(19) United States
(12) Patent Application Publication
(10) Pub. No.: US 2017/0281700 A1
(43) Pub. Date: Oct. 5, 2017

(54) PHAGE THERAPY FOR TARGETING ENTEROCOCCI

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL)

(72) Inventors: Nissan Ronen HAZAN, Tzur-Hadassah (IL); Nurit BEYTH, Jerusalem (IL)

(21) Appl. No.: 15/623,556

(22) Filed: Jun. 15, 2017

Related U.S. Application Data
(63) Continuation-in-part of application No. PCT/IL2015/051227, filed on Dec. 17, 2015.
(60) Provisional application No. 62/092,932, filed on Dec. 17, 2014.

(51) Int. Cl. A61K 35/76; A61K 9/00; C12N 7/00; A61K 45/06

(52) U.S. Cl. CPC A61K 35/76; A61K 45/06; A61K 9/0034; C12N 7/00; C12N 2795/10132; C12N 2795/10121; C12N 2795/10171

(57) ABSTRACT

Isolated bacteriophages having genomes comprising the nucleic acid sequences as set forth in SEQ ID NOs: 1 and 2, or variants thereof, and compositions comprising the bacteriophages in a ratio ranging from 1:10 to 10:1, respectively. Further provided are uses of the compositions in treating or preventing an infection inflicted by a bacteria (e.g., Enterococcous faecalis and/or Enterococcous faecium). As a non-limiting example, the composition may be used in treating or preventing a root canal infection.

### CROSS-REFERENCE TO RELATED APPLICATIONS

This application is a continuation-in-part of PCT Patent Application No. PCT/IL2015/051227 filed on Dec. 17, 2015, which claims the benefit of priority of U.S. Provisional Patent Application No. 62/092,932 filed on Dec. 17, 2014, the contents of which are incorporated herein by reference in their entirety.

### FIELD OF INVENTION

Present embodiments relate to the field of bacteriophage therapy.

### BACKGROUND OF THE INVENTION

Enterococci species are Gram-positive facultative anaerobe cocci that occur singly, in pairs or as short chains. They play an important role in human and animal microbiomes as a commensal of the gastro-intestinal tract and to a lesser extent in the female urogenital tract and the oral cavity. On the other hand, enterococci are also among the leading multidrug resistant pathogens in hospital (nosocomial) diseases and as such have been of interest to researchers since the 1970s. Enterococci are a major cause of worldwide bacteremia, endocarditis, bacterial meningitis, penetrate the dentinal tubules urinary tract, wound, and device-device related infections (Sava et al., 2010) among other infections. Thus, enterococci, and in particular Enterococcus faecalis (E. faecalis), are considered one of the biggest challenges faced by medicine today. Moreover, enterococci are also of regulatory and industrial interest as they are used in food production, probiotic products and for tracking fecal contamination. Specifically, it is the major pathogen found in persistent infections associated with root canal treatment failure. Moreover, the persistence of E. faecalis in the root canal system can result in periradicular tissue invasion with subsequent development of abscess and cellulitis.

Biofilms may pose a severe health threat, as at this phase bacteria become not only inaccessible to antibacterial agents and the body's immune system, but also provide a reservoir of bacteria for chronic infections throughout the body. Most biofilm-associated infections are treated today using antibiotics, for lack of a better alternative. The extensive use or misuse of antibiotics has led to an alarmingly constant emergence of virulent, antibiotic-resistant pathogenic bacteria. Moreover, it is well established that attacking mature biofilms with conventional antibiotics works poorly, requiring much higher drug doses than usual, as all such agents have difficulty in penetrating the extracellular polysaccharide sheath covering the biofilm. This challenge calls for different measures of antimicrobial protection; one that delivers an antimicrobial agent to incapacitate biofilm forming bacteria and one that prevents the proliferation of bacteria in biofilms. Consequently, the development of new antimicrobial means becomes paramount.

One alternative recently regaining interest is bacteriophage (“phage”) therapy, with its history of successful use in western countries at the beginning of the 20th century and in eastern European countries today. Phage therapy comprises a prokaryotic virus that specifically targets and destroys disease-causing bacteria by invading bacterial cells, disrupting their metabolism and causing lysis. The key benefits of phage therapy are: (i) phages are highly strain specific with low impact on the commensal flora; (ii) they multiply at the infection site and disappear together with the pathogen they control; (iii) treatment is efficient against biofilms; (iv) phages are natural products which are usually devoid of apparent toxicity and (v) in case of resistant phages can be evolve or genetically manipulated to attack the resistant bacteria. (vi) Being multiplying organisms, the production of phages is relatively cheap. Moreover, the main reason why conventional antibiotics were preferred over phage therapy was the unknown nature of phages and risk of them having harmful genes. Nowadays, with high throughput sequencing abilities and lot of knowledge about harmful genes, these risks are greatly reduced.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The present invention provides an isolated bacteriophage selected from EFDG1 or EFLK1, having a genome comprising the nucleic acid sequence as set forth in SEQ ID NO: 1 or 2 respectively, or a combination thereof, compositions comprising same and methods of use thereof in treatment of an Enterococcal infection such as an E. faecalis and E. faecium associated infections.

The present invention is based, in part, on the finding that an isolated strain of EFDG1 or EFLK1 bacteriophage have a high and effective lytic activity against planktonic cultures, in vitro biofilms and root canal infecting bacteria. Surprisingly, this effect was demonstrated also in antibiotic resistant bacteria. The present invention is also based, in part, on the unexpected finding that a combination of EFDG1 and EFLK1 showed an advantageous lytic activity against E. faecalis compared to each phage alone. The lytic activity was demonstrated against a naïve E. faecalis strain and surprisingly also against E. faecalis strains with emerged resistant to one of the phages alone.

The present invention is further based, in part, on the unexpected finding that a combination of an antibiotic and an isolated strain of EFDG1 and/or EFLK1, showed a synergistic lytic activity compared to use of the antibiotic alone, each strain alone, or to the combination of both strains.

According to a first aspect, the invention provides a composition comprising: (i) an isolated strain of bacteriophage having a genome comprising a nucleic acid sequence of SEQ ID NO: 1 (EFDG1), or a sequence having at least 95% sequence identity thereto; and (ii) an isolated strain of bacteriophage having a genome comprising a nucleic acid sequence of SEQ ID NO: 2 (EFLK1), or a sequence having at least 95% sequence identity thereto, wherein a ratio between the isolated strains of bacteriophage having a genome comprising the nucleic acid sequence of SEQ ID NOs: 1 and 2 ranges from 10:1 to 1:10, respectively.

In some embodiments, the ratio between the isolated strains of bacteriophage having a genome comprising the nucleic acid sequence of SEQ ID NOs: 1 and 2 is selected from a range of: 1:1-3:1, 5:1-1:5, 3:1-1:3 and 2:1-1:2, respectively.

According to another aspect, the invention provides a composition comprising one or more isolated strains of bacteriophages selected from the group consisting of: (i) an isolated strain of bacteriophage having a genome comprising a nucleic acid sequence of SEQ ID NO: 1, or a sequence having at least 95% sequence identity thereto; and (ii) an isolated strain of bacteriophage having a genome comprising a nucleic acid sequence of SEQ ID NO: 2, or a sequence having at least 95% sequence identity thereto.

In some embodiments, the compositions of the invention further comprise one or more antibiotics.

In some embodiments, the invention provides a pharmaceutical composition comprising any of the compositions of the invention and a pharmaceutically acceptable carrier.

In some embodiments, the invention provides the compositions described herein for use in treating an Enterococcal infection. In some embodiments, the invention provides the compositions described herein for use in treating a subject inflicted or at risk of being inflicted with an Enterococcal infection.

In some embodiments of the compositions, methods and kits of the invention, said Enterococcal infection is selected from the group consisting of: Enterococcous faecalis, Enterococcous faecium, Enterococcus avium, Enterococcus gallinarum, Enterococcus casseliflavus, Enterococcus durans, Enterococcus raffinosus, and Enterococcus mundtii. In some embodiments of the compositions, methods and kits of the invention, said Enterococcal infection is selected from Enterococcous faecalis and Enterococcous faecium infection.

In some embodiments, the invention provides the compositions described herein for use in treating a subject inflicted or at risk of being inflicted with a bacterial infection selected from the group consisting of: endocarditis, bacteremia, urinary tract infections (UTI), meningitis and root canal infections.

According to another aspect, the invention provides a method comprising the steps of providing the composition of the invention; and contacting a bacterial cell with the composition in an amount effective to infect the bacterial cell.

In some embodiments, the contacting is applying the composition onto a surface. In some embodiments, the contacting is administering the composition to a subject in need thereof. In some embodiments, said administering is administering ex-vivo to the subject. In some embodiments, said administering is administering in-vivo to the subject.

In some embodiments, the subject in need thereof is a subject inflicted or at risk of being inflicted with a bacterial infection selected from the group consisting of: endocarditis, bacteremia, urinary tract infections (UTI), meningitis, and root canal infections.

In some embodiments, there is provided a method for treating or preventing a root canal infection, the method comprising the steps of:

providing the composition described herein; and

administering a therapeutically effective amount of the pharmaceutical composition into a root canal of a subject in need thereof, thereby treating or preventing a root canal infection in a subject.

In some embodiments, the pharmaceutical composition is a sustained release composition. In some embodiments, the pharmaceutical acceptable carrier is selected from the group consisting of: a gel, a chip, a film a non-degradable polymer and a degradable polymer.

In some embodiments, there is provided the pharmaceutical composition of the invention, for use in treating or preventing a root canal infection

In addition to the exemplary aspects and embodiments described above, further aspects and embodiments will become apparent by reference to the figures and by study of the following detailed description.

### BRIEF DESCRIPTION OF THE DRAWINGS

FIGS. 1A-M: Demonstration of EFDG1 as an efficient lytic phage that infects E. faecalis: FIG. 1A is a photograph showing formation of plaques of EFDG1 on an E. faecalis lawn; FIG. 1B is a photograph showing two representative test tubes of E. faecalis cultures incubated for 24 hours in the absence (untreated test tube on the left) or presence (treated test tube on the right) of EFDG1 phages (multiplication of infections (MOI)=10⁻⁴), total clearance of turbidity 30 of the culture treated with EFDG1 phages is demonstrated; FIG. 1C is a graph showing bacterial cell growth of a logarithmic culture, as measured by turbidity of a cell culture, plotted against time for different dosages of EFDG1, results demonstrate that EFDG1 kills logarithmic E. faecalis in a dose dependent manner with an MOI as low as 10⁻⁴; FIG. 1D is a graph showing bacterial cell growth of a stationary culture, as measured by turbidity of a cell culture, plotted against time for different dosages of EFDG1, results demonstrate that EFDG1 effectively eliminated stationary cultures of E. faecalis at an MOI as low as 10⁻⁷; FIG. 1E is a bar graph comparing colony forming unit (CFU) counts of E. faecalis bacteria following 24 hours (Logarithmic, left panel) and 120 hours (stationary, right panel) in the absence (untreated) or presence of EFDG1 at an MOI of 10⁻⁴ and 10⁻⁷, respectively; FIG. 1F is a graph showing bacteria cell growth of a logarithmic culture plotted against time in the presence of EFDG1, EFLK1 or combinations thereof (cocktails 1-5); FIG. 1G is a graph showing bacteria cell growth of a stationary culture plotted against time in the presence of EFDG1, EFLK1 or combinations thereof (cocktails 1-5); FIG. 1H is a bar graph showing bacteria cell growth of a logarithmic culture plotted against time in the presence of EFDG1, EFLK1 or combinations thereof (cocktails 1-5); FIG. 1I is a bar graph showing bacteria cell growth of a stationary culture plotted against time in the presence of EFDG1, EFLK1 or combinations thereof (cocktails 1-5); FIG. 1J is a graph showing bacteria cell growth plotted against time, E. faecalis V583 VRE were treated either by phage EFLK1 in concentration of 2.5×10⁷ phages/ml or vancomycin in concentration of 15 μg/ml or a combination thereof; FIG. 1K is a photograph of bacterial colonies formed by seeding aliquots of bacterial cultures of FIG. 1J, on an agar plate, following 24 hours incubation in 37 degrees Celsius (° C.), growth was observed from “Untreated”, “Vancomycin”, “Phage” (Upper panel) but not when treated with both antibiotic and phage (in a circle); FIG. 1L is a graph showing bacteria cell growth of an emerged strain of E. faecalis which is resistant to EFDG1 (depicted “EFDG1^{r}”), plotted against time in the presence of EFDG1 or EFLK1; FIG. 1M is a bar graph showing results of an in vitro fibrin clot model against E. faecalis and EFDG1^{r}. The insert depicts the clot model.

FIG. 2A-E: EFDG1 eradicates E. faecalis biofilms. EFDG1 was added to a two-week-old biofilm of E. faecalis: FIG. 2A is a confocal 3D image demonstrating that the phage eliminated the biofilm almost completely; FIG. 2B is a graph showing quantitative representation of bacteria number within the biofilm layer, in the presence or absence of EFDG1, as detected by confocal microscopy; FIG. 2C is a graph showing quantitative representation of bacteria number, as determined by crystal violet staining, plotted against time, in the presence or absence of EFDG1; FIG. 2D is a graph showing CFU count of E. faecalis plotted against time, in the presence or absence of EFDG1; FIG. 2E is a bar graph showing CFU count in the presence of EFDG1, EFLK1 or combinations thereof (cocktails 1-5), as well as untreated control;

FIG. 3A-E: EFDG1 belongs to the Spounavirinae sub-family of the Myoviridae phages: FIG. 3A is a photograph showing EFDG1 as imaged by Transmission Electron Microscope (TEM); FIG. 3B is a schematic representation of EFDG1 DNA sequence and putative genes (arrows), squares denote repeat sequences, the inner graphs show GC (inner graph) and AT (outer graph) content; FIG. 3C shows a phylogenetic tree of EFDG1 in relation to the genomes of fully sequenced Spounavirinae phages, the name of each phage and its accession number in NCBI are denoted; FIG. 3D is a comparison of EFDG1 genome and its closest related phage phiEF24c by Mauve plugin of Geneious 7.5.1, the shaded box marks similar regions; FIG. 3E is a comparison of ECP3, EFDG1, EFLK1 and phiEF24c genomes, the enlarged regions are the regions corresponding to the region of phiEF24c into which the P2 mutation was introduced;

FIG. 4A-D: EFDG1 protects root canals from infection: FIG. 4A is a schematic representation of ex vivo root canal treatment model: rooted human teeth were subjected to endodontic treatment including standard cleaning, shaping and filling. Bacterial contamination was performed before, during and after the root canal treatment, test group included phage irrigation in addition to the standard procedure; FIG. 4B is a photograph of a root irrigated with EFDG1 phage showing clear broth in the lower chamber indicating no bacterial outgrowth (right) and control root subjected to standard protocol demonstrating broth turbidity (left); FIG. 4C is a bar graph showing number of live bacteria by colony forming units count per milliliter (CFU/ml) in the lower chamber, Viable E. faecalis counts depicted were reduced by at least 8 logs following phage irrigation; and FIG. 4D shows confocal laser scanning microscope (CLSM) images of a horizontal root section of the phage treated tooth (right) depicting low numbers of stained bacteria when compared to the control (left), where stained live and dead bacteria are depicted in the dentinal tubules surrounding the root canal.

FIGS. 5A-B. Peritonitis phage therapy using anti-E. faecalis phages EFDG1 and EFLK1: FIG. 5A is a graph depicting animal mortality after the various treatments. FIG. 5B is a graph depicting health scores of mice in the various treatments. The untreated animal experiments ended after 12 h and the animals were sacrificed due to their poor condition (score <0.2). The treatments exemplified in FIGS. 5A-B were treatment with EFDG1 and EFLK1 phages (“A”), combined treatment of phages and antibiotics (“B”); antibiotic treatment (“C”); and no-treatment as control (“D”).

### DETAILED DESCRIPTION OF THE INVENTION

The present invention provides an isolated bacteriophage EFDG1 or EFLK1 having a genome comprising the nucleic acid sequence as set forth in SEQ ID NO: 1 or 2, respectively, or a combination thereof, having lytic activity against a target bacteria. The present invention further provides compositions comprising the isolated bacteriophage EFDG1 or EFLK1 or a combination thereof, alone or combined with an antibiotic, and methods for using said compositions for treating or preventing an infection.

The present invention is based, in part, on the finding that an isolated strain of bacteriophage having a genome comprising the nucleic acid sequence as set forth in SEQ ID NO: 1 or 2 has a lytic activity against target bacteria selected from the bacterial species of: Enterococcus faecalis (E. faecalis) and Enterococcus faecium (E. faecium), regardless of their antibiotic resistance profile.

In some embodiments, EFDG1 and EFLK1 are effective in killing target bacteria both in vivo and in vitro. As used herein, “in vivo” refers to a process within a subject (e.g., eliminating bacterial growth in an animal or a mammalian host cell), wherein “in vitro” refers to a process that occurs outside a subject, such as eliminating bacteria in a culture or a test tube. In some embodiments, EFDG1 and EFLK1 are effective in killing target bacteria within a planktonic culture. As used herein, the term “planktonic” refers to bacteria that are not attached to a surface, planktonic bacteria are free-floating and are not part of a biofilm. In some embodiments, EFDG1 and EFLK1 are effective in killing/lysing target bacteria embedded in biofilms. The term “biofilm” as used herein designates to be a heterogeneous bacterial formation growing on various surfaces; preferably a bacterial community growing embedded in an exopolysaccharide matrix adhered onto solid biological or non-biological surfaces.

As used herein, “isolated” refers to a material removed from its original environment in which it naturally occurs, and thus is altered by the hand of man from its natural environment. Isolated bacteriophage refers to a phage that is e.g., cultivated, purified and/or cultured separately from the environment in which it is naturally located. Isolated material further encompasses bacteriophage specific for a target bacteria or particular target bacteria isolates, isolated and cultured separately from the environment in which it was located, where these isolates are present in purified compositions that do not contain any significant amount of other microorganisms such as other bacteriophage or bacterial strains. A “composition comprising an isolated bacteriophage” as used herein encompass combination of one or more isolated bacteriophage strains but does not include the bacteriophage as it exists in its natural environment prior to isolation and/or substantial purification.

As used herein, the terms “bacteriophage” and “phage” are used interchangeably to refer to a virus that infects and replicates within a bacterium. The term “bacteriophage”, as used herein, further refers to a bacteriophage which infects target bacteria and has a lytic or otherwise harmful activity against the target bacteria. Typically, different strains of bacteriophage may infect different species or even different strains of bacteria with different results, or may infect some strains of bacteria but not others. The term “target bacteria” as used herein refers to bacterial cells which are susceptible to EFDG1 or EFLK1 infection and lytic activity.

As used herein, the term “lytic activity” refers to the property of a bacteriophage to cause lysis of a bacterial cell. The lytic activity of a bacteriophage can be tested according to techniques known in the art.

As used herein, the terms “lysing”, “lysis of a bacterial cell” “eliminating” and “killing” are used interchangeably to describe bacterial cell death.

As used herein, “EFDG1” refers to an isolated bacteriophage having a genome comprising or consisting of the nucleic acid sequence as set forth in SEQ ID NO: 1. In some embodiments, “EFDG1” refers to an isolated bacteriophage having a genome comprising or consisting of the nucleic acid sequence as disclosed in GenBank (NCBI) Accession No. KP339049.1. As used herein, “EFLK1” refers to an isolated bacteriophage having a genome comprising or consisting of the nucleic acid sequence as set forth in SEQ ID NO: 2. In some embodiments, “EFLK1” refers to an isolated bacteriophage having a genome comprising or consisting of the nucleic acid sequence as disclosed in GenBank (NCBI) Accession No. KR049063. In some embodiments, “EFLK1” refers to an isolated bacteriophage having a genome comprising or consisting of the nucleic acid sequence as disclosed in GenBank (NCBI) Accession No. KR049063.1.

As used herein, the term “genome” refers to the total genetic information or hereditary material possessed by an organism (including viruses), e.g., the entire genetic complement of a bacteriophage. A genome can comprise RNA or DNA. Typically, a genome can be linear (mammals) or circular (bacterial).

As used herein, the term “nucleic acid” is well known in the art. A “nucleic acid” as used herein will generally refer to a molecule (e.g., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

As used herein, the expressions “nucleotide sequence”, “nucleic acid sequence,” “polynucleotide sequence”, and equivalent or similar phrases refer to the order of nucleotide monomers in the nucleotide polymer. By convention, a nucleotide sequence is typically written in the 5′ to 3′ direction. A polynucleotide may be a polymer of RNA or DNA that is single- or double-stranded, that optionally contains synthetic, non-natural or altered nucleotide bases. Unless otherwise indicated, a particular polynucleotide sequence of the invention optionally encompasses complementary sequences, in addition to the sequence explicitly indicated. As used herein, it is not intended that the term “polynucleotide” be limited to naturally occurring polynucleotide structures, naturally occurring nucleotides sequences, naturally occurring backbones or naturally occurring internucleotide linkages. One skilled in the art knows well the wide variety of polynucleotide analogues, unnatural nucleotides, non-natural phosphodiester bond linkages and internucleotide analogs that find use with the invention.

EFDG1 and EFLK1 are both strains of the Spounavirinae subfamily of the Myoviridae family of bacteriophages, which includes lytic bacteriophages that infect Gram positive bacteria. Typically, members of the Spounavirinae subfamily have a double stranded DNA of about 130-160 kilobases (kb) encoding for about 190-230 proteins.

As demonstrated in Table 1, EFDG1 and EFLK1 were found specific for E. faecalis and E. faecium, which are closely related bacteria species. Further, EFDG1 and EFLK1 were found to have lytic activity on all tested strains of E. faecalis and E. faecium. As further shown in the experimental section, the bacteriophages of the invention are able to selectively lyse E. faecalis and E. faecium bacteria in vitro or in vivo. The genomes of EFDG1 and EFLK1 do not contain any apparent harmful genes to mammalian cells. Also, the bacteriophages of the invention are unable to affect mammalian cells, and are, thus, EFDG1 and EFLK1 are non-toxic to cells in vitro and upon administration in vivo, do not induce undesirable long-term effects to cells other than their target bacterial cell.

The term “specific” or “specificity” in relation to a bacteriophage refers to the type of “target bacteria” that the bacteriophage is able to infect. Typically, specificity is mediated by tail fibers of bacteriophages, that are involved in the interaction with receptors expressed on cells. A bacteriophage “specific” for E. faecalis and E. faecium refers to a bacteriophage which can infect several E. faecalis and E. faecium strains and which cannot infect non-E. faecalis and non-E. faecium bacteria.

As further demonstrated in Table 1, the bacterial cell may be sensitive to one or more antibiotics selected from: Vancomycin, Erythromycin, Gentamicin, Gentamicin, Streptomycin, Ampicillin, Chloramphenicol, Ciprofloxacin, Nitrofurantoin, Streptomycin and Daptomycin. As further demonstrated in table 1, the bacterial cell may be resistant to one or more antibiotics selected from: Erythromycin, Gentamicin, Gentamicin, Streptomycin, Ampicillin, Chloramphenicol, Vancomycin, Ciprofloxacin, Nitrofurantoin, Streptomycin and Daptomycin.

As used herein the phrase “bacterial cell is resistant to one or more antibiotics” means that the bacterial cell or at least a portion of a population of bacterial cells actively grows and divides in the presence of the antibiotic. As used herein, the term “at least a portion of a population of bacterial cells” refers to at least 30%, at least 40%, at least 50%, at least 65%, at least 70%, at least 75%, at least 80%, at least 85%, at least 90% or at least 95% of the population of bacterial cells. As a non-limiting example, antibiotic resistance means that the bacterial cell does not lyse or is not otherwise destroyed by the antibiotic. As used herein, the phrase “bacterial cell is sensitive to one or more antibiotics” means that the bacterial cell does not actively grow and divide in the presence of the antibiotic.

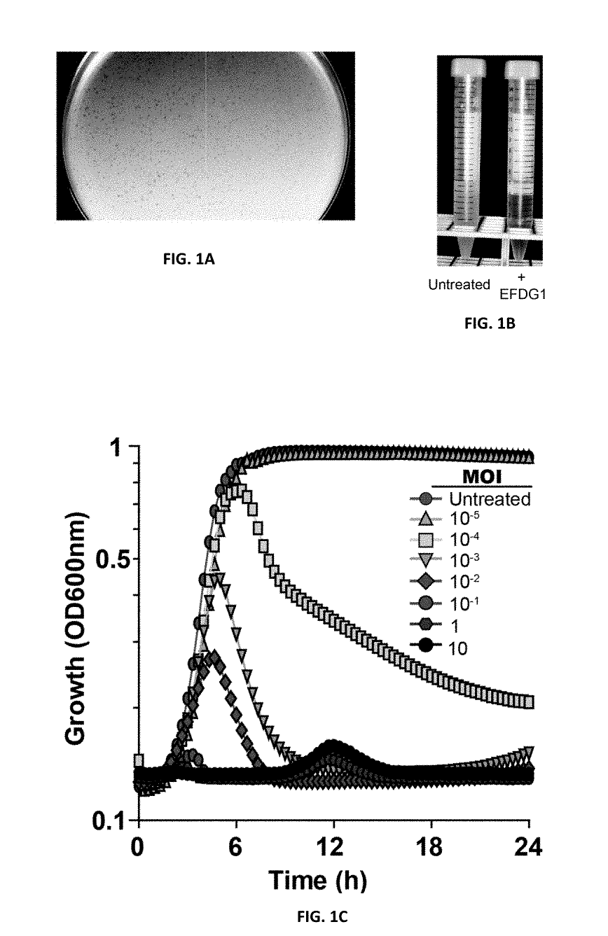

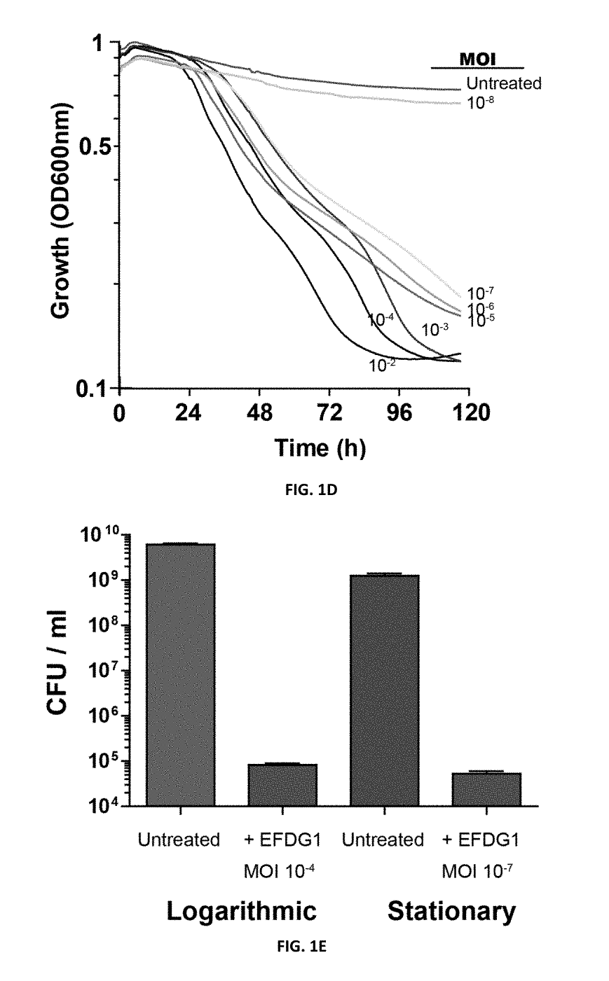

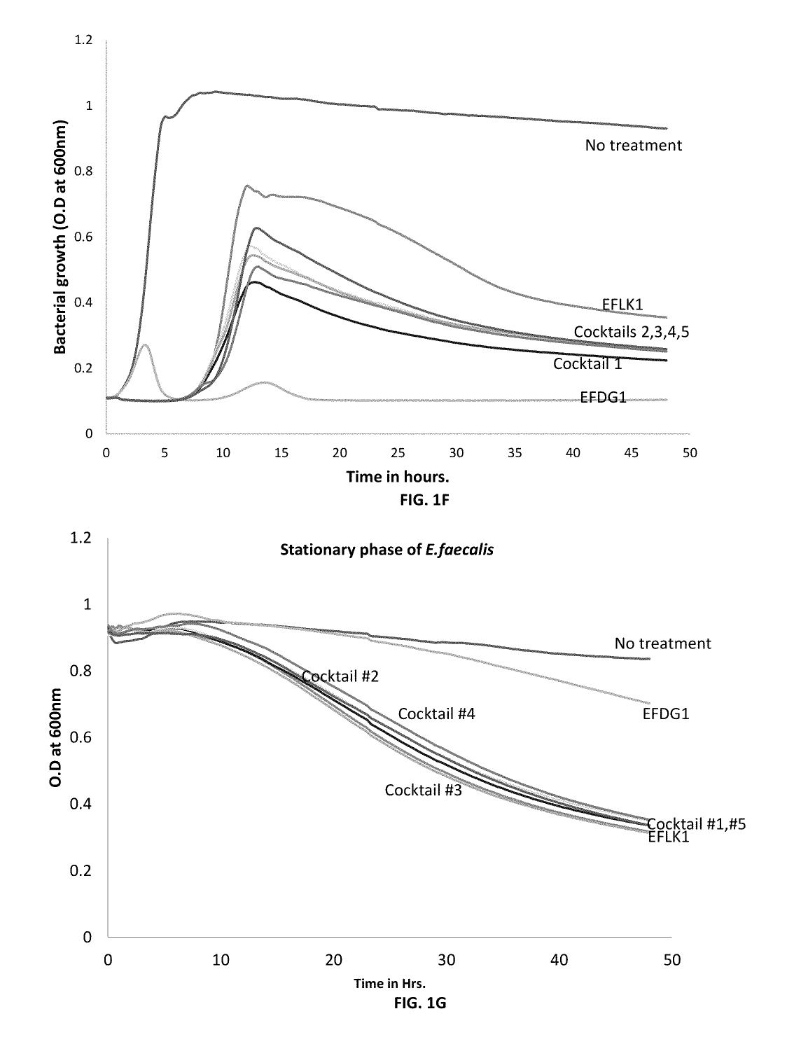

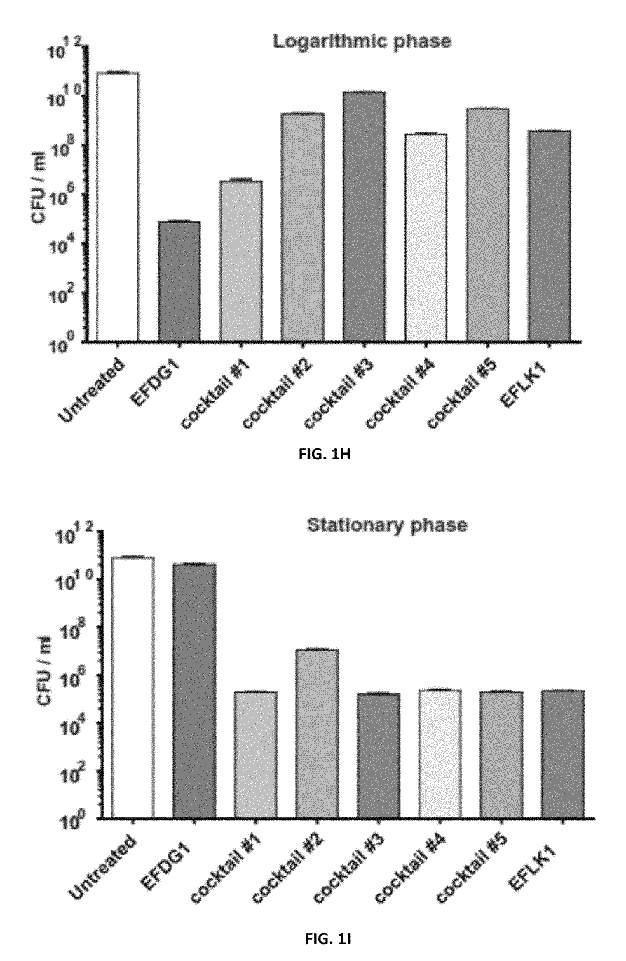

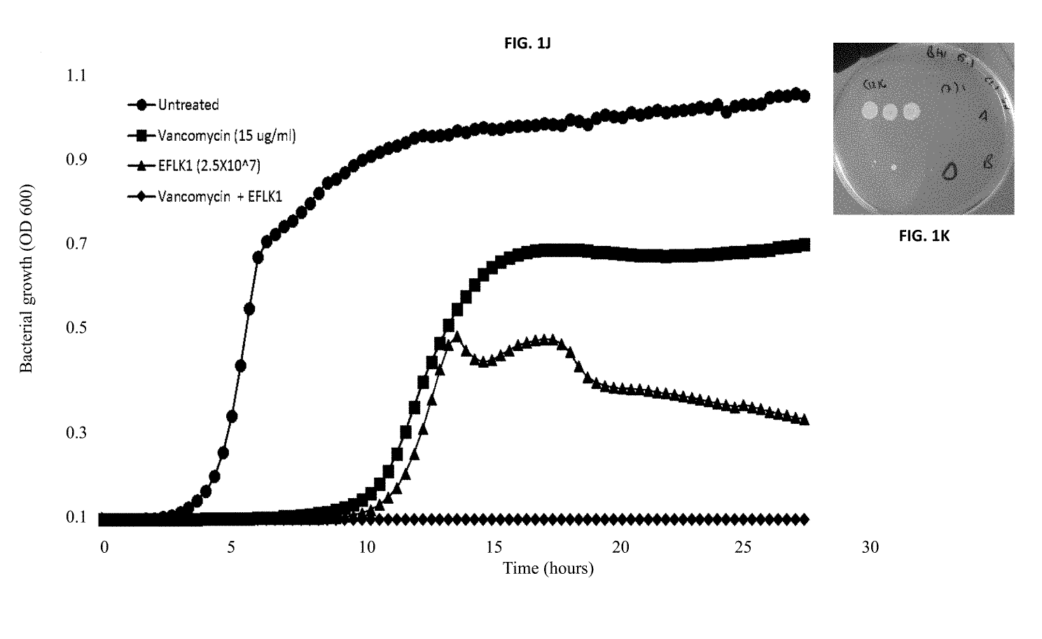

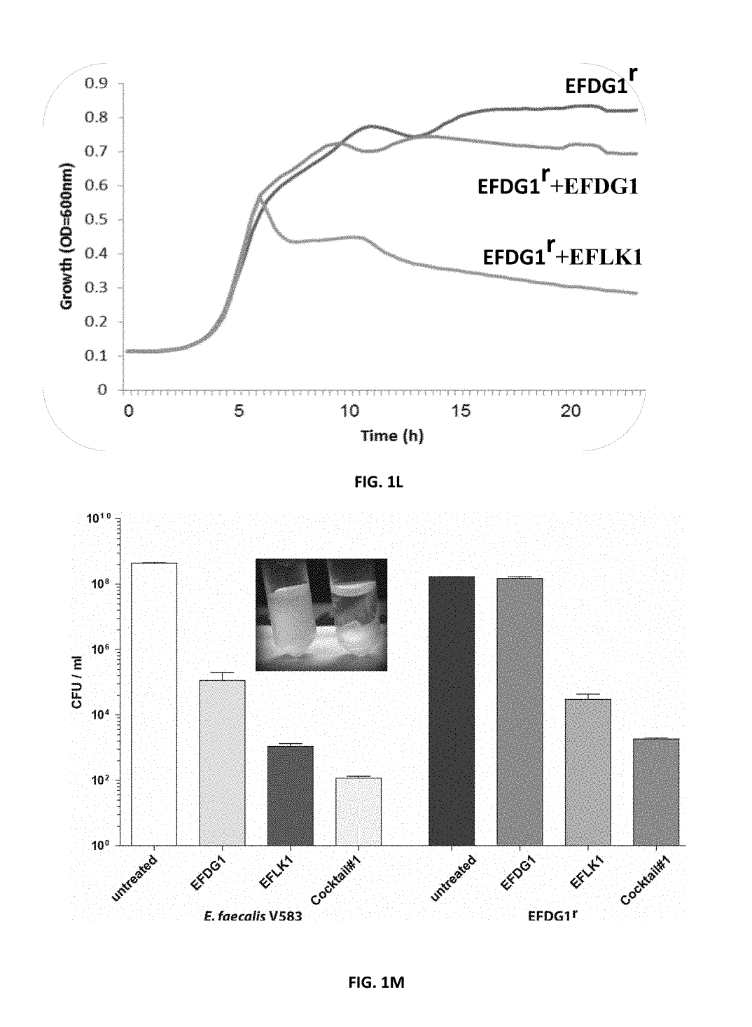

In some embodiments, EFDG1 kills target bacteria in a logarithmic culture and/or in a stationary culture. In some embodiments, logarithmic bacterial cells elimination by EFDG1 is more efficient than the elimination of cells in their stationary phase. In some embodiments, decreased time is required for logarithmic bacterial cells elimination by EFDG1 than for bacterial cells in their stationary phase. For a non-limiting example, as demonstrated in the exemplary section, EFDG1 eliminates logarithmic bacterial cells in an MOI of 10⁻⁴ after 24 hours, EFDG1 eliminates stationary bacterial cells in an MOI of 10⁻⁷ after 120 hours (FIG. 1C), or alternatively MOI of 10⁻⁴ after more than 72 hours (FIG. 1D).

As used herein, the term “more efficient” with reference to an effect refers to a decrease in time for achieving the referenced effect, an increase in the effect (e.g., killing effect) or a combination thereof. In some embodiments, the decrease in time for achieving the effect is at least 1.5, 2, 3, 4, or 5 fold decrease. In some embodiment, the increase of the effect is a complete elimination compared to partial elimination of bacterial cells in a given time. In other embodiments, the increase in the effect may be at least 1.5, 2, 3, 4, 5, 6, 7, 8, 9 or 10 folds increase in elimination of bacterial cells in a given time. Each possibility represents a separate embodiment of the present invention.

In some embodiments, EFLK1 kills target bacteria in a logarithmic culture and/or in a stationary culture. In some embodiments, decreased time is required for stationary bacterial cells elimination by EFLK1 than for elimination of bacterial cells in their logarithmic phase.

Typically, growth of bacterial cells in culture progresses through an initial period of slow growth known as “lag phase”, followed by a period of rapid, logarithmic growth known as “logarithmic phase” also referred to as “logarithmic culture” during which the cells divide at a constant rate and the cell number effectively doubling every unit period of time, and finally, the culture enters “stationary phase” during which the growth rate slows or even stops, that is, cell division rate is substantially the same as cell death rate.

As used herein, the phrase “multiplicity of infection” or “MOI” is the average number of virus per infected cell. The MOI is determined by dividing the number of virus added (ml added×PFU) by the number of cells added (ml added×cells/ml). For a non-limiting example, in a case when 100 PFU/ml bacteriophage were enough to eliminate 10⁹ CFU/ml bacteria cells the resulting MOI is 10⁻⁷ MOI. As used herein, the term “PFU” means plaque forming unit, as it is well defined in the art. Lytic bacteriophages lyse the host cell, causing a zone of clearing (or plaque) on a culture plate. Theoretically, each plaque is formed by one phage and the number of plaques multiplied by the dilution factor is equal to the total number of phages in a test preparation. As used herein, the terms “CFU” and “colony forming unit” refer to a measure of viable bacterial numbers by counting the colony numbers.

In some embodiments, EFDG1 is more efficient than EFLK1 in killing target bacteria in a logarithmic culture. In some embodiments, EFLK1 is more efficient than EFDG1 in killing target bacteria in a stationary culture. As further demonstrated in the exemplary section, in some embodiments, a combination of EFDG1 and EFLK1 (“cocktail”) kills logarithmic cells more efficiently compared to EFLK1 and stationary cells more efficiently compared to EFDG1, thus a cocktail expands the range of bacterial cultures which can be efficiently killed. In some embodiments, a combination of EFDG1 and EFLK1 has an advantageous for efficiently killing target bacteria both in a logarithmic and stationary culture. As further demonstrated in the exemplary section, EFLK1 was effective in killing an emerged EFDG1-resistant E. faecalis strain. In some embodiments, a combination of EFDG1 and EFLK1 was efficient in killing target bacteria which is resistant to either EFDG1 alone and/or EFLK1 alone. In some embodiments, a combination of EFLK1 and EFDG1 was efficient in killing bacterial cultures including bacteria with emerged resistance to one of the phages.

In some embodiments, the EFDG1 and EFLK1 are combined in a ratio ranging from 10:1 to 1:10 respectively. In some embodiments, the ratio between EFDG1 and EFLK1 in the compositions (i.e., mixture) ranges from 10:1-1:10, 9:1-1:9, 8:1-1:8, 7:1-1:7, 6:1-1:6, 5:1-1:5, 4:1-1:4, 3:1-1:3, 2:1-1:2, 1:1-1:5, 1:1-1:4, 1:1-1:3, 1:1-1:2, 5:1-1:1, 4:1-1:1, 3:1-1:1 or 2:1-1:1, respectively. In some embodiments, the ratio between EFDG1 and EFLK1 in the mixture ranges from 1:3-3:1 respectively. In some embodiments, the ratio between EFDG1 and EFLK1 in the mixture ranges from 1:2-2:1, respectively. In some embodiments, the ratio between EFDG1 and EFLK1 in the mixture is 1:1.

The term “relative to” or “compared to,” when used in the context of comparing the activity of a combination (i.e., mixture, cocktail) composition comprising the EFDG1 and EFLK1 to either EFDG1 or EFLK1 alone, refers to a comparison using amounts known to be comparable according to one of skill in the art. For a non-limiting example, comparable amounts of EFDG1, when comparing the combination therapy to EFDG1 alone, may be based on an equal concentration of EFDG1 in a composition.

The terms “mixture”, “cocktail” and “combination” are used interchangeably to refer to a cocktail having two or more isolated phages. According to one embodiment, a cocktail of the invention comprises EFDG1 and EFLK1. According to another embodiment, a mixture of the invention comprises EFDG1 and one or more other phages. According to another embodiment, the mixture of the invention comprises EFLK1 and one or more other phages. According to another embodiment, the mixture of the invention comprises EFDG1 and/or EFLK1 and one or more other phages.

In some embodiments, the one or more other phages have lytic activity against E. faecalis and/or E. faecium. In some embodiments, the one or more other phages have lytic activity against E. faecalis. In some embodiments, the one or more other phages having lytic activity against E. faecalis are selected from the phage: phiEF24c and ECP3. In some embodiments, a cocktail may comprise one or more phages and antibiotic.

In some embodiments, a combination of one or more antibiotics and EFLK1 and EFDG1 has a synergistic lytic activity on the target bacteria compared to antibiotics alone or EFLK1 and EFDG1. In some embodiments, a combination of one or more antibiotics and EFLK1 has a synergistic lytic activity on the target bacteria compared to antibiotics alone or EFLK1 alone. In some embodiments, a combination of one or more antibiotics and EFDG1 has a synergistic lytic activity on the target bacteria compared to antibiotics alone or EFDG1 alone.

As used herein, the term “antibiotic” refers to any compound known to one of ordinary skill in the art that will inhibit or reduce the growth of, or kill, one or more microorganisms, including bacterial species. The antibiotic in various embodiments can be a bactericidal antibiotic or a bacteriostatic antibiotic. Bactericidals can kill bacteria directly where bacteriostatics can prevent them from dividing. Exemplary classes of antibiotics include, but are not limited to, β-lactams, (e.g., penicillins such as amoxicillin, ampicillin and Penicillin G, cephalosporins monobactams such as aztreonam, and carbapenems); aminoglycosides (e.g., streptomycin, gentamicin, kanamycin, neomycin, tobramycin, netilmycin, paromomycin, and amikacin); tetracyclines (e.g., doxycycline, minocycline, oxytetracycline, tetracycline, and demeclocycline); sulfonamides (e.g., mafenide, sulfacetamide, sulfadiazine and sulfasalazine; trimethoprim); quinolones (e.g., ciprofloxacin, norfloxacin, and ofloxacin); glycopeptides (e.g., vancomycin, telavancin, teicoplanin); lipopeptides (e.g., daptomycin); macrolides (e.g., erythromycin, azithromycin, and clarithromycin); carbapenems (e.g., ertapenem, doripenem, meropenem, and imipenem); cephalosporins (e.g., cefadroxil, cefepime, and ceftobiprole); lincosamides (e.g., clindamycin, and lincomycin); nitrofurans (e.g., furazolidone, and nitrofurantoin); polypeptides (e.g., bacitracin, colistin, and polymyxin B); and other antibiotics, e.g., the polymycins, chloramphenicol, or any salts or variants thereof. The antibiotic used may depend on the strain of target bacteria. In some embodiments, the one or more antibiotics are selected from the group consisting of: erythromycin, gentamicin, streptomycin, ampicillin, chloramphenicol, vancomycin, Ciprofloxacin, nitrofurantoin and daptomycin.

In some embodiments, the target bacteria is resistant to treatment by the antibiotic alone. In some embodiments, combining the antibiotic and one or more of the phages sensitizes the target bacteria to treatment by the antibiotic. For a non-limiting example, as demonstrated in the exemplary section, treatment of the vancomycin resistant bacteria strain, E. faecalis V583 (VRE), with a combination of EFLK1 and vancomycin had a synergistic lytic activity compared to EFLK1 and vancomycin, and led to complete eradication of the bacteria.

As used herein, the term “synergism”, “synergistic” or “synergistically” refers to the combined action of two or more agents wherein the combined action is greater than the sum of the actions of each of the agents used alone.

In some embodiments, a synergistic effect of a combination of agents (bacteriophages, antibiotic, etc.) permits the use of lower dosages of one or more of the agents. In some embodiments, a synergistic effect of a combination of agents permits decrease in time for elimination of target bacteria. In some embodiments, decrease in time is at least 1.2, or 1.5, or 2, or 2.5, or 3, or 3.5, or 4, or 5, or 10 folds decrease. In some embodiments, a synergistic effect of a combination of agents permits total elimination versus partial elimination of target bacteria.

In some embodiments, the advantageous effect of a combination of agents permits efficient elimination of bacteria in both logarithmic and stationary phases rather thus allowing efficient elimination of bacteria for both phases rather than one.

### EFDG1 and EFLK1 Variants

In some embodiments, the invention further encompasses one or more variants of the isolated bacteriophages EFDG1 and/or EFLK1. The term “variant” of a reference bacteriophage designates bacteriophages having variation(s) in the genomic sequence and/or polypeptide(s) encoded thereby as compared to the reference bacteriophage, while retaining the same phenotypic characteristic as the reference bacteriophage. Variants also encompass bacteriophages having variation(s) in the genomic sequence and/or polypeptide(s) encoded thereby as compared to the reference bacteriophage, while improving the phenotypic characteristic as the reference bacteriophage. In some embodiments, the variants of the invention are genetically engineered variants (e.g., a result of genetically engineered mutation(s) to the nucleic acid sequence of the reference bacteriophage).

Variants may comprise e.g., silent mutations, conservative mutations, minor deletions, and/or minor replications of genetic material, and retain phenotypic characteristics of the reference bacteriophage. In some embodiments, the variant of the invention retains any observable characteristic or property that is dependent upon the genome of the bacteriophage of the invention, such as phenotypic characteristics of the bacteriophage and/or lytic activity against E. faecalis and E. faecium.

The term “phenotypic characteristic” designates the morphology and/or host-range of a bacteriophage. Methods for phenotyping bacteriophages are well known in the art and include, for example, determining bacterial host range and/or activity against the biofilm produced by certain bacterial strains.

According to some embodiments, the invention further encompasses variants having a genome comprising at least 85%, 86%, 87%, 88%, 89%, 90%, 91%, 92%, 93%, 94%, 95%, 96%, 97%, 98% or 99% sequence identity to the nucleic acid sequences as set forth in SEQ ID NO: 1 or 2. Each possibility represents a separate embodiment of the present invention. Alternatively, or in combination, variants of the invention have less than 15%, 14%, 13%, 12%, 11%, 10%, 9%, 8%, 7%, 6%, 5%, 4%, 3%, 2%, 1% amino acid variation in a coded polypeptide sequence as compared to a polypeptide of EFDG1 or EFLK1. Each possibility represents a separate embodiment of the present invention.

According to some embodiments, the invention further encompasses variants having a genome comprising at least 1, at least 2, at least 4, at least 5 or at least 10 mutations relative to the nucleic acid sequences as set forth in SEQ ID NO: 1 or 2. According to some embodiments, the invention further encompasses variants having a genome comprising a nucleic acid sequence having at least 95% sequence identity to SEQ ID NO: 1 or 2, wherein the sequence comprises at least 1, at least 2, at least 3, at least 4, at least 5 or at least 10 mutations relative to the nucleic acid sequences as set forth in SEQ ID NO: 1 or 2 respectively. Each possibility represents a separate embodiment of the present invention.

In some embodiments, the mutations include deletions, insertions, substitutions or any combination thereof. In some embodiments, the deletions, insertions, substitutions may be found in a non-coding region such as operon, and promoter regions. In some embodiments, the deletions, insertions, substitutions may be found in a coding region such as regions encoding the head of the bacteriophage. In some embodiments, the deletions, insertions, substitutions are silent mutation which do not result in changes of the coded amino acid sequence.

For a non-limiting example, a variant having improved lytic activity against E. faecalis may be generated by substituting adenine with guanine at position 88,790 of SEQ ID NO:1 or position 96,697 of SEQ ID NO:2. Such variant has a genome comprising a nucleic acid sequence having at least 95% sequence identity to SEQ ID NO: 1 or 2, wherein the sequence comprises guanine at position 88,790 or position 96,697, respectively. These substitutions are based on a specific P2 mutation which was found to improve the ability of phiEF24c to adsorb and lyse E. faecalis and on the homologs genes of EFLK1 and EFDG1 and phiEF24c.

The term “% identity” in relation to nucleic acid sequences designates the level of identity or homology between the sequences and may be determined by techniques known in the art.

The variants may be made to specific bacteriophages by chemical, radiological or other methods well known to those skilled in the art. The variants may also be made by homologous recombination methods well known to those skilled in the art. The variants having a mutated sequence may comprise deletions, insertions additions or substitutions, all of which may be constructed by routine techniques. Insertions may include selectable marker genes, for example lacZ, for screening recombinant viruses by, for example, β-galactosidase activity.

According to some embodiments, the invention further encompasses variants having gene annotation as depicted in tables S2 or S3 of Provisional Patent Application No. 62/092,932, the contents of which are incorporated herein by reference in their entirety.

### Bacteriophage Isolation

Typically, bacteriophages are found in association with their target bacteria (host bacteria). Thus, any source that might be expected to contain the host bacteria is suitable for use as a source of host bacteria-active bacteriophage. Samples containing Enterococcus faecalis include fecal, urine, or sputum samples from patients, particularly patients undergoing acute or prophylactic antibiotic therapy, patients in intensive care units or immunocompromised patients. Consequently, samples for bacteriophage isolation may also be obtained from non-patient sources, including but not limited to, sewage, especially sewage streams near intensive care units or other hospital venues, or by swab in hospital areas associated with risk of infection, such as intensive care units. Other suitable sampling sites include nursing homes, rest homes, dormitories, classrooms, and medical waste facilities. Phages also can be isolated from rivers and lakes, wells, water tables, as well as other water sources (including salt water). Sampling sites include water sources near likely sites of contamination listed above.

Methods for isolating pure bacteriophage strain from a bacteriophage-containing sample are well known in the art. Isolation of bacteriophage active against Enterococcus faecalis (‘the bacteria’) from suitable samples typically proceeds by mixing the sample with nutrient broth, inoculating the broth with a host bacterial strain, and incubating to enrich the mixture with bacteriophage that can infect the host strain. Next, the mixture may be filtered to remove bacterial leaving lytic bacteriophage in the filtrate. Serial dilutions of the filtrate may be plated on a lawn of the bacteria (e.g., on agar plates), and phages active against the bacteria infect and lyse neighboring bacteria. The lysing of bacteria, results in small visibly clear areas called plaques on the plate where bacteriophage has destroyed the bacteria within the confluent lawn of bacteria growth. Since one plaque with a distinct morphology represents one phage particle that replicated in the bacteria within that area of the bacterial lawn, the purity of a bacteriophage preparation can be ensured, for a non-limiting example, by removing the material in that plaque (e.g., with a pasteur pipette) and using this material as the inoculum for further growth cycles of the phage. The bacteriophage produced in such cycles represent a single strain or “monophage”. The purity of phage preparation (including confirmation that it is a monophage and not a polyvalent phage preparation) may be assessed by a combination of electron microscopy, SDS-PAGE, DNA restriction digest and analytical ultracentrifugation. Additionally, each phage may be uniquely identified by its DNA restriction digest profile, protein composition, and/or genome sequence.

### Bacteriophage Compositions

According to one aspect, the invention provides a composition comprising one or more strains of isolated bacteriophages selected from the group consisting of: an isolated strain of bacteriophage having a genome comprising a nucleic acid sequence of SEQ ID NO: 1 (EFDG1), or a variant thereof; and an isolated strain of bacteriophage having a genome comprising a nucleic acid sequence of SEQ ID NO: 2 (EFLK1), or a variant thereof.

According to some embodiments, the invention provides a composition comprising: an isolated strain of bacteriophage having a genome comprising a nucleic acid sequence of SEQ ID NO: 1 (EFDG1), or variant thereof; and an isolated strain of bacteriophage having a genome comprising a nucleic acid sequence of SEQ ID NO: 2 (EFLK1), or a variant thereof. In some embodiments, the composition further comprises at least one additional isolated strain of bacteriophage having a genome that comprises a nucleic acid sequence other than SEQ ID NOs: 1 and 2 or a variant thereof.

In some embodiments, the composition comprises bacteriophage in a concentration of at least 10⁶ phages/ml, at least 10⁷ phages/ml, at least 10⁸ phages/ml or alternatively about 10⁹ phages/ml. In some embodiments, the composition comprises bacteriophage in a concentration ranging from 10⁶ phages/ml to 10¹⁰ phage/ml, 10⁷ phages/ml to 10¹⁰ phage/ml, or 10⁹ phages/ml to 10¹⁰ phage/ml.

Typically, phages are relatively stable and may be kept for prolonged periods of time in cold storage (4 degrees Celsius (° C.)). Alternatively, the phage culture may be freezed in −80° C. or lyophilized and reconstituted in a liquid carries (such as saline) prior to application, for example in root canal.

In some embodiments, the invention provides a pharmaceutical composition comprising the compositions of the invention and a pharmaceutically acceptable carrier. The active ingredient (bacteriophage compositions of the invention) in the pharmaceutical formulations may comprise from 0.1 to 99.99 weight percent.

The term “pharmaceutical composition” refers to carriers, diluent, adjuvant, excipient, or vehicle that are added to the active agent and are suitable for administration to a subject, e.g., a human or animal for veterinary use. For example, the term “pharmaceutically acceptable” can mean approved by a regulatory agency of the Federal or a state government or listed in the U. S. Pharmacopeia or other generally recognized pharmacopeia for use in animals, and more particularly in humans.

The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid, citric acid and its salts, sodium EDTA or sodium bisulfite; and agents for the adjustment of tonicity such as sodium chloride or dextrose are also envisioned. The composition, if desired, can also contain sweetening agents, flavors, emulsifiers, suspensions and preservatives.

Other suitable therapeutic agents may be added to the compositions of the invention or administered sequentially or simultaneously with the compositions of the invention.

Therapeutic agents suitable for use in treatment of root canal infection include but are not limited to antibacterial agents such as iodine, sulfonamides, mercurials, bisbiguanides, or phenolics; antibiotics such as tetracycline, neomycin, kannamycin, metranidazole, or canamycin; anti-inflammatory agents such as indomethacin, euginol, or hydrocortisone; immune-suppressive or stimulatory agents such as methotrexate or levamasole; dentinal desensitizing agents such as strontium chloride or sodium fluoride; odor masking agents such as peppermint oil or chlorophyll; immune reagents such as immunoglobulin or antigens; local anesthetic agents such as lidocaine or benzocaine; nutritional agents such as amino acids, essential fats, and vitamin C; antioxidants such as alphatocopherol and butylated hydroxy toluene; lipopolysaccharide complexing agents such as polymyxin; or peroxides such as urea peroxide.

In some embodiments, the composition further comprises one or more antibiotics. In some embodiments, the antibiotics are co-administered with the compositions of the invention. In some embodiments, the antibiotics are administered sequentially to the compositions of the invention (e.g., pre and/or post treatment with bacteriophage compositions of the invention).

The compositions can take the form of solutions, suspensions, emulsions, tablets, pills, capsules, powders, gels, creams, ointments, foams, pastes, sustained-release formulations and the like. The compositions can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in: Remington's Pharmaceutical Sciences” by E. W. Martin, the contents of which are hereby incorporated by reference herein. Such compositions will contain a therapeutically effective amount of a source of the polypeptides of the current invention, preferably in a substantially purified form, together with a suitable amount of carrier so as to provide the form for proper administration to the subject.

The carrier will vary according to the mode of administration for example adapted for an administration mode selected from the group consisting of intravenous, subcutaneous, intramuscular, intraperitoneal, intravesical, oral, nasal, aerosol, rectal, vaginal, and/or directly or adjacent to a damaged tissue.

For oral administration, the bacteriophage may be combined with one or more solid inactive ingredients for the preparation of tablets, capsules, pills, powders, granules or other suitable oral dosage forms. For example, the bacteriophages may be combined with at least one excipient such as fillers, binders, humectants, disintegrating agents, solution retarders, absorption accelerators, wetting agents absorbents or lubricating agents.

According to some embodiments, the carrier is intended to be used in to treat or prevent root canal infections. In such a case, the carrier may be in the form of a liquid carrier such as saline, used to rinse the infected area, e.g. root canal when it is open, before canal sealing off. Alternatively, the carrier may be used as a depot for the active agents (bacteriophages of the invention) to be released for prolonged periods of time (sustained release) and in such a case may be a gel, a chip or degradable polymer to be placed in the root canal when it is open before closure. The pharmaceutical composition may be applied in the form of a powder, liquid, cream or gel.

Carrier for direct delivery to the root canal may be a matrix such as a fiber, chip or film made from a biocompatible polymeric material sized for introduction into the root canal. In one embodiment of the invention, the polymeric material is biodegradable. In another embodiment of the invention, the polymeric material is non-biodegradable. In some embodiments, the non-biodegradable polymeric material is removed after the treatment. Carrier for direct delivery to the root canal may be a hydrophobic elastomer substrate (e.g., materials such as synthetic rubber, natural rubber, a derivative of natural rubber, gutta-percha, balata, silicone rubber, neoprene, isoprene, or polybutadiene) that is sized and shaped so as to be at least partially insertable into an exposed root canal. In some embodiments, the carrier may be filling materials based on gutta-percha or balata. When gutta-percha is used as the carrier, the carrier is neither eroded nor biodegraded. In contrast to soluble or biodegradable carriers, such compositions can thus remain in the mouth for any desired length of time without the root canal becoming re-infected. Gutta-percha is a naturally based carrier, the main component of which is trans-polyisoprene. Other trans-polyisoprenes may of course also be used, such as balata, as well as synthetic carriers based on isoprene, silicon, caoutchouc or acrylate, or various derivatives of the aforementioned materials. The matrix may be formed into a device for treating root canal infection disease in a mammal, wherein the matrix is impregnated with an effective.

In yet other embodiments, the bacteriophages of the invention may be delivered topically to the oral cavity in a composition that includes a carrier such as a toothpaste, mouthwash, or chewing gum. The chewing gum typically includes a gum base of a biocompatible polymeric material such as an elastomer.

In some embodiments, the compositions of the invention may be applied as coatings to medical or dental material surfaces or implants, by applicators, floss, tape, swabs, and sticks. Isolated and mixtures of phages can be incorporated into an inert carrier such as a polymer compositions and other compositions without affecting their intended functionality. In some embodiments, the coating may be applied by mixing the active agents (bacteriophage compositions) with cement and glue materials.

In some embodiments, the compositions of the invention may be administered intravesically. In some embodiments, the compositions of the invention are formulated for intravesical administration.

### Use of the Compositions

According to some aspects, the compositions of the invention are used for infecting and lysing a bacterial cell selected from the group consisting of: E. faecalis and E. faecium.

In some embodiments, the invention provides a method for sensitizing a bacterial cell to antibiotic treatment, the method comprising contacting the bacteria with a combination of the antibiotic and EFDG1 and/or EFLK1. In one embodiment, said bacterial cell is resistant to said antibiotic.

According to some embodiments, the pharmaceutical compositions of the invention are used to treat, eliminate or prevent diseases inflicted by E. faecalis and/or E. faecium. In some embodiments, the diseases are selected from the group consisting of: endocarditis, bacteremia, urinary tract infections (UTI), meningitis, and root canal infections. The methods, compositions and kits of the instant invention may be used for treating, eliminating or preventing any other diseases or medical or pathological conditions inflicted by an Enterococci infection, such as E. faecalis and/or E. faecium.

The term “treat” refers to reduction of the severity of the infection including the reduction in the formation of a biofilm, healing from endocarditis and preventing root canal post treatments failures. In some embodiments, the phage treatment eradicates an infection and prevents microorganisms from infecting or re-infecting the root and/or periradicular tissues. In additional embodiments, the phage treatment disclosed herein prevents acute or chronic inflammatory lesions around the apex, i.e. periapical periodontitis (also termed apical periodontitis or periradicular periodontitis), prevent abscesses in the soft tissue at the tip of the root canal system, and prevent swelling and spread to the surrounding tissues that may result in osteomyelitis or cellulitis

The term “eliminate” refers to complete irradiation of the disease-causing bacteria as the desired site of administration or systemically in the body as a whole.

The term “prevent” refers to application of the composition to a site, where it is feared that infection will be developed in the future in order to prevent development of infection. The term “prevent” includes or reducing the likelihood or possibility of forming a disease or disorder. For example, in root canals the post-treatment infection is caused by E. faecalis in 30%-90% and the composition of the invention may be applied as a preventive measure.

According to some aspects, the invention provides a method for lysing a bacterial cell, the method comprising the steps of: providing a composition of the invention; and contacting a bacterial cell with the composition in an amount effective to infect the bacterial cell, wherein said contacting results in infection of the bacterial cell by the composition; thereby lysing the bacterial cell.

In some embodiments, contacting is carried ex-vivo by applying a composition of the invention onto a surface thereby disinfecting the surface from E. faecalis and E. faecium. In some embodiments, the surface is coated with the composition such as for a non-limiting example, by spraying the surface, spreading the composition onto the surface, rubbing the surface with the composition, or dipping the surface in a liquid comprising the composition. For another non-limiting example, the composition may be applied onto a surface of a surgical tool prior to applying the tool in a surgical procedure, for disinfecting the tool from E. faecalis and E. faecium and preventing infection in a subject treated in a surgical procedure. For yet another non-limiting example, the composition may be applied onto an implant prior to implantation for preventing infection in a subject implanted with the implant.

In some embodiments, contacting is carried in-vivo by administering any of the compositions of the invention to a subject in need thereof.

In some embodiments, the invention provides a method for treating or preventing a bacterial infection in a subject in need thereof, the method comprising the steps of: providing a composition of the invention; and administering a therapeutically effective amount of the composition to a subject in need thereof, thereby treating or preventing a bacterial infection in a subject in need thereof.

The term “therapeutically effective amount” may be determined in accordance with the desired activity (prevention, or treatment) and the desired mode of administration. One skilled in the art will recognize that dosage will depend upon a variety of factors including the strength of the particular composition employed, as well as the age, species, condition, and body weight of the subject. The size of the dose will also be determined by the route, timing, and frequency of administration as well as the existence, nature, and extent of any adverse side-effects that might accompany the administration of a particular composition and the desired physiological effect. For a non-limiting example, therapeutically effective amount may be an amount sufficient for lysing bacterial cells in vivo. For a non-limiting example, for prevention of root canal infections the amount is between 10⁸/ml to 10¹⁰/ml, or alternatively about 10⁹ phages/ml.

As used herein, the term “subject” refers to an animal including a mammal. The term “mammal” includes human subjects as well as non-human mammals such as dogs, cats, horses, cows, ruminants, sheep, goats, pigs and non-human primates, among others. In some embodiments, the subject is a human subject selected from: adult, child and infant. As used herein a subject in need thereof is a subject inflicted with E. faecalis and/or E. faecium or at risk of being inflicted 15 with E. faecalis and/or E. faecium.

### Kits

According to another aspect, the invention provides a kit comprising a composition of the invention and instructions for the use of the composition, optionally together with packaging material.

In some embodiments, the invention provides a kit for applying the bacteriophage of the invention to a surface for disinfecting or removing E. faecalis and/or E. faecium. In some embodiments, the kit comprises a container for storing the bacteriophage in a suitable carrier, diluent or dispersant, and a mechanism for dispersing or dispensing the bacteriophage from the container. In general, any mechanism that provides substantially even dispersion of the phage may be used. Further the phage should be dispersed or dispensed from the container in a manner that does not cause damage to the surface on which the phage is being applied and also does not damage the phage itself. One suitable mechanism is a spray mechanism that is directly associated with the container. In this device, the pressure is generated by the user when the user depresses the pump (or, if a trigger pump, when the user pulls the “trigger”), causing the phage and its carrier to be forced through the nozzle of the mechanism. In another embodiment, the container is a canister in which the phage are stored under pressure configure to be dispersed via a spray mechanism in a conventional manner by depressing a button, or a valve, on top of the canister. In another embodiment, a fogger or misting mechanism directly associated with the container may be used to disperse the phage over an area. In another embodiment, the mechanism may be a roller or brush such as a paint roller or paint brush. In another embodiment, the mechanism for dispersion is cloth wipe, a paper wipe, a towel, a towelette, or a sponge, that may be prepackaged with the phage or phage formulation similar to an alcohol wipe.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); “Bacteriophage Methods and Protocols”, Volume 1: Isolation, Characterization, and Interactions, Clokie & Kropinski, springer publication (2009); “Bacteriophage Methods and Protocols”, Volume 2: molecular and applied aspects, Clokie & Kropinski, springer publication (2009); all of which are incorporated by reference. Other general references are provided throughout this document.

Various embodiments and aspects of the present invention as delineated hereinabove and as claimed in the claims section below find experimental support in the following examples. Reference is now made to the following examples, which together with the above descriptions illustrate some embodiments of the invention in a non-limiting fashion.

### Materials and Methods

### Bacterial Strains

Most of the experiments were carried out using Enterococcous faecalis V583 (ATCC 700802) grown in brain-heart infusion (BHI) broth (Difco, Detroit, Mich., USA) at 37° C. under aerobic conditions with shaking at 200 rpm. Additional bacterial strains used in this work are shown in Table 1. Unless mentioned otherwise, all compounds were purchased from Sigma Aldrich.

### Isolation and Propagation of Phages

Isolation of phages was performed using the standard double-layered agar method, sewage effluent from West Jerusalem Sewage Treatment Facility was centrifuged at 4000 rpm for 5 min and the supernatant was filtered through 0.45 μm filters (Merck Millipore Ltd, Ireland). Exponentially grown bacterial cultures (108 CFU/ml) were inoculated with the filtered sewage effluent for 24 hours at 37° C. After centrifugation at 10,000×g for 10 min the cultures were filtered through a 0.22 μm pore size membrane filter (Merck Millipore Ltd, Ireland). 1 ml of the filtrate was added to 5 ml BHI containing 0.5 ml of overnight culture (109 CFU/ml) of E. faecalis, which were incubated until complete lysis was obtained. The lysate was diluted in BHI broth, plated using soft agar (0.6%) overlaid with the test strain, and incubated overnight at 37° C. as described. Plaque morphologies were observed and clear ones were transferred into a growth broth tube using a sterile Pasteur pipette. The phage stocks were re-plated with bacterial cultures in order to collect high titer lysates then stored in chloroform (40 ml/1) at 4° C.

The concentration of plaque forming units (PFU) was determined according to the standard method. Lysates were serially diluted 10 fold into 3 ml of pre-warmed BHI top agar (0.6%). 0.1 ml of overnight culture of E. faecalis was added to the tube which was placed on a BHI agar plate. The number of plaques was counted and the initial concentration of PFU was calculated. Assessment of Phage Lytic Activity in Planktonic Cultures

Lytic activity was assessed by inoculating logarithmic (10⁻⁷ colony forming unit (CFU)/ml) or stationary (10⁹ CFU/ml) E. faecalis cultures with purified phages at various multiplication of infections (MOIs) (0, 0.01, 1, and 100) in triplicates. The growth kinetics of the culture were recorded by incubating the samples in a 96 well plate reader (Synergy, BioTek) at 37° C. with 5 sec shaking every 20 min. The optical density at 600 nm was recorded. At the last time point, the number of live bacteria was determined by (CFU/mL) count.

### Assessment of Phage Lytic Activity in Biofilm 30

Static biofilms of E. faecalis V583 and EFDG1^{r} were grown for 2 weeks in a 96 well plate at 37° C. to a width of approximate 100 μM. Phages were added (10⁷ PFU/well) and incubation continued for another week. The wells were washed with PBS and biomass was quantified by crystal violet stain. Briefly, fixation was achieved by adding methanol (200 μl) to the wells and incubating for 20 min, followed by methanol aspiration and air drying. The biofilms were stained 35 by 200 μl of crystal violet (1%) for 20 min at room temperature then washed with water. Ethanol (200 μl) was added and biomass was quantified by optical density reading at 538 nm.

In addition, wells were stained with LIVE/DEAD® Cell Viability Kits (Life Technologies) according to the manufacturer's instructions. The stained biofilms were examined using a confocal microscope; the fluorescence emissions of the samples were detected using a Zeiss LSM 410 confocal laser microscope (Carl Zeiss Microscope). Red fluorescence was measured at 630 nm and green fluorescence was measured at 520 nm. Horizontal plane optical sections were made at 5 μm intervals from the surface outward and images were displayed individually. The microscopy slices were combined to a 3D image using Bio-formats and UCSD plugins of ImageJ 1.49G software (http://imagej.nih.gov/ij/).

### Transmission Electron Microscopy Visualization

For the visualization of isolated phages using electron microscopy (EM), grids were prepared by incubating filter-sterilized (0.22 μm) lysate on a carbon coated copper grid for 5 min followed by negative staining using uranyl acid (2% w/v) in water for 30 sec. A transmission electron microscope (Joel, TEM 1400 plus) with charge-coupled device camera (Gatan Orius 600) was used to capture images.

### Host Range Specificity Tests

The activity of EFDG1, EFLK1 2 and a mixture of both was screened against clinical isolates from the infectious diseases unit of Hadassah Hospital and strains from our lab collections. Aerobic bacterial growth kinetics were followed using a 96 well plate reader. Anaerobic strains were grown in anaerobic jars and their optical density was recorded every 24 h.

### DNA Isolation and Sequencing

Phage DNA isolation was performed as previously described. Briefly, phages were added to overnight E. faecalis culture (MOI=0.01) and were incubated for 48 h in 37° C. until total clearance was observed and high titer lysate (10⁹ PFU/ml) was obtained. The culture was treated with DNAse (10 mg/1) and RNAse (5 mg/1) at 37° C. for 30 min to destroy bacterial nucleic acids. Proteinase K (5 mg/1) and SDS (20%) were added for 1 h at 52° C. to digest both phage capsid and DNAseI.

Sequencing was performed in the inter-departmental unit of the Hebrew University, Hadassah Campus as described. Libraries were prepared by the Nextera® XT DNA kit (Illumina, San Diego, USA) and DNA was amplified by a limited-cycle PCR and purified using AMPure XP beads. The DNA libraries were normalized, pooled and tagged in a common flow cell at 2×250 base paired-end reads using the Illumina MiSeq platform (Illumina, San Diego, USA). Quality of the reads was determine using FastQC (http://www.bioinformatics.bbsrc.ac.uk/projects/fastqc) and reads were trimmed and cleaned by FASTX-Toolkit (http://hannonlab.cshl.edu/fastx toolkit/index.html).De-novo assembly, open reading frames (ORFs) prediction, alignment of whole phage-genomes and phylogenetic tree generation were performed using Geneious 7.1.5 (Biomatters. http://www.geneious.com/). Blast, blastX, gene annotation and gene onthology (GO) analysis were completed using Geneious 7.1.5 and blast2go. tRNAs were predicted by tRNAscan-SE v.1.21.

The circular nature of EFDG1 genome was validated by PCR amplification using oligonucleotides which correspond to the flanking region of the putative “seam” (GATGGAGACACGGAAGCTGT (SEQ ID NO: 3) and CGGCTTTCCCCGTATACCTC (SEQ ID NO: 4)). As a control, oligonucleotides that amplify a fragment with high coverage were used, distant from the “seam” (GCCAAGCTTCTCACACTTCC (SEQ ID NO: 5) and CCACCTTTTTGTCAGGTCGT (SEQ ID NO: 6)).

### Ex Vivo Human Root Canal Model

Extracted one rooted teeth were subjected to endodontic treatment including standard cleaning, shaping, filling and coronal part removal by a diamond bur. Standard endodontic access to the canal was performed using Gates-Glidden drills followed by autoclaved sterilization. Canals 15 were contaminated with E. faecalis suspension (250 μl from an OD 600 nm of 0.1 culture) and the root canals were prepared using K-files (Micro Mega, Besancon, France) and irrigated with 2.5% NaOCl and EDTA in a standard procedure. After the third K-file shaping the canals were re-contaminated with E. faecalis suspension (250 μl of OD 600 nm of 0.1 culture). Final cleaning and shaping was performed by two sequential K-files including 2.5% NaOCl irrigation and EDTA. The canals were obturated in a standard procedure using gutta-percha and an endodontic sealer (AH26, Denspaly, Kanstanz, Germany). The phage treated group teeth were irrigated additionally with 250 μl of phage (10⁸ PFU/ml).

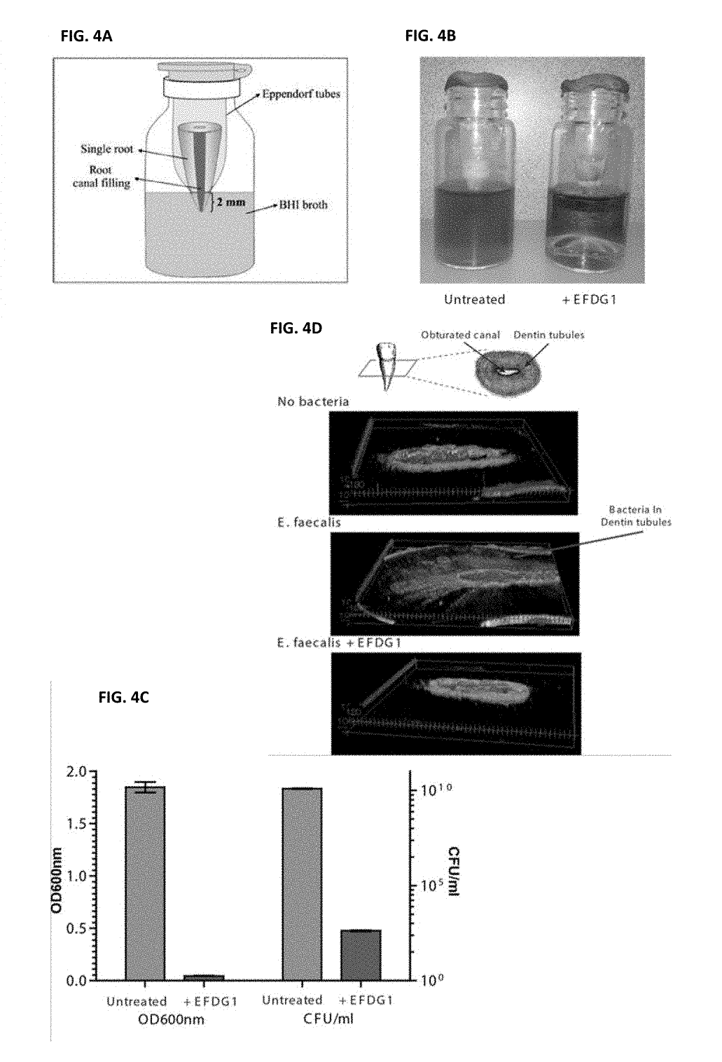

Bacterial leakage was assessed using a two-chamber bacterial leakage model (FIG. 4A). The coronal part (1 mm) of the roots was subjected to further bacterial challenge, i.e. the upper chamber of the model contained E. faecalis suspension (OD 600 nm 0.01). The lower chamber contained sterile BHI broth. To prevent bacterial transfusion between the upper and lower chambers the gap between the root and the upper chamber was sealed using a flowable resin composite (3M ESPE filtek suprime, Minneapolis, USA) and only the apical 2 mm of the root was placed in the lower chamber sterile BHI broth. Turbidity was assessed every 24 h and samples were plated to determine the number of live bacteria (CFU/ml). Then the roots were horizontally split in the center and the internal part of each root was dyed using a live/dead kit (Live/Dead BacLight viability kit, Molecular Probes, OR, USA) according to manufacturer's instructions. The samples were visualized using confocal microscopy as described above for biofilm.

### In Vitro Fibrin Clot Model.

The in-vitro fibrin clot model was prepared according to the protocol described by Enteza et al (McGrath et al., 1994; Entenza et al., 2009). Overnight cultures (109 CFU/ml) of E. faecalis V583 and EFDG1^{r} individually or in a mix of 1:1 ratio were diluted 1:10 with citrated plasma. Clots were formed by triggering coagulation by adding 20 μl bovine thrombin (5000 U/ml) and 20 μl CaCl₂ (50 mmol). The resultant clots were then re-suspended in 500 μl of bacteriophage 5 lysate of 10⁸ PFU/ml and for the control, 500 μl of BHI were added instead. The tubes were kept in an incubator-shaker for 6 hours at 37° C. After incubation, the clots were washed with sterile PBS and lysed with 341 of 0.25% of Trypsin EDTA. After a 5-minute centrifugation at 14,000 rpm, the cell pellet was re-suspended in 100 μl of PBS and used for calculating CFU/ml.

### Example 1

### Isolation and Determination of EFDG1 and EFLK1 Efficacy Against E. faecalis Liquid Cultures

E. faecalis phages were isolated from the West Jerusalem Sewage Treatment Facility which drains the waste water of about half a million people including Hadassah Medical Center. The two phages with the best lytic activity were termed EFDG1 (bacterial strain having SEQ ID NO: 1) and EFLK1 (bacterial strain having SEQ ID NO: 2). These phages displayed clear plaques on double layer agar plates (FIG. 1A) and complete lysis within 24 h in liquid culture (FIG. 1B). Quantitative analysis of EFDG1 against logarithmic phase culture showed that it is effective in MOIs above 10⁻⁴ (FIG. 1C). As further demonstrated in FIG. 1C, in MOIs of 10⁻²-10⁻⁴ a slight culture growth was observed followed by quick lysis and in MOIs bigger than 10², EFDG1 almost completely prevented bacterial growth).

Stationary bacterial cell elimination by EFDG1 was slower than the elimination of cells in their logarithmic phase. Nevertheless, killing was achieved even at a 10⁻⁷ MOI; in other words, 100 PFU/ml were enough to eliminate 10⁹ CFU/ml E. faecalis cells (FIG. 1D). The results shown for both stationary and logarithmic cultures were validated by an endpoint CFU count of the lowest effective MOIs in each case, i.e., 10⁻⁴ after 24 hours with logarithmic cells and 10⁻⁷ after 120 hours with stationary cells. As demonstrated in FIG. 1E, a 5-log decrease of the number of viable E. faecalis cells was observed, after treatment with EFDG1 (FIG. 1E).

The efficiency and kinetics of EFLK1 infectivity was distinct from that of EFDG1. EFLK1 killed more efficiently than EFDG1, stationary cells (FIG. 1G) and less efficiently logarithmic cells (FIG. 1F).

Various combinations of phage cocktails were tested in order to determine the best EFDG1:EFLK1 ratio that would be effective at least as one of the two phages in each condition. The following ratios of EFDG1:EFLK1 were used: Cocktail#1 (1:1), Cocktail#2 (1:2), cocktail#3 (2:1), cocktail#4 (1:3) and cocktail#5 (3:1). A ratio of 1:1 (i.e., cocktail #1) showed superior lytic effect on a logarithmic culture of E. faecalis than EFLK1. Further, a ratio of 1:1 showed superior lytic effect on a stationary culture of E. faecalis than EFDG1. Thus, the cocktail behaved as a better phage than each phage individually (FIG. 1F-I).

A mixture of EFDG1 and EFLK1 killed logarithmic cells better than EFLK1 (FIG. 1F, H) and stationary cells better than EFDG1 (FIG. 1G, I), thus the cocktail is more efficient than the individual phages against different types of cultures. Further, an EFDG1 strain termed resistant-EF_EFDG1 was isolated from E. faecalis infected culture. EFLK1, showed an effective lytic activity against resistant-EF_EFDG1 planktonic cultures (FIG. 1L) as well as biofilms (not shown).

E. faecalis V583 (VRE) is an example of a vancomycin resistant E. faecalis that was announced by the Centers for Disease Control (CDC) as one of the major bacterial threats today. Indeed, as demonstrated in FIG. 1J, adding vancomycin to this bacteria somewhat affects its growth and viability. The phage EFLK1 inhibits E. faecalis V583 growth and reduces its viability by about 4 logs. However, the addition of both vancomycin and phage synergized the effect up to complete eradication (FIGS. 1J and K).

An in-vitro fibrin-clot infection model (McGrath et al., 1994; Entenza et al., 2009) was used to assess the efficacy of the cocktail and the individual phages EFDG1 and EFLK1 against E. faecalis V583 and EFDG1^{r} (FIG. 1M). CFU/ml results following a 6-hour incubation of E. faecalis V583 with EFLK1 and cocktail#1 showed a CFU reduction of three logs with EFDG1 and six logs with EFLK1 and cocktail#1. EFLK1 and the cocktail also reduced the CFU by 5 and 6 logs respectively, against EFDG1^{r}, while, EFDG1 failed as expected.

### Example 2

### Determination of EFDG1 and EFLK1 Efficacy Against Biofilms of E. faecalis

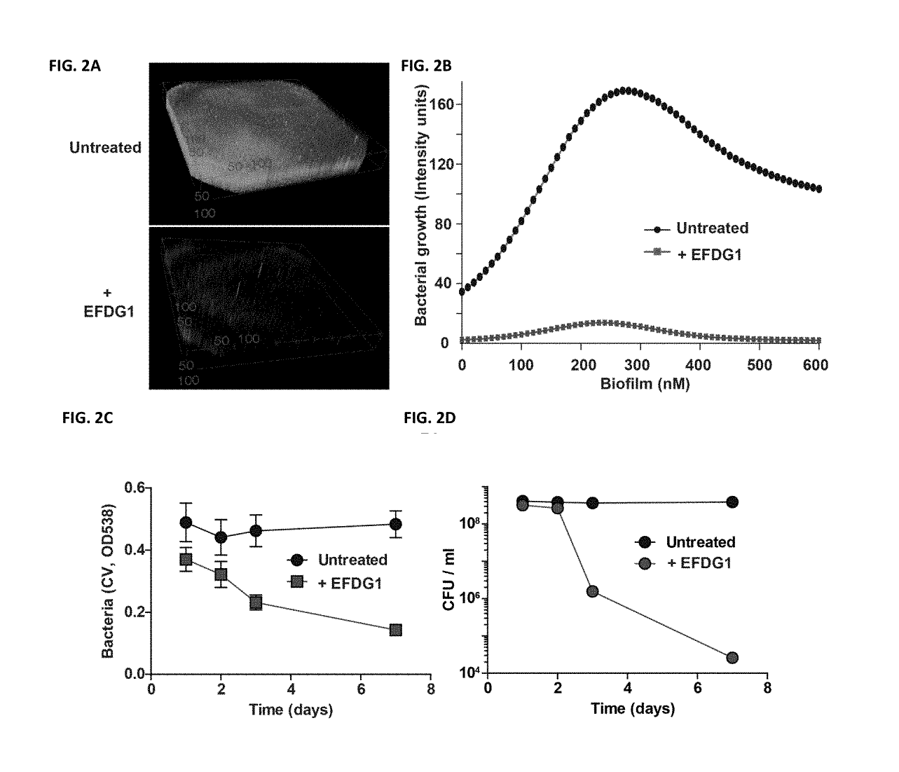

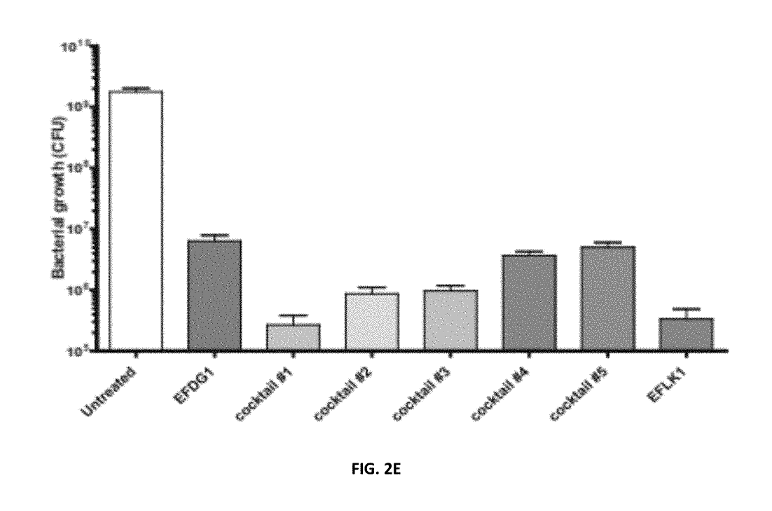

One of the most problematic and challenging pitfalls of conventional antibiotics is their limited effect against cells within bacterial biofilms, which are mechanically and physiologically 1000 times more protected than planktonic cells. In contrast, EFDG1 eliminated and dispersed a two week old 100 μM width E. faecalis biofilm (FIGS. 2A-B). Biofilm biomass evaluation using crystal violet showed a fivefold reduction in the treated samples within seven days, whereas the untreated biofilms were stable and no reduction was observed (FIG. 2C). Viable counts showed a five log reduction following exposure to EFDG1, while no significant change was seen in the untreated biofilms (FIG. 2D). These results show that EFDG1 is capable of effectively eliminating well established E. faecalis biofilms.

EFLK1, similarly to EFDG1, is an efficient killer of E. faecalis challenging biofilms. Furthermore, the cocktail (previously presented in Example 1), like the two single phages, was highly efficient against E. faecalis challenging biofilms (FIG. 2E).

### Example 3

### Assessment of EFDG1 and EFLK1 Host Range of Infection

The infectivity of EFDG1 was assessed on range of aerobic and anaerobic Gram negative and positive bacteria. Strains were grown in 96 wells plate reader for 72 hours. EFDG1 (MOI 0.1) was added at time 0 (logarithmic) or 24 hours (stationary) and optical density was recorded every 20 minutes. Anaerobes F. nucleatum and P. gingivalis were grown in anaerobic conditions and optical density was measured at the endpoint.

Table 1 denotes the details of the tested bacteria, including their antibiotic resistance. EFDG1 and EFLK1 were found to be host specific infecting only E. faecalis and the related E. faecium strains regardless of their antibiotic sensitivity.

TABLE 1
Sensitivity of bacterial strains to EFDG1 and EFLK1

| | | | | Antibiotic | Antibiotic |
|---|---|---|---|---|---|
| Bacterial strain | Origin | EFDG1 | EFLK1 | Resistance | Sensitivity |
| Enterococcus | ATCC700802 | Sensitive | Sensitive | Vancomycin | Daptomycin |
| faecalis v583 | | | | Gentamicin | Streptomycin |
| Enterococcus | Clinically | Sensitive | Sensitive | — | Ampicillin, |
| faecalis aef01 | isolated from | | | | Ciprofloxacin |
| | urine | | | | Nitrofuantoin |
| | | | | | Vancomycin |
| Enterococcus | Clinically | Sensitive | Sensitive | — | Ampicillin, |
| faecalis aef03 | isolated from | | | | Ciprofloxacin |
| | urine | | | | Nitrofuantoin |
| | | | | | Vancomycin |
| Enterococcus | Clinically | Sensitive | Sensitive | Erythromycin | Ampicillin |
| faecalis aef04 | isolated from | | | | Ciprofloxacin |
| | venal blood | | | | Vancomycin |
| | flow | | | | |
| Enterococcus | Clinically | Sensitive | Sensitive | Erythromycin | Ampicillin |
| faecalis aef05 | isolated from | | | Genamicin | Chloramphenicol |
| | venal blood | | | | Vancomycin |
| | flow | | | | |
| Enterococcus | Clinically | Sensitive | Sensitive | — | — |
| faecalis cef02 | isolated | | | | |
| Enterococcus | Clinically | Sensitive | Sensitive | Ampicillin, | Chloramphenicol |
| faecium aefc06 | isolated from | | | Erythromycin | |
| | venal blood | | | Vancomycin | |
| | flow | | | Gentamicin | |
| | | | | Streptomycin | |
| Enterococcus | Clinically | Sensitive | Sensitive | Ampicillin, | Vancomycin |
| faecium aefc07 | isolated from | | | Erythromycin | |
| | venal blood | | | Ciprofloxacin | |
| | flow | | | Genamicin | |
| Enterococcus | Clinically | Sensitive | Sensitive | Gentamicin | — |
| faecium aefc08 | isolated from | | | Streptomycin | |
| | venal blood | | | | |
| | flow | | | | |
| Enterococcus | Clinically | Sensitive | Sensitive | Vancomycin | — |
| faecium aefc09 | isolated from | | | | |
| | feces | | | | |
| Enterococcus | Clinically | Sensitive | Sensitive | Vancomycin | — |
| faecium aefc10 | isolated from | | | | |
| | feces | | | | |
| Staphylococcus | Clinically | Resistant | Resistant | — | — |
| aureus w6460 | isolated | | | | |
| Staphylococcus | Clinically | Resistant | Resistant | — | — |
| aureus w0406 | isolated | | | | |
| Staphylococcus | | Resistant | Resistant | — | — |
| aureus lsa011 | | | | | |
| Pseudomonas | | Resistant | Resistant | — | — |
| aeruginosa Pa14 | | | | | |
| Pseudomonas | | Resistant | Resistant | — | — |
| aeruginosa Pqsa | | | | | |
| Streptococcus | | Resistant | Resistant | — | — |
| mutans lsm012 | | | | | |
| Streptococcus | | Resistant | Resistant | — | — |
| sobrinus lsb013 | | | | | |
| Fusobacterium | | Resistant | Resistant | — | — |
| nucleatum fs014 | | | | | |
| Porphyromonas | | Resistant | Resistant | — | — |
| gingivalis pg015 | | | | | |
| Burkholderia | Clinically | Resistant | Resistant | — | — |
| cepacia | isolated | | | | |
| complex 25 | | | | | |
| Burkholderia | Clinically | Resistant | Resistant | — | — |
| cepacia | isolated | | | | |
| complex 80 | | | | | |
| Klebsiella | | Resistant | Resistant | — | — |
| pneumonia bkp016 | | | | | |

### Example 4

### Characterization of EFDG1 and EFLK1 Genome Sequence and Phylogeny

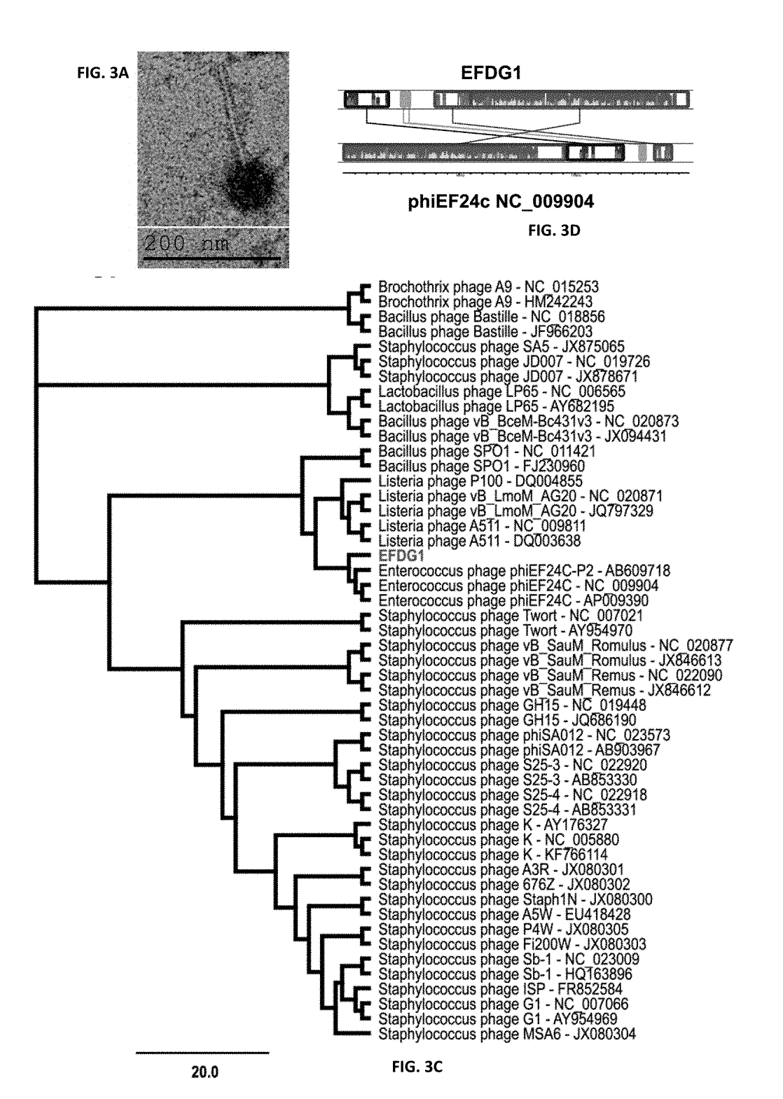

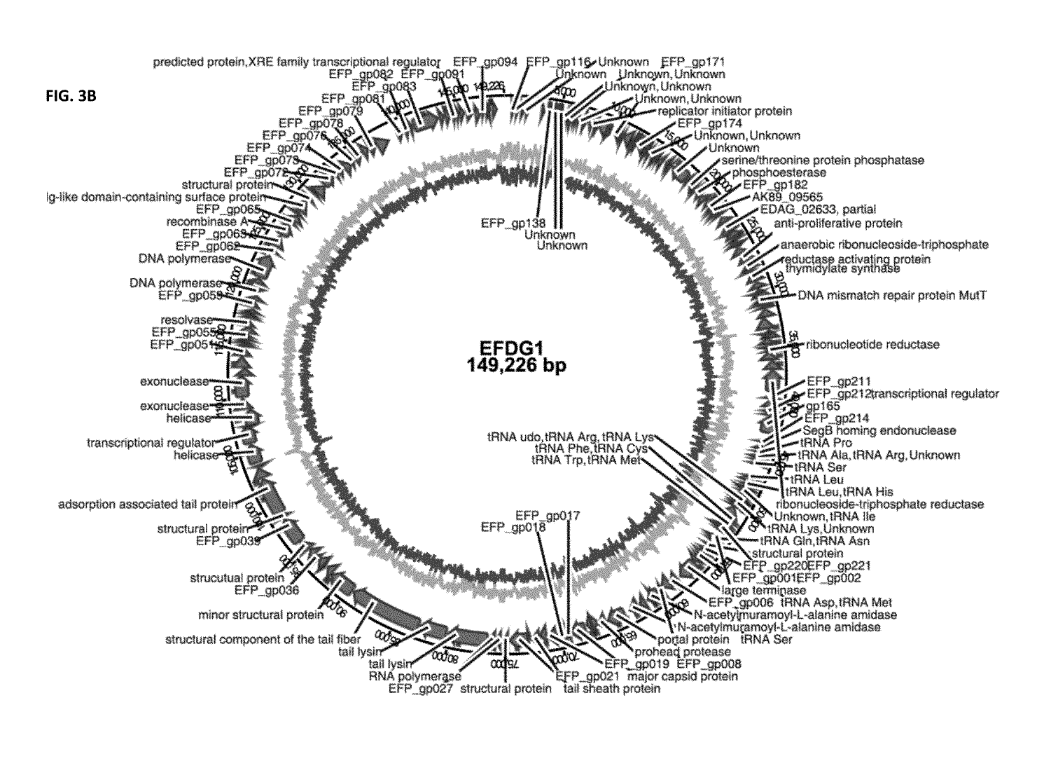

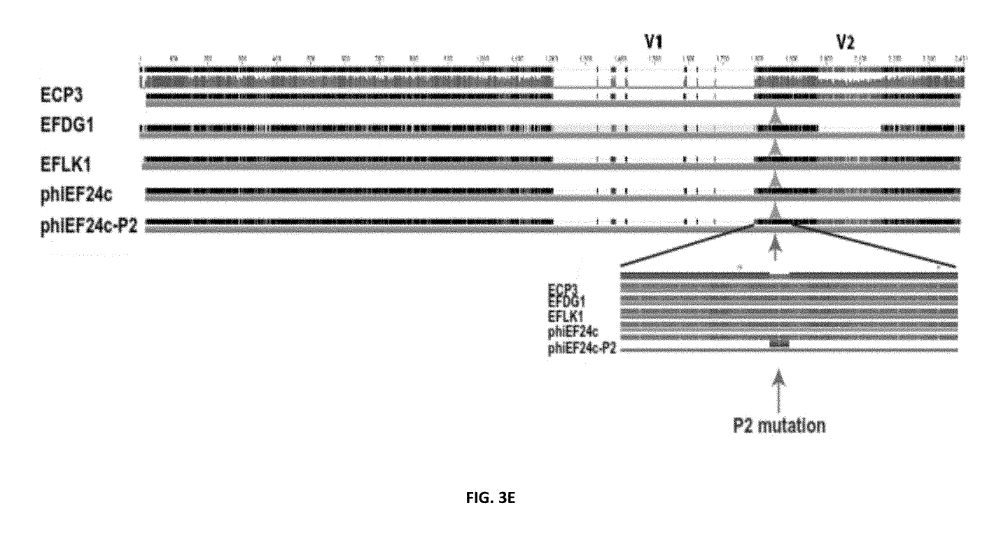

TEM microscopy showed that both EFDG1 and EFLK1 has a round capsid and a contractile tail (FIG. 3A) suggesting that it belongs to the Myoviridae family of phages (http://viralzone.expasy.org/all byprotein/140.html). A whole genome sequencing of EFDG1 and EFLK1 was performed. EFDG1 genome yielded 634,614 paired end reads with a mean length of 244.4±15.6 base pairs (bp), which were trimmed and cleaned. Reads that aligned to the E. faecalis V583 genome (GenBank (NCBI) Accession No.: AE016830) or its three plasmids (GenBank (NCBI) Accession No.: AE016831-3) were excluded from the analysis. The remaining reads (194,186) were subjected to de novo assembly, which yielded 10 contigs with more than 10 reads. The largest and most significant contig contained 149,226 bp, assembled from 186,686 reads (96% of the reads) with a pairwise identity of 99% and mean coverage of 295±81. It was found to be circular (FIG. 1B). Validation performed by reads-shuffling and re-assembling showed that indeed it is a circular sequence.

EFDG1 genome is AT rich with a GC content of 37.1%, similar to that of its host E. faecalis (37.5%). In accordance with the TEM images (FIG. 3A), a Blast search showed that EFDG1 belongs to the Spounavirinae sub-family (http://viralzone.expasy.org/all byprotein/2777.html) of the Myoviridae family (phages with contractile tails, http://viralzone.expasy.org/all byprotein/140.html) of the Caudovirales order (tailed phages). So far the Spounavirinae sub-family contains 50 members with fully sequenced genomes, all of which are Gram positive bacterial phages including bacilii Staphylococcii, Listeria and Entrococii (http://www.ebi.ac.uk/genomes/phage.html). Multiple alignment and phylogenetic tree analyses of the Spounavirinae phages genomes, including EFDG1 (FIG. 3C), showed that the closest phages to EFDG1 belong to the E. faecalis phage PhiEF24c group (FIG. 3D), Listeria phage A511 and Staphylococcus phage 676Z, with 55,730 (37%), 53,730 (36%) and 50,604 (34%) identical base pairs respectively.

Prediction of open reading frames (ORF) larger than 100 bp identified 210 putative coding sequences and 17 tRNAs. BlastX analysis showed that 166 (79%) of them have similarities to sequences in the non-redundant NCBI database, most of them to the phiEF24c phage. Putative functions could be attributed to 79 of the 166 ORFs, with the majority of them belonging to four groups. The first are phage structural genes encoding capsid and tail proteins, and proteins which are involved in adsorption and/or lysis of the host bacterial cell. The second group comprised of an impressive pool of putative proteins involved in DNA metabolism. It appears that EFDG1 harbors fully functional DNA replication and repair machinery that includes two DNA polymerases, two exonucleases, two helicases, as well as recombinase and resolvase. In addition, the EFDG1 genome contains RNA polymerase and a large set of tRNAs genes, but not rRNA.

It should be noted that among the annotated genes no known harmful or antibiotic resistance genes were found.

Beside the annotated genes there are 85 open reading frames (ORF) that are conserved and appear in other, mainly phage, genomes but do not have attributed functions. Lastly, EFDG1 has 59 ORFs which are putative coding sequences unique to this phage without any homolog in the non-redundant database.

In addition to its ORFs, the EFDG1 genome contains 63 regions of repeats (FIG. 3B, red boxes), which can probably be attributed to the genome rearrangement and the differences between EFDG1 and phiEF24c.

The EFLK1 phage contains a circular genome of 130,952 bp, with 209 putative coding sequences and a G+C content of 35.9%. No tRNA genes were identified in EFLK1, in contrast to EFDG1, which harbors 23 genes. According to our analysis, EFLK1 belongs to the Spounavirinae subfamily of the Myoviridae phage family. As such, EFLK1 (GenBank (NCBI) Accession no. KR049063.1) shows similarities with the other E. faecalis Spounavirinae phages, EFDG1 (GenBank (NCBI) Accession no. KP339049.1), PhiEF24c (GenBank (NCBI) Accession no. AP009390.1) and ECP3 (GenBank (NCBI) Accession no. KJ801817.1). EFLK1 contains a significant cluster of DNA replication components, including two DNA polymerases (EFLK1_ORF130 and EFLK1_ORF142), two DNA helicases (EFLK1_ORF161 and EFLK1_ORF159), DNA maturase A (EFLK1_ORF121), three DNA exonucleases (EFLK1_ORF127, EFLK1_ORF157, and EFLK1_ORF158), resolvase (EFLK1_ORF147), and primase (EFLK1_ORF155). Additionally, with regard to transcription, EFLK1 contains a gene for RNA polymerase (EFLK1_ORF176) and a sigma factor (EFLK1_ORF134), which are conserved among many Spounavirinae phages, including the 3 E. faecalis strains and other Gram-positive bacteria.

The most significant difference between the EFDG1 and EFLK1 genome sequences is the presence of tRNA genes. EFDG1 contains 24 tRNA genes while EFLK1 does not contain any. The other two E. faecalis phages, phiEF24c and ECP3, contain 5 tRNA genes each.

The correlation of the codon usage of each phage to that of their hosts E. faecalis and E. faecium using CORREL function of MS Excel showed that EFDG1 is the most similar phage in this term (0.921), while the other 3 phages have lower correlation (ECP3, 0.89522; EFLK1, 0.8954; phiEF24c, 0.8948) (results not shown). All three phages, EFDG1, ECP3 and phiEF24c carry tRNA genes corresponding to codons CTA, AGA, GAC and TGG of leucine, arginine, aspartic acid and tryptophan, respectively. Indeed, in the three phages, the usage of these codons is higher than their usage in E. faecalis. However, it is also higher in the phage EFLK1, which does not have any tRNA genes. Besides these tRNA genes, all three phages EFDG1, ECP3 and phiEF24c encode for the tRNA corresponding to the initiation codon ATG of methionine, of which EFDG1 has two, and E. faecalis nine copies. All of the tRNA genes of EFDG1, ECP3 and phiEF24c are interspersed in a conserved region that is fully absent in EFLK1. A codon usage analysis of this region in phiEF24c did not show much difference with respect to the rest of the genome. However, the bias with respect to E. faecalis became less pronounced for the codons corresponding to leucine, arginine, aspartic acid and tryptophan, and more pronounced for the codon corresponding to methionine.

### Example 5

### Anti-E. faecalis Activity in an Ex-Vivo Human Root Canal Model

To test the activity of EFDG1 in post-treated root canal infections an ex vivo two chamber bacterial leakage model of human teeth was used (FIG. 4A).

No turbidity was observed in the phage treated samples, therefore, it was concluded that the obturated root canals that were subjected to EFDG1 irrigation resulted in reduced bacterial leakage from the root apex when compared to the control group (FIG. 4B). Indeed, quantification of the turbidity and viable E. faecalis counts revealed an 8 log reduction following phage irrigation (FIG. 4C). Confocal laser scanning microscopy (CLSM) images of horizontal root sections showed that stained bacteria were evident only in the dentinal tubules of the group which was treated with E. faecalis. In contrast, no stained bacteria were seen in the phage treated teeth or in the sterile control teeth, demonstrating the significant reduction of stained bacteria (both dead and living) by EFDG1 (FIG. 4D).

### Example 6

### Peritonitis Phage Therapy Using Anti-E. faecalis Phages EFDG1 and EFLK1

The efficacy of EFDG1 and EFLK1 was examined in systemic peritonitis settings. Mouse peritonitis model was carried out on 40 female ICR(CD-1) mice, which were infected with intraperitoneal injection containing high inoculum (10*LD50; CFU-2e10) of Vancomycin-Resistant strain of Enterococcus faecalis. The mice were divided into several groups, each treated in a different time point after infection using the EFDG1 and EFLK1 bacteriophage cocktail (PFU 2e8), both individually or in combination with standard antibiotic regimen (Ampicillin 12.5 mg/kg). The mice were evaluated by clinical and laboratory findings (CBC), and bacterial load counts were obtained from numerous tissues. Clinical evaluation of the mice included activity and consciousness, breathing rates, general and eye appearance and more.

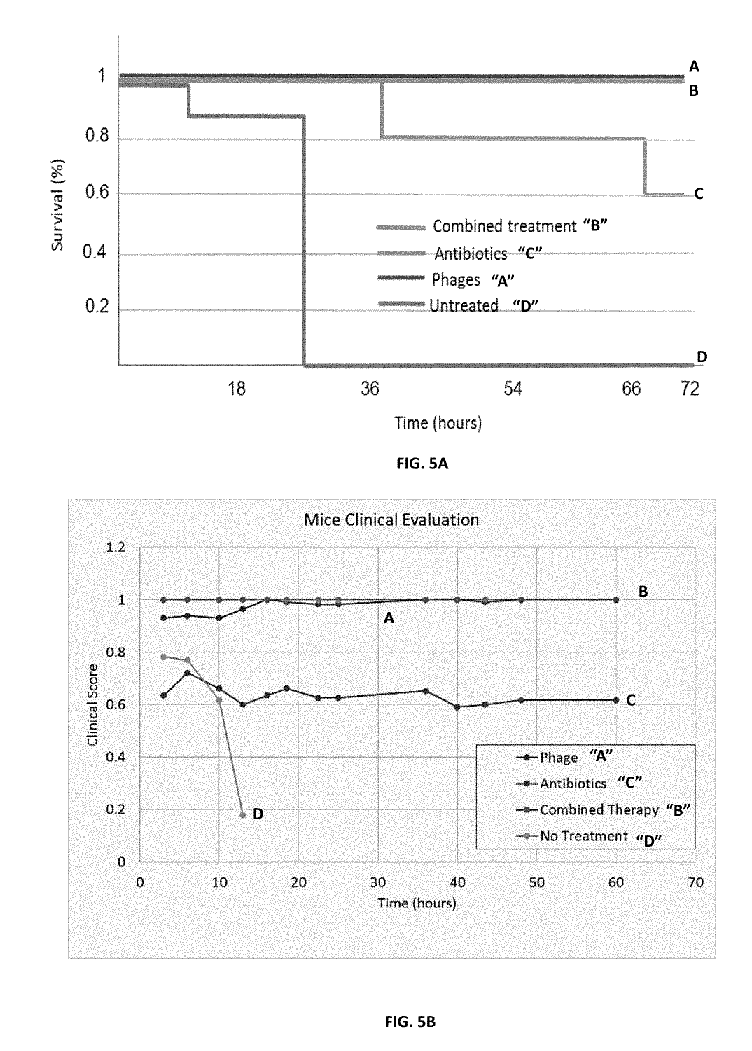

As seen in FIGS. 5A-B, the phages alone (depicted “A”) and combination of phages and antibiotics (depicted “B”) were superior to the antibiotics treatment (depicted “C”). This reflected both in mortality (FIG. 5A) and health condition evaluation score (FIG. 5B).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A composition comprising:
i. an isolated strain of bacteriophage having a genome comprising a nucleic acid sequence of SEQ ID NO: 1, or a sequence having at least 95% sequence identity thereto; and
ii. an isolated strain of bacteriophage having a genome comprising a nucleic acid sequence of SEQ ID NO: 2, or a sequence having at least 95% sequence identity thereto,
wherein a ratio between the isolated strains of bacteriophage having a genome comprising the nucleic acid sequence of SEQ ID NOs: 1 and 2 ranges from 10:1 to 1:10, respectively.

2. The composition of claim 1, wherein the ratio between the isolated strains of bacteriophage having a genome comprising the nucleic acid sequence of SEQ ID NOs: 1 and 2 is selected from a range of: 1:1-3:1, 1:1-1:3, 3:1-1:3, 5:1-1:5 and 2:1-1:2, respectively.

3. A composition comprising one or more isolated strains of bacteriophages selected from the group consisting of:
i. an isolated strain of bacteriophage having a genome comprising a nucleic acid sequence of SEQ ID NO: 1, or a sequence having at least 95% sequence identity thereto; and
ii. an isolated strain of bacteriophage having a genome comprising a nucleic acid sequence of SEQ ID NO: 2, or a sequence having at least 95% sequence identity thereto.

4. The composition according to claim 1, further comprising one or more antibiotic.

5. A pharmaceutical composition comprising the composition according to claim 1 and a pharmaceutically acceptable carrier.

6. A method for treating a subject inflicted or at risk of being inflicted with a bacterial infection, the method comprising administering to said subject an effective amount of an agent selected from the group consisting of:
a. an isolated strain of bacteriophage having a genome comprising a nucleic acid sequence of SEQ ID NO: 1, or a sequence having at least 95% sequence identity thereto; and
b. an isolated strain of bacteriophage having a genome comprising a nucleic acid sequence of SEQ ID NO: 2, or a sequence having at least 95% sequence identity thereto;
c. a combination of (a) and (b).

7. The method of claim 6, wherein said bacterial cell is selected from the group consisting of: Enterococcous faecalis and Enterococcous faecium.

8. The method of claim 6(c) wherein the combination comprises the isolated strains of bacteriophage having a genome comprising the nucleic acid sequence of SEQ ID NOs: 1 and 2 in a ratio ranging from 1:10 to 10:1, respectively.

9. The method of claim 8, wherein the ratio between the isolated strains of bacteriophage having a genome comprising the nucleic acid sequence of SEQ ID NOs: 1 and 2 is selected from a range of: 1:1-3:1, 1:1-1:3, 3:1-1:3, 5:1-1:5 and 2:1-1:2, respectively.

10. The method of claim 6, wherein said method further comprises administration one or more antibiotics.

11. The method of claim 6, wherein said administering is ex-vivo.

12. The method of claim 6, wherein said administering is in-vivo.

13. The method of claim 6, wherein said bacterial infection is selected from the group consisting of: endocarditis, bacteremia, urinary tract infections (UTI), meningitis, and root canal infections.

14. The method of claim 6, for treating or preventing a root canal infection in said subject.

15. The method of claim 14, wherein said agent is present in a sustained release composition.

16. The method of claim 14, wherein said pharmaceutical acceptable carrier is selected from the group consisting of: a gel, a chip, a film a non-degradable polymer and a degradable polymer.

17. The method of claim 6, for treating or preventing urinary tract infections (UTI) in a subject.

18. The method of claim 17, wherein said administration is directly to the urinary tract.

19. The method of claim 18, wherein said administration is intravesical administration.


SEQUENCE LISTING (6 sequences)

SEQ ID NO: 1 - DNA, 147589 bp, Bacteriophage
ctttaacaaacacgtaacagcagaagatattatcagagatggagaatacaatgatgcagttgtagaacatattattggagaacttgaaacaagtgataagaaaaaattagctctattcaaacaatggattgcagacaaaaaagaagatggagacacggaagctgttgaatactttaaaggtatgacagataaagaaattttggtttacatggacttagacgaaattgtagaatactacgaagaaacaggtcttgcagactttgcagatagtttagacattgactggacaggtttactagaaagttttggagtagaataggaggttaagactaatgtatgatatggtaactaaaaagaaaagaaatcaagcaattgtacgtatgagacaggcaggaaaaacgtttaaggatattgcttctgagtataacatttctcaacaacgagtgcaacagatatacaaacgagaaacctcgttaaataagattagacctaaagctggtagaccaagaaagcacgaaattgatgagaaacgaacagacattagtacttttgcagtaacgaagagacagagagaaagaatagtccaacaagctgaaaaggaaggaaagccaattaaggaggtagttgattctattatctttaatgcaatgagtggggaatacaacagccttttccataaaaaaggtggagataataaacaggttcctatggtagggaataaacaactgttagatgctttacgtgcatatcttggtgcatatgaggaaaccccatcagaggcgttaagactagttatagataggatatacgaggatgaatagctgttaggataggatgataggatacatttaggataagataggataagataggataggtttaggataggatacacttaggataggatgaaaggggatatgatacaatgttaagtacagcagaatacgattcacttagagagtatttagtagccgtaattcataacgaaattagtgagtatggaggagacacgcaaagcttagacgtatggaaagaagaggttagtaagtacgcagataatcgaataggttggtatctccgttgcatgaacctagcagatttgttatatgtagatactcccgcttcactggcagacatttacaaagactataaagaggagctaaacgagatactcagagattatacagaggagttatatgcaggggctacgttcgataaggaagctgacgagcgcttagggtggatgtgggaagattatgacccattaatactagaagcacataatcaaggtctagtggctcgtctggggcttctaatcgttgctgaacaacttgctactcaattagaacaagggacgtttacaataccatatgataactaagtaacaggctagcctaagtagcaggctagcttttttcttctaagtagcaggttagctaaaatcttctaagtaacgggctacagtaaaaaaactctaagtagcaggctacaaaaatcctctaagtagcgggctacaaatagcttataaaatcccgaggcgtaccatccgacagccagacttgccacctttgcccgttcgatatgccaccactcaaacccgttttttccttatatatagaagaaaaaagtttttcatcttttttagtgttttctattgacattaagaaataggcatgatatagtatagacagttaaagaaaggagaaacaaacacgaataaaaaaattttaaaaaaagttattgacattaaacaataaacaagctataataaacatgtaagataaataaaagaaagagggaactaaaaatgagtaaaacagttgaaacagttattacattgatgaaaagggcagaaaagaaaggcgtagcaatcaaacgagcaagacaacgggacatgtggggcgtgggagacttacaagaagtgtatcgagtagaaaacaacggtaaaacagtaagcttataccactatgaaacactaaccgcaaaagttgaccttgaaaacatgcaactaatagaggtatacggggaaagccgttcggatagtgatagcgtggcaactttcttatctgaattaggttttaaaaactttgactttggttttaaaccagtaaacgggggcttttggttattcactaacaaagacggggaaagacaagaaacctatctagacgaacaaggagaaaccgccgttataaatgaatttaatcgggtacaggcataacaaggagagagagagggggaaacctctctttctaaaaataaaaaattttaatcttttctattgacattaaacaataaagataatataatactagtgaggaatgataaaaatgaaaatcgaaattatcgaacctaaaaaagaatttaaatttttaactagggaggaacttaacacctatcaattagaagcgggggaacttgtggaactaacttatatagtaaactacacaaaagaaacgcctaaagaattgacgagtgttttcgtgtttgcggataccatcggggcaagctcgtaccttgtagacgtagaaacgggcataatgacaaagacacaaacaaaaaacccgctttctaactgtaatctagctataacaaaagtaaactttttcaaaacaagcaaacttatactagaaaggaaagaaaactaatgaatttaaaagagtggggctatacatgggacggcatgaaagaaatcacacgggaacaagcaaagaaacaatacaaggaaaaagaaacatttgttctatatagtgatgacacagaaagccttattgaatcagaagaacagtttaatataaaaggcgttcggtacggtgtggaaaaggagggggaacaatgaacaaatttctagaagaaagaaaagaagatttacttatacagcaaaaagaacaagctcaaaccttattacactatatagaggaaaacaaagaaccagaccaaaccatcatacaaagacaaacaaacaggcttaacaggttaagcgctcaaatatacgaaattaaaatgatacaaagaaaactaaaaagaatataaggagagaaaacagacatggaaacagtaaaagacattaaaagaatgataagaacactaaaagacgatataaagcataaagaaagctatataaaggaattagacgaacaaggaagaatgatagagtcagaactattgaaagcagaagtaaaaggaatgagggacgttatacaggacatagaacaagctatatattagagagagaggggaaaaggaacaaggaaacagaccaaagagcaaaacaaaaaaaatagagcaaagaaccaaaaaataaaaaaagaccttagagcctgagattttcaaaactcgagcttttcggaactcgcttataagggcgcacactgtttttctatcattctagcacaaacaggaagctagagggcacacaatagccctaggaagccacaggaagcccttagagcggttttacatagatttatatagaattatccataaataaacagaactattctagaatgctttaaaatagcttaaaataaattgaaatatttaatagtttatacttgcatttcatctctttacatggtatagtatagatagttaaaagagagcttcattattcggacgagaaacaaaataaaaaagttgagtttaatgcttgacaaggtaaactatacatgttaatataaaggtgtggttagggttaaaccttacaggaaaacgagtacgaccacacaaacaaaaaaatttaaacaaatctattgacattaaacaataaagctgatatacttaacttgtaagataaataagagggggaacttaaaaatggcaaacacgacaaagaaaccaaaatatttagtggtggataactacgatttagacatgtacgaactaggacgggttaacactatgcgagaagtgagaaagctttgcaaggaacaggtagaggctacagacggggaatgctatgttatggtgctaactttaaacgaggaaacaaacaaatattctagaaaatcggcggaaattgttcagtactaatacattattgttatttaaactaaaaaccgaggtgcaaggcaaaaaacaggaatgctattgcaccaaggtttgcgggactaggtggttaaaacttccctagggcagaacttcatagttaaaaatttaatcgtgttattttaaacttttctattgacattaaacaataaactagttataataaaggtgtaaggtaaagagagggaggaaacgacatggaatacaacacactagacgagttattagacagtaaagaatttaaggacttcatggacgaactagaagacgatggattttttgaccaattcgaggacgaggaagacgggaacacactagcagacacgggacgttatccagaataaggagaggcttaacaatgaaagcaatcatcatcaaacaacttaaggacggttacaaaggcattctagcacgtaaggaaaacggttggacaaaggaggaactaatcaattttacagcgggcgtaagcaatacactatttgaccttatcaacgatttacagggcgggggctttattacagacaaggaagcggaggaaatcgaggaagtattttctaacacgattgacacactagaattttagaataatctattgacattaaacaataaaaataatataatactagtgagggggggcttttccctctcactcacacaagaaagggactgaataaaatggaaaatgtaaaaattgcaatgaaagctatccaagaagaaataaacgaaaccactctaattataacggcatgtaagagaccgccacagtggtttattgaccatctagaccgattacagaaagcacacctttcactacaagacgtagcaagacaaatgccagaaattacagaaagcggggctaactaataatgaccaagcaactaatgaaagaagcgcttgactacctagaaaaagagggaaaacaaatcactagaaaacacgttacacagtacggctttaccatgtatgaacaccctaccttttcaagtgcggttattatggtaaaagatggtaaaattgtaggacaaacaaacaaagctaaaatattagcagacaacagcagaacacgggaaaaattagaaacatctgattttatagaatttacactataataaaacggcatataaagcccgtagcgaggttttaacgggctttatatataaataccttaaggagggcttacaagtgcttacagtggaaaataagggcgttagatggctagtgagtaaagaagcatggaaagcgggctttgctatcgaggtgctaggcttgccagattgtaagattagtacaatattaaaagcgggctatagaattattatttaattatttaatcatatctattgacattaaacaataataatggtattctatacatgtaagataaatgaagggaattgataagaatgaagaacctaatgaatgaaactatctatagtaatattacattaaacctaactaagaaggcgcaaggcattatatcccaatacgtagaccataacgagggctatccaagacaaaagaacagaggtcactactaccgctatacagttacatttatcaatggtgaaacgtttgaaagtaatagcaaggcggacatcgctcactatatttcatctatgacagcggacgaactagaaaccatcatcaaggaagcacaagccgagcagtaagaaataggtatataccagttaaccccaccccgtggttttgtgaccgtatttaggggcggggctatcagggcaccctcgaccctcgggtacctattttttggactggaaattttttctagaaatttggaaagtcaacactaaaagtcaacaccttataactaaccccaccccgttgttttatctagtcgatttcttctatctgcgtgtttaatagatacatagtaccacccatacccgtaccctctagaacgatacactccaaaaatattatcctaggggcactgacatcctctagggagataacttccccatttaagacatccccaccgaccacctgcactacatgtcctagtagtgtcctatactctttacctatcactacttgttctaccataatgctttcctccttataaagaccctaccccgttgatgatgaggtctttttattgtgacgtactccatagttacattcctttattccaacctcccacatctttatgagtatcgaaaccacaatcggggcagtaacctgcttgttctgcatctgttgggtacatgctatagctataatagaaagcatacatccccatgttttcatgcgtacactctgattcctttttcttatacgggtaaggacatgcaggacaggtaaatgtatactctcctgttgaaccaattaaacgttccccgcattttgggcaaatatccgcaaccatcttagtcctcccccactataaatcttgctttagcctttaatggtgcgtagtccttttctgttagtcttacataagcaacacccggcactcccggagagcatacgtaagcaactacaccccagtctttgacttcacttactgtggcaaaacatcccccaaatgtttcatcgaaatgtgggactatttgaacaacatcatataactctgtgtgatacttcatttaatcttcctccgtacttaatgagtcttgtagatagaagtcggagattccccatttgtttatataagtaatttctccttctttactacctgtcacctgttttaacttgtatgtgatttcttccccgtcttcacgagagtttccgtctttgtctattacgaacagtggagtcttctctgtgtctaatagttcgtaacgctcacctgttagcttgtgtatataaatcatgttatccctcctttagaacaatgcacatataaatacaatcaccgtagttttcactgtcatatgtgatatagtctacttcatagtctagtaattgttctgggaaatcttttgagtaaaactctgtacgccatgactgttctgcgcaatagggttccccgtcaaagtctttaatatagattcgaaaagagtcaccaactgataggatgtctctaacttgtctaagtgttaatttcaaatgtcttgcacctccggttctgtgatgtctaggacagctattacgtgtacctgtcctactccttctatatataatggtgtaaataaaccagttaactcgactaagttatcatgcctaatcgtgatagcattaaacttgtaattacacccataaataacttcatagatgcccataaaaaccctcctgtaatttatacttaaagtatatcacttagagggttaattgtcaataccaagcaggcaaagattgttcataatcgaaagaccaaccactagagtttaaaacttctcctatctcttttatactctttgctttcctaatcttctttctagcatcgaggtgttctttccttgttatactactgtataaggtttctcctaaccttataagaagctcgtctttaaactttttgaaggtgtatccttcgtaatagtctgcccattcccatgtaatactttgtgttcctttaaatactttactgaaccctgatatggttatggtgtatttgattgtcattggtagttccttcatcgtcttatctcctatacccatgaaatcttcctgttttgttgtggacgaaatacctctactttacgtgtatgaatgtccctacctataatagcgtaaggactataatctattacttcatacaaacgtacagtgtaggggtgttcttcccgtataaagctactagcttctgcaatactgaaaaccttgttaacaatgcgtactgccttcattacacaccactgtacctttcttgctcttcatagatagccatgtcttcttgtagttcgacttctctgtctatacatttataacaggtttcaaaggtgtagtcgaagtcacaatctaattcactcactagtcttccacaactagcacaacgtacctcattttccattactttgcactcccaatatattgaacaaaataatttgagccttttttagctaaagtaaacatagctgtaacttctccttgttctattacttcatgagtaattaagtctactaagttaatactactcatatttgcaataaaagcctctccacaaggatgcccctctacaagtatatgtgctcctgatgctgaacgacctctgtaagagtctaggatacgaatgtgtcctttaaaatctaccttttcaattacttggtatttttcggggtagtgttcaatataagcatccccatattcaagtaaccgccaatcagttaaccctcttgctttatctggtacccaaatattgtccaatctttctaaataataggttactgccattgtttctcctccttgttaggtattactcccataggaatatatcgtttcttataacaaaaatgtcttctagcggagtattataatgcaactctacattatgattctctttcatgtaggtttttgcataatcaaataaatgctctggacacttaaaactaaatgaaccaccatctgcatctttgcaagctatatgctcttccatagtactattgacaaaccagtttacacactttggtggataacctagcataattcctagtgcataatcttttccgtgttttcctgataatttagaaaactcagctagttgttccaatttagaaacaacaagtatgccatttttgcctacagaagttgcaaaatgctcccgtacaacacgtttaagttctggtgtcccaataaagatggtatctcttagtcccaaattaaatgccacagtttcttctgctaaaataaccatgataatctctcctcctatacctaattactcttttgatagtttgcttcttctaattgtcctccactatttcgctttaaaattatatacaggtttgacaatcttttgaatatctactgtatctttgatgatttctttgatttcgtcagcattcttgtatgcttttggtgcttcgtctagtgtactttgaacaacagatgttgtatagataccagacattgtttgtttaaactcatctaagtcaagcagttcttttgctttagaacgagacaatacacgccctgctccatgaggtgcagaattattccaatcagtgtttccttttcctacaccaatgatagagccttctttcatgtttaaaggaataacaagtctaacaccctcctgtgcatctgtggcacctttacgaatagtgttggaggctacatcaatataattatgaatagagtcaaatgagtcaactacagtccaacccatattagaaacaatcttatcaatgatagttttacgattcatgtaagcataacgttgtgctagttgcatatcatttaaatagtcatccactgcggaaccttctaagtatggcaattgtggtttaacattgacagctttaaaattcttaatagcctcttggatttctgactgtctgccttcttgcttaagtgtctcaatcatagttgctaaacctttgtcatcacgttcatggtataaatccgctttcttctggtgatattgggctactttaagtcctaaagaacgggaacctgtatgaactaacaagtagtaattgttttcgtcatctttacttaattcgataaaatggtttccaccacctaaagtacctaaagataaggtaacacgttcaataaatggttcttctgttgcacctttaaacgtcatagattctaacatgtgcttaatcgtattcggtacagtaggatgaagttcgtccaaattttctctaatgttaaacccagaaggaaccactttgttaatcactgcatccaatttagcccattcggtatctgtcgtgtcttctggcttctcaatttgagctaccataataccacaacctacatctacaccaacaatagagggtacaactcgtttaataccgtctggataatgaatcgttgtaccaactgtagagcctttaccataatgtgcatctggcataattctaacaatgccaccgtctgttaccggttcattaatcattgttgtaacctgaccgatagtgccttcatcgactgtattaataaacattttcgcatcggtatacttaccgttaagttcaattaagttattcatcagttttctcccccactaatactttcttcatcttttcatctagtctctgtataatctcttgttgttcctgagctagctttgcatggtgttcacgctgttttttatacttagcataagcaaatagttcttctgtagttagttcttctaatttacgtgtaaatggtgtagcttcaaattccattacaagtacctccttgttttttatacttaaagtatatcatgtaagataaaaagaagcaagcagtttttacactacttgctatattttttaaacaaattcttctcttacttctccccaactttggtctgggtctggtaagctaaaaggtagtttctcctgttcttccttagataagccctgataacatttctcatgtactgcaatggttccagaactaccctgagcaacgggtaacgcactaatcaattcccccatagcttccctaaactttttatagtcttcgtcatcagcgttatcgactaggacatacacatcgcattctgctttacgtgcatttgcaatgttttcctcactaggagcatcatagaaattttttctttgtaattcaagatacttaactgctttaattagtcttttagcatagtttttagctatcgggtcaacatcaataccattttgtaaatattctactaattgatacttcgcatcccctgatgagataccgaatagagataagcaacttgtagataaaatatctccacttggaagtatcttgttagaaccaaaatggtgagaatccgccctaccaaactctgaatcacatatttcagaatgactattggggtttggagtaccatccactttacggtcttcccctctatagtccttatcttccataacaccaccatactcgttgtagtgtcctacagtttctccaattaactttccctgtctgacatgttttaaccgacaaagctccccaccgttaaagcagtcacccacaatctgtgtattacatactggacaaatataaccaaaacatcccattagttctctcctcctttttcacttgggtctacaaacggaatatctattgcaccaaccgcttcatagtaagggacaacccgatatagcgttcccggacgatgcttactagaatttttcatagcaccttctgctctaccttttgtcgtatgcaccccttttacaataatttcaccggtaactctgtgtttcatttctaatacatataatggcatcttacattccccctacaagttggataccttcgtttttaaaggatacttctctagctttttctatttgttctagtatacgcacagcttgtgaagcaccaccaaagccattgttgatattcacaacagaaataccggaagtgcaactagttagtgcagtatttaacgctgtgtaaccatctgctgaaataccgtaacctgttgaagttggtacagcaataatgggtaaatcaattaaacctgcaagtactacaggtagtgctccctcaaagccttgtacagatactaccgcattactctttgtaatagcatctgtagaagccattagttttttcaagtcatttacaccaatatcatgaaacgacacaggaacatatccatttaaaagtacggttaagtaagcttcgtctacagtgtctctatcagctacacccgcagatgtaattgccacagataggtcactctctgtaggatttgcacgataaaacgctgtctgactagacttacagtatacgaaagcgctaccctctccccaactatctacccaatctggttcttctaactcctctgccttgtttataacagcaccccaagataatacttgtgttgtgccaaagggtagcccattaaatacatcaaaaggtaactttgtcaccataatatttgtatctgcgggtatctctgcaatctgactggctaattcctgtgcagttttatttccaccaaatagtgttcgtaatttcattatttaatctcctctaaaaatttagttttgtctactggttctatcgttccatcttcctcggttccttgaactttaaacaacaagaactctggcatctcacagacaacagcagggtcatatgctgtttccgggtcacacgccatctcaaacatgttagtatctttgttatagaaaaaacataagtccccaattctttcatctgggtataaaaagtcagcaatgtaaaacgccatatatttcatactagtttcctcctgttattttattatatatcttagtaagaaaaccaaccttctccggttctggctctggcggaaaccatgttattgttagacccctgtctcttcctgtcactctgctaccgccacaaacataacgtacagactcttctgaccaaatagttactttataacctttggctgttagtacctttttaatagctgtaagctcctgctcagttaactcattaccagaaattctgcaagagtacccaccaagttctattgttttattgatactaggtagtactttattattaatgaagtctgcggatttccgagctactttctctttgatttcttgctccttatcagctacttttttacgagcctcctctgctgacatcactatgcaccaactccaatttcttcatctagctttaaggatttaatcttattactaaacatgttagcctgctcgttatagtgtaaagctaataacatcttaatgttattttcaaactcagatgcttttaaatctacatatgcttcccgcactagtgagtacattgtgatgttatctttacaaaatgcccaaagaggtgtatccaactcactttttagcacacgagttttaaacagatagtctggctctttactacggttctcaatctgcttttctgtaattttcaagtcttcttctcggattttcattacacgagtggtacatgtaaagtctccttcgattacttccgatggtctaacatctactcgtacaccaactaaatataaagaatcctctcttgaaatatatacaggttctatgtccacaatcttagtgaatgggcactcgtaaccattcttagcaaaccaatctgtaattaaggcttgtaactcatcttgcttatcttctggaatttcatctgttaggtagtagtcctctgcaacttctccaacttcttcatagaccatctcttgtgtacgttccaagaatgaatcaacatctacagggagataaaccggagccactcttccaactacaaaactgttgattggttcttctggtagaaatcctaaaatatcctctacgtgttcccagatttcctctacaaaagttttcccatttctaatggcatctacggctagtttacctgcatgaatagcctcttccttggtatcgaaataagcacaagctctccaaacttccacatctgtatcacttgcacacatccattttcctactaaagattcattactcattttttctttcctcccattttatccttatctagaccccatatatgtgcccatttacgacaaaagtgtttatcataatctaagtccataccggtagcaccttcgaagtctttaaactcttcataaaattccgattctttaaactcatcccactttttcaattcaaccatcctatacataagttggtcaaattgatagtctggtattttaattgcatctaattcataatacagataactgtgtagtagtaaagcacgctctgtcatatttataaaggacaggttatctagtatagtattatttacaacggtcacttgttactcccccggaattgaaattattacttctatatctaacacagccataatcttaaacaaggacggtaagctaattacagttaacccgttttcatatgtagaaatggctttttgtgtaatgcctgcctttgtagctaatgcacgttgagatagaccttgttcttctcgtgcactatgaatatatcctcgtaatgtatcagataaggcacgtgtatattcttctgtgtcatctttattaacacccagttcagcaactgtttcggttaagttgatttccatgttttctcccccttgcctttctatactcatagtatatcagatacctttcagtacgtcaacaactttaaataaaaaaatcctagtatttactaggattttttctctctttttattgcgtctgtgagcatctcactaaaacaggttcccccattatagaaggtaaacaaaaaattatacaggtcgggttcaccctccacaggagtaagtaaagacacttcatcattatctaagatagtttgttcgaccgcatgataaaggtcatcccctactttgacatagctatcgtaattctcttctaaaaaccactccatgtcactatcatagtaactaggtcgttctcctctactttgtgtaaacaagctaactgcttgatactgttctgcttctgtaagcttacgtaccttgcctacacgtgtttccatttctgacattatattcgctccttaaatatggtctctgtctttaaacgtttcgtaagaatagtgcacctgtgttctaacataaaagaccttatcacaatttggacaagttgtttcccaaattgccccttcctccatattctctgaactatagattccagaatgatacaggtcttcctcgtctttcatagtttcttcacagaatgggcaagtgggatatatttccttctcttctataatgtatcctttatacataataactccccctacttaaacgcttcgtctaaatcaagttcacgttttgcgatttcagtgatttcttcaatggcttcttggtattctcggatagttgtaatctctttaccatttcctcctaagacccaatgacgttgaatcatgtgtaaagcttttgtaaggttcgtttgataaccgacaagcgcaacattctcgtttccttttttgtcgtaagtgatttctcctttgtcgttacgaactacacgttttacagttacgttgtctgcatcgctagtaatgcggtagtctaatactttcatgtttaacattatttgtctcttcctttcattcgttggtttcctgtacgaggtttaaatttacgtggtaaaacacgagtattgcgtttagcttcattgtatttacgtttaagccgaacctttctgtctgactgatgttcaaacatgtctgcaagagcggagcagtaatcaatctctgcacgtactttttctcccacatatctttctaaatagaatacatcaagagctactttactatagtcaatatcatatcgactaatagtaacaatgcgctcaactgtgtatctatcacaaatagagaatcgtaagtaaattattaactggtcaaatcttaagtcataatagagtggtttaagatagtccaacttaatacggttatctatttcgtataggtgtttttttgtatagtctaacagagcaataattcgattgtctgtctcattcatttctaacgtcataccttcgtcctttccggtgtgcattgatagagtctacaggtacatgtcttagctgaaagcttggcgtattagactcggataaaagccccttacctagacctgtccgctaacagcggaggatattatctaggtaagggctaaggataaacacaccctatacataccgttaatgtgacacgcccggttcgttactgtcttaaaataaggtctgttggtagtactgagtagcgtttttcatccactggatttaacccatctttgccgagaactgccactacaagtttgtcttcatcgtatcctgtaactaccaagttagcccccattagttgttcatttcttgcataaagtcctgccggtgaatagagaacccaatctccaatgttaaatactcctttaaggtacttaccgcacatcggacagtacttaataaatgttctagcctctgcggtcattgtcgtacgaacgttcccgtaactatgaaagctaaactgtgcacgcaatacattatggtctacacctataccaataccgcctgattcgtcttctaaattataaagtaatgctccaccctcacaccaattacacatgttgctcctccttcgctttttggatttcactttgtaaaaaattaatgtaccttggattttctatattaaacatatcttgaacatatacggctcctagttctctacgatacgcatagtccacaccaatacgtctatagaactcatggttaacatgatacataaactgatggaatcttgtatgctgtcctcttaaatcatacagctcctgtaataggtcacagatttgtacaaattcttcctttgttccacttgccatgatttatctcctccttacctgttacctaaagtatatcaaaacccagagcatctgtcaacactctgggtaacttttttattgtacagataataaggaagctacgttaatgtaagtaatcaaacctttttctacgtcctgtacaatcgggtactctgctttgtcttcatacatgatgttatctcgtacatataacatctctgattcaacataacggaagatagcatctgaactacctgttgttgctaatcgtatccgtttcttatccgtagggtttgctatgactgcctgatttttacttaacttacataacaacttaaacattcttgtaatgttttcatcttctaaagccggagaagatgttgttttcttcttcaatcgttgttccatataaattggaattgcttgtgcagataacttcatcgtagtatcactatttaagaagtactctgtagtagttaaaacaaccttcgattgttctagaaacttaagctgtaaatagcatcgcaaatcatcttctgagaatgatttttggtattttgaatggtctgcatttatccattctacgatgctacttaagaaggttacaggtctcttaccagaataagaaatactaatatagtcagatgtatttactatctcttttgcgcaagagatttcatatatggtagccccctctaggtcactgtcttcaccgataaaaatagtagttaagaagctacttgggacaaatcctacatggataaattcttgggtttctggtagatgtgactcaataaaactacctaaaagttctttccataagaactgctcgtccttgactagtgcttccttcttttgaagtttatagaacagaaggtcaaacgcagtataccaatcttcggaaatctttagtttagttaattcttcatctaccggaacatcaatagtaacagacgtgtcttcctcaacttccgtactatctaaatcattatcccataaatcaaatgttttacggagacaaaactctctccataatgtatcactcgggtcttctccatattgcaataacatctgctttccaattgaagttagtgtaaccttacctcgactattgataccttcgttataatcttgaatacatcctttataccgcaaagaaagtaacatagtgttcgttgctttatcttcaataagttttaatatttgtttatagccgtactccatatagaaaacagggaagttcttttgctcaacaaaagcttcgtgctccacctctaacccacgtttttgtcttcctagtttatcaataatacggagcgcttctaattctcgttcctcaatagttacaacatactgactaattttgttttcgtaagctaaagcggtgttcatctcttgcaaaatctgtttaaataaatcgtttgcatcttttgccttctgagtcttaaatgttctgattgtcgtatcatagaaacgttcgtgactcgaaatagcaaactgcccaatgtatcgtttaccttttaaatttaaacggttttcgttacgatttcctagggtaaacgttacgtagacagactcactagacttagaaaaatagatgtcttgaacaactatgccgatagtttcaagctcgtttatacgtttagtaaaatcactctcatattgtggtctgtttatatttttgcagtgctttctatagtgtactgcaacagttttcgattcttttgccacatccaattacctctcttatctctcttcttttgcaacatatgcttctaatacaatagttctaatcggtgcttcttcatgtacttcatcttttaccttcttaattacaccttctcttagcagtccttctcctaacttatgcgaagcttctagcattcgtaagtttacaagttcatcactgtcaaatacactcacatctaagcttactctgtaaggtaatacttgttggtatacacgagtgactgttaggggttcccctataagaggactcttcccaactcgagaaaatgcccagttatccaactttttattaaaccgcttctttaatctgtctaacatgttatcacctccatgtaaacagtatacatgttaagccttacaaaagtcaacaataaaataaaaaaacatacaattaagtatgttttttcggtacaaaatatttctcttggatattaccgtctttatctacaacaataccgtttaactgtccgccataggcgcaagcaccatcaatgtatattgtacggtctcctaaataaatatcacagttattgcctttatgaaagttaactgtaggtgtatgtcctacaataatagttttacttgtctcgttcttttggaatgggtttggtctagtccaaagcatttcgtgttcctctgctgttttccagtctggttgtctccatcttggaatacccccatgcacaaatagcacgttaccgaactcgtagtaaggttctaagagttccaaggtgtcttttacattagagttatcaagaagctctgtacgcaacgctagggcgctaagagaagctgtcttagttcctagaatagaattagcagtctcttgacctccttgataataccacactgaatatatctcttgcaagtcctcataatctggttctaatcttaatgggaaactaaggaaatctaatagcatttggtcatggttccctttaattgctgtgccaccagaagccatatgttcgtaaacaaaacctagaacttcttttgattggagacctctgtctacataatctcctaaaagaaccaattcctgttcttcacgattccaattttttaataactccataaattcatcgtacataccatgtatatctgatactacgaacgctttctctttgctcattaatcgtcctctactttctcaaaatatcgaaagaacgcataccaatcatatcgttcagtaacctgcttactctcgtgcataaatacaaactcaacgtatttgtgtttaactgcatgagggtctattggttcccaaggaagcctgcgaggataatcatagtaaactcttgttgggatgacttctcgcattaacaatttagagtaccgctttttaggacggtacctctttcctaaaacatgttcgtatgtcggtctcattctattgtaccatgtttcgtataagttaagccggactcgttaaaatgtttttccttctcttcaatcaggtcaatacattccttaattgtgtatggtgtgccaaatggatgtaaagttaattccggtgagtcaatacctacatttagtaaattaggttcttgataagccatctcatgaatatgtccatgtaagtttactcgatttcctcgactaccaataatgagtggatagtgtgttaaatggatgatttttctttcctgctttagtattacgccaacatcactccaaaatattcgttcacgtagttcctcatcccgattaattgtttttaaaagcttggtactgtcgtggtttcctttaaccatgataatgataccgtttagcttacgaagaatctctagagtatcctttggtttctggttcatagcaaagtcacctagatggtacacagtgtcttccggacgaactcgtttattccatgtcttgattaagtaatcattcatttcgtccacatcagcaaacctacgccttgtctctacaaagccatcttctccacaaatattgtgatggttgaagtgcatgtctgaaattacgtatgttttcattctagcaaccctcccaattcaaagttatcgaaacactctgcattgtatacattttgaatggcattccgatagaactctacagtctctaggctaactggggtgaagttgtgtacatctactcctacgttaagtccgtcacgtttcaccttttgtttttcgtgaatatgcccaaataagccaaaatagccatctttgattttatcttctgagtaactcatatgggaaggctcatggacaagaactaatttttggtctacaagagtgacaatttctccttttcgaacaaattcaaagtatttttcttgttccggagtaataaacccacctttaccatcccgttcatagttgccaaataataaatgaattttacctttaagctgtttagctacttcgaagttaccgaaatctcctaaatggtaaacagtgtcttcttcatctacaacatcgttccaccgttctatcatagctaaatcagcatgttctacattatcaaaatacatacctcgcatagaatatttgaaagtacgttcttgtccaaagtgtgtgtcggacgtaaaccaaatcttactcatttacatcatctccaaacgttcataacattttttaattaccaaggcatcccacattgcattatgctttacaccctccattgtttcaccattaccaagaaggaacgcaaactcttcacgagaaatatcagggtcaacgcctttaaccttgaataacgtgcaaatgtcaaagggaatatagtacacgttactgggtatagagagcgcatccccaaatagatggttgaacaacacccaatcatatgaaagacaatctgaccatacttctacagaattaaactgcgctagccattcctgtagtttttgtgcgatatatgctgtgtctccatagtactgattttcttcttctcgtaggttttcttcccctactaaattatcaagcacatgcgcctgtaaccattcgtctacttgtgtttcatcgtaatcgtttaactctgcatagaatgatacaccattttcagctataattccaatactaattagtgtagtgtctttatgcaatcctgtaaactctgtgtcaaaaaaaattttcatgtcttactcctctatgcaatttaattatgattatgttgtgactattaatagaactaacaattgaagataatgaactgtttggtcaattgttagttgtgttttggggtctggtactatcgaaggaaaccatgtactttgtgctttgaggtagtccattacaaaatgtggtaaccatagaacaagtaaaatgtgtgctaatgtaatagacaaaccaattaggtagcctgtcatagcaattgtaaaagtccaaatagttgcatggataaataacacatataagtcttttgttttttctcttgcaatatattcactttgtagtgcgtaatctcctacagcatgtgcccctactaacataacaaacatagtaatatatggtgtcattatattctaccaccctcatctaatagtttctctaacggaatgaactctgttgtccgtctaagaacaatatctcccgattcctcaattgtatcaagccaacgtttactaccatcactcattcgaagcagtcttaaagggtatcccatttcatcatattgtatgatatttgtcctttcagatacgtaaggctctggcttcttttttataaagttaaaccagttcattcctgcacctcttcataagtgtctgcaaagatgtctggtttacatggataaacttcaccttgaacgccttgaatgatgtagtctcccacagaaacgtgcatccaaccttcaagtgttttaatataaagttccccttcttcaaaactataatctacatttaatatccattctgggacatagtaatttcctgtggaatccataaaatctccatcaaatttaaatgcctcaattactactggtttctttctaaatttcattttaactcctcctctattctgcaatcatgggtgttccatctgggtttaatcttacagtcataccgccagctctatccccatgaaaaactaaatattgcacttttgtttccgggtctttatataccgtcatatccctagagtctgcattatcaattttagcagtatccgcattttcaacagaagagacatcacatgcccctaaaaataacaacggtaatagtaacaacaattttttcactttacttcctcccatatttctgtaatactttcttagattttgtcattaatagcaccttcaatgtattttaactcccgcataagttcaagaaaagaatcggtatctaaaataatagacatgtcatcatttctgtcttgtatagtaatttctacaactgtatcctcttcatctagactacggcggtcttcttcataaactaccttaacaccactacgggtgccatcatcgctatataatgcgtaatactcttttgtacttatttccatttcaattatttcctttcttttgttggtcttgttccattaccttgtacatttgttcctctatacttaatagcacagtcagcgtgctgtagtctccaataaccattctttctttcgaaatgtccttgacctttctctacaaaggtaccgcacctgtagcaataacccggatacttgttgcgcatagtagtctcccctttacacaagttctaacgaaatacttagtataccagaatatttatcttttacagcttcttctacttggttaactgcttcttccagtgaaatagttgtattcgtatcctcggaataagtcttagctaaaatccgtattgcatgaacttgggagacctcttttagttcaacagaggtttcatcttcatcatccacgtaagcttgctctttgtatatctctaaagctttgctaggactataagctgatataagagcaaaataaggactagttacttcatagaacttaattcctgttaagttaaagaaaggtgttacgtagtccattacgccttttttcatacgtagttcatttgaatgttttaaattatcgtagtgctctttttctactacaacaacctctactactttaccattttctacttcatttagggtacgtagtctatctctagctccgagataactacacattagtgcatctttaactatcgttccagaagcagtattataccctgttttataaaagccatcttctcttactgcaacatataattttaacggctcactcatcttaattttcctcctctgtaatagtatcatagaaaatcgtcccaaccgactcccacgctacgcataaaacaagacttattacatagtaagtatcttttatgatactcatttttaaaactcagagtgctcccgcactctgaacaatataacttcacttttttatctgggtatccatcgttgagtatctcctgcttaaatctcttaattccttgtcgtctattcatctataacttcctttccactggcacagcaaatgcccataaatctgcattaatttcttttatctcccgctctgtaaaactgttcttccaagttctacaatgcttattatttttaaccacggaaaggtgagtataagtctccccggacattgttttattttgaataagataatatgtttcattaacaaccgttaacttaacatgatacaacggttcttcaaccttataaccaagaatacaagccctctcaaaatcataccgacagtatgctatttcaaagtatttacccaacttttctaattctaaagtatcaggtaaatttactgtatcaggtgtgttattctgaaattcttggattgcatcactgggtgcccaattacgtttcgttacccaatcaacaaactccgccacaaaatttggtataacgggtttgtctacttcctctatctttctcattattgtaattccggcatttataccctcgttatagcaaaagtctcttatatcagttcccgacattctatggctttccaattccttaatagcttcttgtttatccataactactcctcctctaaattttctctcttggctaacaaccgaaaattttcataaaagtaatctaaagtttcgtcaccgatagctccatcatcttcatcagtataaaattcatctttgattccgttaactaaaagtaccccgtcatcatctaccccttcaacaaagtatacctttgcttcatgtttacctttacgaataactaaatcaccaacattcacttcccatttaggtatcggctctacctcatctggtaatttaattattgtaaaatgcgttttgtactctgggaagccatcgtctaacggggtaccagaatatggtgcttcactaataggaaactcccaccaaagaacatctcccatgtcttcatgccatttatctgcttggtatgctataaccttctgaggttcgattgataaccaagctcccataaactcttctgtattgttagcaatccactttagtgctactgtttcttgtagacttccggcaaaaggttgggacttagttttataactaactagaatttccattatagtatcaaagtctgtgtctgggctttctttccaataagtgagcaagtcttttacttcttctggcaaggagtaacttattataacctgctgtttttttaacgaacgaatagcattttgtaagtaagcttcttcgacatctcgacttttattaccagttaaaacccgctcaatctcatcaattgcatgttcaatgtgattcattattattattcctcccttaaatccccaaattttctgtctaattcaacaccaaattcaaattcatcagctttctttgataatttgcacatatcttcataatttttcttgaagttcttatgaataatcggagagcaataacctgtctctatccgtgctaatgaatacttgaccagtctagcaattttatactctaatgtttggtaattaacaataatatttaatttattttttgggttgtaccattcatgtgtatatggaggcattgtaaccctattctcaaaccccaaacctgtcaatagctttttgactgcttctgtattaacttctaatgtgtccatgtctacctccaccagttaggttctttaataacttgccacgctctttttagttgttcttcgttgaaatgtccaaaatgacagtcctctttaggaataccaagcttacaagctaacacactgtacagctctcccctacttacctgtttggatttccatacagggtcaaataaactgtgacataattttttccatcttttcataggtggtgtagctaaaatacctagcggttcttttggtctattcttatgagtccctacacttgcaccacaacctgtacaataataacatttaccattaccgtaaactttcccatataatacttcattacttgtccaaataactttagctccacagtaagggcaattatctggtactaacattttatccatgatttgttcacctctcatgtatacagtatacccctatggtatcagtaagtcaacacttttttacaaaaaaaagaacagcatttctgctgtcctaaaaatcacaccaatcatctttatcataagaatcatctgtgtagtgttctttacctttaatgttcttaacattaacatgctcgtcttcctctggttgctcttctttgtttttcttgacgaaataataaccaatagagaaagctatacccccaagcaacagtataataagagcgataaagaatttagcaatgaagaaccccattagtgcatcactccatgttcattgtaatagtcaatagcctcacatctagcttgataagcttcatctaacgtgttgtacgtacctaaatagtaaaccttaccatctcgctttaacctagctgaatagtggtcacctcgtttgtatataccaacatgtttacggtgccttctgtttaaagcttgtactttccagtcagtccatcggcagttatccggagaataatctccgttcgtatcaattctatctagagtaagcccttcttcgtaaccagaagacaaagcccagtctaggaacggtgaaaaggtagcccatactggcgctaccttaattccccgtccaccatagttatggtagtcacttgacttaggattggaagtcctataatggataccttgccatactttataaagtttcttatgtccaaaacttttattcttaagacatccacagctcttgacttcaccagaagtgagcttatcttgcggtttgactacagtgtttccacattcgcatagacatttccagtagctacgtctagctaagtgtgaaaattcaatcactgttagctttcctgtagtaaaccctatgagattttttactcctttagggagtttattttccgtcatcgctaccgtcaaccattacggagcttttatctcctacaatagtagtgacccaaccgttttccttacgagcttgtgcttcttcgtaacgaactaattcatccgtaacagattctgcaattttcttgttagcttctgcttgtgcattagctttgtctctaatagcttgggcttctgcttgggctttcaaagtaacttttgtagcctcagtttctgcttgtgttttttgagttttagcatctaactcagcttttttagcttcttgtcctgaacggatgattgcatcaattgatttttgtgtttctgggtcaacgtcaggtacacctaccgttacgttttcaactaagaaacctttaccttcaactgaatcagagaaacgagctagcacttctccctcaacctcagaagacttacctgaaagaacatctagcaagctgtacttagagtaaacttcacgagcagtcttttgtagacgagatttcaaccaacctttttcaatgtcctcagatgttacgttaccgaactctttgtacattttagttgcttgtttagggtctactttgtagtcatacttaatgttaactacagttttcttaccatcagatgtagaaaccgcaacatctttagcttggatagtttgtaaacggattgggtattgtgttactttatcaatacctacccatttaacgccgggctgtaatgcttcggatttaacaccaccgttaggtgagaaacgtacacctacataaccgttgtcgattcgttcgaagaaacggaaagcaccgattgtacctccaatcaataaaataactgcaaccactccggctacgactaggcgtttaaacgtcttctcggtcataaaaataattcctccaatatttagttttttatgtgtaagttttccccttacaaatagtagtataccttacactgtataacaagtcaacaactttttttattttcttgtagaaaataatttaatgggtgtatggtcttctccttcaagatacttaccatcttcccacaagaccactgagcctttctctaggcttctctgcacatcaatgatacgttgattccatgaaccacggaaaacaaggttagggttgaatttatcctgctcaaaacgaccatctactagtacatctacgtaagataataattcaagcttatcttcccagttttcagcgttagaatctgtaagctcttcaaatgtgtaacctgaccatgaccagattgttttacctgttagctccgatttaatacgtttagctaaaggcaataagacttgtgtatttaagaaaggttctccacctaacaaagtaataccatcacaataatcttgtgaaatatctttaaggatttggtcttctacttcttgggtgtaatctttaccccaactaaagttttgcgcctttttattgtaacatcctaaacaattaaacggacaaccagagacgtagatgcttccacgtaccccgataccgtctaccattacaggaaacttgtaatcagcatatttacccttagacaatctttctgctgtccatgtcattgggtctttactcatctgtatttccttctttcttaacatgtaagccttcgtcataccctgattgggtaaacttagcttggcaaattgcacacttataggctttgtaccctagattattatacggaaccactttgtcttccccatcttcatcttggtgggtacacgctttttgtagcgcatttagtatttcgtttcgttgcttgattaatcgttctttctttgcaaggtgtctacactcttcatgtagaattgcagactctgcaatggctaaaagattttgtaaagaggaagctgtactcattaaattgccacctcgtaattaaatcttccgttatgtttgtagccttcaaccttaagttcagatagtgggcactcaaagaagttagtcatgttagcctcttcccaaggcaatactagcttaggttctgcgtttagaacttcctctgtcataggagcagttacttgttcgacagcttgtaccatatgacggtcgtagatatgtgcattgtcgatattccagaacattgtacccaactctaagccagataccttagacataactacatgtaacgcatgatactgcaatacgttaaatggcagtcctaagcacacatctgcactacgctgtttaacgtgtaagttcaacttaccaccctgtactgtccagtgagtagcccatacacaaggttctaatgccatttcatctaagtcttccacatcccatagagttgtcataattctacgagaacttcgattgtattttagttgatatagaatagtttctacctgattaaacttaacgtagcacttatagtcttttgtttcagggttaacatcaaccataatatcctcttcgggaaaaaagtcttccggagaagctacataaaattcattattcgtgtctagttgcactttacgttttttattgcgtaactgccaaccataagcttttccgattgtaccatcttcttgttcccattcgtcccaaatagttactccacgttcttgtaaccacttgacattgttagacattgcttgccaaatccactctaattctgtaaatgcccacttagagcctacaaacttactcttaagaattggcagtcccatgtctgatgtaatttcaaagcttacaccttcaattgcatatgcaggagcaggttccccatcttcatagtgtgctcgtacttcatcgggattttgcaaaatacccttttctttgatggctaacacaaggtcagcataaattttgtcaaaactcatgtgtgttctccttttctatgttcgtctaactatattgatagagtctgtatacccatttcttcgtacccactctgtataactgttgtgctcttcttgcgcaatcgcttttaaagaaccataccacttcttgacttcttcccgttttttacgtttgaatactaatgatttgtttttatctatgacgttgataatcttacttactccaccagtattatctataagcattaatcgtgaaacggtcggttttggaggaaataactttttgcatcccttacacaactcctccggctcttttttcctacccagtgcactcaaatctggtagcgttctaccacaacgtatacactcagttccataagcctgctcaaaatattgtgcattaatacgaggaaacttttgcgtaagtcttagattagttgagggtgttttaactctttttcttagtactgttttgtttttcttaaccttaacactaactgggtcattgccatcaaaattaaaagaaaaactaattgttccagaagggttaattaccggagtaactacattgcgttcttctcggtctgctaatacccgaacatctgttatacgaggacgggttttctttgtgaataatcgtcctcgtgtgtaatctataccttgcatattacaaacctagccatgaacgtactttgctgaaaggaggtttcttttcaggctccttgtactcagagtttgtacgttttaatttatcgttgtagttatctgcaagtgttaacaattcatcaggatttactgtttcacaataaaaagcaatctcttccccagttagctctctattttcttcaatcaatttatcaagagtcaaatcgcaatactcatcttcccagtcagctaagaagtacatacgttccataacataatctgatgtttcatctaggaatacaccaaatagaattgggtctttgtctcgattatcttttgctacttgttgacctacctcatctgtatagtctgtaaacatgataactaagcggtcaaatttatcttgtacacgttcaattacaggaacaacttcatctggaatttcacgaggaaaatctttaaggtcagctagtttaactgaacgattttccacattttcaataaagttttcaatatcttctgaatacacaaaagttgtaatgcccatatctctaatttctttttctaattggattgttctaatatgatggcgtaacaaagaaatctgtctatcttgctttgttttaattgccttttctaacatacgtaatgcgtttgcgtaataacggtctaggtcggcatcttgattttcctttttattttgttttactcgttcgaagaactctgtaggtgatagttcttctaatggtttcgttgttaagttttcagtcatgatacatcctcctaaaaaaatattccttacaggaaataatatatcacctgtaaggaataaagtcaacactatattaaaaaattgttgtaagcatttgactgtggtctaacgctaatacagcatccccagtaacacgcccatcacggtatagaataattgtagtatcatcggttgagatgtctactttagcctcatgagaggtgatggtaaaccatacgtaatcactgtcagattcgtcttccttacgttcagcatctgaccgcatttgagctatatacatttgtgcaagattatgcccagaagggtctctattagggtctccaaacactgtagatggactaataccttcttcttgtccgtctaatgaaactgtttctaaaatactctcagcaagcatattaggggtatctgtggctcctaaaaaagcattaggtaattcatacacaccttcttgaactcggtgtagtaaaatagtagcacctacttctaattggttccttaatacaactaaagaaacgttaaatcctactttttcaatcatgttctttcctccatttttccatttcatttattttatcaggcaatgttcttcttgttaaacagccagtagtacttacatgaagcctttgaactccattattttttcctagaacttcgtctgctttcttatagagcatgtctgtatgttttggacaagctatctcattaaaagatgctccgttatctgctacacttacaatcccgtttgcacagaatcgactacgacaacctatacattcattcgctagcttcttcatgagcaacctcccactctgtggcttctcttaactgtagctgtctagcttcttctaaaggactgatataatcaggtagctcatctaccggtgtaaaacgttttgtcccgtcttcaagtatgccataaaaatggtctgccggacgtgcaaaaactgtgttagcatttggtgaataagggttaggatacattcttcgatacaatactaactgctccccagtctctgtatgaagtacatcatcatattctagtgtagcataataagcacccttataatgacgaaaccacgttccggctctatgcttatattccattcctcttccccctaaagaaaagctagcactaggctagctctttatttttacctttggttaatttaccactttcatcataccacgcataaatggcagttacaacatttgtttgatataaaacatctctaattgcaaaccagtagttaccagaaccgaaatctagtaagaaccagaagaagttacttgcaaaccatccccaccatgaaaagtcaattcgcataatcattaagaattgagatacgattcctagcaagtaagcaataatgttaccccaatacatgatgttacccattagggaaccatctgtagtaaagccaattttatctccgtacataattaatacaattaaacctacaattgtgaatgctaaagtaaatactacccaaaaggcattcatcttacctactttaatacgcccatcttcatcttgattggattcttttgtccactcttttaacccgataatgtgtgtaataccataataaatacctgcaaaagccaaaccgaagttcccaaacatagtattaataatgatttcaccaacgttagaactaatacctaagatatttcccattttaggataacggtctgctaaagataccacacaaggtacacctaagcagtacgctgtgatataaaataaccagttagttttatctgaccagtcggtgacaaacacccccacaaccagtcccagtactaccaaagtgattgcgtactgtttgcgccatttcccattagctagtgaaatattgtccataaatgttttcatattatttctcctccttgttagtccaaatttcgtcaacgtataattgcaacgtgtcaactgtagttccttctaggtacttttcgataattttcttgcaaagttcataacgagtttcataatcactcgcagttacttccacatatgggatatgattctccgcatataatttcttcaacaggttttggaagtcttgtctacgtttttcatcaccgattaaacgcataccatcgtctacccatttagtcgaattatctaacaaaataaccaaatcaaagcggtcattatcaatcttatcttgtacagatggatgaggttttccttcgtacgtaatacagaaggattgtgtggctacgtagtctgtatccgaaatagtaaatctattagcattacgtcttgcaaatcgaacgtcttgatagtgaccattagcaattgcttggtagtcactgtactgtaatgcactctcatctccacctaattcaagttcacagtagttgcgaccgtattcccaaacagtcgtagtagcaaagtagttacctagcttatctaccatcaaagttttaccactagactctcctcctgcaattacaattgtaggagccaatgcttcccggctagcacgaggtagatagtcccagtacttaatagggttgttacgtacttttgttgcactaatgttaacattagaacgctctgggtctactaacattgtaggacagccaaagtatttttggtaaccttccgcatcctgtggttcacttgaaaagaatgttgcttcttcccaatcaaaacctctttcagccaacacacgattacctgttacagtctctttcacaaggtttgcccatgcttcccatccgttagggtactctggaatgtttgattcatctacgagtacaggaatgataacatctcgttgaacttggaaagtcttttgaacaattcgtagcttgtcccgtgctgttagtggacgtttcatgttagaatccatgtataaacggtcatctctactgtctgagtggcttaccactacaacaagagtctctacttgtgtgcttgctttttgaatcatatctacatgtcctttgtgtaagggataaaatttcccaactactactccgaactttttcataccttattcctccttttaggtattttttactataattaattataccacactttactagttattttcaatataaaaatagccataaattaaaccaattgttagtgcaatccacccgataacaattaataccgtagaaaggattcctttagacaatttggactcaacaaatttatcatatgcagtataggaaacgttaatagcgctgtctttacctctgactgactgtaacccattttcactagcatccgcaagaaaagttaaatgtaacttcttaggtacgccataataggtaatacgtacatctgatgataagtacttagtagttatatacttgccatcgaacccagtaaacttgttaaagtcatatatatggtcgtgtactttaactttatcaacctgtttagtcttagagccatagtatacccattcccagtaagtttctgttttagtttgtgtattacctttctcgtctacataactaactgttctagtccgcatctcatagtgttcctcttctgcaacaactttcatgtattccccaccaatacgaggaaacgttactggttttattgctatgaaatcactatgcgtataaaagataccttgttgtgtatctaacacgtaatcatacgtttctttatcatctgccggaatagcagtttcgtacatttgattagattttatccggttctcttctacttttccatgtacccatattcctagtaaactagttaaagtaacaattagcataggaataatgatgaaccggattctaccatccacccgcatcatttgctaaacaggtctgtatgacctttaggttccacagggattgttaacaattcataatcttgtacttcgtatccttgcagtgcaagtaacttacgatttaagaaagttctagtgaatcgtttataatctgaaatttgtcggttataagctttacgatgactagcaactcgattctctgtaatactgaactccttcattgcttgcttataattgtcttgtgatttgagttctgggtatttttcgactactgcatttaaagtcgttttagctgaatctactttaccagtttctgcttgctgtcttgctttaacaataagttctaacgtactcttttcatagttattatagctctcaatagcatctactaagttttggaacaggtctactcgtctgttttcctcagtatttaaatccccataagcttgctcaatagatacttcgtaactgatagctctattttctacatgttttactccaaaaaatgcaacaatacctaataataaaacccctactagtgaaacaataattgtgtttgctttattcatgtgcttttcctcctaagaactgtttaagaatttctctccaattgacatattctgttgtatgtcctttgtcatcaatgtaaacatctgcaccaagcttacggctgttatttccatatgctttcatcaaatctggatgattctcattgatgtagtggtaattaataccacattctgccaaaaacctttttgcttgttctaactcttcgccttcacgacatgtccatataagaatttgttttcctgacttgtataggctgttaattacttcttttgcatgtggttgtagccctacaatatttggaaattcacttcttacaatagtgtcatcaaagtcgatagctatgtatttgcactttgataaatctttcatttgtttctcctttcaaataaaaaaggtatacccttctttgctacaagaagaagtataccttatgagtatcactaagtcaacactatcttacaaattatttgtaagaattttttatttctttaatttttcccatttgtacacctgtaaaactgatacctgtagacgtttctacgtaaggtaatgaggttacagacttactaactaagtaatctctagcggatggttcattatcaatattatgctctgtaaactcgattccttcacctttaagcatattttttacaaatgagcaaccaccgcacccattcttagaataaactgttactttaaccatgtgttacttcctcctgtttaaaccattttgaacctgtaaatgctacaacatcgttagctaaataaagcatgtaagttaacattaatacatacacagcgtgtccatgaagagctgtttgaatccatagtgctactgacattaaaccttggaatgtccagaagtaatactgtgcacggaaccctttaacacacaatactgctccaactaaaccaatcattgcacttgtagcatctaagaatgcttgtggggaatgcagaataactgtatccaacagatacaatgcaacccaaaatacaacaataagtaccaatgttttaagcatatgtgctcgattcatcttgcgaggtttcaaaccttcattccaacgtttagagaagatgattggaatatccagtaaaataatatatgctgattgcataataatatcactatagttgcctgttacacttgctactccaataagtagaattgcggaaataaacccaagtaaaccattaattgatttacccattgtaattgttacggtacaagtaaaaccaattattcctgcaatagtagcagttaaacctattacagtaataccttgtccttgtaataagggtagaataagtcccgtaaacacttgaataaccattcccacaatccataaaattcttgcaggtttagaccatccagttagtccgtctctataatattcatatgtgaataattttttaatatgttccatcttgctgttctttttagcttccattaatttgttactcctccaatgtgtattaaatatctttacgctctatatcccagaaatgtgctacaacatgataaggtacatcaattccagatgactttgcatgattatagaacttcataactgcttgttgattgctttcttcttggtataattctttgtatgcgttaacggcttgtccctcagatatacagcctaattcaaataatttatctataatttgacttacgatttgctgtctatgttctgggttgttaacaaaatcaatctctgtaacatcgagaataatttttggaaaacctaaatctctttccatccaattttgatagttctcatgatgacgtttgaagtactcgtataattcggtaccttcttctactttttcaaattcacgagcacgagcattgattcgtctgatttcttcttcaaaagaacagtttaaaacaaccattaaatctggtgttttcttaggaagtggttctaactccttaagcattctctctagtaaacgatggtagacaccttgttccatttcgttcacatgtccttcttgtactaattggtctaagaagatactatcttcaaatatagaccggtctaaaataccgttatccacactcattgcttcttgaatcatttcaaagcgtttggataacatatctatttgaaaaagtagtccgtattttgatttgtcttggtaaaacaattcaagcattggattctcttctacaggttcataaactgcttttgtatctaataggtttgctaatatttctgtcattgatgatttacctacaccaataactccggctaacgtaattatcataaccaactcctcgggatatttattttttgacaatattaagagcctgactagcaggctctaataaaccttacatatgttttacacgtgcatctatttctttcttacgtccatgtgcattaggacgagcttgtggttgacctaaataaccacattgtcttttaacaacatcacatgttgcagggtcattattaccacattgcgggcattcataaccatctgatgtagggttaaactctccatcaaaaccgcattcatgacatttatcaattggggtgtttgttccaagataaccaactttatcgtagctgtagtcccaaacagcttctaacgccttaaggttgtgaactagtttagggtattctacataatggataaaaccaccggaagcatattttggaaactctttttcaaaatcaatcttttcaaaaggtgttggagctttgcgaacatcatagtggaagctatttgtataatattctttatctgtgatattctcaataactccgtatttttctgtatccatgcgacagaatctatcagttaaactttcacttggtgttgagtagatagagaaccagattcctgtttcttttgtccattcatcctttttatcataaagcttacgtagaacgtctaaagcaaactcttttgcctcttcatgatgtgggtctgttttatcttgccagtcaccaccatagaacatgctagcaacctcgtacagcccaatatagcccatagaaaccgttgcacggttatttatgaacaattgttttacatcgtcatcatcggttaaattagccccaaaagcaccatgtttgtacaagataggagcattctttggttgagcttcgtaagtacgttcaattcgataaaccaaagcatcatgtaataccttagctcgttcttcaaaaatttcccagaagacttctttatccccttctgattcaatagcaatacgtggtaagttaagggttactactcctaaattcatacgaccagagtttacttcgtttccatgttcatctttccatccttgtaagaatgaacgacaacccataggacatttaaatgaacctgttagctcaacaattttgtcatagttcaaaatatcagggtacatacgtttagctgaacattctaatgctaactgttttacttcatagttagggtcttctggtttacggttaactccgtctttaatagtgaacaataatttagggaagattgctgtacgtccttctgttcctagaccttggatacgtactttaaaaatagctttttggatttctctttcccaacgtccagtacctaaaccaaaaccaatagacgtgaaaggggtctgaccattcgatgtaaataacgtattgatttcatactctaggctttgcattgcatcgtaaatatctttgtttgttttctcgattgcatatgtttctggttcgggaatacggtaacgtttagcatccgccatatgtttttgataattcatttcggcataaggtgctaatacttcatcaatgcggtcaaatgaacaacccccatactgactggaagccacattcgcaataatttgtgctgtttgtgctgtagctgtttgaatggaatgaggtgaaccgatttctgcattacccattttaaacccgttcttcaacatatattcaaagtcaatcaaacaacaatttgtcattggacgataaggttggtagtctaaatcgtgataatgaatatctccacgtaaatgtgcgttagctacatgaggagggagcattttaagaccctcaactttgttaactgtacctgctgtaaggtctcgttctgttgcaaaggtatttgagtccttatttgcattctcattgacaatagttttgtccttttcttgtagcttacgtagccttacattaatgtctgtatcacctgcacgttcagcatcacgtaaagaacgaaactcaatatagtttctagctacgtttttagcaaaagatttcattaattcctgctctacaatactttggagttttgcaatatcgacacgtcccgtatcgctgtaatgcttcactacaaagcgtttaacacgttcagctataatctctcctaaatctgttttagcgcccgtagcatccttctcagctcgtcctacggcacgtacaatccttccttcattaaacttgacagttcttccgtctcgtttaactacaaaaatctgttttgtcttaacatctacatcttgtgttgcttgcactcaaaattccgcctttcttacaatatctagtatacttagtatagcataacaatacttacagtacaagatgtaattgttaacaaaaaataaaagactacaaactaagttgtagtctttttcaacaaatattacaaattgattaattttttctcagattagctcacctaagaaatcttcaccatcatctggtttcttgtgcttatactcatccgttttgagcattaggtacttgttacctgtttctacttgtaacctctcaaaagctctacggtactcctcagcactagatgtagcatctagtaggaacttagcattaaggatagctaaaaccgctagtagctcctctcgactaccagacatctgtaccttaactttaccatcctcagtcgtataagctccgattttattcttgattgccacagttacacctcttaatcttttaatttaattttaggcatagcttcaagaagtttattattgaactttccagaaataacagcttccccaagttcgtttgttaccacggaaatccagtaaggagacttcggtttcaaacgattgttgttaacgtctaagaatgctctaaacaatcgagggaaagaggttactcctactaatttactagcaaggtcatgcgcaataactttcttagtgaaacgctctgcattgaatgagtctagcgtatacaacaagaaataaactccctgcaactgtgtgtttgacctaaaatctgttgaatcaataaacttttgatatgactgagcaaacttgacatattctttatagttacggaaacggaacgcaccatcacgtacaacatctgtttttcttcctgctgtactactataacctaacgcagtagaaatagtagaggacacccgtaatgccggatacgtagacatcaagttcaacagttgcttataactttggtatcctgattcggcatacttatggatgtagtcttcgaccttccatgatttacttgtactattcaattctgcaacatcaatgtcactgtcatcgaacgtataagtaatgggtaacccaagttcacgagctacagctaaacggtgttgtccgtcaataacttccatctttctatttactttaatctcagtaagaaccccacgtttcgcaattgagtcccgtaaagccttagaaattttaactggtctgtttccttttagttgtttgaatttagacaggtctgctgtttcatagacctgttttactgctttatcttgcttcttttctttagacacctgtcatctctcccttaattattttttgtattgaatgctttgacttacctcagtgattttcttaacctttttaaatacattaggataacatttttccaaggtatttcggtctacaccaatgcgttcataggcgctaataagtttgaatgttccgtattgagattcaacttttgttttcttaagttgaatcatttgttcaagaatttcttttctaagttcgtctctttgctcgtttaaagaatcaagcaactcctgaacacgtttatattccttaaagttgcgctctaattctttttcttttacttctgtagttgttagtctcatgaccatctctccctttctttactatactcaaagtatatcattacatattataaaagtcaacacctatcttacaaaaaattgccaaaaaaatatttggcaatcagtttttgtcatcaggttctaatagcttgtccgcaggcatatcaaaaatccgagctagtttagcttgattatttgctctgggttttttgattccatgctcccaataggatacggttgaccaatgtacacctagttttttagctacagaacgcaaggattcatgcttttcttcacgaacacgtttcagcaccaagggttttggcttgctttccacgattctcccctccattctatatataactacagccctaggaaaacctagggcaagtagattatttaagtgcttcttttacaaagtcatattctggtttatttaacacagacaacatcatttcttctggggaagtaatttctccacctaacacttgtgttaagatgtttgtggagaacccagatactaaagctgttcctttttcgtcaaaacgtacaggtgtattatcatgacgagagtttacattccagaatactacagatggtagctcgtaacctgcctgttcgaacttaagttttgcttgttcatagttagtgtaacctgccacaccttggtcaaattccatatcggaaataataacgatttttgaaggtaattcattttgtggtgtgtggtttttcaccgcagtagataaaatcaagtcgaatactttttgtaaatttgtatttctagcattgtatgaacatttatcataagccttcatcttatccataagtgtacctgacaatggtaagtttactaaacgaggttgttcagaaaatacgatgaaatgacctttgaatgcgccacttaagcgttccgcagtgtagatagctaagccttctgcaacttcccaaggggtaacattttctgtaccaccccatgtcatagaaccagaagtatccgcaactacaattgcattatcttcaatatctttcaacacatcgtccatagatttccatgttgcttccagtgaacggtctaatgttctgtagtaacttctgctaccataagcccgaattacttcatgaggtaacatattctttgcattcatttttacttccccagattctactttagaaagtaaatcttcatattcgtctggcatgtgtaattgcaaagctctacggtatttgaacaatgctttagaggatacttggttcaaatcaatcttatggtattccttgcttgctaaccgaacttcaattacatttaagtaatcacgtaatttagcaatcattttacgataacgagttagacctgctttaccttcaaaacctaagtagttagcaatacgggtaccttgattacgtttcttgctatttggagaacttacattagctaaccatttacctagtaatgatacaggcttaccttctgacatatttttaatatcagatgttagttggttcccgatgatacgtgcgccttctgtatccacaaagtgaatactacgtttgtgtgcatactccatagtttcaaataagtctgtccaacgaccaacatatgccatgtactccattaaagtaacgattacttgcttgttttggttgcgaccatccactaaatacttgattgcttctaggtaagcggaacgttcacccaatccacctaaacggatattacgtaagtgtaccaaattgcgtaatgctaactctgggttttcatcgtaagctttagcaaacatgtttcgtacatcttctaaacgaccacgcatagcccctgcttgtgcaaagaagtctaagttagcgtttaaagttgatgtataagttttagctccattttctgtagagccaacagttgtttcttcttttaggttgtctaaaaatgacatctgacatctctcctttgtatggtaatgttttatctaacaccactataacatgtaatattatgtaagtcaacactttttgtaaataaaaaactcatccgattaagaatgagtttgtatgtaatatcaagaaccgcaggcttaattaaattcaatagttttaatttattagtagttgattgctgtagggttctttatgctctggcaacctagtaggttagagctatggcaaaataccaaaacctttttaatatgtaacaagaatctattttatacgttgctctaaccaaactgagctacaggaggaatcatcctcccggcaggactcgaacctgcgacctacggctccatatgcaataaaaatcgctgttagattctttatgttccggcacccattaaggatggaactatagctatactaaaaccgttttatttatgtaacaagaacgcattaactttaagctttaccaattaagctataccaccagtggtggcagttggattcgaaccaacaactcacatttttcagatgtattagttttaaaagtttgctgtggcgttctttatgttccggctacctgttaggttggaactatgacatatgtcaaaaccttttatcttgtctttataatagcataagctatttaaactgtcaagtattagtttaaatatattgaaacttatttaaaagctccaatatatttaacggtacctttgcatttacgtgcaaaagcttctagctctttacgagcacctaaacggtctggttgagaagactgtactgtaacttcttttggtacagtaatttcacgagttttcttaactatctttccgttttttaacttttgttcgtactcttctgtacgagtagaagttgttgaaataattccgtaaaatttagacatgttacgtgacctccggtcattttattttaatggggagtgatggagttgcaccacccgagctttcgcaactgatttacagtcagccccgctactacttacggtataactccccgtatatttttgtaatcagttgccttgtatggtaaaccataagacttacaccattttctaacagcgttatctgatacattatattctcttgctactgctgagaagctagcttcgcataatttgttaaacaatatatctttatcaggtattaatgcttttctactgaatcctgatttttcaaaacattgtttacataggtaagtttttccttttccgtgcaactggttattgcattgtttacagaaataaattttagctttattcttttgcccaccgattgctctattatacccatcaggagctaatgtattgtacttcttaatgtaagcacgctctaattcgtctaggtctttttgttcacaagtagttagcacttcccaagaaaagttttctttaccatattttcttatggcttgatgtactacacactcatacgacctatctgttttataaaaggatttccaaaaatgctcttttacacgcttttcaatattaactgattgtccgatatagcattttccgttaatattgttttttattaagtaaattccaatcatagtatcagacctgcttacgaattaattgtaacaaattatatagaagttaataggcgggtacccgacagcgaagtatcattcctgtcttgtcattctcctattaactccataataccttgcctagagttgagctaggactcacaattaccgaaggtactaatcgggaaagcaggactcgaacctacgaccccacgctcccaaaacgtgtgcgctaccaactgcgctatttcccgttaacttatacatatattataacacaaacttacatgctgtcaacaactatttgtaagtttttttaatttatccattcgggacagtgtgctttaaccattcagccacccgcaggggtaacccgcaggacgggattcgaacccgtataccactgtcctattggataggcttgtgtacctgtcatcttccgattacacacgcctagtctagttaccgtaccaatagatttgacaggaatctagggtaacgcccaatatggtgttagttcacctcgtattctgttacaggtttgcaaccctgaacaaggatgaccaatcctaatggagcatagcggacttgaaccgctgacatctaccttgcaagggtagcgctctcccaactgagctaatgccccgtgtaaaacacctccgctgagattcgaactcagaatgcgggattagaaatcctgtggtttatccaattagcctacggagatatgattgtttacagataaggtactcctgtaaacaattcaattgcttcaatttcagtatgaaagttttcccattctaaacattcatagaaatcttgtaagatatgcttgtctttcctgtaaacagtctcacctttatgggctttctcacagatttctctaacagcatccacattatacttaccaccagttttatactgctctaaaccagaatctaataaaactcgttgtacttcttcacttttatctcctgctttccaagaatcaaaagctttttcaaacaatgtgtcctccatgttttcaccttctacttccatgattgattgaataaatctttgccacttagaaggctccttgtgtttacctttacgaaaactcatgattagcctccattatctttaatgaaccgaggaggagggactcgaacccccaaacgcttttacacgtctacatgatttcaaatcacgctgaataccagttatgatactccccgaaaaagaaattgattgactagtactgttagttgactagccaaccggaactacttaaaccgaactctttaaacagctttagtagtttatatttcaaatggaaggcatgggattcgaacccacacatgctgttacacatctacttggtttccaaccaagcctctttaccgtttgagtaaccttccgtcttgggagcaggcgggtttgtgtcaagtacacaatacctaatcttttgtgcatgttgttccctaggctttatgcacaccctactatctgcatactaacagacctgccaattttctaacactttttctaatgcttcgtatggacacattactgtcttaccgtgaacaccatgtattctcaaaataatataatgttcatttatctcggaggtaacaaagtatatcttgtccttcactaaacctaaacttccgtttaaaccaatgaattttaaatacatcatgttacctcctaactatgtataacccatgaagcatcatggggggactgtagtatgcccaacctgcggggatgctccgctctgaatccgtagaaatcagccacaattatgtcactggtaaggatttgcaccttacatgcacgcttccattagttagtcggcaccttcgagatagggacatacagcttcaattttctaatggatttataggaatctagccttacagttgtttacccgacttgttctagactagcctgaatcttcccctgtgctatactgtgttaattagcgtttacctattccgccacagtgactatggagtagacgggactcgaacccgtgtcctaacgtagaaccatacaaaattctacgggcgtagttactagttttaatctacgttaaggttacaactaataactgaaattaccttaactgagtggtaaagcctctcctgagttatccacttttacctcagcgttgtcggttgatgagttctctaggtagaaccgaccctagtctagaggggtaagtgtgtagctagtctaaaactaggctacagctaataattcttcgtttttagcagttatatttactgcgatgattaagtcatcactctgcccgctattcatatacttcttacgccagtcgaaaccaaaactaccccaacataagaatttcttttgactaaccctacccttccggcagggtctaatcaaaagtatacaattaaaaaaatgaaaatgagttacgaaaaatgtaacgtctccaccaccggactataagggtaagttgttccggtctttaacctaagtactttacttaaatctaacaggacagtcttcattacgtaaactacttaaccatgtaaaacgtaaaggtcaatttccgttgacctaatacaagaggagggattcgaaccctcactgaatagattctaagtctacttcctctgccagttgggatactctcgcatagttatttttgttacaaaaggtctcagtctgcgaatgacaattcggacataagtaccttagattatcaagttcattatttgtgttatccccgtctatatggtcaagctgtaatgttagcacagtacctaaccattcaccagtgtttccacaaatagcacaagcatatggaattaaaccatctttctttacacgtttactcagtgcgcttccacggtaaatccctttaacatagtattgttcgtttggagtacgagaggctctcgaagcgtgtacataattcggctctaaatagtctctgtaatcaatccccatagaatcaaatcttcgttttgctatgttacgtgtatgttgtgctcgtcctgtagagtagcctaaaccttccaaaaacgattggaacgtagtagatgctttaagtctgttcagaagctcttcgtcagaatatttgtccataagtgaagtttttaccataattaatcatcctttcatgtactaatatagcacacgtacacaagatgaccaattagtgccacatgtctaccaattccaacactctcgcataataacagtcctacccacctactgttctgctagcatatagcttgcaggctttttatctgtgttataaaaaccagaaaggttatacaggtaacaggagtcgaacctgcatgtcacaaaggacaatggattttaagtccacagcgtataccaattccgccatacccgcataatcactttttaaactagattatttatgatttcccacttctgaaaaaagttcattttacatatcaatatttttcttatgcttaagttaagatgtgctttaacgaggaagaggctaaaagagtatttcctcaaaattttcggtcaagttaatctagtaaggaaaagtgtaaccttatgtgactgctcagaatcgaactgagattcatgtttccacagaacactgccttaccattaggctacagtcaccatcatgtgttccttacaagaattactatacagcatcttgtaaggaaagtcaacacttacatataaatttatttaaaaaagttttgatgaatctaattgagagaaactttcgggtagagaacctagctcttccatgtactcttggtcaatctcgtaaacagttaactccgttacttcattttcaatagtaagtttcattgcttctgctgtttgtatactgtgtccttctacttgaagcttatcatcaagataaatgccttcccagtctcctgcaactacataatctgctttcaagatattacctactttcttctcaatttctgataaaaattattatccttgtaatcttcctcgtgggcataaaattttttcatatcatatgtccaacaagtttcttctactcgtttatgatgtgtcaatgttacttcgtcatcaaaacgaccttgatttaatttatgcctgtaatttctgttaaccattctttttatgtctcgaacagttgagtccttcatacaaacataacggtaatagggttttctgtaacttctactcattgtaagtcctccttaatagatgcttacaatttagacaatactttcttcacaggaaccactccttattaataaaagctacctcccgaaaaacgtttcttcaccaaagtgtaagacgtaaactaacgacaggtcatagttttccaatcaaggcaaataccagtgagaggatttgaacctctgacacccgccttatcaagacgatgctctaaccaactgagctacactggtgtgacccatgtgtcaggactttcacctgttctcggggtgccccgcctctctactaatctcctcattcggtgcccctactagagtgactagagatagttagcaataaaaattatatatcatgaatcaaatttttactgcatcctatatacatgataccgtctacaggatttgaacctgtgacatccaccttgtaagggtggcgttctcccactgaactaagacggtgaaatatggaaccctaccagaggatttctttcggtattacaatcacctcggcatgaacaaagccagatattgaggtctggacttctcacgccccgggagtcgaacccagtataaacaaaccatattttatacggaagacaggatttgaacctgcgatataggagttttagtgcactcattatctcccacggtatccgttgcgtattgccatcacgccacttccgtatgctaaccgacagtaaaggaatcgaacctctatcactagattcgtagtctagcatcctatccaattgaacgaactgtcgctctaggctagcaattacgcagactgactatgggcaccttcgtcccgtcccctgtaagtgccctcctacaggttgtccgtggcaccgcaatgtactaaccgatgtccccacagggatttgaaccccgaaccccccgattaaaagtcgggtgctctagccgattgagctatgaagacagttgtgtttttcaagataaccctctcgaaaaaccgttactaatagaaaaactaagggtctcgactgacagatttgaactgcctgctcctcctaagaggcgtgttaccaaagccatacaccaactcttcgagaatatcccctacaggattcgaacctgtgaccccctgattaagagtcaggtgctaccaccaactgagctaaggagataaaattaccaatagctagttaagccttacggaatcgaaccgccatgaggttatgaagctcacatagaccactagacacaaaactagcttattgataaaattctatatagataattgtatgcaatgttcgcactaactaaccagataactagccaatcattcaaaacattaatttgtgagtttccgctcacaatgctgattgagagaatcgaactcccattcccgggttacaaagccggagtaatagccgttatactaaatcagcgtaacccctcaatagaggggaattattatactaagttgccatacccgtaggtatttgtttgagcttgtttaaacatgatgctcttgatagttaagtcgttatacttttcatcgtcttcatcatcaaggtacacgtcaacgttaacgttcttaccggaaataccgattgtttcgtctttaccaactgttaagtctttactgttgaaggatacatcaaagtagatgctatcttcttttgtacgactaccgaacgaaactctgtcaccaaataaatccaagcttcgagagttgattacaacaggttgtaacccgccttcgaattgaatagttactgtgtaatattgattttcaatattaaggatgtttaagtcagcaatagcttcaccaaatgggataccaaagcttaattccaatgcaattgctcgtaagcagtcatagtttaacttaattctacggctaaacttaacaacactgtcaatttcaccatagtaagcttcgtctaacttatcttgcaagtaagtgcgaatttcatctgcatcaggatattcgaaacgtaaatggtaatggaaacgtcccggacggttaagcataaagtcatttacacgacttaagctattaacagtaagagcgtagatacgtttacgttgtgataaaccatcgaataagctcaatagtttttcttgtggttcacgaccgtcacggtcattgaatactttttcaaattcatcgaacaggataagagactcttggtcaatactgtcaataaattctgcaatccccggataagcttctgtaattaagataacaggcatatcctcttcaatagctcgttgagataagatttgagtaaatagtgatttaccaatccctttgtctccacttaaaatgattcctaagctacgattaattgttttaaatgtgtgtagcactttttcaattttagctaaacggtcaccgtaaactttaggttctttcaattcaaaatcgtctgttactactaatgagaacccagacattggattgaatcgaactttataagtttgcgcaggcaacttatcataagttttaaggtcgtttgcataaatttcatagttactaccatagttgattactttcataaatattccccttattcgtgttttattagcatttgctaatggagaggggtggaattgaaccaccgacccctagtttttcaggctagtgctctaaccgactgagctacctctccgtgtcaatttggacttatcccccacgtggttactaagataagtcctatgcctttgaagtgtggatactcaaaaatgagtacaggtactaaaaatctgatgacaagtcagatttatgtcataagcagaaggtgagattcgaactcacgttcaggggtttgcaatccctagccttaacccacttggctactcccgcattggcatccctctaggtttttatctgcatcgggactcacataaactgataagtgaaaacagggcactagtctgctttacacctgcgctatcagaacctagactcggcaacactgccttagatgactttactcttgtaaacacgtcacattcttcaacaattaccagtagagtgctacataccagtaattcgttcctgtgaaggatagtacactataactatcaccgctgtccaagaatacacgccgtgaaggattcgaacccccgtcttccgggttggagccggaaatgatagccactacaccaacagcgcaagattcggaggggagggactagcaaccctccgaaaaatgtaagataatgagagattaaaacatgccactattaaaagtggaataggtaattaaggattcgaaccttacaagcccttactgcttaccttgtaaacttatagtatcatgtgttgtaccgaaagtcaacactttttataaaaataatagggagaaatatttctccctaaaaaatggactacttgggaatcgaacccaaacctgtcagttatgagctgacggctctgacctactgagctaatagtccgtgtaataataccacgaaataaaccttagtaagtcatatagtcccagaaagggagacatatgaaactagtgttgaatgccataatcgtttaataaatgtgactgagagagtaataaatttcttaggcatgtccatatgcctcctttccaataatgatagttttaagatataaacagaataaataaaagagaaaataataatgaataatagcgcatgagagagtcgaactctcgattccggttcgaaagaccggtgtcttgaccgcttgaccaatgcgccataaacgagctatatgggacttgaacccatgaccttctccgtgacagggagacgagcactcctgactgctccaatagcccaatgtttccccaaccaccgccacctaggggattcgtacgtctacgtgagttttttggttatacattattgttcaagacaataaattgtacgagtggaataccaatttaccactctgcggttcctattgcggtaggtgggagtcgaacccactaaagcttggttatgagccaagtgcctaaaccgttcggcatctccgcagtaatacaagtctggttaaaaactcaaaacttaatagattaagggcttttcttcctgcttcatatacactcactgcattcggtcacatactcagagaggctgaaccatcgggagctacccaatagcttctctctgcctttaatgtatggtctagcacggtggctaacaacgttcacctaaacattcagcattaaaagtattattttacatataacccatatgtcttctgtaatattttcatccaacgctgacttaagctacttgtaccaaggtctcttgttaaacctgtatttctacagttctcgtcaaagggagtcttttgagttttccaaggtctgccaagcttctcacacttccacctaacttggattcttttccacaggaacgtctattgtatcccccaagagtcttgtaccttacaatccatgtatactccacatagcacttaactgatattagcttttactaatagttacttacatgtcaccatgtaacctacctacaatcaaagacactatcgcaccttcaaccatattcagaattatgccatgctatcctagcaagctgttcaaactttctgcactaggttcaaaatctattaagttttcaatttttaaccactaacttgtggtgtaatactaatatagcataacattacaaagttgtaaacacctttttgtaagtttttttgatttttttttgatggaaggtaagggggtcgaacccttacaccggatttctccgacctaactgattagcaatcagccctcttaaccatttgagtaaccttccaaaagcaaaaagggaacccctgcaattaaacaggggtaagagagatgtctctaaaagcctacaaccattataacatagaaacatagtttgtacagccgtaactgtacaactgtcaggacagggttcgaacctgtatcgcaaccttcaaagggtcgtgtcctacctgttagacgacctgacaaaaaggtggaacctccctaaatttggggaagagggaggttctcatgctattaacaaacacgaatttgctaaccaagtaatcttgactagcaactcacctagcaggacttgaacctacaagaactggttaacagccagtcatgataccgtttcatcataggtgaattttagttctatcaacattataacacaaaagctgatagaactagcaactttttttacaaacttaacaacttttttcttaactactgattcagcaacttactcaatagttaaagacttaaagaaagaggaagtaattgaggtatgtcaatcactaactaaagtatacaacatttttatcatcctgacaacaactaaatgtaaaaaagttcaaaaaagattataaatagttaaattatcagaatattcaaattaatgtcctattttaaatttttgacctccttatatacttaatactatatattaaatctatatatctaaattatataaatattagactatatattattaatatatataagacttgcaaaaaattaaaataggacatttttctgacaattcacttttagataaaaagaaagagagcaaaatgccctcttaccgtgtttcaaagtattccttgattgtgtactctctagcttctttagcttcctcagtttcatctttaggagctaaatacaagtcagtaattgtagtaggtacctctaccatcttgtaatcaataaaagtagcttgacgtagatttaagtatcggatacctgttgttctatcagtacctgtatgaacaggcaagaaagttaattctttacctctgacagagccattagcttgtgttagtgcatcaactacagttccgcccatatagcgtggaaataggtaaaccttgtcagtacctactgtaatttctgcgaccttttcgtgtgttggatagttcacttataatttctcctttcagaatatgctataattacagtataagtataacacattctatggtttaaaaatgctgaacacgtaagtcacaaccgcaccgagaacaatcatgaagatgttttccacgatgtctcttttatggtcaaactctttgtcgtttacaccttcgagagcatcaatggtttcttccagttttgtaatctggtacgtaagatgtgaatatttctcttcatgaacagctaaccttttatcaagactgttaacgatgtccttcaattcactaactgcattattcagttctgtgttgtcctgtttaatgcccttctcagttagttctccatgttggagcttgtcttccaatcgttgcagtcttaatactaggtcacctacttggtaatcgttcatttgtgctccccctctcggttgtatagctcaatctcccggtcagttagaaaaccacatgtaaccgcctatgcccagaatacatccgacaaggtaaataattggagtatacagcccaatgaagagcataaccacgctaccaacactaagtccgtataatatcaaaagatacttatacgatgccaaaggacttaagtctgcttgcagttttttgtactgtatagctcgggtgacatagtaggatgctgagtaaacgaacaactgtagcaataacatagcaataaatgctagtcggtacattctatcgcctctcttctttcctagagctatctttaatatagtacaccaaagcctttataatcaggtgtgctagattaaatatgtaggctaaaaaatataaacggaagagtgagaaaaagtgaaggtacggaaaaaattattatacaaatcaatttttatcaccatctctgttatcatcatgatttgtgcaagcaagctgattgcagatatgctaggcttagaacaacactttacaacaacaatgcttgtaggaggttctttgtgggcttggatagctagtattgtctttgattcttatgagaagaggaaaaagaaagataatctacagcaacagcttcatgaagcactgcgtgatttaaaagagataaaagcgttaatcaaaaaataattaataaggggtggaggatatgtcccttacaagagaagaaataaagcaaaaaaggcagtatatttttagcaaagctcaaaagacgacaaatatacgaaaaggggataaacgcatagcaagtgctacacggatgtgtgcagtatgtggaagaccgttgtccagacttgttcttactaatggttctgtaatagttactacagcacatgtaagctgtaagatgtcagatttagtacgattgaacatgtgcgaagacatacggtcttgctatgcgtatacaagtaaaaagggggaggaatagccacatgagtatggcagaccgtattaaagaaaatgctgaacaaaagaaaatggaacgtacatccgaacaacgattacgtgacacatttaataacgcttctctcacgttaattaaccagttcatggctaaagtcatggcaggttcaatagatatagatgacgttggggatttaacccgtctattccaaatctacatgcagattaataatatcaatgcaggaatgcaagagggaactggtcaattacctgctcttagtgctacgcaaaaagatattattgcagacaaggtaagcactgaaaaggctaccatagacggagaggaggaggaagttgtttcgttggaggaattagctaaattatcggatgaagaaattgataagatgctattggataaggaaatccaaatgaataaagaaaacgaagcaacattttaatgacaaataaagcacaacatattgctaaaatggcaaaagaaatgtatgggacggacaaaataacaactgcacagcttgcctacatcacagacatgttaactccgtccacatatttactaagaaaccactccgttcgtaatcatccgattacatttatgatttccggacgtgaccaacaaaaagcccaagcgcatagaccttggcaagtaaaaattattaatgaccaacatcgaacaaaggcagttattaaatcacggcaattagggctttcagaaatgggtgtaggaactatgctacactttgcagatacacatagttacgatgcagttaagtgtttgtatacctttccaacaaatgaacaaatgactaaatttgtacaaacacgtcttgacccagtattgcaacgtggttactacagtacaattattgacccagaagtagactcattaaaggctaagaagattcgtaatagctttttatacttccgttcaagttctaaaccgggagctgtagaaggggtcgatattgattacctatctatggatgagtacgaccgtgttcctgcattagcagaagcttctgcattggaatctatgtcatcttctccatataagattgttactcgttggtctactccatcagctcctgatgtaggtatccatgctttgtttgaacagtctgaccaatactggtatctacacaaatgtgataagtgtaatcattataaccaaatgaactatgaagactatgtaccagaggcaccagtggaacgtagagggaacatcctttgtgtaaatcctaaaggggtagacccagttgctaaaacagttgtggatggctccttccaattcgtttgtcaaaaatgtggagagccattagaccgttggtacaatggtatctgggttcctaaatatccagaccgtactaaaaatggtctaggtattcgaggatacatgatttctcaaatgaatgctgtttgggtatctgctgaccaattaaaaacaaaagagctaacatcactatcgaaacaagcgttctataactatacgttgggttacccttatgctgaccaaaaacttactgttaatgccggtgacgtagaacgtaatagacgtggagacctcccagagtcccctagagaccgtggagactacaagtttatctctgtaggtattgactggggaaacagacactgggtgtccattcatggcgttagaacgaatggtcaagtagatttaattaaattgttctctgtaggtaaagctaacccattaaaccctgatgcgattgatacagatattcagtcaattcgattacaattagcaccatatgaacctgatattatcgttgccgatgttggtgactctggtgataaagttgctaaactgattcagatttatggcaaagaccgtgtatttggttgtgtatatccatctacacctaaatctacaggtaacttagtaccaagttggaatgttcaaggtaacaaggtatctgctgataagctaatgcagaataaacgatacatttcaaacatgaaagatggagttattggtttctaccacaaaatggatagtgaactaatgctttacattgaacactggggaaatgtagtaattcgtgatgaggaggatgaaaaatcccctacaggattccgacaaaccatcggacgtaaaggtgatgaccactattcacaagctagtgtttattccatgttaggttatgaacatttgatgaacgtatttacaggggcagatgactacgggtttaactctgattggatttctactcaattgaaccctacacctcctgatatttttactcagtttacttaaaaaagcaaaatgtcctattttaatttttctacgtccttatatattaatactatatatctatatacctatatctagttattaagtatatatagtataatatattatataagggagacaaaaatttaaaataggacatttttatcaaattagctattgattgacaaaaaataatatgctattatactacttgtcaataatagcactagttatttttcttgtcataacaataaaggagacgtttacatgaatgacaatcaagaataccaagctcttgttgataatcttcttagctctgtaccagaatttaaacctgtagaagctacagtcagtaagtcctttttagaaggtatgctaggcaagaagttagactacgggtacgttatatcagaaaatgaaaaattatcaaaagactattcacatattgtgaaggcttatggttttaactccttttatgacctatactgctatgctgatagttgtgattcactaaatgactatttaattaaaggcggtcaaaaagatttatcaaagttgaagccagttaaaagacgagtaatgagaaatggtaaaatgatgacgactactatctacgaagacagttccggagaagatgatgataaaaacccattagataaggacactcaaacagaggaaaatgttgagccaagaaatgctagagatttaagcaaaacaattattggagatgaccataatggtatagacccaaaacaaattgctaaacttaaaactgaggcaggtaatttaaatgggacattttcaactgattgttcttcatacttagttctccaaggagaaaccggagaacttggtggtgtggcaggttatcgtaaagagggtagctatttatatttagctttctaccaatcagacgaccttacaagtggagtggcttatgtagcatttacacaactattgttacgtgctcgtaaacttggcttaggtgctaagattgatgttactgacgacccgttagcactagaattatttaaagaatacggacttaaaaaaagtggtagttgttatataatttccaagagttccctactggaagcgcttggggagccatgatttcaacttttgagttatttttaattttagccttctcaacaaccgtgtttatatgctataatctttttgtagctcaaacagtcaagacaatttttgatttgagaaaaaaggcacaagttgtgctaactgaaagagcaactgagctaacgaagaagttaacaaacatactgaaaggggatgtacaggagttgccagagaaagaaaacgtaattattaacgaacaaacactctctgacttgttagtcgaaaaagcaagcgatgccgactataaattgttcattgagaaactgacagagattgtaaaggatttatattcaaattatcaatttcttcgtcaagacccacaacccgatgatttcatgtcaggttattttttagggttccaagtttttagagaggaataccctgtcgaatacgagtatttattccgattagctattgaccgtgggttagacgatattaaaatcaataaactctttgtcgaagctttacaaaacggagaggtattaccattaggggatgccattgtagaccctaacttgtccggatgcagttctatccttcaagcacaagaagtacgtgtaaacattgtattcgcacctaaagcatacatggagcgtaaagaagagctgattgaagaaaaaactgctgaaatggaaaagcagaagaaagagcaacaagaactagcaatccaaaaagataacgtggttaaatctgcacgtcttgctgaggctgtcattaacgaattagtggatgaagtcgcagaaaaacacgaactacaagaactcgttaaacaaattaaagaaagcatgaggtatttctaaatggcaaaccctacaaccaaagtaacattaaaccaagcattaatcaaagaattgcgtgcacttcaaggaaaaagagagtccttgattgcaagcaatctacatgtaccagaacctcagtcagttatgattccaattcctaatgttttagtaacagatggtgacaaagaggtactaaaatttattaaaaatattggtttaattagagctgataaatcttttgggttagatgtggcaattgaagcaggagaaaaagtaacccttacagatggacgttcttccactatcttggaatgtattttctattacacaggagatacaaaaattgatgaaattgagcaattcttagaacaaaactaatatttcctgattaaaaaggagcttttttttacttgtgctatattaacactgcacaggaaaagagagcttctttttttgtgacttttttataggaggttgcaagatggcaggagaagtatttagtagtttgattacaagtgttaaccctaatttgatgaacgcaggtagtcgtaacggtattgctattgaccgaattattctacatcacaacgcaacaacaaataaagatgttgctatgaacacatggttattaggtggtagcgcaggtacatctgctcactatgaatgtacaccaacagagattattggatgtgtcggtgaacagtactcagcattccatgccggaggaactggtggcgcagatattcctaagatttctaaccctaaccaacgttcaatcggtattgaaaatgtaaattcaagcggtgctcctaactggtcaatagaccctcgtacagttacaaattgtgctcgattagtggcagatatttgtaaacgttatgggattccatgtgaccgacaacatgtcttaggacataacgaagtgactgcaacagcatgtcccggaggaatgaacgtagacgaagtagtacgtcaagcaaaacagtttatggcaggtggaaacagcagtacagctacagccactcgtcaagcattcttcgatgttgtgaacgtaaactctggtgcattcaacgtgcaaggatggttcatccctagcaagtctacaaaaggacaatctatttggttgtactttatggacaagaaaacgaataaagaaattgctcgtttccaaggtaaacgtgtagttcgtgaagacgttaagaaagtatatccatctaaccctaacggagcagatgttggttttaacgttgatggtgcaactccacaaagtcttatgggcaaagactactacttcttgttacgttacaacaacgataacaaagaagaacttttcgtaacagataaagtctttaaagcacctgcactagttaaccgtggttgcctagacgtaattagaggtgacaaaggtcaggttactttacatggatggcatttatcttccaaacgtagaaactctgacaaacacttcttgttcatcatggataagaaaacgaataaagagattgtgcgttttgacgttacaaaatcttcattcaaagcttccccagatattcaaaaattgtatgacggtacaattgcacaacgaactaattgtagatttgcttttgctcatgcactggatgctaaatcccctgcacgtggtaaagacatctatattttaagtcgttactgctctgaccctgcggggaacaacgggatttccgaccagttacaacttggtggaactcacaaattgtaacattacattgacctaccttcgggtgggtcttttttattactgtaatataatgtaaaggaactgtaatactataggactaactttgtaacactaatttgctatattagttcctgtaagcaacacataaaactaaaacaacccaaaggagaacgacaacttgaagaaaacgacaattgcaacattaggacttttaggactaggactatcattaggattgggagctaaagctcacgcagacgaaatccaagaaaatggacaaacttactggcaagtagaatccggagatacattatcagctattggtaatcgttacggaattgattttaacttaattcaccaagcaaactcagacaaagtttcagatgctaacttgatttatgttggagacaaactattattaccattaaacggtgaagttcaagctcctgtagctcaaccagtgcaagaagcaccagttgtagaacaagcgcctgtagtagaagaaacacctgtagttgaacaggcaccagttgttcaagaaccagtagaacaagctcctgccgttacatctaactcagcaaaagaatggattgcacaaaaagaatctagcggttcttactcagcaactaacggacgttacattggacgttaccaattagatgcttcatacttaaacggtgactactcaccagaaaaccaagaacgtgtagcagatgcttatgttgcaggacgttatggttcatgggaaaacgcacaagctttctggttagctaacggttggtactaaaataataaatactaacatattaaaaaaagactctctttttagggggtctttttcttatgctatattataactgtaacttaaaaacaaacaggaagtgatagctatgagtaacattaacatggaaaccgcaattgcgaacatgtacgctctaaaagcacgaggtattacttactcaatgaactactcccgaacaggtgcagatggcacaggggattgctctggtactgtctatgattctttacgtaaggcaggggctagtgatgcaggttgggtacttaatacagatagtatgcacagttggctagaaaaaaatggttttaaactgattgcacaaaatcaggaatggtcagctaaacgtggagatattgttatctttggtaaaaaaggtgcaagtggtggttccgcaggtcacgtggttatcttcattagtagtacacagattattcactgtacatggaaatcagcaacggctaacggtgtttacgtagataacgaagcaactacttgtccatatagcatgggttggtatgtgtatcgtttaaatggtggaagcactcctccaaaaccaaacactaagaaagtaaaagtacttaaacacgctaccaactggtctccatcaagtaaaggtgcaaaaatggcaagttttgtcaaaggcggtacatttgaagttaaacaacaacgaccaatttcttattcttactctaaccaagaatacttgattgtaaataaaggaacagtacttggatgggttctttcccaagatattgaaggtggatatggttcagatagagttgggggaagcaaacctaaactacctgccggatttacgaaggaagaagctacgtttattaatggtaatgctcctattactactcgtaagaataaaccaagcttatcttctccaacagctacaccattgtatccgggacaatctgtaagataccttggttggaagtctgctgagggatacatctggatttatgcaacagatggacgttacatccctgtacgacctgtaggtaaggaagcatggggtacatttaagtaacatgacttaagagggctaaatagccctcttttttgtgctatattaaattagtagacagtgatgaaaaattgttacagggctacctgtggtatacttaaactagtacaaatgcttgggaggtaagaatacgtggcatacttacaggacgatacatggcaagcagtaaaacagttagctaaacaaaacggttttgtaggagactggattctaattattcacctctactatgaaaacggtggaaacaatgtacaagttcatactgctatagataataaaaattaccgcatactacgaatcttagataacaaacaggttttattagttgatagagaaggaaatgttgctgtagaggactatgaagtagtcaatgacagtcagaaaggcttcttttatagtgatatgcacaaagtcgaactaacacttccggaaggttgtagcttcaacggtaaacaaagaataaaaatttatatatgaggtaggtgtaatcaagttgccaaaatggttagataaagtgttgggtgtagaagaagatactgacatcgttaaaagcgaagaagtctaccaaaccttgcttaaagaaattcaagcacagccgttaacagaaacggtagagaaaggtatgaatggtaaaccagtagcatacatgcaacctataattggtgacatgtcagtaaaccccggatttaagacaaaaccttctattagaaattctcaggatttacacaaaatgttgaagaagtttggtaataatatcattcttaattcaattattaatacacggtcaaatcaggtatctatgtattgtaaacctgcaagaaactctgagacaggtgtaggttacgaggttcggctgaaagatattgaacaagagccatccacacatgacattgcaaatattaaaagaattgaaagctttttagaaaataccgcagtattcaaagaccaaaatagggataactttactgctttttgtaagaagatggtacgtgcaacttatatgtatgaccaagttaactttgagaaggtattcgataaagacggaaattttatcaaatttgataccgtagaccccacaacaatcttcttagctactaacggtaaaggaaagattattgaaaagggagaacgatttgtacaagtactagacaatcgaatcgtagccaaatttaatgaacgtgaaatggcttttgctgttcgtaacccacgtgctgatattgaagtaggtcagtacggttatccagaactagagattgccttgaagcagtttattgcacatgaaaacaccgagacgtttaatgaccgattcttctctcacggaggaacaacacgtggtatcttacatgttaaagcaggacaacaacaaagtcagcaagcattagacattttccgtagagaatggagaagttccttatcaggtattaatggttcatggcaaatcccagtagtatccgcagaagacgttaagttcgttaatatgacaccatctgctaatgatatgcagtttgaaaaatggttgaactatttaatcaatgtaatttctgcgttgtatgggattgaccctgctgaaattaacttcccgaataatgggggagcaactggttctaaaggtggttctttaaacgagggtaactcaaaagagaaaatgcaagcttcccaaaataaagggttacaaccattattacgctttattgaagatactgtaaatactttcattattgctgagtttggagacaagtaccaattccaatttagaggtggagacctaagtgctcaattagacaagattaagattcttgaagcagaagttaaagtgtttaaaacagttaatgaagcacgtgctgaaaaagggttggaaccaattaaaggtggagacgtaatcttagatggtgttctgattcagtctatcggtcagttaatgcaacaagaacaatttaactatcaaaaacaacaggataagctcaacaggctcttagagctttctggtggcgaagcagatgattctaatggaattagcttccaagacaaacaacagggcttagatggaacctcagctagcgtaaatggcaaaggagagtcatctgttggtaaagatggacaaattaaaggtaagggcataactaacaccaattctgctaaacaaggtatgaaaggtgacaaacctaacgactggcaaaaataaatagaggggtaacaccctctatttacaattgtccaactgtttagtggtagttctgctatattaaaaacagatgtaagtttaggtggtgaatgaacaaagatgcaagagaagtataatattttcgttcccattgacgtagcaggttccgtagagaaatctgaacaggcaaacgatggtgaatggtacgtacagggttatgctacaacgcctgaccttgacttgcaaggagacattattttaccacagggaattgacatttcttactttgttactaaagggtgggtaaactacgaacataaacaagatgcagaatacattattggtgtaccaactgacaactgttatgtagaccttaataaaggtttgttcgtggaagctaaacttatgaaagaaagcaagtatgctcaatcaatgtggcagttagctaataccattcaaaaatcaggaatctcacgtcagttaggtttctcaattgaaggggcagttacaagtcgaaatgcaagagacaatcgtattattgagggtgtggctatcagaaacgtagcattaactacacatcctgctaacccacaagctacgtgggaaacacttgttaagtcttggacaacagggtatggaattactcctgagacacagacagatgcaggagcattacgtagagagatgtttaaagaagacatcacaaatctaacatatgcagtgaagacagtagctaagttgtatggtaaaaaacccgaagataaagagtttatattacgtgaagttgctaagagttttgaccaagaaacctctgaaccagagttaagtgcatttatgttacaattaactagaggggtttccttacaggaagctaacaacttcattgaaagtagaaaggagctaaaatagtgtctaaaacaattaatgacattatcgaagatttcgattcaaaagtgaatgtacaggaagtatctaaatctattgaagatgaaaatgtagagcctgtacaggaagaacaaattgaaacaccagaagctgttgaagaaggtgtagaaaatgacaagccggaagaacagcctgttgaagctgaggaagttaaagaggatgttgaaccagaatccgaacctattgaatcagatgaaaaaccagaaacagaaacagaatccgaagtcgcagaagaagctactgaaccagaagtggaaaaagtagaaaaatctgataaagaatccgataaagaagatgaggaaaaaaaatcttctgatgatgaggacgacaaagaggataaaaaggataagaaagataaaaaagctgacaaagaagataaagatgaaaaagataaagacaaagaagatgttaaaaaatctgatgagtccgaagacgtagaaaacgttgaaaaatctgctatctcttcccaagatattgtcggtggattcgaagttgtattcaaaaatatggcaaacctttttgaaactcaaaaatctttaactgatactattgcagaattaaaatctgaaatcgtaagcttacgtgaatctcacacacaagctatcacaatgaatacggaagatgttaacaaatctattcttgtagataaagttgatggagtatcagtagaaggtaaagcagtaggatttgtttctaaatctgtagatgtagaagcagatgtgacttctgaacaagaagacgttgttgaagtagtagtagaaggtgccgaacaagaagaagctccggctaaatctgatgaagaacaattgtttgacagtgttcgtggaatccgtgatgacttaatgagcacatatactcgtgttgcaagtaacggacaagcccctcgtggtgagttagaagaaattcgtcaattgtggggacgtattaaatcccaagacgaactagctcgtgcacaagcattcattgataaatacaaataaatttactaaaaggtaaatttttttaaggtgtactgctatattatagtcagaaccaatgagagacctgacatttcccccctgaacctccacagggtcaggtctcatctttgagaggaataaaaatgaaaggtgatttatatataatgccagaagacaaaaaacaagaagctaaattaaatcccgttcaggaacaagtaatcaaaggatttacaactggttatggtattactcctgacacgcaaactgatgcggcggctttaagacgagaatttctagatgaccaaattactatgcttacatgggcagaaggagatttgtcattctatcgtgacatcacaaaacgccctgctacatctacagtagctaaatatgacgtatacctagcacacggtaaagtaggacatacacgtttcgttcgtgaaatcggtgtagctccaatttctgacccaagcttacgtcaaaaaactgttaacatgaaatacgtttctgatactaagaacatgagtatcgcaactggtttagttaacaacattgaagacccaatgcaaatcttaacagacgatgctatctctgttgttgctaaaacaatcgaatgggcttctttctatggagatgctgacttatctgaaaacccagataaagattccggtttagaatttgacggtttagttaaattgattgaccaacataacgttattgatgctaaaggtgaaagcttaacagaagcattgcttaaccaagcttccgtattaatcggtaaaggttatggtactccaactgatgcttacatgccaatcggtgtacaagctgacttcgtgaaccaacaacttgacaaacaagttcaagttatccgtgacaacggtcaaaatgtaacaatgggattcaacgttcaaggtttcaactctgcacgtggtttcattaaattacatggttcaacagtaatggaattagaacaaatcttggatgaataccaaatggttagtcctaatgctccacaaaaagctactgttcaagcaactgtagaaactggcaagaaaggtacattccgtccagaagacattgctactcctgctgagtacaaagtagttgtagtgtctgacgatgcagaatctgctccatctgacgtggctactgctacaattgctaacgctactgacggtgttaaattagcaatcactatcaacagcatgtaccaagctcgtcctcaatacgtagctatctaccgtaaaggacatcaaacaggattgttctaccaaatcgcacgtgtacctgcaagtaaagcagaaaacaacgtaatcactttctacgatttgaaccaacaaatccctgaaactgctgacgtattcgtaggtgaattgactccatctgtagttcacttgttcgaattactaccaatgatgcgtttaccattagctcaaatgaacgcttctgtaacattcgcagttctatggtatggcgctttagctcttcgtgcacctaagaaatgggcacgaatcaagaacgttaaatacattccatctggaaatgtaattaacgaacgctaatcttagttaaactaagaaaaattgaatagaaaacggaatagggaacggtctgactgttccctatttttattataacgaattaaatggaggaatacaaatgttaaaatctacagtattacaaaataaaaaagtggcaacatcttttggagaagctgaatttaaccatttaggagaaagtcataacttgccagaggaacatcaaaaagctttagctgaaaaggtgccttacttgacacacattcctgataagaaagaaaaacctaaagaggaaaaagaagtaaaagaagaaaaaccaaaagctaaaaaagcacctgctaaagctaaaacagccaaaaaagataaagagtaggaggtcatcatatgtacccagattacggttatggtgatggtggaaaccaagatgtataccaaccatatgcacatggtaacccaaaacatattgacattgcagacctagataagattactcctgctgattacggttggactcccgacacgttaaaggcttatatgtttggggtagctgtagttaaccctgaaacaggtgaaccattaggagatacattctatgagcatgtaattgacacagcaattgctaaagctgaaaagaaactggatattgcgattttacctcgtttaattcaaggggaacatcacgatttccaccaatctgactttaactcttacttatacacgcatgtatttaaacgaccactaattcaagctgaaaagctacaattagaaattaatggacgaggaatctaccgttaccctgcaaactggtggaaagtatataccctagcggggcatatccaaatgtacccaacagcaatgatgcaaacaggtacacaatttgcctatgattttgcattctctggctacccacaattagcaggtatgccaccttcatctggtcaagtagatgctccacaaatgattcatattgactacgtagcgggtatgttacctcgtaagaatcgtggatacaacgctgagtgggagtgccctgctgatttagagcaattagtaatcaaatatgctattaaggaaatcttccaacaatggggacgtttgattattggtgccggtattgcaggaaaatccttgtcaattgacggtgtaactgaaagtatacaaacaacacaatctgctatgtatggtggtgcatctgccgacattaaacagattgacgaagatattgatagactagaacgtgacctagcttcctacttcggaatgaacctaggaattatttaaggagggatgactcatggctaatgaaccaacacgtttcagcagttcaggttctacaggtaaccctaagcaatacatgaatgtaagtagactagagtttgaaacaaaaggtatggatacctttgtaatgaaccgtggtataaacattctgtgggaacgagcttggctttgtacatgccgaaatccgatgactcttgctcctgactctaattgccctatttgtaggggcagaggtattgcctatcaacctgctgttaaggatataatgattatccaaagtcaagaaaaaggtgtatcaaatcaagatttagggttgttcgattctggtacagctattggtacaactccaattgactctaaaattactttccgagacagaattactgtcccagatgtggaaatataccaaagtttcatttttaatgtaaatagtcgtagattagagacaggtatgtttttaagctatgatgttaaacggctagaggatgtgtatggtgacaaaggtcaagtccttgtggagggtaaagactttacgatggactatagcacgaatactttttatcctaaacaacatttgttaaacacgaacatctctatcaacatggcagtaacattacgttatctggttattgatttactcaaagaaagtcgttaccagtatacgaaatttggtgtaaaagaaccactgtttgagaaccttcctagaaagctcctattgaagcgtgaggacatctttgttgatagtgaaccctttgtattagaagtagatacgaagtctcgtctagaggcactagagaaggaaaaagaagtaacagaatctaagatggtagaccctaaacgtacaggttcgagtgcgggaggattctttggaggtaaactaaatggctaagaaaggacaacgacctgcactgttcaccagtcctaaagaaatgttaggtaatctgacacgagcaatggtagatgagattctatctgatgcacaagaccaagcgttacgtgcaggtgccgatgtccaaagaatgcctaattatttacaggtaacagaatcaagaatggctaagagcggtgtaattgatttaaaaccgttcttcgctcgttctagtaagaagaagtacaacaaaaaaggtgaatggtacttatatattcctattcaaattaagacaagaaagatgtctagaagattgtatgacgatttaaggtcttttcctacaaatggtcgtcccgtaacagttaagatggactatctttatgatagaagaaaacaaagtcctgctgtacaaagtatcaactatagccctaagtccaaaaatgttacaattataccaagagagtggggagaaggtacacggaatacgtatgttacattccgtacagtaaatgccaattctccggctaacagttggataatcaaccgtagtaaagtgaactcggacgatatgagtaaaacaatgttagctaacatcgacaggttaatgaagtggaaattgaagaatttaggagggtaaaagatgataccaagtttagacacttacttgtataaagaatttgaagaaagactgagaattatcttagaagagtgctatattatagacgaagctttgaaggagatagattcccaagctttagagacatttaaaaatacttattgttcaattgacggtaaggctccaaagaaagaaatcgaaatgtcgtattctttcccacaggagaagcagaattttaatgctcgttatgtgattacactaggtgggagtgatgaggagaataagtcaatgggtggtgtacagggaggctatacttaccgagaaggtgatatagttaatgaagctgttcaacttgtccgtaatggaaaagatttaattatgaacacaacaaagcctgtagctgattttttgaatagcccagatattagtttttcagaaagcgaccatttcagaatcgaaaacaacaagcctgtatttgattattccggtaatgaacacttagaaggtaaatcttttaatattgtttatactgtgaaagagtctgatgaggatgtagcaggtgtttacaaagggtaccaatcctccgataccgtaaacattgtcggaattagtacaaatatggacactgcccgctgtctagatgccatcgcaagactaattcttattacgatgcgtgacagcttagatgagaaaacaggatacatgctacagacattacactttggagacatgcaacctgtaattgaatcaggtgagacagtagtatttggtagaccttgtacaatcgggtataaagtgactaattctattagttttgatttaaaccgacaaatacgagaaattattaccaaaaggaggatgaaagcctaatggcaaaagaaacaacaaagactaaacaagagagtccaaaggaacaacctaaaaaggttccaaagcctaaagggtacgttcatgtagatacatttttgggctatgcacaagtactttacggattgagtaagtatcaagtagcaggcttcaaagcattcatggcagggagagagtatcagcacgatgatgccgactttgttccgtttttggaaaagtatataggaaaggaagttaaataataaatggctgtagaacaattcccaagaaaaaaagtgtcccgtccacatactgaaattactgtagatacttctggtatcggtggtgcatctaacagttctgaaaaaacattaatgttagtaggttctgctaaaggtggtaaaccaaatacagtttatcgtttccgtaactaccaacaagctaaagctacattacgtagtggtgagctattggatgctattgaattagcatggaacgcttctgatgtgaacactgcatccgcaggtgacattttagcattgcgtgtagaaaatgctactaacgcaacaatcaaaaaaggtggtttaaccatcgcatctactatttatggtttagatgcaaacgaaatgcaagttgctttggaagacaatagtttaacaaatactaaacgtctgacaattgcatttgcaaaagacggatacaacaaagtattcgataacttaggtaaaatcttctctatcaagtataaaggagaacaagcacaagcttccttctctattacagaagataatgttactaagaaagcaaacaaacttattttaagtgcgggtgctgaggttgaagaccaaactcctgtaatggaatatagcttaggacaaggtgtttactctgatacaaacgtattagtaagtgcaatcaacagtttacctgactgggaagctaaactattccctattggagacaaaaatgtgcctactgaaacatttgataaagtagaaaaagctgatgttaaaacaggtgagaaatatgttgaagctctaggcggagacattgctaaacaattggaatataacgactacgtaacagtagaaattgaccgtgcaactccaattcaagactttgcgttaactaacctttctggtggtacagatggaacaatcccagaatcttgggctgacaaattccctctactagctaatgaaggtggttactacttagtaccattgacagacaaacaagctgtccattcagaagctttagcattcgttaaagaccgtacagacaatggagaccctatgcgtatcgttgttggtggtggaacgaatgaaacagttgaagaaagcatcgcacgtgctacaaacttacgtgacccaagagcttctgtagtaggattctctggtactcgtaaaatggatgacggacgtttgcttaaattaccgggctacatgatggcttcacaaattgcaggtattgcaagtgggcttgaaatcggtgaagcaattacgtttaaacacttcacagtaacaagtgtagaccgtatctacgaaagtggtcagttagacatgcttaacgaaagtggtgttatcgctgttgagtatgtacgtaaccgtaccttaactgcattccgtatcgttcaagatgttacaacttataacgataaaacagacccagttaagaacgaaatgtctgtaggtgaagcaaacgacttcttagtatccgaattgaagattgaattagataacaacttcattggaactaaagttgtagatacaagcgcaagcttgattaaaaacttcatccaatcattcctagataagaaaaaacgtgctcgtgaaatccaagattacacaccagaagaagtacaagttgttcttgaaggggatgtagcatctatcagcatgactgtaatgccaatccgtagcttgaacaagattactgtacaactagtgtacaaacaacaaatcttgacagcataacaggtcgggggcttttagccccctataccttactaaaaataatctaaaataggagtgaaacatagatggctagcgttggaaatcaaacagtccacacaggtaacacagtttacttgatgattggtaacaaaattatcggacgtgcacaatctgcatctggtgaacgtcaatatggtacacaaggtgtatacgaaatcggtagtattatgccacaagaacacgtatacttgaaatatgaaggtacaatcaaccttgaacgtatgcgtatgaagaaagaagacttagcaagcttaggtattactgctctaggtgaagatattcttcaacgtgacattatcgacatcgtaatgatggataacttgactaaagaaatcgttgttgcttaccgtggatgctctgctgtatcttacagtgaatcattcacagctaacgaaatcactagtgagtccactcagttcacgtacctaacatcagctaaagttaagtaagtagtgtaaaatagaccctaaaaaatggggtctatttttttagttttggtattgacaaattatacatgtatctatataataggtattgtagttggtatacattttcgaacttaatctttcttatctttatcttacacatgacctagcaggagtgctaggtctatttttttgctctcatagaggcaactaactagtgtgctataataagtatagaatgctatattaacttatgaaagacataggaggaataagtaatgactgaaaaagaaaacaaggttgatttccctcagcttaacatgactcgtgaacagcttatcgaaaagattaaccgtggggaaaatctaacagacgaagaaattaatttgttaaaatatcataatgcagaagcagaacataaagaacttaaccgaattattccgggagttaacaatgtattctcaaaacattatgatttaaaagaatttggtatggaatttgatattaaaattaaagcaccaaacgtggtacaacaaggtaaaattcaagcaatgcgtgaggcttacttagaaggtatgggtatggcagtaagtaactttattttccaatgctaccatacactagctactatccgtgtttgtggtgtagaagtacctaaagagctagaaaaagatgaagacatctataacatctacgtcttaaacattatcgggaaggattttgcggagtggttaaactcctttcgctactaatctcgaaagtttagggggtataaaaaccctcgtcagaaatacatatagccgaaacatgtgggctattatgaaaaaatttaatgtattacctagtgaccctgcatggcaaaatctaactcatgagcaagtagaatggattttgtataacatggaaagagatgttgaagaagaacaacgcagagctaaaggtcttgctcttgaaggtgaatacgaagacttggatgcttcttggtatgatgtacctcatgaagagtttaaccctgtacgtgaaggtcatgatgaagctgagattgctcgtaagcttaatgaaatcactacagaagaggacatggctaaacttaaagctcgttgggaagctagtcaggaagtcgatgcaatccgtgccgaaggtggtacaacgattgaggaagacacaatcaatgaacttattgctaacaatgtacagaaagctattgaagaagctaaacgtattgagaaacatggtggtaataagtggggagacaaaaccccaattgaattagaggaagagcgtaagaatctagaatttaaatctcagcttaagcaagatgatattcaagatgcaattgctctgttcaatggagaaaaagatattgaaccaacatcagtagacgatgattttgtaatataggagggtagggttttaccctaccttttctttgtaaaggaagtgtggaaatggctaacaactatcggttttacgttgatgcaatgactggggatgccattgcaaaattagaagaagttaataagttgatggataaaattgactctaagagtgcaaaaggtacccagaagtttttccatacaactcaaagagaaatagacgaagcagtagccgatatgcaacgtttaattcgaatgaaaaaggaactagataaaaacttcgaaaaacaaagttctatggcagaagctacaggtagcatgacagattttaaacgtgcacgtgcaaacctagaacagttacaaagtgagttccaaaaaacacaaaacgagttccaaaagttagcaagcatgaaagctaacccgaactttacgaataaaacagctttgaaagaacaaagagcttaccgagaagaattgactgagcaggaaaaagctttacaggcaatcaagaaagctcaacaagaattaaatcgagtaaactctcgtgtaaatcatagagcacaccaagctacctctacaggtagaatgacctacaatcaatctgaaagcatgaagaaggatttaggtggtgttgggctatactcagatactcaacgagaaaataaagaacgacaagctcaactaagagaccaataccgtagaagacaagaggagctatcacaagtacgttctgatacaaacttagatagacaggttcgtaagaatagagaaacatctatcttagaagaaatcaaaggtatcgaacaagagattgaagctcgtaagaaattcaatcagaccttgaatgacactatcgagaacttaaagtcaaaaacggaacagttaaacaatagtaacgttaaagtagaggcggataggaactccactgcgggcgttatagcttctcgtgccccatctatagccattgcaggtttaggctctgctgtggcggcaataggtggcttatacgctaagggagctactgcgaatgcaggtatgcgtgattcaaccatctctttaggacaacgtacaggtaacggagacttccgtgctattcgtaaagaagcccaaatgatgggtattgaaaagcaacttgggtataaaggagctgatatgcttcaattccaagaagatgcgttaagtaatatcgggtttaccaataaagaagacctaacagctaatactagagctttagcggaaggttcacgtgcagttcctgtcgattctgaaaccttaagcgggtttatgaatgaccaaatgcgtagaggtgctgtgagcggtaaagaccaaatcaaagctattcaagaaggtttcttaggagctatccaaaaatctggtatggtaggtcgtgaaaaagagcagttaaatgctttgaaaacattaagtgaccaatcatttagtggacgtaacggtagtaatgaggaacttaaacaacaaatggctatgcttacactgttgaacaaaactggttctcgtgccgtccaaggtgaacagggtgccgagttaatgactagtttatcagcgggtattcaaggtagtgtttggaacaacaaggcttctttattactaggtaagggtagtcaataccaaggtttaagtggtatgtatgacttagcaggtaaacaagaagagggagctacccctgaaaatctgcaaaagattattggtagtgtacaaaaagctgttcctaatgatgaaaaaggtcagaaatatgtccttaaaagtgctatgcacgaactatttggtacaaatgcaactaatgaccaagtagatggtatcatgaaggcttacggtaacggtaatctaaatgaagaaaatatcaataagattatgaatgaaagcgaatctactggtaaagataaatacaagaagaatgcagaagattatactaactcgaaagagggtatggctaatcgttccgaagcagtaactgagaaacaagcttccggtatcaacgactatggtgatgctctacgaggaattaattctaaattaggaggaatccctccggctatgtatgctttaggtgcaggtatgggtgccctagcaactgccttgattacttcgggaactatgggtggtctccctagctttgttaagaacttgacgagagggacattctctgccggagctacaactgctacaacagccggagcaactagtgcaggtggttcattctttaaaggtgtaggacaagcctttaaatcaggtaaggctactggtggatggtcagcaggtcttaagagtgcaggtactacagctaaagacttagcaggtgctaatattgccggagctgaaactattgcaggagctacgaaagcaggttctgtagcaagtaaatttggtaaagtagcaggtaaagtagcgttacctattgccttagtcggttcagcaattgatattgcaagctccgatgacaaggtaaaatctgttggtgagaatgccggaggactagcaggtggatttgccggagctaaattaggtgctttagcaggtacagctattttaccgggtgttggtactgttatcgggggtgcattaggtgctcttggaggtagcttaatcggtaataaagccggtggtggtttagtagacggtgttcgtaagttctttggtggagaggaagcctatgcagacgaagtagatacaggtacttccgcaggtgggcaagaaacgcttaaaggacaagagaacaagtctggtaaacaagaagaacgagacatgacgaataaacgtgtcctttctgaaaaaacacgtgctgagaataacgctgaggaagcaactaacttatccatctactcaaaattacttgatagagcacaacgaatcctaaatcaagctagaaatcaaaatggtatctttggtaactccaagggtgctaacagttctagtgatagtggcggtatcggtgacgaccctgcaagtaaggattttggcggagactgggaaaaagccattcgtcaagcttctaaacaacttggggtagacgttacagaccaagatgtagacacattgcttaaattgattcaagcagagtctagtggagatgaaaaagctgttcaacaaatcattgatgagaacaactttaacggttctggtggtgccaaaggacttctacaatacatccaatctacgttcgatgcttataaagtaaatggacacgatgacatcatgagtggttatgaccaattactagcattctttaacaatagtaactggcaaaaagacttaaatgcttggaataatcgttaccagaatggtagtacgggttggggtccaacgggtagtaagacccatgcgatgggtggacatatcacttctccggaacatgctttgcttggtgaggtgccgggacaagatgaatatgttattaacccacatcaacctacagctcctagattacttgcagaagcttctcgtaaaactgcacagaaattccgtttgtttggtaatggttctggggacgaatggtcttcccgtataggtggaattaacttatccggtggaggtagttcagcacaagcaccaacattaaacaatacaaatgacgttaaagttagcgtaaccgtacaaggtggcggaacttctgaaagtattgctaatgaaattggtaataaaacagcaagtaagattctgcaatcagccgatgatgcaatgagtttctttacaaaagaaatgaaacgagtttaagaaaactaggggggactcgttccccctttatttattgaaggaggtaaactatggcggtagaattacgttatcctagatttaacctagcgttttatacagaaaaagacgaatatcatattacgtatgatgcacagacaggtgaatctaatacttctgtagtaaacaaagataatgattcggaagtttctaataactttatgatagaatccgttatcagtttaacaacaaaaaattcaatggaagatgacagtgctgtcttttcttttgttctagccggagatgtatactgggatagaattttaaaagccaatgatgcagttgtattacggctttaccccgacataaccagtgacttacctgttaacccagttttattagttgggttaatctcagaagtacgacttgaaggagactatggggagaactctaagatgtatcgtattacaggtcaatcatttgctaaggctttaatgcaatttgacctaggtgttattcaagaagttagtgttgtattaactgatattggttggttacctgatgatgcccaagagggggttaagatgacaggtagaagtgccagtcagctagcagaaggaatgatgaaccgtttcttacagtatatgaaatttgactttaacggagaaggcttagagcgctttttagaatgggaattagacagttggacagaagcagagcggttgatagataataccccgtatattaactatgagggttccctaaaacaattgttagacgacatcacagctaaaccatttaatgaattgttctttgatgctacaccgggaaacaaatgtcgaatgattatgcgtagaaccccgtttgataaagcagactgggagaatttaactacgtatactgtaacatctaaagaagttatttctgagtcagtagcaacaaatgatacagaagcctactctatctttaatatttctataaataacctttatggtacagactctatgatgctcggttctaaaccacaggtattccctgctttagttaataagtacggatacaaaaaactagaggttgataacaaatacttgcagggtgcaattgtagataacagtgataatgcagatagtgcggatacaaatactgacggtgatacagaaacgaacgaagataccactacagaaagacgtacaactagaaaaaaagatacgaatgaatctagtgcattcgacacgacttacggtacagtaatgaactatctacgtggttacccattagacatcttaagagtgaaacgtggtaatgttcgagctaaaatacttcaagtagacagacgtattacaggaacaatggcagataaaatcattgaccagtatattacaacacagagcttgacaaaagaacagttcacagatataactggtattaatgaaaagaatgctagcaagggaaatggtaaggaaaaacctacttataaaaaacttgtgaagttcttggaagattacaaggatgaagcagagaacaatgagactaccacaatcaagaatgaacttatgaatgagttcaaattaacagataaccaagcaacatctattgctagtgagtttgtggctaataaaagcttgcgtaaaactcaatatgacgaaatcatgaaaaacaatcctagtgatagtacaacggtatctggttctgatacaaaatacgtaaaagaatttacgaaacgtttagctaactggtactgcgaaaatcctaacttctatagtggagatattgtagttaagggtagtccagaatatcgtttaggcggacgactattcgttatagatgaacagaacaacgaattgtgggaatactatattgaatctgtagagcatactttttcttacacgcaaggatacacgacaactttaggagtaactcgtggtcttaaaaacggaggtaaagaccgattcacacacctttgggacaagtcagaagacttctcaggaggtatgttgggtgaaaagaccttagaggctttactagaagagcaagaaaagaaaaataatgagaacaatagtagttctggttctagtggtggttcaagtggagttaacccatctaatattccgggtggtacagtagcaatgaaagctgtaaactggggacgttctcactctaaaacagaatcttcctttaggtctgcgtatgactggggaggcgggcgtacaggtagagacccatttgaaagctcacctattgctacagactgttcgtcatttgtatggtggtgttttaaacatgcaggtgtagaattgaatggtggagcaactgggatgactacatggagtattattgcggatacgaaacttgaaacaatagctacccgtggtcaaaagaactccgctatctttgataagatgaaagcaggagatattatctggttccgtaactgtgaacatattggaatttactgtggtgaaggaaaaatggttgcatgtaatggttcaggtaacatgaatgaaagtcctacagcaggtatcattgtgtcagatatgacaagtggatactggtgggatacatttgatggagatgtaaaaagatacaattaacctcgttaatagcgaggttttttgttgtaaaaaacaaaactagtatgtgtgctataatattaagtagagacacatacaagagaaaggatggtatcaacttggcaggaggacgtagatttcaggcagggttaggttccgaacataaaagattatacaaagaaggacaacaaattaatactttgttgctagcccaagttatccaagttaattataaatacaatacagttgatttactagcattacagcataaagaagttttccaaaactcctatgccaatgaaggtagattttctgctcgtttaccaatggaatttggtggacgaaaccttgcaggacaaccatatggtcaagtaaaccctattgccgttggtactgttgtcctagtaggttttattaactctgataaagacatgccgattgttattagtgtatacaataacaacgatgtaaataaacaattatcccgtacacgttttgctaatgcagaccctacagatatggctttagcaggacaaatgtatcaaaaatttagtttgtacccatctttaacttacgatagtattgatggggatggtaaccgagtagttacattctctggtaaatcatttattgcattcgatacaaaagatatgcaaaactctccaatgacagatgctagttatggttctcgttatgaagacctaggaacatcttactataacgatggagaactaattgaaccgatgaaaggtcgagcacctaatgtactattcaagcaccaagggattcttgacgatgataataaaccagatacacataatttcatgattcacattaacccagatggaacataccgtacatctatgatggatacggaacaagactggcggacaatgtttgagatgacaccggagggtaaaatccgtttacgtagacaaggggatactgtacgtttaaacgatggttttgaaattggtgaactaggtattaatgaagaaggtatcgtttacttacgtaatggggacatggacttagaagttcgtgaagatggtatctattcccaaggtaaactaatcacagaaaatatcaatcttgatgatatttatgaaaaattagctaatgttacttttgaaattaataagacaaatgagtccttacaaattttaactgagaaatcagaactacaagatggtaagattgtaaatctagaaacagagattacaattgtagccggaaaagtagaatctaaagtaagtgcaacagaagtacaagacatgattgatagctctattgtagacatgactgatgctattaaaaaagcccaagaagatgcagatagagcaaatcaagttatttctgatatggctagtgataatcgcttggctcctagtgagaagctagacttactaaaagagtgggacatcatcaagaatgagtacccaacttatttagcacaagctgaactatacgaagtagatagcacaacttacactgctaagtacaaagctttagaaacatttgttactcctttattggaagacatggaagctactagtgtagtagacggttctattatgcgcaaaacatttagtgcatactacactgaacgaattaatctgttaaatgcaatcactaaaggtttaaaagatggtttagaagaagcaatgaagaaagcttctcaggcttctgtggatgctacacaagctttagctgattctgcacaggcacaaattgatgcaaacaatgcaaagcaattgattgcagatattgctagtgacggtaagctgactgcttctgaaaagtaccaattgaaaaaagaatgggatgttattgttaaggagtatcctacaacaattgcccaagcaacaaaatataaagtaaacacagataattatactgctaagtataaagctttggaaacatttgttactcctttgtttgcgaacatggatgaaacaagcgtagttaacggggaacaactacgtgcagtattttctgattactatgctgtaaagattactttattaaaagaaatcacagatattgctcgtgatgaattgactgattatggtaaccgtattactgtagcggaaacaaaaatcacacaaacatccgaagctatcacgttaatggcttcccgagtggaaactgttgaaaatgatgttaaaacaaacacagcgcaattgaaagtacaagcagacctaattagtcagaaagtaactgctagtgaagtaaaagatgctattgataatgcaattgataacatgtctattggtggttctaacttgtttgtaatcaaaactcaaacagcaggactattgaatgaaaataatggtactgtaggaacagccgtagataaatctgtagtatctaactatatcaaggtaacagctaaaatgccttatgtagcttctttatatggaaacacagggacaaacagtattatcattgcatggtatgacacaagcaaaacgtttatctctggtcaagctgtagctgattcgggtgatttccataaaacttatgttgcaccagaaaacgcagtatatgctcgtttaagctataaaaaagctgacactgttaaaatgaaatttgaagtaggtacgaagccaactgattacagcccgtcatgggatgatattaaaggtgaccagactgctttagaggaatacattaaacaggtagaggaacaagctaaacaagcacaacaagaagcagaaaatgctaagaatgaagcagaaaatgcaaatagtgctattgctgatatgtctaatgacaacatgttaacagcaaatgagaaacaacaaattttactgcaatgggaagaaattaaaacggaatacccaattaatttagaccaagcaactaaatttggtgtttctgctacgcagtatacaacagcgtacaatgctctaaaaacatatttagacccattattagcagatatgacaacaacttctgtaattgttggttctactatgcgcagtacatttaatacgtactacgaccgtagaacaacattgcttaaccgtgtagctgaactagctaaacaagtagctgaccaagctaaggacacagcagacaaagtagacgatgatttaaacaacatcggtggatacaactacattggattctcttccggagaccatatgtaccctcgtttaatgattaagaacgttggttattactatgttccttctacaacaagtgcagaatttgtagatgatatggtttgtttaaaacctaaaacagctacagctacaagtgttcaatatgacatcggtaactcttatgttagtgtggctaacgttggacttgctaactatcgaatgaaagaagttaaaacaggtcaatggctgactgcttctgcaaatttaaaagttgtgggaacaggtactgcttatctaactatttttacactggaaaatggttcatggaaagcttcatacagtgatagaattagtgcaagtcaaggcgtaacccgtgtagttgctcaaagacaagttacagatgcaactacaggtattttagttcgagttgatggaagtaacataaccgaagttcatttcggaaatacgcagttagaagttggtatccgacctactccttggaagaaatcagatattgacattcaagaagacatcaacaatgttgcagatgacattaaagattacatcggtgctcgttcagataacctaattacaaacggttttggtgagttagggaacaatacgaacattggtggtatctttgatggtgctgataggattgtcggtaagggttccttccgtcaagacgaaggaaacaaatcactattattcagtgaacacattgttattgacaataagaaagtttataactttgattactacatgcacacactaaagggtgttggtagaagttatgctatgatttgtccttatgatgtagatggtaaacgtattacgtatccttctcttggaggacgtaattacaattctactactcctgttaaatttacgaaactggcaaaaccgcttaaagtcggggataccgaagtttatgtagaagatgtgagtctatggaatggtcaagcaccgcaagactaccaacgtagtattatcatgtggggttataaaaactcatttggttatacttatcctgatggaacatacagtcagctaatgcaaatgaagacgtatgatattggtgcagttgatactacagctaacaagattacattaaacaaaccgtgggcagtaactaaccctaatagttcagatggaatcttccctgtaggacatacacttagcccaacctctgacggttctacgtatctatatcttaacggtcacgtaaatattcaagtaccgactacgtacacaagatatagtcatttgattagtggttcttctgagtttgctaacacaacgcttatcccagtagaaacaggttctatccaacttggattcctattgaatagggatacaactggtgaaaaatcttggctaaacggtttacgcctacgtgattatacagacacgtataagttaaacgatgatgttagagaaacacaagaaaatgtggataaagcccaagcagatgcagataaagctaaccagtcaattgctgatttatctaatgataatctagttactcctaacgagaaactggatttgaaaaaagaatgggaaatcattgttgctgaaaaacctaagaatgatgctcaggcagataaattcggtgtaagtaaagtagcttatggtactgcgtatactgctctagatacgtatttaaaacccattttagcaagcacaacaacgaactctgcgattgttggacaaactatgcgggatacgtttaaagcgtactacacggctcgtacagacctgttaaacgctattgcatctaaagctaaggatttagctgataatgcacaagatacagcagataacatagctgtcggtactcgtaacctcttaattggaacacaagacttttccaaaggtaaatatccgggtaacgccaacgttaccattacagatgaaaagctgtttggaaatgcagtaatgaagaacgattacactacaggtaccggatactcagatatgtaccaaatgactacctcaattattccaacaggaactcagtatatcttatcgttctatgcaaaggcagacctagaaggaactaaaatgtcttgttacttctacaacccaaatactacgacaagtagtgtaaacagtcaaggtggcaaacttagttctagtgacggtcgtactgtatttgttttaagtacagaatggactaaatactgggtaatttggacacagacacaggcagacaagcctaagtcagtaattattggtcgtaagactggcggggaagaaccgaactctgctttctatatgtcctctcctatgatggtagaggggaacaaaccacaaacgtggatgaaagccccagaagacattgaaacagctataaacggtaaagaaggtgcttgggtttactccccaacagcaccaacaaatccggctgttggtttagtctgggtggattcttctaaaactccgaaccaaccaaaacgttgggttggtggagaaactggatgggttgccttaacaccagaagaagtaaaagacttaccttggggtgaagatggttctagtcttgctgactgggtagcacaagcagagcaaaagatttcttctgatgcaatcattagtactgtactaggttctgaggactttaccggtatttttgactccaaagcaaatacagaagatttaaataaccttgcttcttacgatgacttagatgctatgcaagctgagtatgaacgtctgttaaaagaagggattgcaggaatcgacttctcaccatatgtaactaacacagagctagaacaattgaaagatagcttcactttctctgttcaacaagccggtggggttaatatgttgaaaaactctctaggattctctgggacagacttctggcaagcctcttcgggaatagatactactcagaatgaacagttatctaaattaggatttggttccggatttatgataaaccgagttcaaaatgcaacactagctcagacaatcgaattaccagaagcaaaacaaggtttgcagtatgctttatccttctatatgaatgtagctacttttggagatgtcactgatttccaatgtggggcacatatctacgaagaaggtgtgttgaagtatactgttggagtaacagatgctacacaagaaattccaagtgactatcacttgtataaacttgtgtttgaaccagagtctccaaatactgtcattgaattatttgttacaaatggtgcacaagcaactgtaatcatttcaggggttatgtacaatattggtaacattgcgttgaaatggcaaccgtacccaagtgaaatctataacacgaatgttaagattgatattaatggtattactgttaaaaacaatcagacagatggttatacaatgattactccgcaagagttttcaggttatgcacgggttaacggagagatggaacgtatctttaccttaaacggtcaggtaacagaggttaagatgttacaagcagagaagcgtattacaatggaacctatttccgtgtttgcgatgaactctaaagaaaccaatacaattggttgggcgtttgttgcttccggagaggttgcacattctgccatagcatctcgtacacaatagaaagagggagccttatctgtaaggctctcttttgttgtgttgtgctataattatattatgagaaacaaaacaagaggtgagaaaataaatggctttaagcggtagtaaatacacagcttttgcacggcataggcttgtgttagaatggagagcctcccaaaatgttgcaggcaactattctgatgtaagtatttggttatacttgcagtctatggattcctacggtgctttatatgctcctgctgtgggacaagcacaggtaacagctaatggagttaagcaaaccgaaaatgcaacatcacaattgaacgcttatcagaaaaaactattgctagccaaagtttggcgtgtaaatcataacgcagatggtagcaagtctttttctatatctgctagttactttgtaaacgtaacctattcaggtgtttactacggaacaatctcgataccgtcattctcggtttcattaaataaaattccacgtaaaagttcgttaaaccctgtgccagatttaaatattcctaataaattaggggtaagtattacccgacaaagttctagttttacacataatttaactgtttgggtagccaatagaacaaaccctacattagataatgactcccattgggtgtatttaaacagtattgagaacgtaggtacaagtggtacattcacatttagtgtggcaaacttcacagagatgttcaagaggatgggtacaagtaccgaatgggtaggtaaggtgaagctctggacgaatggtttaagcgagtcagtgccgagccaacaacgaaccttccgaataaaacctccaatgaacgcacaggcttctgggggtaaattaaaagtaaaagcaggagaaaagattactgttaatttaagtaacttccaatctgatggtaattttaaatatacaggtgtttttaactatcgaggagtatccataccagtagcaacaaatgtacatgctaattctatgactcttacattaacccaagcaaacgtggattctatcttgaaagagtctccggatgatgcaggaacatgggggcaagtacgagtaacaagttactacaatggtgtacaatatcgaactccttggactggacagcatattgattgtaccattcctaaagaagattatctacctgtaatcaacgggacaccgacttatactgatttaagttccgaagctactaacgttaccggaagtaaccaagtagcgttacaaagtaagtctaatatccgtgtgactattccggctaactttgccacaggtcaaggattctcaacaattaaaaccgtgcaagcttcattaggtggggctacaaaaaccgctaactataatgcttctggttttaacattgatattggtgccccgaacgaaggtactgcttctacattagttgttactgttctagacggtcgaggatttagtacatcttggacaaaaacagttcaagtatacccatatagcaatccagatgttaactatacgattgctcgtagaaacaactttgaagtagatacagagttagctgtttcaagtagttgggcacctgttactatcggtggtgtaaataagaatgcaattacgtcagtaacatatgcaaccaaacgttcaggtacaagtacatggggagcagaaacacccttaactttctctacagatggtgctaaagttattgtcccaacaaccgttgttcaattaccaaatgagtatagttggaatttcagactaactatcaaagataagtttggtagcttctctaaagaagcgaatatttctgccggaaaacctatcatgtttattgatgctgttcgtgagtctgtaggttttggtaacttaacaggtacaggggaaaacagaaaccttaacggtgttattgaaattgaagctgaacgttatcatagttcgggtaaaactggtatccagatgaacaatagtgatatttctggtttgaatggaatatttttctctaatgacaggatggataatgatggagaaggtattcactttattaaatcaggtaaagacgttaactctcctaatcaaggtcaagattacgaccgattttacatgagagataatggattctatgttaaccatgatggtactccaatctttaaagttactgataacggtcgtatgtactatcctgcctctcatttgtggtctggtggttggtatatgtccggtacacaaaatataaccccatcgaaaccaatgtcagagtgtagaaatggatgggtactcatgtgggctaagtatcaaaatagcacacagacatggaatgatgttgtacaggtgtatttaggtaaggacttggtaacaacacatagtggactaggtgtcagattattatttggtggttatggtaacgttgttattcataaatatttaacgattactgatacagaggttaggggtaatgcttcaaacaccaatgctagtttaaacgctgacagagctattttagtccgtgttcacgaatggtaaacgaggtggaaacgatgaaaatatacgtagcagttgacgaatataacgtattgactggatggagtagtagttacgaaaaaaatctaattgagacagagattgacgaaaatcacccacttattgtctctaatgatttttttggtaattataaactcatagacggtaaattagttaaagatacatctattgaattgttaaatttaaaagacatgttaaaggtagaattagaccaacaatgctcagaagctattattagcggttttaactacccgttaacggatacagaaacacttcacgtctcttattcacgaaataaacaacaatactttgaagagattaagtctcttttcgaacgtaaaattgttactcgtattaactgggagtttactactgatgatggggatactattactaaagagctagatgagatagaatttctaaccttgtacacttttgctagcttggttaaaacagcaaaaatgactaagcttaaagaaaaattatatccactagttgataaacaaacaagtaaagaagccctattgagtattacatgggattctattcctgatgaaccattaccagaaaaacctgttattgatttaaatggtgacggtgaaataagtcaagaagagttcgatgctttggttaaggaaaataaagaactaaagcaaaaagtagagtttaacgaactagctttactggatgcgattaacatgctatcgagcatgattatgaattaattttagggaggtgataacatgtatccacacttagcaatgctttacgcaacgtatgtcatgaaagaccctgaaacatggacgatggacaaagttcctgctatgattcgagaagatgtacaaagaattgtagatgaaatgacaaaaaaagaacaagcacccgagtagtctttctaacactaggggtttccattggtttagtcgcaggactagctattgggttcttcctataatgctcggggagaggaagagggtggcttaggctgtcctcttttttcttagttaattgtactatattaatggacgaaaggacgtgataagtgtgggacaatcagatggaatgggcggaacattaaaaagaattgctattcaagttggagacgacccaaacacaggttggtatagatttcaagttaacccaactcaatataaatacagcaaaccacaccgtgtaacaatctttaaaacaaagtctaacattatcactgaggactttggtaaagatattgaaaccattcaattctccggtacaactggatttagaaaagactctagaggaatgaacggtgctgatagattaagagaactgtctaatttgatagatgattatgcaaatcaaggtggtaatggtaaccgtccaaaagtagagatgaagttctataattttactgatgaccaatattttgtggttcatctagccccagaaggtctcactatagaacgttctgcggaacagccattactatttacttatacattaagtttagttgtattaagaaaagcaggggagcctgctgaacgtgaccaagtaaacccagaaattgggaacaaaaatccaagtgtaggagacaaggataaagaaacaagaagtggcataattcaaactcctgcacaagtactacatgaacagtataaaaattctgtgttgccaaatatagcagtaaaccctagtggtacatcaggtgcctataactacggtttagttgaactaaaaagattaattggttatggaggtggtcagtaatggcaactgtatatcaatctgcggacttgcttagattctttcgttacttaaatgtaaacgtgcaaggagatgtcgttagtaatgtcatagacgaccaacctaactttatttctagattttacacaccgcatacaagagtgaataaaatatctagtaccttattggatattgtgaaagacaataacattagtgaaacaaacaaagagttatccaaggattcactaacctataagttcttgaagagtgggttacgattaacagctcctgacatttaccagttagctcaaatcgtagtacttgaatcttttgctttgatttatgcaattgaagaaaaacctgaaatgttcaaaatgattaatgaatcagatgtgaaacaaacaagagaaaacgtaaaatatcttgtggactacttaggggagaaaaaagactatactgatattgtaatggatttacactctatggatattgcccttggttatattcaagaacagattccattaattcaaggagggttaccagtaaatggcacgatataaaaaacatacaatcgtttatggagaaaccatgcaatctattgctcaaatggaaacaggttcagtaaacgactggataaaaattgctgagtataactccttggtttatccatacatagtagatactatgcaggagaaaatggctaacattgaacatttagtaacaccgggggatactttggtaattcctgtcgaagctaacctactagacatggacgtaagtaatctaaatcaaagagacatggattttctattaagtttatctctaggtcgtgatttagacatgacaagtgagacagaatactatgaaaatcatggtacaagtgatgaagtgttctcattaacccataacgggcacggagaccttaagacggctagtggagcagataatataaggcaagcaactatctctcgtctaatgaccgctaaggggtctttaatgctacatccagaatatggtagcaacctacacctactttttggtaaaacaactattgaacaaatgaaattgattagcttagaagtatgtgaaactgtattaaaagacactcgggtagctgagtgtgtattggttagtcattatatcgaagaagaccattatgtaggaaactaccgagcaaccatacaatcaacaaaagaccagttcgagtttgtagtacaaaatgatagtgtgggcgcaattgttatagaatagaaaggagtatccctgtgagactaaagaagatttcagaaattttaggtagactaattgacgtaaccatgattaatactcatgagttaaacgacttctctgtagggtctacaatccgttccatttacgaagctgtatcaatggaacttgaacaatactacatcttaggaagagaaaatattttatggggtatcgaacagggggtactcaacgcttttgattttagaaaacgtgaagctaaacgggcttatggtatggttaccctagaatttcataccgtaactcaaacccctgtttatgtgccgacaggtacgacctttgactctagcttatctggtgcacctagtaacttgacatttcaaactatgcaagactatgtgattcctgccggagttgttactgctaaagtagaggtttactgtacaacagttggttcccaaggtaacattgccaaaggaagaataaatcgggttattaacaacatctctaacctgaaaaaagtgtataatgaatatgacatacttacaggtactgacgaagaaagtatcgaatctgtaaagaaacgtttccatgctttcgtagaatctcgtggtcgagctacaatcaaagcattagattatggtacacgacaagtcgaagaagtatctggtgtgtatatcaaagaggaaatcggttatgtacgtatttatgctcatgacttaaacggagatttgaaagacgaaacattagctaagattaaaacagctattgaagactaccgtcctgcgggaattaaattagacgtgttccctgtagtcaaattaacagtaccaattgacgtaacaatcacaattagtaataaaaacagaattaacactgcattagaagaacgaatcgaattgactattagaaactacttaaacagtcgcacagtatcagaaacattggtaaaagctgatttacttcaagctatcatgaatattgatgacaacttaatctatgactgtgaaattaataatctgagtggtaacttggatattagagacgaggagattattcgtgcaggagacgtaacgatagaattaatctaggaggtaacattgtgagtaattttttcagaaatatacacccactcttgcgaagaagtaaacgtccggagaagtatgacgatacgaacttcgctgtgttgaatgcgttaaattacgaattaacacaggcagagcaagaaaccattgctagtaaaatacaatcctccttagagtctgctacagatacgtacctagacacttggggagattggttcggggtgtaccgtaaggacggttgggacgatgaatattatcgtgcacgtatcatacgggaactacttcttaagcgtggtacaattcctgctatcattgatgcgttattggatttccttaacgacaatgatgcagtcatccaaatctacgaaccttggagaaacattttctatacaaataaatcaaaattaaacggtgacgaccatttaatgggttactactatcgttttgcaattattgatatttcaattgatagaccgtttcctcctgaaattgtagagattattaaggctttcaaacctgcgggagttttattctatctaaggttagacacaagtttaaataagaataaaacaactgtagaaagtccatatgtatatctagacgtgacgaataagacagaattagagttccttaatggtttatattatgacctacgaggtaatattaacctgtctgaccaacgtacacaagttgtagagaacaatatcttccatacgaataactctatgttaaacggtgaagatgtacttgcaggagcatttgaccacggaagaggttacattcacttagcaagtacaacattgcttgattacacaccaaaatctactgactctatgagtgacttaaaaacagctctaggagaatcaggggctgacatgtataatcaaacaaaagaaaaggatggaagaacagcttctattcaagtacctgcaacaaagaatgtccatacgttatactcaaatagtatagattttggtgactatgattatagcggaaaccctaatgtagctgtaaccgatgcagatacgttcaaatcaggaactggtggaactcttacctataataatggggagtacactgttttgatggatggaacagctagattaagtaagtataataccgatggtaaaaacagtgtatacttagaagataaggtaacctatacaatttcttgtgaaatgtatgttggttctgattataccggaaatgttgaccagatatttgctaactatgcgtatcttgatggtgggtatactgtactgcaaacatcaaggctatctgcaaatgctcctagagatacttggataacagtaagaggaacatctactatgaattatcaagggagagtacctaaagtattatactttacttggcaaacagttgctagtgatgttaaccctactggtacacttaaaatgcgaaatatgaaaattgagcgtggagaaattaatactccttaccaacctaatttattagtagagccattccaattatctaatgttcctttaaatgataatttaggagacccaaacgtatctttcccaattaacacacaaaattcctacttagtgtatgatggcactataaaagaaaattggcaagctaacacggtgtataccttaacattaaagggtaagaaattaaatacacagagttggaaaccgtatattgatactaaaaatacttccacgcttagtaaacctcttttaactcctgtggaggggttaccgaatgtttggcgcagtacatttatggtctcccaaaatgatatagacactggacgggttcaaaaacagttaagtatctaccaagcacctagtgcaacaaagggtacagcttcaattgaatgggtgaaacttgaaaaaggtgctattgctacaccacctatcgaacagtatgcctacacaggtactcttgtttcagatacatcttctaaaaaccctaaagattatacatggggaagtttctcccctgttttaggtaagaaagcatatatggtgtttgatattaagacatttatcgaagaaaattatgctatagaatttgaaaaacttattactgacctaggagaagaccaagcattaaatactgtgtttgaaaactttaacatctctacagcacttaaggctttagtaagtccaagttcaccaattaatttttcgattgaactatacgatttttctacgagtgcatggcacaaactaaacacggatagcttagacctacgtatgcgtacattcaacttagtagcaaaccgtatcacagactatctaaatgattacaagctattatttgttcgttacgtttttgataacgaaacagataaagatgtaacagttgaactagacatgctaaatgtactattcaagtatcgtttaggtgacggatatagtttagggctacaaagtactgttgagtgtctaagtgaattagaataatatgctataatataaatagggcacagcatcgtgtcctattttttaattgaatgctatattatctaatgaaaccatagaagaaaagaggttataaagtggctattgcaactaataattcccgagtgtatgcttcattacaacttaaaaataaaaaagacagcatgtacttagttatcggtaaaacctcaccttggacaaatgaagatgccccacctgctacagaccccaatacttcaacattacaagaagttgttggttacaaaaaagtaagtaaagcatctctatgtcgtgaatacgttgcagatgacgaaaacaagtacccagtaattaactatggtactcgtaaatttactttaataccagatgaggatgcttataaagaaagagcatggatggtatatatcgaatctgaaatcgtaggtgatgaattacctttaggtacatttagacaagtaggacttcacactgacctagtaccaaaaacaggtgtggacaaagatgcgttactacctacagaagtaacaaacgcaggtatcctacaattctttgagaatagacaacaacaaaacagaactgccgatgtaacattgagagaaaaatttatcgttacaatggaaaacggcaaaacattaaaagcgtaagaaaggacgataagcattggcaaaagaaattacaaatgatgatttaaagaaagcgccctaccttgaccgctttgaccctaacaagaatagaacacgagtgctattcaaaccggacaaacctctccaacaagcagaacttaatgaactacaagcgatgcaagactatgcgttatccaatgtagcagagtccgtgttcagtgacggggacattcaaacaggtatggaatttattcttcaaggtacaactcttactgttaaaaaaggtaaagtgttccttggtggtaaaatgcgtaactttaatgaacaaagtattgaaatcacaggtacaggtactgagtacattggtgttaaacttgttcaaaaagtaattaccgcagaagatgacccaaccttgttagaccaaacaagtggcgttcctagtcatttctcagaaggtgcagaccgtttagaagaaacagttgtactaggagtaaacgatgatgaagcttcaaatatttatcgttttgaaaatggtcagttatacattaacccagatacaccagaaatggataaaatcaataaagtactggcagaacgtactttcgatgaatctggttcttatcgtgtacgtggatttgacatgtatacggaagcacatccaacagaccctaataacaaaattcaactagttattgactctggacgtgcgtatgtattaggttttaaagtagacaaacctacaactactcgtattgatattgataagtctcgtgatttagaaacaatcaataacgaaggtttctattacagtaacgaaactcgtttgaataaattaggtaactcacctgtatctagcgtagaccgtgtaacagcccaagtggaagttgcaaaagaacaagtgtctcgtggagttgttggtgggggtactgattaccttaaaaatacttcggtaacaaaagttgttcgtgtatggacagaaggttctggagcacacgaatacaaacagggagaagacttccagttagttaacggtcaagctatctcatgggcacctacaggtcaagaaccacctgccggaggtacttactttgtacaatacgtttacaacaaaaaaatgattgaaaacactgattataaagtaactgttaaaggtgaaggggataaccgtgaatggtatatcgactttaacgaaatgacaggttctaaaccagtagacgaatcattagttaacgtagattacaaatacttcttagctcgtaaagacttaattgtgttagaccataacggaaactttactgttcataaaggacaacctaacgcaatgcgtttagtagattcccctaaccatattgacccattggtactgaacattggtacagttcttgtttacccagattcaagtacagcagaagctaatcaatggacaatcactcggttaacgatggaagagctacaaaagttatctgtacgtgtagataacatggagtataaccaagcagtgttctacctagaccaacctgcgatggcaggagaaaacccaatctatttacgtggggtattctcagatgcgttcatctctcttgataaatatgatacaagtcacccagatgctacaattgcctttgactttgatacagcagaaatcacactgccttatgcagagattaacaaaactgtaccatccatcatcgaaggttctagtgaagcacatgtatggggacggttagtaactgcaccatttaccgaagaagtcggaatcagacagccatttgctacagaagctatgaatgttaacccatacaatacgttcaacaaacaaggtgttctaacactaaaccctagtgcggataactggattgaagaagaacgtatcactatcaccaaagaggagacatctacaatgactattcgtcaatggtggagacacggtggggcttcttggacaaatgacgaaatgaaccttgtatctaacgtagacttagataacggtatgagttggaatgacttaaaagatgactaccgtaactctggtttatctggtacaactctatctagtggtggacaacaaacaaaagagtctatgattgagttcatgagacaaattgatgttgaaatctacgctgaaaacttagagccaaatgcaaacaacttagttgtatcattcgatggtttacgtgttcctgttacaccatcttctggttaccgtaaaggttcacaagagggtactggtatggcaaatgctgacggtacgttcaaaggtgtattcaaaattcctgcgggtgtacgctgtggtactcgtgaagtatctatccgaaacgatacgaacttagcaagtacaacgtttactgcacaaggaacaatgaaaactactgaggacatcatcatccgtacccacgtaacaatcaacctagttgacccattagcacaatcattcagcttcaatacaaaccgtattgctacaagcttcgatgtgttcttcgcttctaaggatagcagtacaaacattatctgtcaagtgcgtggtatctcagaaggcggtcaaccaaacaaaacggtttatgcagaacgtgtactaaaaccttctgaaatcaaagtgtctgacgatgctagtgtagcaactaagattacatttgatgacccattgatgtgtaaagcaggtcaagaatactgcttagtattcattactgactctgataaatacacaatgtggattgctacaatgggacaaaacagaattgatgacccaacgcaaagtgtgaactctaacccttacttagaaggtgtgctttacagttcatctaatgcgagtgcatggtctattcaccaaatgtctgacttgaaatttactgtatacacagctaagttcaatgaagaagctgtcctagagtttgacgtaatgaaaaacattaacgtagaccgtgtggttcttatgtccacatacttaactcctgcgaatacaggatgtaaatgggacatgaagattgttcttgacaatgaacctgccggaactactgttaatgacaaaccgtgggttccaattgcgaactacgtagacttagatgttaaccagattgctcgtgaggttaaacttcgtgcaacatttaaagcaaatcaatatatctctccaatgcttgcattagacgatattatgtttgccggattcttaactgctctgaaaggtagttatgttgctcgtacaattgacttaacagaagcaccgtataacactattaaaatgtcttatgaacagtttactcctgccggaactactgttgttgcaaaatacagtactgacgaaggtaaaacatggaagacatttacaacacaacctacaacaacacaacgcacacaagatttcgtacgtgtggactatgttgaaaaggttaatacgggtggaacatttaaatccattaagtttcgtcttgacatgtcaacccagaactcattccttcgcccgagggtgagacgcttaatgacgaatatgaccgacaaatagaagatgtaactgtaatgtaacatttctatgcctcttactgtggtatacttactgtatacgaaatagtaggaggttttttaatggttaaagcattacaattagcaggtactacatttggtaaatggtacgtaaaagaacgagatacttctaagaaaggtagagcatactggatttgtgaatgctcttgtggaagaacagttcaatctattccgagcggtactcttactacaggcgcatctgtgatgtgtaaacaatgtgcaaatgagaaatctttagtgggtaagacttttggtaggttgaccgtcattaaagattcaggtgaacgagcaactaatggtagtatcctttgggagtgtaaatgttcctgtgggaagacaagccttgtcaggggttcagagttaatagggggtcgcacaaagagttgtggttgttactccacggatgtactcaaaaaagtagccactaagcatggattgtctaaagtgaacggaaaacccacaaaattattccgggcatgggcttcgatgaaacaacggtgttacaacaagaaccatgcgagttataaagattacggtggaagaggtataaccatatgttctgaatggcgtgaaaactttgaagctttccatgattgggctatgactaacggattttccgatgacttgtctattgatagaattgacaatgacaaaggctattcaccagataactgtcgatgggtagatgctaaaacccagattcgaaatagacgaaatacagttacttatgagtggcaaggaaaagagtatactcgagctgagttaagtaggttgacaggtataaatgagcaaaccattgcttcaagattgaacaaaggtttttcccttgaagaagccctttcttcaaaagtaggcacttctgtattacttatgacctataaaggggagactaaacctgtcaaacaatggtgtaaagaactagggttaaactatgcaactgtacgtaacagacattataagggttggactgatgaagaagctttgactggtatacgtaataaatagtcaaccaagttgaatagcctctatatgctatactaggcatataggggttattttttatgtaaaggagacttattgatatgccagaatcatttagacaaaaatcatcaggagctttaatttttagacctacagtttctgagaaaatccattctaaccagatgaaagctcttgctaaagataaagagaaattgaacagagacatacaagaggttaataacttaaaacaagaattgactaaggaattagaagaaattagagaactaaaaaacaagttaaaaggataaaatgtcctattttaaatttttgacctccttatatatttaatattatatatttaactatttatctaagttatatgtatttatatattatatatttatatataagggaaccaaaaatggaaaataggacattgacatacacttacagtactggtacaatagtattgtaaggtgtgagacattattaggaggacttatgagactagtaatagatgtaatgcacactcagattagatttgatgaaaacgaaggtagccttcgtagcactatacataaagtaatgcacgaagaacttggagtaaaagcagacggctatcaatttagtcctgcatataaatcaggttactgggatggtattattgatttttataataaagacaacgatacatttcctacaggtctagtacctaaagtagaggaaatcctaggaagactacaaactgctatgggtagtagaggttacatgtttcaatttgagattattgatgacagacctgacaaattcatggaagttgaagaaatggactcagaaattaaattgaatggtgacaatggagaaataattactctacgtgactatcaatatgattctgtaaaaagtattatcgagaagcaaacaggtattatcaatgttgctactaacggtggtaaaactgagattgcttctggtttaatacagcaaatcctacctgctttagaatcgggagaaaggattgcattctttacaaacagctcctctatcttcactcaatctatcgaccgtattgaaaaacgattaggcattaaagtaggtgcctttggagcaggtaaaaaggatattcaacaagttacatttgtgatgattccaacaattacctctgctatttcagcagacccagaagctaaacttaaacttacaccgaaagagcgtatgtacaaaaagttagctaaagagattgctcctaagttccttacagggtttaatcaaaaaggactacttgaaggttacatccgtaacttccaagtgaaaacaaaagctgacttacagttgaaacatgaattggaagaggcttactacagttgtggaactaacaaacaggtagtaatgaaaatgcgtagctatcaagctgagtatgaaaaggtagtcgagaaaaagaacggtaaggtcttgaagaaatacaatgaagcaaaagagtttttagaatctgttgctgttatgattgtagatgaggcacaccatactagttctgatacttggtaccaagcactaactgcttgttctaatgctcagtatcgtatggctttaacaggttctatcgacatgaaaaaccatgtgttatggcaaagaatgcaagccatttttggttccattactaccaaggtttcgaatgacaccttaatcagtttaggtcactctgcaaaacctaaaattaccattttcccaattattgcccctacagacatcgaaaactccaattacatggatgcttacaagatggggatagtggacaatgtatacagaaactctttgattgttaaacttacaaagaaaatgtatgagagtggcagtggtgtactggtaattattaatcgtattgaacacggggaaacgattagtgagttattagatgcagaaggtgtaccacattacttcattcacggggagcttgataatgacttacgtgatgaaaagttacaagaaatgcgtgacggacatttgaaggtgatgatttcctcaacaattattgatgagggggttgacatctccggtattgataccttaatattaggtgcaggtggtaagtccttacgtcaaaccttgcaacgggtaggtcgtggcttacgtaagaaaaagacaggtgaaaacaaggtatctgtttttgatttttacgacttgacaaataaacacttgaagaaacattcggaagagagaagaaaaatctatgagaaagaacaatttgagattgttgacatccctcttccaacgaaataaacgaggaggtacaagatgacaagtcaatgcttaacaaatgaaattcgagcacaatatagtctaggcaacggaattgttgagttcgttgaaagattagctaaaaaagcacagcaatggggagaaacatttgctacccctatacgtaaaacaactgtagctgaggagatgggacgtgacacacgtactgttactcggtatctaggacaacttgaagagttgggtcttgttagtacagaagcaaaacgtggtagaaatggaggaactgttgtcgtgtttaatactgatattttaaattttgaaccaactgataacccgattacttcggagacaaaggaagctaaggagattcgtgaacgtgtctttccaaaagcaccaactccgaaacctaaaagacggtaccgtacaaaattagagattgcagaagctcgcattttagaacaaaagcaaaaaagctttgaagaacgcttaaacgacttacttgaacgtaccttcttggatagagacttttttgataatttcgaagaacctcgtttgtatttccaagggtatttaattgcacaaatgtacaatgcttatgcagttattttcccaaagaatagacacgagttctttaaagatattgatgtgaaaaaatcagaggaaggtttacgtagtatgaacaaagctaaatcatacaacgtgttacctgctcgttttgtaggaaccccacagtacaacaaatttgttgaagtagctagatactgtaatgaaaataatattaacccattatcttatctaactgttcaatttgagcgtgcagagcatttagccgacattggtaaagctcgtgtaggggctattccttatgtaaacactttactgtgtgaagaagctcgtaaagcttattctgacaatgtaatgttttatcgtaaaatgcgtaatagctttaacttattcggtatgagtagtagttctgttccgtataaaggggctaaatatgagattattgtagctctacgtactgcgtacgaattagatagaacaacaagagatacgttcaactacttattagatgaattagctagtggtgcacaacaatcagtgaaacaagcaaccttgttaggttattacaacactactttaaatgctttatcagaaagtgagttagtagacgaagaccaacaattaatcagagatttcctgaaagagcaagtattgctatattcacgtaagaactcattgagcagtactatctatgctttagctttcccattacaaattaacgctgttaattcagttgcgaccctaaaaggtcttgacaaagagatgtattacacttatattggtaacatgtacaaggttacagatgttaacgatgatgaatacaatagtttcacggaacgtggtcgaacaattgatttctcttacaatgctaacgatactttctttagcacaatgcgattaattgcagattgtaagggtctaggagttccggcaggtaagctaggaagtgccttacaaaaattcggtgaagaaaaagttccgttagatacttttggtatgttagatattgaacgtatctatggtaaattgattgattcagatgagttagccaaagaccgatatattcaagacaaagatgcaaccatgatgagtatggttgtgactgacgaggggtagggagaaaagcagaatgagtcaaattcacaaacaaaccatttataaagccattagtgaacctttgttcgcaaaggatgtttttagtcgattaccaattgaagattttaaagaggatggatacgacatgattgtatccaccttaaatctttattatcgtacacatgatgctccactagaagaacaaacactattaactcttgtggaagataaaatgcttaaacaaaataaaagtttagaagcacaacaggttgcatttaacttagtaagtgacttgtataagttagaagaagcagaaactgactcagaggtaatcagtgaaagtgttcagaattatgtgcgtaagactttaacacgtgaagctatcatggaagctgtaacgaatgatggagcattaggttctgaccaaaacattcagagccttatggagtctttgcgtggaatcatgacaatcgacacaggcggacatggagcagaacttttagatttctttgctgatattgataagaaaaaagaacatctacgtaatttacaacaaaacaaatatccaacaggtttcatggctattgatgctatctctgatggtggtttagctcgtggtgaggttggtatggttattgcacctacaggtggtggtaaaactacttgggcagtaaaccaagctagaaattatgttgtacgaggtttaaatgtattgtacattcctctggaagaaaaaattgaccgtatgattgttcgttttgaacaacttctgtctcaacaaagtaagaatagtatcctagttgatggggagttaaacgaacagttatatgaccaaattcaacaagcctacggtgcaggtaaagaacaattaaactgggggaacttatggattcgtaagtacaaaccacaagaactaactcccagtggtttgtcccaattgatttctgatgtaatgattcgtaaaggacaacaaattgacgtagttatcattgattatcctgatttgatgaagaaccctcattcaagtggtggtaatggagagtcagatgcgggcggtaaattatatgaagacattcgttccattgcccaagaatatgattttgtttgttggactctatctcagttgaaccgtgctagttatggtcaagaagtgaagaatgccggagcaattgagggttcaaaacgtaaaatgaatgcggtagaattaatcttcacacttaaccaaacaccagaagagttccaaagtgggtttattcgtgcttacgtagacaagttacgtaataacagtggtgtagcttatgataaaatgttgtactttaaagttattccggaaacaatgaaaattcgtgatgagacccaagaagaacgaatggctcatgcgaacttgttagaacaaacaatggaagtagctagggataactacaagaaggaaaacaattttacaccaaaggatgcccaaagtaaaattaacagtttaaacgcacagctagcgggaggtctaaaataatgaaacacattattaacttttctgattttcatatgcacttttttaaggacttctctaagccagatgctatttatgggactgaccgagcaaaagaacaaataaaggttcttgaagaattaatgaattacgctcgtgaggttaaaggagacgttttatttaatggagacctgtttcataaacgggtttctattgatgtcagaatattcaatatgatgttcgaggtatttagttcatatccagatgtaccaatcattatggttagtggtaaccacgataaggtaacgaacgctctagcctctgacagcgctctagaagcgtttaatgctttacctaatgtaactgtcatctctacaatggaaaagcttgtacgggacgaatacacgctctatggagttagttacggtgaagaagtagacgaaatgaaggaatggattgcagaggaagctaaaaagctagaccctaacacaatcaatattctttgtgctcatatcggtgttgacggttcttctactggacagtattcccatacacttagtggagcattcaaagtagctgacttgtaccctgataattttgatattgttacgcttggtcactatcataaacgacaattcttaggtgggctatctaatgtattttatgtaggtaacaccttacagacttcttttgctgatgaaggtcaagctaaaggcttcatggacattaccttagatggtaaagaatggagtatggaatttattaaagcaaactataccccatttatgactgtaacagcagatacagtgccagaagacttaagtggagcatttattcagtttgtaggtaatgttactgaaaccgaagctgttaaaaagcttaaagaagacaatgacctgtctaacttacgaattaaagtgcagaaggactattatgtacctccacgtattgagattaccgcaggttctaccccagaagaagttgtacgagcattcactgataagaagtatcctgacttacaggacaaagctttagaatgtttacgtatagctacagagatataaattaatagattttttaagcccagaatgttctgggcttttttggcatacacccttgacatgtaagtgcttgtaatgtatacttatgttagtaacagacatatacttacaggaggtaaaaatgttaaagtttaaacgtgtaagcgcaaagaactatctctctattggagaagtatctatcgacttagataatcgaggacttgttttaatcgagggaattaatgacacgaacgaaacgtttcagagtaatggttcaggtaaaagtacgttgctttcaactgtcacttatgcactatatggcactacacctagtggtttaaaagcggatgctgtaattaacagacaggtaaaaaagaacatgtctgttattttagagtttgaaaaagacggcattccatacagaattgaacgttatcgtaaacatagtaaatttaaaaatacaacgaagttatttcagggagatactgatttaacacaaaaatctgtagctgatacagacaaaaaaatcttagacattttcgggattgactaccttacatatgcaaatagcatcatgtatggacaaggaaacgtagaaatatttgctactgccacagataaaggtaagaagcagatattagaaaatctggctgatattggtgtataccgatatgcacaggatgtagcaaaagaaaaagaaaaagaagctaatgccttagtagaagaacttggtagacaacatatgaccaaggtagctgaaatcgaaacattaaaacaggtttattctagtgcattagagcaatatgctaatacagaaagaatgattgttgctcggaaacaagagtatgaagaagccagagtaaaatacaccgaggcagaagacatgctcactcaaatggaacaagaagtaacaccaaagattgaacaactaactaaagagttggaagcttgtgcagttccagaagatagctttgcaatatcagaggaattgcgagaacagcaggctaacctgtttaagttaaatcaagcaaaaactactacacaacaagctattaataaagcacaagtagatttgagtaacactgagcaagctacaaattgtcagttatgtggtgcacctttagatgcggaacatcggcaaaaagagatgttacgattgcaacaagaaattcaagagaaacaagctttcattacacaattagattcagcaattgttgcttactctgatttggaacagaaagctaaagaaaaacaggcaagggtacagcaagaagttaacgaagttgttacaaaacaacgcaatatacagtcagctattaatgaactaaacaatcgtttacgtacggctgaaaatgctgtagcacttacacggaacaatgtaactaatctatcaggagccttagctcacttagagggcattccgaaaccagaatatgacacggaagcggaagctcgtattaaagcagaaatggaagacattactaaacgaaaagaattagcagaacaagaagcaagacaataccatattttagcactagaagtcttctcaaataaagggattcgttcggaggtattagacctagtaacaccattcttaaatgaacgagctaatcactatttgtctactttatccggttctgatattgaaattagatttagtacacagacagaaaatgcagacggaagtctaaaagataaatttgatttagaggtaattaacggttctggtggagacacctaccaagcaaactctgagggtgagaaaaagagaattgacctagctatttcttttgccatccaagacttggtacaatcaaaagcaaacattgctgtgaacctaggcttatacgatgaatgttttgatggcttagattctatcggttgtgaaaatgtgattaagattttgaaagaaagacagaaaaacattagcagtatctttgttattacacataatgacaatctcaaaccattgttcgaaaatgtagttactatgcaaaaaacagaaggaaactccttcttagttgaaaagaagtaaggatggtaattacatgaaattatattcagtagataaagggcgtaaccttggaacggtactcgtcccaacaaatgtaggctacacacaacgtttagacttcccgcttgacacattgtttgattggtacccatgttgcccaagatatgattacaagtacagtgatacacgagataagatgtacattgtaatgattgaggaaaatgaaaagcttccgttacgttacaatgttccgtctacaaaaaaacgtgttaagtcaaaaacagcttgctttattacagatacttggtataatcaagaagtagctaatgacaacaagaagttatttagctttgctgaaaactatgacattgtaactcctaacaaagagaattatactctggaagacataaacaagagtattgaacgtgtacaggacattttagacatgctgtatacagttcaagaaaaacaagtagaaaaagacttgattgttcgagttggcactttggaaaaagaagctcctgattcagaagaattgaaggcagtgcatcaggaaattaaagaatacatgcagttagaccgagagggaaaagcttcacatgtattaagcttattgccccaacatgccgaagagttatatgcaaactttggagaagtttacacaatgttaaatctaatgaagaaagtagtgtaggaaatgttctcagaccttctaacaaatgaactaggcataccaaaagacattgaacacgaattgcgatataactgtccgttctgtgaaccaaaccatgattataaactatatgtcaaggttagtgatgatagtacgaatggattgtggatttgttttaagtgtggtcgtaaaggtaaccctatatcgtttgtaatggaatactttggtgtctcttttgaagaagcattagagattctagagggttatggttacagattccagaataagaactacattccaaaagatgattcattaacggatgaagagtacttattgttactacttgatactgtagggaaacctaagacggtagaggaggaagaagaaaaactcacgccccctcctttaccacaaggattccaactattatctcaaaatctatacaatccggaagcatacccatttttagtttattgtcataaacgaggttttactttaaatgacattatccgacacaatattgggtatgtactagacagcacagtcaatcttccatcaggaagacagattaggttgaagaaccatttagtatttcttacacatggtgacgatggacagtatcagtattggaatacacgtgcgattgctgatagttatataaagtcctttaatgcacctagtaaggacggagagtactccaagaagaatactatattcaatcttaacattgcatgtaaaactccacaagtagtcattactgagggagttcctgatgcactaactttaggagaatctggtgtaggtacatttggtaaacaggttacagatgcgcagattaatttaatacttaagagtgttgctcctgagcaaccaatttatgtgtatctagataaggatgcaaagaatgagataaaaaaacttgcagagcgtttgtatgaacgacataaagaaacttatattgttataagccctacagacaaagacccaaacagtattggtaaagagagtgcttgggaaattattaagaaccattctgtaaaggcagacggtgtaggtataatcaaattgatgttataggaggtacgtatgagtaacattaagtatgaagtacattgggtgcatcaccaattaggggtagataaaacacatggtgtatttggaacactagataaagctattgagtccatctatgattggtggacacagaatgactacaatccaccatatgttcgtatgtggacagtagatggtgtaactacattggactatgggtttcaccacatgttttataaaatcaaagaaataaaggagactaatgaattgaatccagtagttaggtttactaagagtgagggagccattgccccaaaacgaggacgagaagctgatgtagcacacgacttgtttacagacaaggatgctgtaattttaccgggaagactgggagcaacagtagttactacaggaattagaacagcttttgaccctaacttgtatggtctgtttatcaacccacgtggtggcatgatgaaatacccattaactctaggtaatacacaaggtgttgttgaaggagaataccgtggggatgttgggcttcctcttaagaatactttctctttacaattcggagaatatactaacgctactcgtgttttagttattaccgaagacggtaagcttgtaggaaaaccagttaaagaagtacgtgaaagctatccagaatttgataagctttatgaagaacagctaactaaactaggtaatgagctagatttaatctatgacaatatatttactggtgtttctgaaacccatgttgttgcaggaactatctttattccaaaaggtactcgtttatgtcaagcattcttgcttccacgttacgctactaattttgttgaagtagacgaattagacgaaacagaacgtggtgaaggtgcatatggttcgtctggtgttaaataatggctagtcgtataccggatttaaagatgccacttctttatcttgtaggaagtgcttctattggtactcgtgagcgtggctgtgatgtaacagttaccttacgagacttttatgtagacacccctgatgacaagttagagcaactaataactttgacgtacaaagaagacaaaattgaaaattttacacgacttagtgcagataaatgttcatttcgtgttatactaggagagtaggcaatgaaagaatcaccaagtgtaaaacttacagaaactcatacagttactcttgtaaacactgacgtacttaaaataggacaggttattggctttagagttaacagtcctaagcaagtaggcgcagaagtagtagatgtttattccgaagctgtcggtgttattgaacgtatctggtcagaacgaattgaggtcttaaacctgattacaggtggtcgtacagacctatgggcaaatacgttcgattggagagaaattgaaatactgtcagatgcggaacatctgttggaggcgtataagaatggaatttgctgagtatttcaataaaatgcaagaacattatgcaaaaggtgaaactgttgaggtagaaaagcttgttaaaagcgatggttcaaaactagttacctatgctgagtttgaggatgctttagctactgtagcacaggttactacacttgctattgaagaagcattccaagcacaggaagctaagtttggtcttgtactaaaaacattaagagaacaaaaagttatctcagaagatactgaaaaagctattcttgctgagtttaataatataaatgaacttatggaggacgacattaatgagtaaaaaagcaaaacgtgtaggatttatttctgaggaagacatgagaacatggacagagtatttagctacaggacatgttcatgataaaaaccatgcaaaacagttagaacgtttaagtaaacgtgatattagcttaggggacgtggctactgtcgtagatttcatgtcaagacgtaatgatggttatatcactgctctaatcgaacaaaactctgttaatgaaaaattacttaataaattgggcgtaacagatgaaatgcgtaatgaagcgaaagccgaatacgaagttgaattgaaacaagcacaggaagaaattaaaaagttacaagaagagctagctgaaaaactacaaaagggtgagtaatatgagcacacatgaggaattacagaaactcggtctagttgacgaacaaggtagcttaaagatagaagctagaaaaacaatggaaactccgttacaagctttagccgatttattaaacgataaggtatccagtgattctggggtatacgtaagcgactgctttcaaagtgttatgattcgagaggaaagaccttatcgagtagttaaggggttgcatgatattcctacgttggtattttcgggaacagtttacccattggatagactaacctgttatccagacattcataatattaactatcaggatgtaagtttatttaaacctgttaaagaggagcatgtagaccaaataattttcatggtacaaaacctttataatttgaacaatagaaacaacaatttgtatatgcagtttaagagataccaatgtggaaaagttacctatgaagaaataatgaacacacttaagtctaatatggagttctggtctgtacctaattatgtagaatcattagttgaagagtattatgtgaacccacatctattcgatgttaaaacacctgtagaacaactaattgaacaagtagaacttattgagtctagacgtaaacgtgttaaagaaagggatgacgagggtatctatgactgattacagtgctgttggaaaacgtagtaaaaataaaggtgcccggtttgaactagaaacagctaaaaatttatctaagtggtggggacatgactttcaccgtgtccccgcttctggtggcttacattggaaagggagtaacaatgttgttggagatattgtagctcctatagaggcaggatttccttttgttgttgaatgtaaaaaccgtgaagaatggactatagaaaacttattcctcaataataaagaaataaaaaattggtgggcacaggttgttggtgatgctaaagaatctggtaagattcctatgcttatctttacccgtaatcgtgctaaaacttttgtgatgatggcttacaacgaagacctcatcaaagaaatcgaagaccgtggttatcctcttatggtctctaatgtagagtatgtagacgggtataaagatacccattgttataagacatttactactgtgttagaagctattacaagctttaaacctcgtaagagccaaggggaagaacatctactccattattttaacccaaaagagtatgattggcaaaaatctggtcttgtccgagaaacaaaaaagatggaagaagcaaaacaacttgatgttgaggagtctttggacttacttgttaattcctatttagaaggagaaaaataatggcagaaagaaactttccagaagctattaaaacttactattcttacttacgtgttcctgatttaaaagaacaagtatccagtatctctattgattttgtagagaactggacaggtcaagaagaacggtatgtacttgatacgctcacttctgaaattaagcctcgtttatacagcttagaaacagcgtatatcattgagggcttttcactaccggaaaaagaagttgatgtaaacgtagatgaattgagtaaagaagtagccgagttctttaaaaaagcatctacagacttgaaagaagcaattgaggcatctaatgacaatcaaccaagtcattcagaagatactgaaccgattaacgattgaccaattagaagtcactcctgcgatggtagacgttgttctcgaatatctacccgcaggattgcttttaacagccaataacgagggttgggatgcggtagagcctgacctgcaagaagtggttatttccaatttggaaaaaatgagaggagcatctaaatgattgaatcaagtaatgtaaatcatccaaaacactatacacagggaggtacagaagtaattgacgttattgaatacctaacttctaagtatcccccatcaatccgttactcattaggtaatgtgattaaatatgtttgtcgtgcaccgtacaaaggaaaagaggaagaagacttaaataagggtcattggtacttgaatcgcagtcaatcagatattactgcaagcgtaagtaaaactacgtgctctattcctacttttgacatggaagctttcatccaagatactgcaaaaggctatcgtgaagaacagcaaccattctttaaatccattatgtacatgttggatgatacagaaggaactcctgtgttccgaatattaaaccttgttgagcgtgaccttaaggctcttaaaattgcatagttactactaggtagtacagagttttactccttttgcttgtaaggcgttcctgtcagttatgttataatagagataggaaaactaaatagcaaaaggagtttaattatgagtagtacacctcgagtaaagcggttaaatatctataataccgaccgacactttaatataaacttaatgaaacgtgaagacattgcaaataaaattaaagtgaatcggttaaatgaaaaagaaattgagaaagagatggatgaactaagcgaagtatcggttgtcacaccagtgtggacaatgaacatgaagaatgaaaagtcttatctcttatatcaagaaaagtatacaaatgatacattgatacagaaattattcaaacatgcaggttctgtttcttactatacagatacaattgtaccttattatgttattgaacagattgctaaaaatctaacaagtgaagtaatttatccaacaaaacctaaatatgaaaatagagagattgaaaatattcagctagcatttacggcttgtcctgtaaccattgattgtccggtagttattccagatgttagtccttatgacgttttatttgctttacatcccttaaaaacaagtgtagacaaagtacaaatttcatttccgtggctgtcagaatcagaaatatctgatagacatagacgttattatcataaggttggagaccattatgaggttaaagcaaaatataaatatcaattctttaaatatgtccagacctcactatccatctgggctatgaacatttggattgtttgtgatagtgatgaggattttgccaatgtcgattactatgtacaaaaagaaaaaaataaaagaaatgtcaatagagaacgtgctctccaacgaaaggagaagtccgatgagtaaagataaaacaatgaaccggactgatgttgcacgtgccatctctcaatatacaggttatcgtatgaaagatgtgttagctatcttacaaatggaagatgaagttatcacacaagcaatttcacaaggaatatcaattaaaaaccataaattatggaaattgaatatcaagaaaaaacctgagaaggtcgcatgggatggcatttctaagaagacgtttattcaaccagaaaaatatgtagttaagtttgtgccactttctaggttgaaagaagctatagatacttacaatgaagagaatgattagtcattctcttttttttgcccttgacataccttacaaaaatgctatactgaaatagtagaattacagaaaggggaagagagcttgaaaatactgttcttacaagattatatcagagagacccatgtacataacagtcagaatggacaatatgtagactataaacgtactgacatgggtaaaaagcttacaagtttacttaacgacataggtttaacaggtcgtgattatatgattgactacgactatgacctaattcctgaaccacagaaggtaaataataaaacaggtaaagttattaaatacaaggagcctgtattaaaactccggaaagaacctgaaaaacgtttattaaaacgattaatgaaaacaaaaccagacatcattattcctatgggaggaatgggatgtaagaacttattgaatagtacttctattacaaaagcacgtggtgttcctgtacaaaaaacaatcactgatgaagaaacaggagagttttttgatacatggattctaccaatgtttagtatggaatacttgtcaatgaaccctaatattgagaacttggtaaaggcagacatttctactttatctcgttttattgcagaaggtgcacaagcattcgagccaaagaaagtcgaatatgagcttgttatgacaattgaacgagtgaaacaaatctttgagttcttgtctcggtttaaacctcttactgcatgggacttggaaacaaacagtctacgtggagatgtattaggtgcgaaaccactagttatgtctcttagttgggaagagggtcagggtgttacgattcctttagaacatcatgaatccccttggaatgaagaagaactagctattgtatacaaccatttacaagcttttgtagctgatgcacagcaaccaaaggtaggacacaacatttaacaacatgggtgttgtaaaatccctctcatatcggtgaacgctaagggagaaatcctatgccaataccgagcctacattaacggtgacgttagatggggtgtagagactgagccgagtccttgcaggacaaggtgagaagtggtgtatcatgacagaaaggttgtgatactatgaagaaatgtaaagtatgtggaaaagaaaaggatttagaccaatactacacatataaaaaatatggcaaaacctatatcaatggtaagtgcaaagtgtgtactctagctcaaaacaagaaatatagagatactactggttatgacagagcacgttaccaaaaagataaggaaaaaattagacaactacaaagagagtacagtaaaacagaggaagggcgcaaagctcgactagaagcttcaagacgttatcgagaatcagaacacggtaaacttaagcagaaagcacgagcagtagttaaccacgcaattagagatggtaagctaattaagccagaaacctgtatggattgtggtagaagcactcagttagaggcacaccataaagactacaataagcccttagatgtagattggatttgtaaaaattgtcatgaaaatagacaccacttaaatgagggaagtacatctgagtaggtacctgcaagcaaacctactgaattaccacaggtgtactaaggtatagtccactcctttaggaaactagagggtacagtgcagtttgatattcgtttcttaatgaacacaaaagggtttgcttattttgagaataaccgtgatacactgattgggtattacttaatcgtagcacagaaaattgaaagctctaaacgtttgtcagaccttgcttatgaactaactgatatgggcggttatgataacccattagaagattataagaagaaatataaggaagactacattgctcgtaagaaagctgaaattgatgagttaaaagcttctgagaaagcacgtgtagagaaggagtataaactagcacaggctaaatacaaagaagaagtggcaaaagctaagaaattaggtaagtctaccaaatcaattttgaaacctgtaaaagaaaaagtagttgttccttctaagacagaagttaaaactgtgaatgaaattgatggtggagactttaactatgattggattccattagaaattatgcacccatacgcaagtggagatacagattgttgcttacgtatttataacgtactataccaacgaattgctcctcatgaaaagattctagctctttggacagacttctatcctaaacttacacggacacttgctcatctagaagcagaaggtatcttcgttgataaagaatatgcgaaagttcttgaaaaggtatatgcggaagaggaagaacgtcttatcgaagaattacgtaagttccctgctgttaaagagttagaagcagagcatatggcactttataaagcaggtctagaagaatggaagaaacccaagtcagaacgagacgaagaggttgctaaacttcgtgataaatataagatttctgataaggaaaacaaagttaagttcaatcccggctctgctgtccataaaggaaaagttctttataagattcttggactaacattaccttatgacaaagagtccattaaagaaaaaccttttgataatggtgttcctgaaaacgagctaacttgggaagactataagacagataaacatgctttgggctatatcgcagagcacgatgaagatgcaaaagaattagcagaaatgctactagaatactctaaagtaaacaccttgaagaacaactttgctcagaaacttccgctactagcttcaaacaaagatggaaaagtacatggctcctttaagattactggtacagaaacaagccgtttatcttctaaagaacctaatatgcaacaagtaccatctaaagttggagaccctaaacgttttgactatacgtacccaattaagcgtatgtttaaaacaagctttgaaaacggagctttattacagctcgactactcagccctagaaatgcgtattctagctttagtagccggagacgaagcgatgacccaagctttcttagatggagaggacttacacaaatcaaccgcttctattgtttggaaagtacctgtatcagaagtatctaaggatatgcgtaagaacgctaagagtgttaactttggtattgcttatggtgaaacacctttctctattgctcctaaattaggtgtaacaccagaagaagccgaacgtatctttgaagactactttgcaaacaaaccacgtattaaatcctttattgatgaaacacatgagtttgttaaagaaaacgggtatgtagataccttacaaggacaccgtagattgattcgtgatgcgttctctaaagaccgtaatacattcaatggtgctttacgtaagtcagtaaatacgattattcaaggaactggtgcatacttaacaaacatgtctctggtgtatattgacgattatctaagaactaaaggtatgaagtctcgtattgctattacagtacatgactccattgtaattgattgtcctcgtgaagaagtagacgaagtagcaaaagtagcttgttttatcatggaaaacctaccaattgatttcttaacaattaattggaaaggagaacaaatgagatttcctattgtagctgatgtagaaatcggagaaaactacaatgacatggttgactatgatgcagaagaagtgaacaaatttgcttcttataaaggatacgttaagtattacaaagaccaagctaaatttgaagactataagaacgcaggtatgattagtgaggaacaaatggaagccggtattaatgctgttaaagcttgcatagaacagtacaaactaattgcgtaagtcttacaaatttatttaaaaaaaattacaaaatgacgttgccctaacatgtaatgtgtgctataataatagagcagacaagttagggaatgtctgtaatattggaaaggagaataagacagtgtgctagacttacgagtgaacgaccttgaatttcgtaagatacgagttaccgatgaaaacggtgaaactattgtgtttgaccttacagaggaactacaggtaaacgagtttagtgttcggcaagcgttcttagaacagcctgcgaaatacacgtattggacttctttacttgaacgcttacgaatgtaccaagaaaactacgagttacaagctgagaagcgtaaagcagaactgtatgaaccttctagaaagtctttgattgagcaaggggtaactaagccaacaaaagaccaaatagaagcacagattatgcttgacgaacagtattatgctctgagacagaacataattagtttaacatttaatgttagacaagtgcaatatgtagtaaaagcctttgaacaaaggaaagacatgttgattcagtatggtgcagacttacggagggatttcgagtatagccagaaagtgtctatgccagacccactaaaaggtcaaaggacaagttataacaatccatataacacataaccttagaacctagttgaacaacacataattcgagaaaccaaaataaaaaaacaaaagaatggagaaattaacgtgaactttcaagaacaattaaaacaacaacttgcccaacaaaaccgaggagaacgtgccgaggtagactatccttccaatcatcttaaacacaaagagctatttattccaaaagaaactaatcaagtgttagttcgtatcttacctcctagtgtttctggtgaaaactataaccaaatggttcgtgaaatctttttacaagcacataaccgtaatggtaaagaactaaaattaaatgctgtcttatctgcattccctaacgtagaagacccattagaccaagcacttattaactggaatgcacaaaaccgtgttcctaacaaatggaacaaaaatgcaactccaactaagaagtatcttgctaatgttatccaattaattagagacccacaaacaggtcaattccgatatgaaacggatgaacaaggaaacttgatggtacgcttgttcaagctatctcattcagcttgccaagctattaatgctaagttaactgatactatgttaacaccaaactttaacccagatgttcctgattctgttaaacaatacagcttcatttcatctgcggaagcattcccagtaatgattagcaaacccgctaaaggttctggtcaaatgtcttacggtgttgatgtttatagtaacatgcctctaggagctttaccacaaggatgggaaactcaactagaagacttagcttatcaagctacaccttcatacagctacaataaagagtacattgattactttattgatgtagtaaacggtgttgaacctgtgagccaagcacaaggtggacaacaagtacaaccacaacaaggttaccaagctcctgcaatgggacaaggctttgcttctgctccacaacaacctgcttacaacatggcacaaccacagcaaccaatgaatactggttttaaccaacaacctgtagctccggttcaaccacaagctacaaacatggggactccttcatttggtcaaacaaatatgggaacagcacctacaatgaacggtggatttgcgcaagcaccacaacaacctgtacaacctgctccaattgcagaccctatgggttttcaaatgccaatgaatcaagcaccagtaactgacccatctactatttcagatgctgatatgctatttaacatgcaagcaatgccagatgtaacaccacaagcaacaagtgttcctgagcaaccagtagttgcaactcctgaaccaccacaacaagctcctgtagcacctgtagaaacagctacaaatactactttaccagatgttgatagcttgctacaacaaatgcaaggttagtaatagaggaacagggggctgaaatagccccttcttctatttaaatttgatatgtaattcattactaggaggaacaaatcttggcaagaaaaagacaaagtgaaacagtagaagcaggtacaattgatttaacaaaagaattgggcttaacttcttttagagatacaaaattttctagtgtagccgaccgactacctacaatgattcctcaattagactacatcttaggtggaggacttccatttggacgtatggtagaggttatcggaaagaactctagcggtaaatctacttttgcagtacaccttactaaaatcgctcaattattaaaggttccaacattgtggattgacgtagagggaactgctgaccctgaccgtttagctgaactgggagtaaatcctgatgcaggggatgtattcatggtagagcctgctttgaaaaaagacggtactaaggatacaatgacagtagaacgggtagcagaagagctacaacgtatcctaccagtaattaataagacaggtaaacctattttaattatctgggattccgtagcacagaccgccgctgaaaaggaaattgaacgtggtgtgggtaaccaacaaccgggtattaaagctaaagcaatggcacaattctcacaaatcattgcacctttaatgactaattcgaaagcattatttgttgcaattaaccaagctcgtgatgagatgggtagtatgttcggaggtattgattctccgggaggacatgcacttcaccactgggctagcttacgactagaagtccaaaaagcaagtaagattgaaaatagtgaaataaacgcctttggtgtagaagaaaaatcttatatcggacatatcatgcgtattaaaacgttaaaatctaaagtatccagaccacagcaaaaagctgaaatgtacttaatggctgattcaggtttagaccttgaagaaaacatctaccgtgcatgttttgcaactaataagcaatatggattcattagcggtggtgcatggaaagcttacacaacagatgcaggtcaagaaatcaagttcaattctgaaaagaattgggtagacttcttgaaatcagaagaagggcaacctgtacgtgatgaactatttgctaaaatgatggctcgtagtttccctaatggatacgcaccttataaaaatgaagagattgatatttgtcaaataccattatataaatacgttaaagattacatggaaactcataaagaagaagcacatcctgtagaagagcaaacagctcctgtatctgcaacagatgtttcagacttattagaccaaattaagtaaggggcgttatgcccctttttataagagatggaggaaacctaattgcgtaaaactacaccagttaacaccgtactagaagccttagaaatgtttcaacaaggaacagcaactacagaggttcgtacgcctatttctttacaatctgtacgtaaacgtttcttagatgctaaagaagcaggaaaacgtgtattgattgaaaactctgatttaagtagtgcaaactctgttgttgttgatattgactatgtgggtagccgttggtgcttaggttatcaaaaagtattatactttggagaagagatacgcattcctcatactatacacttttgcgatgtatatggggcttacggtaacgaagcacaaagaagtaaacggcaagtgaaaatcatttttgaaggagagaacccttttgagtagagaccttaacaaagaggaacgtgagattagaaacggaaatcgtttcattactgaggttcacggtaagggggtctttccacgagatgttgatagactgtaccaccgttatgccaacttacgttacaaggtctataacactcataaagactcttttcgaagtgaagcctcacgaaaagaactaagaagttatattgatgaacaatttatcaagttaacgaaagaatatgatattaacggagaagttgattttccgggatatatcaaaaaggctcttaatctaagagttagacatagctttgtgaaaggacgttttcgtgatagtgctcgtgaacgtctgggagcagaggacaacgagattgaaacattactgggaacagacacctcttctcaggaagcaattgaagatgctgaactaattgatgatttactatctaaagcgaagtttagccctgtagaactagctgtattcagctacttgctacgaggtaaggtaaaggatgctaatatcattaaagaggtgtcagcgaagtataaagtgtctcaaaagtccgttagagatgcaatcaaaaacgtgcgggagtacgttgcgttaaatcttaaagaccgatagaattagttaaaatcctccttgtgtgtatgctatattagcagtagaaacacaaaggaggatttttgtaatggaagaacaaaacaacactggcaaatacgctccatttattcgactaattgtaatgggtatctccttcgtggctactggtttaacaactatgtttggttgggaaccactaccttttacagacgaacaaatgaaccaaggtttaatgcttgtactttctgttggacttgctatctataactggtacaagaacaacgctgttactaagtacggtaaagctaaagagcaagcaggtaaagaagttgtcggaactcgacaagacttcaaacaacaaggctaagtttgagggggtaaaacccctccttagttgatattaaaggagtatgacaatggaactaacaaatttaagtgctaaaacactacccgaattaaacgggaagtttcttgttgtagagactctatcagacggatacacaggaaccgtaaacggtcattataactatgaagttgttactaaaggtgaggactatgaaatctatcccacactttggaacaacgaaaaagcgcagtttgtaaaagctgacgaaatggttgtgtatacaaacggagatactgtattctatgtaacaagaacaacaaaagacccctataaccatgctgttatcagtgagttaatcgttgaagaaggaatggataaggataagcgcacccttcaagctttccgactattcgcagacgatttattctctatcggaaactataacgtgttcttaacacaccaaattgatgtagtagacagcccaaataacgtaaaagttgaatcagtttccttagacaaagaatccggaacattatatgtgggtgacacagtagaattaacagcaactgtactaccagaaaatgccacagtcaaagacgtaacattctctgttgttccagaaggaattgctactgtaactgctactggtgccaaggcaacagttacagctagagtaaaaggttcaactaatgttatcgtaactacattagacggagataaaacagctcaatatgctttaacagtgaaggaacacgttttagtttctggtattactttaaacaaagaagctatttcaattgttaaaggtgctacagaaacacttgttgcaacagtaactccaccagatgcagaaaacaaagctgtaacatgggcatctgataaacctgccgtagctatagtagaccaaaacggggaggtcacagcggtagatggaggtacagctaacattactgcaacaacagttgacggtaagaaagtagcaacttgtgttgtaacagtaactgtgcctgtaacaggcgttacactagatacgaatgctattactttagaaattgacggtacacaaaaattagtagcaacagttgctcctgctaatgcaacaaacaaaaaagtaacatggaaatccgataaaccggaaattgctactgtagaccaagaaggtacagtgacaggtgttgcagacggtacagctaacgtaacagcagaaacagaagatggtaagaaagtagctacttgtgctgtaacagttaaccctgcgcaagcataatatactaggaaaagaggaatactaaaatggctaaagaaatcgcaaatattgcagacttattaaaaccaagcgtaaaaactgtcgaactagacggtaaatatcttgtagtacctaccctatcagatgggtatacaggtacagtacagggggcttacgcttatgaagtgaagaaatcaggaacagactacaaagtgagtgaacttatctatgataaagacaagaacacgtttactcctgcggatgctccaattatcatgacagatgatgatacaattttcttcgttactcgtacattgattgacccttacaactacccagtaattagtgaggctacagttaaggctcatgaaattaaagaaaaacaagtacttcaagcatttattgcttttgcagaagacagattcaaactaggggtatacaacgtattcttagcagatgacccttatatcttgcaagataaagcagaagactagtttgttctagtacaaaaaaactgtacaaaatttaaataaagtgttgacgacttacacctctcttggtatacttagactatactaaataagagaggtgttttattgtggttaaagctaaaaatcctgtacaaagacgtagaggaacttacaatagttgtgatgtgttagttgaagaagagaacggtttcttttactatagtgtacttaactcagatggtaaagaaatcataggtggctttagggataaaggctcccaagacatatctacattttacaactacttgcttaagttagtagatgactacgttaaatgtccagagggttttgttgaagatgtttaaacgtaagaaatactatgtagtccaactagttgttcaaccagatagtaagccttggtacaggtcaatacttgtccaaagagaagacgggtatctgtttacagaaggttacaacagaaaatgtcgggtagagccaatgtatacacgtcttggtatgaaatataaaaaagtttttactgaaaaagaaatatgtgcaatcaacccagattacatgtattttgcagtggaggtgaccaaggatggcagacctattaatgataactaaaaagaatgttcagggttatgtattaaactacttatttaagcaattagctgtaaaaagtgaacataactacacttgtgagtttttagataagtttaacttacctgatattaaaaattatgcgaataaagcagatgtactaattatttcaggcttccctttttatgagagccaacgtactactgtggatgaagcattacaggtaatgaacaatccgtttagtcaattctttcacttagcaacttttggggatacctatagcaacgagggcagttttcattcatatgtagatgaattagtaagccctgtaggtaacttccctaaaataatcaaagaactagagaacttcttgaaatttaatgatgtaattcggcacctgcttctaagtgatatggaaagtatcatcgaagcaaccgatgcgtataatcgttatgaggtaacagatactattctagactgggtttctcttgtagaaacgtacagagagcagttatacgcaagatacggagtaggtacagacttagaggaaatacttgataaggataaaaccttagtcaaagcattgcgggagaaaaaagacgattatatcaaaagaacacttggcagaacatctgcacaggtaatcaatggtacagttgtatgttttacttacgcagaggaatatgtaaatgaggtagcccataaactgattgagttttatcaatcatacaactatacaaaagtagctgtctttgttggacgacataccaaaggtgatgatatgttcagcattcgaacagcaggtatagatgcgggagaacttgcctataaggtaaacaggggtagaggaaaacctactactgcgactgttttcttaggtaattcaggtcaatccacattcaacgcattacttaacacacttgctcaaattgtataacaagtaagtgtgttataataatgttaggtataaaacaatacacagggagtgtgctacttgaaaaagaataaaatgctagaagacctaatgaaacaattagataacccatacgtaattggggcaattattgcaggaactttgcaacatgactttaaccgtatacacgttagcaaatataataaatatgctcgtgagtttggtttagaccgagagaacaaagacagcttagaagcactaattgaacatttcgattctgataagttattaaaggaattgtacatgtcaagtattgcaggagaaatcaatgtggacatgcttcgttcatttaaagacaaaacgaaaaaatctgcattccaaactttatctccattcgtggtagataaccaagctgttttagctcgtaatcgtgagttcaacaagattcaacgtgagggtgcttacttagaccacttaatcgaaggcttgaaagagcacttatccgaagaacttgaaaatactaatacacttaaatatattgaaccagaattacgagtaaaaggtaagccaagtgaacgtgaaatgattctatgtttatctgactggcatattggagctttcgttaacaatattgatacaggtggatacaactacgatattttccgtgaacggttaaacagtctattgtcagaagctttccgtattgtgaaagaacaaaacattcgaacagtacacgtttaccatattggagacattattgaacatatcaatatgcgtaatgtaaaccaagcgtttgaagcagagtttccggcaacagaacaaattgctaaagggattcgagtattagttgaaacactaaatgtcctagctaaagcgggtctagatgtaacctttggtatggtaggtggtaaccacgaccgattccaaggaaataaaaatgataaaattcataatgataacgttgcttacttagttgtagaccagttacactttatgcaagacttagggtctctacacaaaaatgtaactttaattgataacagacaagacgtttacagctttaaagacactgtagcgggtaaacgcattaaagttgttcatggagacaatgaaggtaagaaagtagacgttaagattccaaaacatattaaagaagaagttattgaatacttaatcatgggacatatccatacaacacgtatcattcaagaagattattcacgattccatgtatacgttgggtctcctatgggtgcaaataactattcagcagaaaacaatctaccaacaacaagtccggcacaattaattatggtcttagaccctaaagtggatagtccacaatttatgccaatctttttgtaggagagtgatagaatgaacctgatttactttttattatctatcatgtatgtattcgagtttggacaagcattcttgaatacgaaacgaaaagagcgatatatgattgaagatggtggagaacctcttcctagaagctcttacgtgttcttattcattcaatatgtattgcgtactcttttcgcaatcttgcttatttttattgtaccagagagcctacagttcaacgtaggaggtattactgcctttatagtagttttgcttatcattccatttatctctcggttcattgaagttggtattcgagtattgattattaaatacttgcaagccaagcacataaaacgtattaaggaacaaggggacgaaaaagagactaattagagtctcttttttgttatgttatattaagtattgaggtgaaaagaatgaaatatacagaaatccaagtaggtgacggacaaagcaccttattagatgtaaaaagcaaacctgccttaattcgtagaggcacattaacaatgaaagttaaatcaagagatagtctaatcagtaaggtagaagagaaacctgtatatattgttgagctagcagaagaatcctatggtacaagcgttgtttctgtttttagtgcagtacaggagggaagcggagaccatgtagttgttcagaaggcttacacagagaatattacgcaaccatcgtttaaaggcaccacatttttaggtaaggttgctcaaaaggttcatgaaaagagcttaaaaccaaaacgggtaatcacaactaaaccccctgtttttctagcgcctgttgtcatgggagtagacacctttacacaggagcatggactagggttttatgaacgagaaccagataaatttgttatgaaagacgggcatcctagtattgagtatggaaagaacacaggagtctttatttgcttaaattctattaagtgggataaagcttatgtcaatgcaggacaactgattcaagactatgtagcagacaaacaactatggtttaatctgggaggaactaaaccacaattcaagagtgaagttaatcaatgaccgataatcgtttctatgaggcagacatacaagagttaatcttgaacaaaaaacgtatttttggagacattggggaaagtgctattgtatttgagaaagcagttatgagagggaataccatttgtgattgtttaatctttacagaaaaacgaggcgttattggtattgaaatcaagacagaacgtgattcaactaaacgtttgaataagcaacttaaggactatgaaaaagtctgtgattttgtttatgttctttgccatgatacacatgtaccaaaagtggaacagattttaaatagatataatcatagacatgttggtattttagcttatacggagttcatgggtgaagctatgttaggaatatacaaagaagcaaccccttcaccaagtaagagtgcttatcatatgctagacattctttggaaagaagaaattctaaaaatgttaggtacgttcagacattacggtattcgagtagagactgctacaggaaccaaatttatgcacacagctaaccgtagcggaggtgtgagtggtctttatacgaagtctgcgaccggtaggacgatgcgaaagccagacttgataagaaaccttataaatcgtgtagggggtgctgaggaggctactagggtcttctgtgacgtgttcattcataatagacagcatccagagaaagctattaagttaagacactttaaacagcaaccaataaggagtgataacaatgagtaaacaaggattgccttttggtacttgggagaactccgcaggaagaaactacacagggttaggtaatacagataatggacataataagaaacgaatgtccacaagaggatattatcacgtagctctgacaaaagagtataggggactaacagagagagactttaaactaaaacttgaatatgggaaagaccttgtttctcaatatacaggtgtaccgattgatatgcttgttcttcgaaaaaaagcagaaaaacaaacactttcgtctatagatagtgtatactatgtttgtcgaggaaaagaacctatcggtaagctgtctgtaagggcacagagacggtttaaagaccttggactcacatttgtatacctagagaagaactacgcccctaaagtggtgagaagggtgtctaagggacagcgtagccttctaggacaaacgaaatcagataaacgtaaagcaaaccgaaaacaaaagaagccaaaaacaacaaattaggaggacaatcatgctaagtaaggggaaacaagttacacttagaaagttaaacaatattattacaaataaggagtacatccttataggtgctcgttacacgaacgattctttagaacgagatattcctattacaactgtaattgacgatatacttatagatttgaagaaagagacaatggcaggttgtgttttctttagaccagtaggagatgcacaaagaaatgcgctatctctattaaaagatgcgtgtatgcactttggttacggagaatttttagatgaagctattgaacgtcctgcattgtatgcggggctaaaagcaaacgttatgctacctgacacagctttctcaaatatccaagaaattccattcaatttttatttcacagaaccagaagtattttgtgaagaagtaactatcaagggaagaaaacatattcagtacaccatcttagacagaatcagtttagatgatgtacaacatgtagaacaatcccttgttgttcttttccgtaaagcagagtatgatggagaaaccctgttaggtgcttacacggatacaaaattaacaaagacagaagcaatcaaaatccttcaatttgttagtaacggttccttacttgaacaggtagttggggcagttactgtattctcagataaagtagtgtctacacttattcctttgtttaatgatgtagatataagctttatgaaatcaggagaaggaaatcctattgtctctattacagcagatacaggaggcattgcctttgatgaaaaagatttggatggtatgtctattgaatctcctagtttcggagcttaccgtcttgtgttacatgtagggaaagacaccgtaagtattttaattggataaatttttttgtaagggagtcaaattatatattgactcccttatttttgtgtgctaagataagttcagttgttgtacaaggttacaatgacaacatgtacaggagggaaagaaatgagtgtagcatcaagtttagatagagctgatgaatggttgttgtcgcatttgtgtgataagcgtttagagtatttgtctcaaaaagaagagttaactgcaaatgaaaaatcagaaatattactaattcaagagcttaatatgaaactaatctgtaataccttacaaaaagaaacacctgttcaaaccgtacagggttctgacgttactagtggcttattaagcaaagcttacacaaactctgttaatattacatcgcaagaagcgtggtatacatctttgttgaaacgtacgcttgatgttgttgatggaataccttgtgtagggaatgttactgtatttgtggactcagatgagattagtatttcgattatgggacatgtacgacacgataatgatttagcctgtgatgtctacgaaatttcttttgaaaacctcggaggtatcgaagattttctaattcatgttgagggagtagaaatcgctttaccagactatttagagtttattaccgaggaactacccgccattttaaaaaaatcattataaggagagagaataatggatttaaaacaagcttacaactatgcagtagaacatttaaatgaaaatgccaaaacagaaagtgaacgtatctatgtattgggtgtcttccaactaggaagccgatatgccggatacgatattgaatcaagtgattacgattttacagtcgtttacatgccgtctgcttatgatttattaaaccgtgagtatggacgtagcaaaattaagtttgaatacaatgattacgaagtagaaatgtctttcatggatattcgtaaatacctaaatcgtttattatcaagctcactagaaacattacaaatggtgtttgcaccatctagcatgagctactatgctaaatttggagacgataatttatcattacgagcagaagaacttgtagattttatgctaaaattacgagacaatcgtaaatcctttgtctatttgaacacagataagctatatgattcaatttcaggaagagcaaagtcttctatgggtaatgctagcattagttacacaaatgatgaatatggtcgaacagtcaagtatgcgattggcgttgaatacatgcaagacctacttcaagcattatactacaatgaagacattcgagaaggtttaactgtttcacctatgcaagcacctgtttataaggaacatcgaaacaatcctagctatgaaattgctcgtttgtaccattctcgtgctgttaaccttattgagaaaatggtcttaaacaaagaagtggttcgtgaaactttacgaaaattacagccgaccgaagatacaattaaggtagtaagagaagtatggacagaagaatttgtaaacattatcttgggaggttatgacgactaatgatgacaacagcagtaagagaaagaacattagatgaaatcttaaccaatcctaagaaaccaatgacagagaaactggttgaagctgaacaactaatcaagaacagtgagcatgtttatgactttgggttaggtacaattagagaaactatggtgatgtctggaaaaaactggttagctagtcacttatatatcaaagaagttactacttatggatttatcttgcaagccgttgtatacaaacagtccttagttgagcgggctattactaacgatgaaggtgaagaaataaaacccgcttcttatgactatgagctaaaccgtgtctacgaattagagtacagtctacgagataagtatactagactacttaaaaatggtagtcccctgaaattctcagagaacaacatcaaaaacatgttttctggtcaagaaggtgttattcaaaaattagattcatgctgtaacaccgagatgtattcttctatgtatgacgtgtttagttgtattgggaatgaatcagtaccccaagtagctcgtttccttgttagatttatgcaattgaacgtggttgaactaatctataaaagtggggttcctattccttttgctaaagactttgctagagaccaagcaaggaaacttgaaaaaggtaatggttggtatggtagtccagagcctaatgctaactgggtagattgcacagctactgcacctgcaaaggttcttgatattccaaagtctttatttaaaattatttgtcaaggaaatgttagtttcagtagatatgtttcaatgcacagaactatagaaagagctgaaagtacgggttttgaagaggaacctcgtgaggagtggaacagggaactacgaatgtatattaaacccgaagaagacgaagctattgtacaaaaaaggaagaagtacaatgagaaatgtctaaaacgaaataaacagggaatcattcgagcttgtggtgcactgtatcatttgttcacctttgctgtggaactagatagtgaatatggaattgaaaaagctaataatttagtttctcatgagttcgaagatatgttatacagggaagtagtgcctgcccgtgtaaacagcctacagaataactcagcagtttgtattgccttttctttatgattggagttaaatcgtgtcgtcagatacatgtactatcaattacatgtagaacaaggatttactgatggatattataatgcaacttaccgtgactacctacgtatgatgcgacaactagagattgcggataaaggtaactatcctaaatcattgaaaacagtgcatgatgtagctgaaatgaatactagaatgattaaggataaagaactggcagataactttgcagaacaagtcaaacattaccgagagtgggaagacgttaaagttcgtggtagcaagtatattatgcgtgcacctgagagtatagatgaccttgccaaagaaggttctagtctaagtcactgtgtattcagttatgctcgttcagtagccctaggagaaactacgattatgttcttacgtactaaagcttgtccagatgatagcttggttacagttgagattaagtacagcccgagtcgagaatgctatagaattgtacaagctcgtggtcattccaatcgagcattaacaactgcggagaacgagttcttagagaaatggtgtaaaaaatcaggaaatgtagagagaaaataggctattttaaaatgaaaaaatactactagttagttcattttcttaagttagctaaaacgattcatttcaaataacaattttccacctacaaatgtttcatatataattcacagaaaaaacacaaaaaaccctacagttatgtggggttttcttgcgtttcatttatatctgaaatttaacagttatgtgtgctatattaacagtagaacctaagaagggcgggctttatccgtgttcttagaatatagacatgttgaaggagtttgtatttgaaatggctaaaaaagacttaaataacagttcagtattgctgaacctatatcataataaattattggtatccaaggttgatgaagccctagacgagggcaaaccttatgattttattattgctttttgtaaagagaaatttgactttgaaatcagtaagcctgcattatctcggtataaagaaaaacgcagagaagcaatcgaacagggtgtagaccttgaatctctacttgacaaaagacgaaaatctggtaaagttatagacctaaagggaaaagaggttgaaactctaccaaacacgaacacgtcttatgaccaaacatttaatgcagtagagcagatttataatgacgttgaagtgttagacactatcattcaaaaaggctttgctagcttgaaagaagtcgattatgtagaagcacctcttgccatgaaagctatcgaggttaaagcaaaagttacaggtaatcaactgcaagggttaagtctagtgggattgcgtgaactaagattacgtcagtctgctaaagaacaagcgatgaccgaagttatcttacgtttcatcccagaggaacaacacgaagaagtttaccaagcaattgaagaagcagaagcagagttctatgaaaacttagacctaacagcagaagatgcacgtatcactagcgcactcgaacaagcgggcatgaacattatctaagggggctattcaaatggcagaaaatctagtggaagtagactacagagggctgactttacaagaagcactccaagcgctactagacaaaaaagtattgctagtcaaaggactagaacaaagtcggaatttagatgtattgatacgtctttacaccgaaggagtcgtacctgttactcaaatcagttatgatgttgttcctgctgatggacattggagagaagaatactggcagatatttgatatatctgttaatgctctaagtacgtatccgtgttacgtgtatgatgctgaaacggctaataaagcacctaagttcctgattggaacaacagtttattacaccagtaagatggatagcactcgagattccgctatcgttattggattgtacaaagacgatgagggcaattggcattacaaattaagtagagaccaagaaatttacgcagagacagagcttacagcagacaggttgtaagcctgtctttttttgtaattaaagtattgacaataaacagtaagcatgatatacttatggtataaattaataaggagagattcatatgaaagaagaaaagacttatactttagaggaaattagagaggaaattagggaaaaagaaagtaaagctaaagaattagaagagttgcttgcaaaactaccagaagatgtcttatgtaaagaatatgatgcaatgacatacgacatgttttatgcttgggaaaatgattcttgtggacaagtgcgggagttattgagtaaacatgagtataacttggactcaattcaattggttcatgacgtattctatacactgaacaatttggtagaaggggaaatagaatatggaaaataagtagttgacagtactagtacaagatgttaacatgtttatgtacaagaaaacaaatcaaggagagatgtacaaatgaaaccattcgaagacatttttgatgtgttaaacaaagcacgtcaagacaaagacaaggtaacacttacttttattgaaggactagataatattgctaaacttcaaattgaggattcttctgctacagcaggaatcactcgttatgtaaagttctacaacaatgacatccgtaagttccagtacgttgaccaagctgactttgaagaaatgtatgcaggcttagaaatgaaaacaggtaaaccaaaagaagaaacaattgagtttccttctgaggaagtaggaattatcagtgctttaaaaatcttgcaacaaagtggtgtctttagtgatgaagacggaatgcgaactctaggaattaacacgtctacattgaaagatgctcactatatcttacagtatattgagcctgcaattgaccgttacttcgaatctttgaataaggatattgaagaattagaccgtgtaatgattaaccaatcagatgaacttaaacaaaaaatctgggaaagctttgtatttagcaaagataatctaagcaaaatgctagatgactttgaattggcaactctagacacgttcccaaatgtaccgtctaatttgattgttgagacttgttcagaagtgaagaaaactcttgaagggttattgattgaagatgcggaagaaaatcctattgagtgtcatgattaccttgttcgtggtttcgttggtgctgttatccgtattgaacaatttattatttctgttgcaacattatcttaccgtagaacagaattagaacttagagaatcaagagaagcttatatcaaagagcttgcttcaaatctatttatctttgcagaagctattgctcaggcgtatactagacttgtatgttctatcttaatggaaaattctattgatatgcacaatgcttcggagtatatccaagttatggaaacaaaagcggttcactgggtaaaagaagaagcactaggtggaactatgttagctaacaatgaggaagacctcgttgagttatcccaatctttaaactctatgacactagatgacgtacgtgaaacccaagaagaccctgaaaaggctgagtttaaagaagctttggaagaactgcttaatgagttgataggtgttttttcagaagacgaggacgaagaagaagaagttgacgaaacagaagacgaagatgaggaagaaataaaacaagccttgttagaatcattagaagcatttgaagacctgttacgttcaattcaagcccctactatggaagaagaggaagaagacaacgaggtacctttcgaaacacctatggtagacatggatgacctctttggacacttattatcaggggaacctaaagaaaaagaagaacctgtggagctttcagacgatgcaaaagcagagtttgaggaatactttgcagatgaaatcggtgagttacaagaacttcgtgaaaaagggaaagatgctgtagccttgtataataagattaatggggaactaccgttaaccgagataaaatcaatggctatgaaaaatatgggatttgcactgtcaactctgcaaaaaatgttagatagaacagagcttgactacactaagaggggtctacttgattccttagatagattaaaaggaagcaaatacaatttatttgtagaatatattgagtttttgagagaagcatataagttgtataaaacagtaggcatcggtattgttgcttacgcacatgcaattgttgaagatatagaaaatacggttgataacgtgatggacattgactatgaaactgcttaccttgttaagcttgcagttaaacagattaatcaagacattaaagcctttgaagaaaatgatggatactctgatttattagccttctctttagcatttacactcgttgttatcatgagagactatggagaaaccgacttaagtccaattagtgtagaggtagaagactttgtgtctaataaaaagccttccgttgttgtcgaagacgaagacgaggaagaaaaagaaggagctattgacttagctaaagagttgaaagacttgattcgtgggactgcattagacacagacaattctttaaacaatagtgtagacgaagaagccctgattactgattataagaatggtgtctctcaaagagaactcacaaaatgttataatataagtacagggaagttctactcaattctacgtagacatggtatcgcagtagacagctctaaagtagctaaaaaggttgcgcatgtagaagaaaacccggaagtactcgaacaggttcttagagattatatcgaaggtaaaacattagcttacatttactcaaactataatctatacaaaaacggtttgttctatctactagacaagtaccaaattccacgtagaaacaaacgttaacttccaaataactttaggcagtggctactatggtcactgcctttaataaggagaatatttgttggaagagacggtttataattttatacaaggcttaagcgggacacaattaactatattagaagatacatttgctttactaggactaatctttatacttataggaatacattttttagtagcagatacagttgaatgggtgaaacttgttaggaatccaagagtatctacatttgcaacaatacacatgtattttactttttatctaccgatgtttattttctttttggtagatggtagtacggttactacttttgttgtttcattactgatgtttgctattgtgtatacatggattgtctggcttcgagtgggtaggtacacagaaaactggtcagtagtacgtccatttgttacctatattaaagcacaagaccatgtatgtacgctaattattggttcagcacttttaagaacagtactattttttataaagattaggataggttaagaataggataggatggaggataagatatatgaatgaaataatgagttatgtttggttgggtgtgtttattgctatacccctaattggctttatctacgtatataactacaatagaaaaaacccacgttggtaccttcgcaaaagctattgggcgccactagtgctagatttagttgtttttggtgtttgggcaatggtaggcgtaattacactagctgttggacatacagaaactactactaaaacatttaatattgaaccacttacggtttataattcaccagaaaatatttatgcaaagataggtagcacggatgacagctatgttaactttgcaattacaggtaaaggaactcactctgcaccacttggtaacactagtatccattacgttgagtccgaaggtagaccgaaagtcacggttacttactacaaattagactcctctttctcagcctttatggggatgcagggagaacttgtaactaaaaaaactcgtgtagacattcaagtacctgtaggtggagtggatgttacaaatacaggtagtacctttgaagagcaatcagtaaatgatttacttagataattccctaagtagcgggttacaaaaatccgctaagtagcaggctagagacatcttttataggtgtctcttttttattgacaaacctgtacagctatgctaccatatacatgtaaataaaatgagctaaaaaatttgagcttacctattgacattaaacaatgtacaggctataataagtttgcaagataaaacgaaggaggaatttacaatggaaaaacaaaaacaagtaattacaagaaaacaatatgaggagattatggcacgttataacaaaatagactatgctatcattgcaggttttgttatctttggcacagtcatctttggttttgggggatttgtatttatggctattgttggtgcgtttacgttcccacgtatgaaacaaaaaagcattaatttaatggaacaagactacattgttagagatgacattcaacagtaagcttaattgttgactatggaaattacaggtgttactataatggtacaaggaggacaaagaaatggcagaagcaaaaaagagctattctgacatgacagaggaggaagcacaatatgcaatggtattaggtgctagaatcgctcatgcacgagaagggttaggtattagtcaaggagaactaggtaagttagcaggtttcaatcaaacaactatctctttaattgagacaggtaaacggctacctaacttaaagacccttatcagattgagtaagattcttaaaaaaagtttgaaaattacattggaatgaggtagagaaaataaaacagggggagatttattatgacaacgaaagtaaaagaaacaaaatcaaaagtaaagaatggtgaacgtacaccgaaggaatttgtagcagggatgaaacgtagtaagaaagacttgtcacaaacgcaagtggtaacttctttaatggaaaattatccagaacttacaccaaaagacatccaagaaattacaggcattaaacgtggaactatctatacaatccaaagtcgtctacgtagtgctcatcgtaaggaacatacaaagaccgcagatacgccacatatggataaagtagagtctattagtgaaaccatgagtccggaagctattggtaaacaagacttattcaatcgtttagctgatgtatacttaaagaagattgaagaaaccgaagatattgaagaaaccgaagatattgaagaagctagcaaatacaacacgttacttattcaagtgatggaaagcttagataaatgatagcaggattctatttatataatttaagagtcctgattctaccgtatatttaacaggaatgtgcggtagaattttatttacataaacctattgacaataaacaatagataggctattatatacatgtaagataaatacggggagatgtttaaatgcaaacagcaactaaaaattttgaagttatacgtaagacagatgatttaatttattatcactttgcaaaggaaagtagctacgctatcagtaaggtagataaacaagagtgggaagaggcaatgactgaatatgtgaatgattttcttgaacaggcttacggaaacatcccacctatttcagtgtatatcaactctaggctttctagaacattaggttggtttcaatattatttgaaagacaacggtaagcctccgtatattgaagtaagtagcaggctagtaaaaagtgcgattctgctaacagatactgagcacaaagaacttgcatacacagtaattaacgacctgttaagacatgaagctattcactatgcgttgcacgtactagataaaccttctagtgatggagacgatacttttgagaaagacttgtttgtaacagggacatctcctagtctaagtacaccagagtataaagtatataaaaacgagtttcctagactactatgtaaatactatgcaaagtgccctgtatgcgatactgtgcttgcttatggaacaagaggacgttactattgtattcatagatgtgtagaagaggggaaaacacatccagtcatctttagacctaagaatgaattaatcattggggtacagccccacgtatcttacgggagaaaactactggaaggaacatatgatggagcagttaaagagtcttacagaaaggggataattagctaatggaagttaaaatagacttaacatcgggtacagctacagttatttttaatgatagtcctacagcaatagtagaacaataccgtcattctatctcatatgatgaagtaagtaattaccgtaaaagggggcatacagtttactacgctaaccctgagtatcgagacccctacgtacttaaagataaagttgtaggttccaattggaaagaatacattgatagcatgaatgaattgcgggaacaaattactctagcagagaacataggtattacagtcacaaataaggaaagctatgaaaaagtatacaaacgagctttaaaacagtatgcacagttactagaaaaaggaaagcaagaaaataaacacagacgtttaattaagttactctcttctcaggactacgaaactttagtaactttaatagaaacagcttcttggcatctgttagaccactacgagtacgaacatacaccaatcccaccaaaggagcaagctgttattgacataaaaactaagtgggatacaataagtaggatagagggataatgtttccctctaaaatttaaacatatctattgacaataaacaatagaagagttatactatgtatgaagataaggaaagggagagatgaaaatgttaaa

SEQ ID NO: 2 - DNA, 130952 bp, Bacteriophage
aacatggattttgataaaatttataaagatttagtaatggatattattaagaacggagtaacagaattaccagaagggactcgtgcagtatatgctgatgggactcctgcaacaactaagtttattgaaggtgtaaactttacaatcacaccagatatgggggctcctttattacgtagtaagcatgtaggtattaaatgggcgctaacggaattgcagtggatttggcaagaaatgtctaatgaggtttcgtggttaaaagaacgaggtgtaactatttgggacgaatgggagcaagaagatggcactattggaaaagcgtatggttacgcattatttaacaaagaacgttttattcctgtttatagtggagatgttgagttagctaaaaatcgcaaagcaggttcacttattccaaaagtcgtacctaaaaaaggtggttggatagcaggaacacatcaaccatacctaaagctaaatcaggtagaggcagttattgaacaacttaagagcacaccaaactctcgtagaattatgactactttatggaacgtggaagatttatacgatatgtcattagaaccatgtgtatggacaacccactggacagttgtagagggtaaattaaacttacatgtgaaggctcggtcttcggatgtttgcttaggacttccatttaatattattcagtatcatgcattacaattggttatggcaaatactgtagggttagagttaggtaacttatattggacgagtgataacgcacacatttacgaacgacatttagagctagtagagaaacaggtaaacgcaccaattccagaagaggttccaaagcttgtcctaccatggaagaaggggacgaacttctttgattccccattatcagatatttctgtagtaaattataaacacaatggaaaatttaactatgaggtggcaatttaatggcaaataaagtaagtttagagaacctattagcgatggcagtgactgcaaaagagcatgagatgtctcgtcatgctaacaaaatgagacaacttgagaagacagaagcaaatatcaagaaacgaattaaggagctaagcaaggagggctagtctatgggacttactaaaattgaaacaagagagtggtacaatagtgacctagacaaactacacaaagaaggtagagacatctatgtaaaactaaaaataggtaagagactgacctatggagagtatgaacgagaaccaaataatgggtttcaagtaattaataatgaagacaccctcccattaatcttagataactatgaggtttatacagagactactactgagctaactgagaaacagctaattgtattagtctggctaatccactatcttgaacgtacaaagctaactattgcagatagcttacacaatttaatatctggtaaggctaacgatgcgacacttcaagtttgggcagacctctctagtgtagaggaggcagaggttattagagaactggtagactacgtaatagataaaggaggtagcctatgagtaaggagttaatatcatgcggggcgtgtaatgaagtgttcagtgagatggacgaggttgtgcggttaacggacgattcgatgtatcataaggactgtgtaaccctctactcaactggatactgtgcattcttagatgatgagtacctaggtgacactgaaaatggagagggagaacctgcttgctttttcttagaagaaggggaatatatagaagaggaggaatagagatgttttataaaataattgaattaatggctgagcttaccaaagaaattaaagaaataaaagacacgttaggttgttcaatgcctatagcagaagaccgtaaagtgacaagaattgcatacttatctgaacagctagatgatttaaacagtttactattagatattccagtaaactaccatgagacatggttattagatgaaccattaacacagaatactccatatgagtttcatatagaagtaatggtgttaccttatggtgaaatgggagtagacgttaaatgttattatgaagaggtagcttcaagtatttcactaggggtagacgaatttcgtacactaccagatacggtagatgaactacaagagcgtactttccacaactttcatggactttcccgagaggaggctataaacaaatggagcttatgatagaaggtgttagccatggggagttttccaaacagtttatcaaggtaagccagacaacggaagctactgtagatattgctgtaataaatcgtacagtagaggtatttagaccggactgtgtacatgagcaaacagtaactctgacaaaggaaaatttagatttactggtgacagcgataggctatactaaagaaaggatgagcaatcatgggtaaacagatattaaaaggttgggtaactattgataacagccccggtgcagaaacaaacgaaccttacttaataaaagaaaaagtaaaagaaattgagtatcaccatgagcacagagccttaaacatgcttatttgtaatgcactacatgcgttaggggctgaggacactacaacagaagaggagtacgatggaggcatttcttctaagtatcatcttgataacgcagagattcagattttctctgctgtagaacgtacttcattagaagacatcactaaaaatatagtacttaaatctatgggggtcttggactttgaagaaggttggtacggttggtcttcctttactatcgaagggtttgagacaaacacatttaagctaggcggtcatgacatactaaacattgttaggcagttagaaggtaaatatgtttatatcactattgacaaagtataacaaaggaggtaggttatgaataacgaggtagaagtgccaaaacaggtaaaagagtttattgataaacaacgagaacagcttatggataagttacaaattatcttggagggacagagatactcatgtgagtaccctaagtcagagtttagtaaatggtttttcgcaaatacagatacttttattcaagcagtggcaaacactgctagtttacctaagtactatgtagtaatgcctgagaaacagaagagagactacgggttactattacgcaaggacgggaagttagcagtaagtatactgcctagaaacgagccattgagtcaagataccctatctaaattaaaagactactatttaacagaggcagagataaaagaaattgatgaacggtattggttatttaagaaaacacgagaggagctttcgtatgaattacagaattgtatcatatgagaacagtcaggaatggctagacgctttcaatgaatttagcggaaatagctttaagatgaatgaagccatccacccagatatatatgaacctattaaaactgcagtattgctctttaaccacgaagtggcagaaggagaggctattcgtttagagaaaatagacgaatcagaagaagatacgtgtgaccatacttatgagacatatgcagtacaaacaggttactcttcaatagatattgaacaagcaggcttttgtacaaagtgtgggcacgatacacataaaggctataaagagtacacattaaaataggaggactactatgacaattattgactatggaagtaaaccactaaacaactgctcgaaaaatgaactgttagacattatccagtctcaactagagaacgaagtaaaacttattaaactgattgaacgtactacaaaagaggtagaggaagagaaggaagtccctcattacaagaactatatctcccctgagaaacgtagctacttagagggcatggaacgtgtagaaaacattgtaagaaacttctatggttttaacaatgaataaaccagtaagagcaaccagtgagcaagtagtataccgagaagagtacgctaatggatgggcagtagaggtaattcattctttaaaaggattcgggcatatttatgtagagaacaaaccctattcagtggctattatgaaacctgcagaaaactttgcggactatagcacagtgggcacagcagaagaagtactgaaacttattgaggaagtagtagaatatgaggaggacatgtgatgaatgaactagacggaattagtaaattagaagctgatattattaaaactactcagctactagaaacacaacaagaactaaaaaagaactttatccgtaacgaaaaattgtctaaacggaactatgagttgtctatcgatgtgttaaaactaagaaacactttattagatgttatggaggacttagataatttgatggaaaatgtagaaggcatcaaggaggatgcgaataaacatagcaaagtagaaaaagaaatggttaaaggttggcttgaacggactggtaagttagctaacgaaaccagaaagaatattgaagaggtactagaaagttatgagaattagaaaacatgcaaataaaataatcgggtggctatttatttttttagcggtattgacaattgttaaagttatagtacttggaaagccacttgacggatttgatattgtactatatgtactactacagttactatatggtatggaaaagttgtcaaacgatgaataggagtagatttttctacttctttttttgcctgttgacagaatcataatagtagggtaatattatatcatagaaaaggggaggaaaaacaatgattagaaaacaactattatttgagttagaagtagaagttgacgaaccaatggttggacgtaacggatatagaactgtaatgcaaacaacaaggttatacgcatattcacttgatgatgcttaccacgcatttagagagaatcacagatatataatcaagtcaatcaaagaagtggaggaagaaataaatgacgaaattaagctcagcatggttagtgaaatttgagtatagagcggtaaaaatgagggctttcacctcatatgataaagcagtcacatattataatgaaatggtacagaatattgatgaacaaattgaagaaagctactacgagacaaaaacaacatacacagatgaccacaaaccacttctaacacaatttatatactggaatgaagaccgtactttccgcaatatacttggctttgtatcaataaaagaaattgaattggaggaaaattagaatgacagtaaaaattagatgtacacttaaaacagagcatatattcacagaaggtaaagagtacgatgtattctatgatgatgctagaggttacttccttagagatgatgagggagacattatagaatcaggagccactcctgagctactattagctgaattaaataactattggcattctaagtttgaattgctcggtgcagaaccaaagaaacttacactctatctcggaattgataaggggacaggtactaacatggcagttggtacatcccttcaaggagtactagaaaaagtaaaagaactagatatatacttctcaagtgactcagatttctatgcaaactataaaagccaatacatcattgaggaatgggagacctactaaaatgacatacagaatgaaatgtgtaattacaaacccacaagaaccctttttcacagaaggcaaagagtataggattgctcacaacaatactatagggtactttattaaagatgatgatgggacaactgcagaatctggaaaaacacgtggtgagctactgaataaattaaatgaatactggtattctcagtttgaactgattgagattgaaccgaagcagccaattaatgaatgcaccgagattagaaaaaaatttcaacaaacccttattcaatataatatggatattggacacttagaggggcgtttggtgggaaaaccaagtaatatcatatcaacacagattgcaatgttaaaagtaaagcgtgaagcagttcaagaattatacgatgagctgttcaaggaggatagctaaatggtactaatcttagcagtattctggttacttgtgataggcggtatcttactattattaattgatggagggaagttataatggttaaactaggacatttattacaacatattgaatttggtcgcacagtcacattagtagagcacatgacggattaagaaattgtaacattcactttatcagaaacaaaaccatttattgaacaatacgaaggtatcggagaacacttagacttagaagttatgctgatttctacaacatcaagtgcagggctatatcttgaaatcatgtaaaggaggcagaacaatgaaactagtaacttgcccagaatgtggcggaaatattttagagggagcacctaacgaatatggatttgaatgtgacacttgtccttacccatacaaagaggaggacttaatttgaattacttatactatgtaattatccttacaatggctcatgcagtaggggactatgccttacaaagtgactatatcgcaaaaggaaagcagactgatttatatctactaatcatccatgtaaacatctggacgtatattattgtcgcaactacattgttcttaggaacaccagtaaaacttgggctaattgtagtatgcttatgggttccacattttatcatggactatttaaaagcacaaagtgcatggttcttaaaaacagtacctaataaaaagacacaactaattatcgaccaaacagtacactatctacaactagctgtattcgtatggtttgcaacacactaaggaggagcaagtatggggagagaatatgtaatatctgatatgcacttcttccatagaaacatttgtggggaagcctcttttatggacacacggaaacaatttaaagatgtggaagagatgaacgagtatctaatcaatgagtggaacactacagtacgtccagaagacgttgtataccacctaggggacttcgcaatgggtgccaaggtaaatagtatagctaacgtcttagagcgcttaaatggcactatatggctaataaagggtaaccacgataatagtcctctacgaaaagagattaaacgcaatacttccctgaatgatagggttcattgggaagacgttggtattattttaaaacgaatgcataaggttgtgcatatgacacattacccactaattttaggagatagaggtaacctaattaatctacatggacacattcatgagttagccagacccgaaccaaacttactgaatgtaggggtagactcaccagaattagataaccataaattaggaaaacctcttttattagaagatgcaataagattagtaaacaaaaaacaagtaaactttaacaaagcaagtagagcatactctattcatggtgacttacaatgaaacctgactactcaagtataataggtaaaagatttataccaaagaaaagataccctaggatacttatgaaagaagtttatcccacaagagtattctatgatttcccaagagtacttccttgggagaaagcaccagatataccgttaagagttaaatttgttgagttcacctttatacatgagggtagggtagaaaaagaacgatatacctattattctttctttacccattttgaaaggagtggctaaattatgactatgcatacagaaaacttattagccgtaacaatatttttacttatacagatggcagttgttgcttttatttttactaaagggataatagtatatgatagactactttcagagtacagagacgtagtcggtatagcgtttagtatactcctttttggtaatggcgcaatgcttataattcaccaagtgattgcgatattcagcatatggtttaataactcgttcagggagtctgacgtgtttctagctcaaggctactttatcgtattgcttttactatatatagcactgaacgtatatgggagcactttgtttaaaaatattagagttaacataaaaaattatataaactataatgctaggagtaaaaattataagttagcactggttatagtaataacatttattgtcaatactgtactagtactggctacttactggctagcagaagtagttaagggggctattattcattgaaagataaagtgtttattgtatcagatgtacatggttgttatgacgagctaataatgcttcttaataaacattggaatagtgatgaagaggacttaatacttttaggtgactacgtagatagaggtagcaaatccgcagaagtactgggtttcgtttataagcatatacttaatggaggaactgctttgttaggaaatcatgaccaaatgttattagattttttaacatttccattgataagagaagcagactacgaagacctaagtgagatgtacgcaatgtggatgtacaatggcggtgataaaacaataagtagtctattggatttagatgaacacctacaggatgcgtttaccactagaaagatgttattagaaaaaacaaatgtagttgaagtaattagtttattagaaccatactatgagcatggagacaccttatgtgtacacgcaggaatacctgtttatcgtcagcctgattggagaacagctactaaagatgaaatgatttggacgagaccactagcattacagaagaataaaacagataagacaattataatgggtcacacacctacaattaatttgagaaaagagaacagccacgatgtattcttaggtgacagaagtatttttattgatggtgcctgtgcatatggtggtcagcttaatgctgttgttgtcgatagtaaaggtaacttactaaacacgtataaacaagagagcctaagtaagtaaattactaggttcttttttattataaaggtgttgacatataacaaagtagatgataatatatagacataaacaacaaggaggtagcaagtggtgagggacgaaaaagaggtaaatcactttatagaaactttagcagaagaacgtaaaaaaaagtggaatgattatttaacatcattaagagacagtgtaactctaagtgaaattaggacaagctattcttgggtgacaggtgcttatatagtacttctatatatctataattatgatttaaacaaacgaacaacagtacaggttatcatagatgaatggcgtatgcctcatgtgtatttaggtgagtttgagtatttccgagaattagggaagatgtacgatgataaagtaaaagaagcgctagacttatatgtaagacagataggtcgttctcgtccagaagctaaggcattaggaatccgtagatacaaagaagtaagtagaaacaaccgaacacaaacacgaaaatttaaaccaagaaagggtaatataaaatgaatatgttagagcaatatattaaagaagtgcattcagtaacaccatatacagagagttggacaaaacaatacggtaaagatttcctaatggtagacttaacagtaaactgttatggctctctgtctcggaatgagcgtttgttttctgtagatgagtgggaagaagctaaagagaaaggatactacatggcatgaataatttgacagcacaagatttattagacgaattactagaactaaaagaccaaggtcacacactagaggactacatagttgtgggtgaaccagttaacccatatgaccactatttagaatttgatggatggcacttagacagtagcacaaatatgattatattaaaagggtagggagggcaagacaatggaagataaagactatgcgttagcactagaggaagacacagatgtatggaaagcatgtgagacgttccctacgagagaagaggcagtgtctgcaggaaaacttgcattattagctattaatgaaggaaaaacatatgttgatagcttagggatgtacgtagaggatatatttggaacattacctgaaaaggggacaacaagttttgcagtagggcagttcaataaagcagttattagtgtagatgtagtaggtatgttagaatctgtagggggacagctatacagtgaatatggagaggtagcggaggactactacccaacggatgaagtaagcgatgaggctatagacgaattacatgacattatcactaactggttagataagaacttgacacaacccccatcttttggtagtgttgatgacgtagagaaaattacattagaaggagaaaacaaatgaagaaaagactagaagagctaacaccagatgaattacagcaatatgcaaagttaaataagcaaagagaaaatcataaagagaaaattaaagagcacacagaaattattgaacatattgaagaaaagattgaaaatatcctatatatctcagaagaggcagaacacaatagtgacttaccattctaactaatataacatttaagataatttagggaatgagaaaaggggtggctaagtggagtttgttgagaaagcagaatacgtttttccaaagcctattttagattttaatttaggtcatcgtgtagcaatcgtattaaaagaagaagaactaagttgtttggaacgctcttattataaaacgaggttatatggtagtatatacctagtagcatcagataagataagtatagaagtatacaatgaatcaattaagctagggtttaatgacgaggaagttttagggtgtgttattggttatcctgtagagtgtgctaagtggtttgctaaagcttcgagagaagagttaagtaatagtggtgtaatgatgagtggctcttacacatttaaatgccctgagcaccttgtagattatgctaaagaatacatgttggaacattatggtttaaaggcatactatgatattccttgtgaaaaaattgtaactttttcttaatgtatattaattggaggtagataaagtgatgtgttttggatatatctgtcctgtatgtgagacagctattgtaggtgaccctgataacggaggggagctctgtcagctaacccatattagacattctaaagtcatagggacaactagaggacattataatggtcttggtggagtagtagaagattgctattttggaggaaggtctgggagaaatagtcaagaagaaatttttcgttccgagtatgagtttaaagactcttatcgtataggtaaacgaagacaagcccctagtgggagagaagtaagagcatatgagccacttactgatttaactgtaggctctccagaaagagaatgttttgagattgttatagaggggcttaacaaggagatgaaagggtacgcaaatactatcttagctcttgaagatgagctcctccaatctgcaataggaggactttcttgtatggggcaagattactatgaaaaaatgatgattttagctaatatgaaaagtagtagcaatcgttattggctagtgttaagtaaactgtataatgactatatacttacacttccggacagcatggaagctaagtcaggggtcattgcagtacatgtggcttgcttaaaaacattgagtaaggaagagcgtgcaagcttaccatttagtaaaccagacccagaccaaagtataggaacagttagagaagcatatagggaggataacacatgattaaagtaaaagttattgatagcgatacaccttatggtttatctaaggaattagagaaatttttagcaaatcataagcacaattcaattagttattcagtagcatacaaaaacccctcaaccatttatagtgcccttgtcacttataaacaagaagacgtttcgatagacagtttagcgcccctaaaagctaagggattccttaaaataattagctttgataagtatagagtactggaagaatgtcatagaagagtgccattaagtacttataaattaaacttaaatgatggagataaaataccactattttttaaatataagccagtaatagcaattagccctactggtgacgagccttattatttttatgagcacccagagcatatagaagctaatggtgggttacctagtcctcattttgcttttgagcataaaggaaactactatgaatattgggaggaacattagatgagtaaatatattttattttgggggcatactccaagcaaacgtaataaaataggaaaagagtgcttgtcacagtggtatccatcacaatttagtgcacctattaatggtttaggagaagacattattttcccgacagcagagcactacatgatggtacgtaaagcaatgctttttaatgatgttgctacagcaaaaagaattttaaaaacagagagcccaaaagatgctaaacgtttagggagacaagttaaaaattttgaagaaaacttgtgggtaaagcatcgtaaattaatcgttttagatgggaatacttataaattcacacaaaatgacaatttacgtaactttatgctatccatccctaaaggaactaagtttgcagaggcatctccttttgataaagtatggggaatagggctacgtgaatcagacccacgctctcgtgatacgagtaaatgggaagggcttaacctattaggagaggtgctaacagaagttagggaaaatttaaattaaggagggctgtgtggtgatagagtgtttaatgtgtcaagtaccattagaggagacatgtggtgatttttatttagatgaaatgttgtgtggcttgtgtgctacgcatttattactagacgggatgaaacaaagtttaatgacggaaatgtacgtattagatttactactagaagaggaggcagacatgttatgagtaactgcattgaagaactactcagcaaagaggctatttacgtagatagatttattgtagctagggataacagttatttcgcaaatattgttatgcaaagttttcatacgaagaatgtattccatgtaaaacgtaatgatgaatactttatgggaattagagcaaataactttgtggtagagcttggagaacttgattttgaagtaccaattagcaaagaagaccacgaactactatcatctgtactcggtactaagctagacacggcaacgttcgtattagttacaggtaaccataaaggggttcgttacactacaatatgggctatccctaaaacaactttagataaagaactattgtaatataaaataattaaatataaagtgttgacttttaatctagctgatgatacaatgtatttgtagatagctagatttatttttttaatatgtaaagtgaaagaaggtttaacagatggaaaaagttaggcttgtgaatgatgtgtttagtatagcagaatctaagaactttttaaaagaagagtgggtagaaggtcagtataagatttatgaaattgttggagtttccccctatgcaattattggaaaaagtattaattcacgagaactggaatattttaaaccaacagagaatcgaaaactattttgggtgtaggaggagctaaaggaaatggtcattaacagatatattttatgtattaagtcgttaagtatggaagatatgaggtacgtggtagagaaggtatcttctaaaacagattttggtgggttatcagaagactgtcttctacctcaattgtacagacccattacccttgtatttgtccccagtaagaagtatgtataccaatcattaggggtaactacctataaggacgagaaagcgctagtgtatagctatggtactaatggatatgggctagaccaattattaaaagatatagaggaagcaaaaaaggtagtgacctttgaagaggggctaaaaagaagatataaccgtcaaattaaacgtaaaacaaaaagaattaataatttgttagagcaagataagagagggtatgggtggcatgaactgggctatttgcagggggttgtcacaacattagaagatgtattagacgatttggaggagagacaatgaagtatacttgcataaaaccatttaagacagaaagagtatcagtagatgtgaatagtgtatggtatggggtagatgtaaataatatgtggtatgaattagatgccccttgttaccccgatataaagtgccctaaatatatacttataggcggtagtgcaggggcatctatttgtattgatttgcacactcttctaaataattttatgaaagtagatagctcttatgagagtgtagagattaagttaagagagcggggtatatttggtaaagaacacactattacctttgactatatgggttatgcccactttaaacaaagattccttaaacgatttggtaatattagttcagatagaaaactaaaaaaagaaattgtaaataccaactcgttgaatgacctagaaagggtattcagtaacaacgataactggtcaattaagattatgaaagagagatactaattatggataataagtttgcaacaaaggacgacagatacctagtacaagttattgtaaatggagaagtaaaagtatctaaaacaataaatggactagcacaggcaatgacggagaaggaaagacttactaagcataacagaagaaacttagttattgattcagaggaatatagagaccaacagaataacctttatgggagcagaggaggtagacctactgtaatatcaataaatttacaaaaattgaatcatcaggtagagggtaacgaattaaacactatcatttttgatttagttagtaaattagaagttactcataacctcaagattgcacctacgtactttaaagcagttaaggaaggtagaaaactatttgaaatacgtaaaaatgatagacaataccatgtgggagatattcttttattaaatgagtacacatgtggttcgtatacaggagagtttattaaagtagaagttacttatatcaccgattatgcacagaaagacgactatgtagtcttaggtattaaactatgcgaaaaacaaatgtatatattgagacaacttgggcaggtattggtgaaatagagacagtagaagtacccgataatgctacgaatgaggagatagacgaagtagctagagacgtgttctcttcctactgtagctttggttggggtgacgtagaggacgaggaggaatagtgaatgagtagacaaagatataacgaaataattgacagagtttgttatatgtatatggaagcaaatggtgaaattaatttccctaatagttttgagggagaccttttaaaaatagcgtataagactgcagtagaggcagttaaaaagccagaactgaataaaaatcagaaacaggtatttgaatggttaaaagtaggttactgtagtaaagattccctattcgaggcgctcgaacgtattggggagacctatgaagaccctaaaacagaagaggcatattactctttatcagtaacggaggaagcgcaagttattcaagcatttactgattggtttttagaacaggagggagtacaatgattaaatttaaagaatttgatgttggggatatacattacgaagaacaactagaaaaatttcaaaatgagcacccaagcgccgaatttgtaacaatcacaggcggacaaacaacatatgagaaaatttggttcaaatacactgatgataaggtagcccaagtagagctaagtgataatcaacaaattgttttgggttggttaaaagaaaaatatacgttaacatatattgaacctatagaactattttggagactgagagttaattctttaaaaccatgctattgtgatagacctgtttataaaaattatagatacatgacagaaattgcgcagttgcaagtcttacaagcattcagccagtgggctattgaaaggaaggaaactaaatgaataaacaagaattgattaatgcattagaagagttaaagttcagtcacgtcccagtcgttcaagagttatcaaatcggagccagtgctacaatgaggcaatcaaggcggctattttgctagcaaagaaactagacgaacctaagaaagtaattgtgccaaagtttgttgcggaatggtacgaggaaaataaacatgcgttagacctagctatttttacacaaattagaatgttagatggtaatagctacccacatgattcagagttcgattgttggttagaccaaagtaagaataaccctattgaaactttaatacgaatgaaagatgggtacgaggtggagaaagagccactttaccatgtcgtgtttactggtcttggtaataaaggagaagattgttatctttgggttgacacatcccacaataatgaagtatacttctcaaataagcctaatatgggtaagaacaaatttactgaacaagaaataaaagaaattgatgaaagatactgggcttttgctgtaccagtatacaaataggagggttttataatgggctatgaaattacttatgatagcaatactagtgtaaatatacaagaaaaaattatcgcagtcaataactcagcactttataaagtatgtgttgagaagaaagcttctcgtgtaaatgagaaaacagaagtaaagtacactttagaagtcagctctgcagaatcacaggagagtacaaaattagagttacctcatgaggtgatgctccgactatcagaacttattgaaaacagccttgagttttaaggagggaattaaatggataagaatgaatttattaatctattaggtgatgagattttcactcaagtagggtgtgatggtacgttgaaccctcatgctctaggctacaatgcagggatagcaaaagcagtctccttagcatatggtttagatgagcctgaaaaagttgttattccttataaagtaggggaatatattgacaagtgcaaagaagagaacttaacgtttattaacatgattgacaaaggaagctaccctagtaagatgtacttttggttagctaataacccagaaaaccatgagaaacttgccaaagcatgggtactgggtcatacggtagaagaggaagaagacgagtactacatcattatcgcaaaagataaagatggttggggatacagttatatcgaccgattagggtctcctgactttacaaactacctaaaagatatccctacctttacagagaaagaaatccgcaaaatggatgaacgtttcatggcatttgctgtaaaagtaggtgacgtataatggggcacttaccggacgatgtagggagttgggtaacccactgtatcaaccttgtatgtgatggtgaactgttagaagataagaagagtcagacttatacttgtgaggactgtggggaagttgttaccctagatgaactaaacgaacacttagaaaaagaagcagaaagttacttaaagttctttggatataaataggaggaagagtataagaagaactagacttatacttgtgaggattaggaggttgtataatgattgatttagaaaagctcattaacacgatacttgtattattaacaattatagcagtaatataccttatatacgttttctatgtgtcatatgtaacagctggatgggtcggtgttttgctgatgttagcttttttctgtattttgtatgtattatgattaaataggagggagaatagatgactaaaaaagtagagatacctagttttgttgcacgagcaattaaaaagtataactcaatgttgagcttattagctaatgagtattttgacccatacgatgaagcaccagatgacaagctagttgaatggattgatgaaggcaaccaatatgtactagcaagggcttacatgtatgggtatacgataaaaagacagtttaaggttattggagacactcgttatgaccatgggtttacaatcggggatactgtatatcttgaaaaagtatacccagatggtgtttacgcagtacgaggaaattctcgtcacagtctaaggacagtaacactagatgtacacccttgtgacctagaagagattacacaggaggaagagtaaatggaaaaaccatacgtagcagttagaagtatccacacaggtagagtagaatactgtacagaagattggctcttctgcgggagtgagaaaaatgctatgccttgtaaggaaattagatatgtagacatgttacactgtggcatggagagttactacgaagtgacctttaaagctaatcagtacacttacacagaagaactccctgtatatgcctttacaggtatcaagatgattgagaaggaagagaactaagggggcaccatagatgaaagtagagcaaggggagctagctattacacttcctcaaaatccggaggaaactgcgactgcgactatttatttctcagttgatgattatggagacattgcaattactacaacaggtgaagaaaactatacacagtttgacttcaactttacggaggaacagtgggcagtcattgttgatacaatatccgtgcaactacgagcacaaaaattaagcagttgacataaagtagatttaagcatatactattaaagaggagacaaagatgaagttaaaagatttagttaagttaatgaagcccggagtggacgtactcattacttatcccatggtaggggctaccataacagttagaacaaagataggaagtaacaacgaggtactagaaccatacttagactatgaaattgttaatgtatccaaaacgcatcattatatcgcaattgacatcatctaaagtaaaagaactaatgttgacttaagcgcatattttttatgccacttaaaacagttgactaaacaccagtcttataagtaccctatatgcctcttagggcggtgataggtattctacctattgtcaccctaaataactattgtaaacgctaggagagaacataggactggtttaacactaaactataacaaatataaaaacggagggagacatacgtaatggcacagtgctaccacgaccagttaacaatagaagaagagaaacaacttaaacgaaaatacgaggtactacaaagtggaacatgttggcactgtaataagcctctatggatgtgccctccggatagagtagtcaatttaactacccaattagacttagatatacctaatgagtacacagtacctacacgattacattataaccctattacaggagaacgtatgggtcttgtgcacgttaggtgtgattactacttaaaagagctgaacaagagcttacttaatcattcaatggaggggaactaagtgaaactatctgattacctactattattggtagggggtgtagctatactgctaaacacactcatggtgacatatagcatttattgggggctaactagcggggaagctactacaagctactggataacaatcgtgttttgcacaatgcaggcagtattagtaattggcgcatatatcttgaacataattacttttgcaagcctaaatagaattaacaaaaaaataagaggtaaatatagctattattaaggaggaaaacgatggataagaatacaaaagaagaattggcaaacgcattaggtaaattttttgggactttagttctaatggtgttgacccttgttattggagggtatgttacgatgttcttgtggaacgggttaattgccccaacatttggggtgttaacacttacatgggcacaggcaattgggctagatgtatttattagttttattacagctaagactactacaaatacagaagattctattttgatggtatttgcaaaagctactgtatcaacattgttatttatgcttattggatgggtagtaatgttctttatttgagggcagtaactagatgttaaatatttttttataacgtataaggagaaaaaaataaatggaaacaacattaatagacaaagagcggttaaacaaattgaaggttggggatacaatcttcataaaaccagaacttcgtgacccgatactaggttgtgataaggcaacagtaactagtgtgactccggatattgtatggatttctttaaatggtacagagtatgaagtccataaagacgagaaaacgaaagtaccgctttaccgcaatcaatcaggagagattgtcggggagtatttcttggacttcgcagacttccataaatactggacatacttcgcaaagactatggcggaacaaattagtcttagaggggagtaaccatatgctaaatattttttataatgtatggggtggatttataagtcaccctgtactcattactctaagtattgtaggtatttatgtcttgggctatgctctagttgaatttatcgttggtattgttgttttgtcgaatagtttaaatactaaattggtgatagcctctgcgatattcactactgtattgtctattgtttttactatatactcatttttagtggaaatttacgtagtcttagtcgtcattagtggcttggttgtgctattaacctaagaatactaaataaaaggagccacattaacatgctaaattcaagaggagagtctttgctccctatcaacacacaagttagacttaagaacttcaccttcgagtgtaatggtagaggtaggagccctatggggagagtaataggttacgataatagtgtgtgtaaagagttcactgactactacattgtggagagtggtgacttacaagggtatgcccattacactaatgtaaccgaggtcgtgcttggcggaacctgaacctaataaccaattataaggcagacagctaaataaaaaagaccctatccttatcaacggggaggggtcttttttatatgggagtatttaggtgagcctatataggagccctagggtgagcccttatgttgactttatatgtagggtgttgactttctaaatatataaaaaataattttcctagaaaaaatttgtaccctagggtcggggtaggcaaatagccccacccccaaagtacggtcaaaaaaccacggggggaggtcaatcgtaaaacaagcctaccatactgaggaaaaccatcaagtctccctcagtggcaaatttaaattcagttctttcgatgtaggatattaagattctgaatagctctttcaagtcctaactttttacttacaagcccaacccgcttactatgtgtgcaacctattttatatagttcatagttagatggtgtgcttaatatccaaaaatctttatccttatatacgatgttagatgtattatacatgtgtgttacctccttatctttataagcctaccaaggcacctatacgtctatgctttccatgtcttttgtatatatcataccctcattagctaccgtgtactcgtcccctcctctggtatctcttacaagtgccgtgcccttttccttgtcatctttaatcacgtatacacaaacgccgtcttttgtgaatgctgtttcaaaacctctatactttctactcatcccaatcactctctttcttattcatcttacatttaatatactacactagttattgttatttgtcaatagattagctcaacaattctagcagtgcgcaatacccgctcttgtatgcttgttcctctctatatctgaatacaagttcatgtaaatggcagtcctctagttcctcgttattgtattctccatcgtattcgctataatgtgatacctctaagatgaaatacgagttattgactgtgtacccccctgtatattcttgttctaaggtgatgccctcttcctctttcaattcctcgattgctcgtttaaacttcattttaaagccccctattctttcgtataagcgtttctagttatttaccttataattacactaggaagcttgtagctctattgtataagctccctagtgccttatatgttataatgcaggtttcaagtctttaaactcaattccttggctagttaggtactctgctacgctctctttgtctaagtccatacaagtttcttgttcgtagtctagttgtaattcctcgttgttatcgtcagttatatagattcgtacatattccgaggtagccgaaacatctaaactgattgtatattccttgttgtcgtgtttaaaagcatagtaaccttgactataaaagtagttacctaggaactcttgaaaatcgtaataagtttcaaagataactgggattgcttttgagtagcccgccctcacatctacccccgtatggattgcaaagaatgcaatagtcgtgtctgaattttctgcctgaatagttttaaagtccatatcacgttctaagaaaccgctataattatacgtattgtcctctgcttcaagattccatgtatctttctctagttcctctagtaactcttcataagttgggaactctgattctgcttcgttatttttaaagaattgaggtaaaccgattaagtcattgttaccacctaataaaggtttgtttgtgctacctgtaaaggtaacttgtttcatctcccctaactcttccatcagtgtatcatagtcactaacaccaaaccaatcattaagtaattctagtacctcttctttattcatattagcaaattctgtcatagtaaaaacttcctttctgttgtgtggtgtaaagaggtttattcctccttacacttattatactacactagttattgttttttgtcaataactttatttaaaaatttaatatatcgatagtgtttgaaaatacttcttcgatttcttcggcttcctcttcactgataacaccattgtcttgtaagtcgtttataagctcgaatagtgtattgcttacgcctgttgtgtagctgtttagttcctcatctgcccaaccgttgttcctacgttctaaaatgcctttgtaaccgtccttgatttgtttgatgataattgatttcattatgaaattcctccctctctcttacacctattatattatactaattgttgttatttgtcaatagaaaagatgaaactttttaaatatattcatatgctaacttatttagccaattctgcatttttaaatagttgtcgatttcgttccctacctctttatttgtttctagctctcccgctacgtccttacaagcttgtaaataagtaccctcacaagaaaacgggttcttaatcggttcaatgaaaacacctgattcattaataaacgaaaatgggctatcatttttagttaggttcatttctgctagttctggcgtcatagtaataacttcctctctttatctgatatgtattgtgtaagaaagaagtactcattttcctgttaggtttaaccctttccctcttacacttattatactacactatatgttgttatttgtcaataacttttttttaattttttgttccgtcctcgttcattgccccgcaaaactttgctaagtccattagtgaaggcatgtcgttgtgtttctcaaaaaattcttttacttcctcagttgatagctcttttccgtattgtttgccgtagtctttcacttgttcgattgtcatattaataactccctctctttatcttataaacctattatatactagttattgttatttgtcaatagataagatgaaattttttaaattaattcggttgttttgtatgattcaccgttaatatacatgtaaatatcattacctaactgaattacttctaatttattaccaagcatttcttgttgtttcacttctgcgttaatctgttcttttgtcattatgaaattcctccctctctatctgataaacctagtatatcattaggggctgaacttgtcaaccccttttttcaattttatttttcaggatagcgccccgtgtctgctagcgtattgctgttatggctttgcggttgcatttgtgggacgtgattaaggttgttcatttgttttttaaattcttcactgtctagcaattcgtctagtgtgtcgtattcctcttctagtaattcgtctaatgtgttatattccatgtttgtaacttcctctctctatctgataaacctagtatacaatatagtttgttatttgtcaatagaaaagatgaaatttttttataatgcttgttctacgtcctgaacaacttccctcattccttttacttctgctttcagtagctctgctcgttcctgtttcccttgcccctctagttcctttatacgctcttccttatattctatgtcctgtttaataatgcctaaaacattaataatatctgttactgtgtccactgcaacccctcctattatcttataaacctattgtataatagttattgttaaaagtcaatagataagatgaaaattttttattaaatgttttatggtctagcgcattcataaacagcgctacaagatacataaaagtatatacctgttacaacgtcacgcacacgatactcccccgctagtctgtctatacttacaatctgacctgtccaagttcttgcgccgttgttacctcgtacaaatttgcctacttctagcattcttattagtccccctcataagccctcataatgtatttatatataaagcccgttaaaacttagttacgggctttatacgccgttgtgctatagtcctagtcctgacaataattctttttcgtttggataaataaaacttgcatttttatagacaaacgctagctctgttaactccttgttttctagttcctcgtttgatatttctacgctgtgagtcccttgcatataagcaattaaaacgggattctggtctactatatctcctctagttagctcatagccccctagctcgttatatgcctctaatgtagcgccttttgattctgctagtttgataaattcttttaatttcatttctaagttctcccctctctttatcttacaagttaactatacaatagttattgttaaaagtcaatagctaagatgaacttttttattaaataacaaaagaattttatacccttttaaaatctccctaggggaaaagtacacccctatttcttataaacgttgctacaatagcattcttattttagctagttaacctagcccaatcagtaaaataacaatgtgcatctgtcccccgtttgtttttaacagtctaatctactaactttctgcccctcctccttgtgtattctaatatatgtataaatagcctatgcatttaaagccccctataagcccgtataagcgttcctagtcgttcccctcataattacactataaacgatataagcgccccctatatagaatacaatagcaatgaatgcagtaataggctagatgattaatagtataagatagctaactatataatcgaataaacgcccctattttcagatatttaaggataatcgtatataaacagtaataactatacttatttcagattattttgaaatagacttatgcgcccccttgtcgggtttctggtgaagtcgattctttaaaatctcaggtaaacaactttttttcctttttcctctctcctttccctccctctcttattcctttattcccttactattgcttgttaggggctatctgtatataggtacataggtgtataagtatataggtaaacaactttttttctttcttttccctctttccttttctctccctctctctctattcctttacactcttacccttacttattatgtggctatctgattctcttatccctttacctcctcccctccctctatctcttcccctataagtgtatatagttttatagttctatagctctatagtcctttacctctttatatatttatttcttttcgtcctgtttattatcgtacactattccgataactgaaaagaataatactatccctaagattgctagtaattttaaactgtatacgctcattcttttatattcctttccacacacttatcacctttacatctttatccgttagcgttctttttaggatagctctattccctttttctcttaaggtaacctttttcattacagtatagtttgctagtccatcatactcattcacaagaataagactgataaccttttcccctttttctaggtcaatagatattaaagcaaatggaaaaactttattttctaacctgattgtataccctttttctaggtactttctaacttcctctgaaaagcgtttctttacgttttctcttcttacttcttccactagttttatttctttgcttctttttacctgtttagcttgtttattcataaaactattcatctttacccctccttatctgattacatcaaaacttaacgggttttcttgtgagtgtttagcattttcatcatataatgaaatatctttaataggcaaagagtttccattttcaaaagtataaataggcaagtccttattttcttctggcacttcttctagtttcttaatcaattctctaattgttagcccttttagttgttccattcttcttgttcctccttaatagtgtttgttactacccttttacgaaatgaaattcgttgttatttgcatattctttcaatgtattaatcattttagcagtttctactttttcggtgtctgattcttttgaaattgacttataccattcttctaatgtttttgtttcatctccccaaaagccttgtacatcttttctagtgagtttatctgattttttgatattttttaacatggtgttacctcaattggttcaattccattattttctagtaattcatttacttctaaaatagttaggtttgtaaagtcaatacctgattcttctaggaagtcggctagtgtttcacctaagaaagccgttcccccgttccctgtttcatcatctcttaaaattgtgtttagttctttttctgttagtttcatgtttataacttcctctctttatcttataaacctataatacactatgctttgttttttgtcaataacttttttaaacttttttttacattttttcaatagtgcattcactttttaactgtgatagaataccattgtaagttgcttcccaagcttcctcatatgttttatagtagttttcccaatcttcctctatttcttgccccgttaggttttcggctgttatttctagtgagtcatgcaacttcttttctatctcttccacgttttccccgtagtaaacttcttctgtattgtggtaactgttagataccttaaatagtaattgcacgggcttttcaaccccaaagcgtaccccctctgtttccaattcatctaaggatataactaaactctcggtattatctggatataatagaaaagtgtcaaccccgtttttaacgtttttctctgcttcttcttttgtaacttctttcattccttgccatgtataaccgaaattctctaatgtttccatgtttagcacttcccttattttatattttttctatgtaaaggaggttaaccctcctatttatactaatctactaaccaaggcacttctctttttaagcatttaataatagccttgttcacttcctcggtggcttcctcgttccataagtctttatctaattttagcccctctgcaacgtctgctaaaacatcattaaaatctaatctatcaaataaaaactcgtgacctttaatatagtaaagcatatcagcaactgccgaagcgttcccgaggtctgttagagttgcgccgaagtgttcctcttcgtattcctgaatttctccgattgcgttaaaaattccatattcttctaattcctcggtatctgtatttgtaccatagttaaaaacttcattgtgcaagtcgcagtaacaaccatcaaaaccattttccaaagtttcgataatttctttcattagttctttttcatttaaaaatacattgttttcttttgtcatgtttataacttcctctctttatcttataaatttataatacactagttgttgttatttgtcaatagttttttttataaactttttgaccaagtcaaacccttgtgtatgtcttttaagaaaaaaggttgtttattgattaaaataaaattgtcccctgtttcttcttccttacgtagtttagtaaagcacactgcatgccgttccccacagtttaaagcatagtatacccgatttttatttaagtcaatttctaaaatctcataaatactgaatggtgccacgttgcctagttgctttttatcctcttcacttgttgcaaagattgcaaccccaattgcttttttatattgtagttctgtttttgtcatctttaccattcctttctttatcttatgaattaagtatacaatagttattgttatttgtcaatagttttttttaattttttttatctgctaacaatttaaaaataaatgtaatttttacaagtaaaccaagcaaagccaccacgctcattttttaccctcacttttttttctaaaatgcaagaatagctctatttcttccaatgaataattaccgttgaaaataaattcaggtggttcacctctttttctcttttctagctttctgattctttcatattgcaatttttttattctattattaatagtaaccgccacctctctttatattataggttataagcaagcattttgcaagccttttttataatattttttgagttcttttctttcttcttctagcgtttcaatgtcaatagttacaaggtatgtgcatttttccattggataactagtataatagcttaatacgttccctttaactttataatctgttatacctctattctctgcaatgtttagtgctttctcctctgtcgtcatctcaagcccccctagtctatgtaaagggaggaagcccctccctgtttttctattcttcttcctcttcttcctcaaattgtgcaataaactcgtccatctcaaaccataaaatatcgtttatagtcgtttctgttacctcttccgccccctcaatcatcaactcgatttcttgctgtgcttcatctaaaatatccaattctttaattttttctagtcggtctttcccgccttcccaagccttaaactctcccaaagtaatttcttttgtgatttttaacatagtaaacaacttcctttcttatctctataagtctataatacactagttattgttttttgtcaactattatagataatctttttcaatttgttttttagtgtctgcttttagtccgttttgcttaaaaaattccttaatatgtcgcaatgttgtctgtgagtacgtaccgaaaacctttgctagtccattagtgtaaattgtcgcaacttttgtatcgtatgaataaagagaaataccgttctcctcttctactaggttagcttttccataaaagctctttctactgtcgtaaatacattctaattctgtcatgtcaataacttcctctctttatcttataaatttataatacactatcttttgttttttgtcaatagttttgattaattttttttatttgtttttccctgtcccttgttcccaaattccttttaaaccgaaaacataggcaaatagcaaaccactgcaaaccgtcaacgttctaatatcccaaccgcctaacgctctaaaaacttgtaagcaaatcataatgctaaggtagccccaaagtagtacaacgctaacaagcaaccccactagaataatatcagataatttcatatttaaaacttcctttctttatcttatgaattaagtatacaatagttattgttatttgtcaatagataaaacaatttttttctatgtaaaggaggttaaccctccctagtaatgaatggcgctttctctgtattgttttacatctctttgtaatacgtttctagcgtggcagttatgtgcaacaattcctctagtgcttgccgtcatgctgtaaagctcgtaaaccgtgccgtctttcattgtaacatactgataaacgtaacacccgccgtaaactctcggacgtgactcaatataataagattgtgattggtcgattaaactagtgtctaagtagcccccactacgccattctgttggttctaataaatgttcttttttaaattgttgttctgtcatgtctataacttcctctctttatcttataaacctattatagtttatcttttgttatttgtcaatagttttgattaattaattttaaatttttatttaatgtttctagttcgtttattgctttcttttgtatagtgataacttcttttacttctttcttgtatcgtttagctttcataagtctttttagtccgttcatcatataaagcccctcccttgtttacatttatatgttatcatggttcttttaacttgtcaatcttttttttatgtaaaggaggaaaattttcctccccttattctactgattttaaaaagccgtctaggtattcttttgcatatgctcgttgttcgttatctcctaaaaataaatgctctaaaaagttgtgggttgtaataggtagcacgttatcaggtgcaataacctttgctgttagctcgatacctgatagcattccttttagttcgttaaaagctgataactgccctagcaaaccacggtttaccattttaacctttaaatcgatttctttataaaaagtaacaataaccttgtttagcacttcttgttgttcctcaattttattatcccattctttcactgcttctaattcattcatgttcataacttcctttctttatcttataaatttataatacactatcttttgttttttgtcaatagttttgattaattttttttctataagttataaagtaactcaaaaaataattgtcatttctttaatagaatgatataagttwkmagatataacattcttgtaattttcgttactcactcgaatagtgtctaatactatcaaaattttttctttttcattttctagctctgtatattgtagttctttatcaaaattttttgttctttcttgaaagtctgtcttattatcatttaatagcttcattgtattgttgaaaattttaagttcttcattagataaatttttgcggtactcttctaatcttttaaacatgtttataacttcctttctttatctgacaagtttattgtagtttatcttttgttatttgtcaatagttttttataaattattttaacagttcaataattccattagaaagctgtccccaaccgtcacacactcgaaaacgtccgtcccctatcggttcggttttaacggttgccgttgcgtattgtccgcctgtttctgcggtactgtcaaaaataacagtatttaagaaaaccataaatcgaggttttttcttttgtagctgtttagtgcttaagcattctaccgctaaaacaccacaccgattttttaattctttttctttttcttcctctggtaggtttttaatatctgcataggtaaatttatacttgttcattttaatagcgcccctttatctttattttttttattgttgttgttccttacacgctctatattatactagcttttgttttttgtcaatagaaaagttcattttttttttaaataatttttcttactatataaagggagaaaaccgacaatctggggacggtagctattaaggtgaacccgcatggcttggctctccaccgctagccgggcgcatccggattttgtagcctgctacttaggggatttctcattttgcaacctgctacttagaggatttttcgtttgtagccagctacttaggagattttgtagcctgctacttaaggtaaaagaaaaagctagccatgaggctaactcaatcttggtaaggtattacatagtgacccttacagaaaccatttttatatgtgtctcttgcttcaatgataatacctactagcattgcataagctttgttgttatcctctaagaataaaacatcgtcatggtcaaaactgaacccttgaattgtagataagttatcagagacttctagcagtttacctcctcgtaaccacccattcagttcttctttaaagtggttgtagcctgctactaggtcttctctagttaactctaggaatggtttatcaggtccatctgttagaacgtaaccttcacttaaggcagacaaagccattccccaattatctaattcgtcaatatcatgaccatctcttagtaacacatcataaatttctctatctaaccaatctccaattgtttcaaactcattaacgtaactcatagttataccctccatcctatcctaaaccaaagtgcatcataagtctatcctaagtttatcctatcttgtatcctatcttatcctaagcctatcctaaacaaaagtgcatcctaagtttatcctatcctatcttaagtctatcctaacatagttaaacataatagtcaatagttacctcaccaaaatcctctggttgaatactggacacctcactagtaacaataagtgcattttctgggatgtcatctactaagcatttaataatatccttatcaacaacaaacccgtgcttgttcactgctcttaacgttacagttgtcataatgtgcatagcatctaggacatcacctagtttaagacctgctacttgggtagtgtaaggttttgtagcatacactggtacgcatacttgtttgttatctcgtgtgttcaataggtaatggtcgatattcttattagaaaaagccttagcaatctccattacttcttcattataaataactttaccacctactgctaattgataaaaatcagggttcttatttactgtgacagcttcctttagtgctttctctaaatcactatgtgtgctcatctggatgctcccctatctgttattatagattgtttcaatgttttttgttagcttctctacttcgttggacacctcgtctagctctttgtgaagaaggtttaaatcatattttatctcattcaatatcagacgtttagtagaggcttttttatttaacgtagcaagcaaatgctctaatgcaaaatcagggtcagctactttatttagtggaatagcaatcgctacacaccaataagaatcgttctctttaacagtgaacagcccctttgcaatatggctcatatctttatcataatgaacaactgtacctgctagtgaagccatttcaccggtaatgtttaaacactgtacttctgcgcctgctaagcctcgaacacctttcttaaggtcggacacgactagcaatgggtctccttcttttaattctaataagggttcttccatcacagcgtagtcaatgcctaaagcatccatttcgattaaggttacccgactaccttcgttattgataatataccaaccctcattggtggttttatacaatggatatgtgatacccaaactcaacctagcatccggataataacctcctacagccttaagaaggtctccttctctataagtcttttcttcttttttatcttttttaaacattagtatttcatctccttaaataaaatctgatagcgacagcccaatacttctcatgtaacaatactcaatcttcactgtatctttctaaagagaaccctttactaacactatgccaaatagtaagttttttaggttctttttgcataagatagtgccaacaacttgctaaattcaatgtttcctcatagagtcttgattctttgcactctgcaataaacttctctttatcaagacctaagattccacatacctctttaataatatctctattctgctcgttatccgcaacgtctacatacaataccatacctgaatcgtaatctgaatctacaacttctacctcatttaaaatagctaaaactttcttcattcgttttttaatctgtctctggttcatgttgttgtcctcctatttaattcttccacactttttacactttaagtacccatgactatctaatgttttatatacacctttaaccgtgtagtcatgaatacagaatgtttctttccatgcttgtttaacccataaaatgaagtcccctaccatcataattcctccttaatctagttcttttgcgcttcttgattttcaatataggtgtagcatgtatcaagtgccgcctcaataacagcttcctctgtatgagggcgcattccccactctttccctgtaaccttagttagtgtacggtgctgtccgttccttacaattacaaaatacatgtcttgttcagggtaataagtaataataatttcaaattctagccccttatactctctgctatacttcttttctgtttctttcactctacgctacctcctaatattgtttgttttttatctatgtacctactatagcatggactatagtagttgtcaacaacttatttcaaaatactaatataaatcttcccactagcactaaaagttaaattaaatactctacactctaataaaggtgcaagcaccccttcatgccttgagctatctacctccaacgaccatttaaacgtgtgacttcctagtaaggcatatgttttcttgttacgctcttccactgtaacagttgtcccataggttaacgtccacagtaaatcttctaatgttgctcctcgtacagcccctagtttagtttcataagtgatatagtgacaaccatccatagggtctagttcatgagatactattcgttggttagttccaagcgcctttataaatgtctttaggttgtcgctaccctcaattatctgtacatctaacttatatttatcctcactaacagacttatgttcctctaagctacgtggtttactgattacccctagaaacgcaacattatgatactcatgtaagttgtctactaaacaaaatagtgtatcccctgtttcaactactttaccattatcgtcattaataaagaatgtagttcccccaccagagttatcaatctgaattttatatatcgcaccataagtgaaatagtcttcttcactccacccacattgaagatgggttacttgagctaaatccctatctgatattttaagctttcccatttaaatctcctcctataaatattgtttaatagtatacaatgcttcttctaaagctattgactcatcctgcagttcaattgctctagtacttaactcttcactcttctcgaacaactgataagactcttccgacaatgcagtaatcttagcctctatcttctctaagtcagacatgtcatggtttagtctaggttctgcttctgagggtgtttcatctagtagctcaaatttgttaccgctactattaataacgtccagtagttcttttatggatttgccactgctaccatcttcctcatcatcccagacccgatagcgaccatcagatgtaaggtttatctcgtagtctttaccttttttccaaaaggggtgaactgcctcaacacatctaacagatagtgtaggttcttcttcaatcttatctaacatgtcctccgtccaacaccaatcttggtgtataccatcgatgttaataaagtagcgtacgttactcccccaaacacgtgtaatagtagcctccttgcctgcacagcctatcatcttagaggttatgtgagtcttatattctaccccgaatgcctgtaaatcattagacaccttcactcgttcccctactttaaacctaggcttattcattgcctcttcttctactagtgtaagctcaaccttattagtggcatactcctgctctatattcttaagaacacctcgtgctgtgtcactgctgaatctgacatgaccatagttgtcaataatagcgtagctaccagttgtcataattctacgaacacgataacgcttatctgcactccaacttgatgcaaaatcttctaaaaaattaatataataaatcattctctcatctccttaatagatgtaatcataaaaacacttagctacatgtactcccttagtacctctaaagattcttctaacttacgagcttttgcatttaagttaagtgcttgctcatttacatggtcacgcttagcgaataggcactctgcctccgtttttagctgttcaatatcagctagaatcttagcttctacatctagctcttctacgtctttttctccatcatttgatggctcatctacaaagacaaactggtctgggagtaacacatggttccctagtttactattaaggtaaatacgcacacgtccctcagggaactctttaagtactttgtaacaatgcccaacctctacaagacctctatcaggagggtatacacaagttaccttagcaaatcgtacaggtttttcctctacaacttcaaatttaactgcatcgtcaccatttaatgctcttacaatgaactctacggttctgtggcttcttacgccttcccctgcgataactaagccaaggtctttatcacgtgttaccttataaactctatcagcctcccaatagcttacattagcattatcatctgtgcaacgtagtttcataccttctttaatatccttctctgtatacttcttcactgtgcccctgcctcctcagcttccttctgtattacctcagccattcgtactgaaattttttcataacgttttatacaatccgcatagtaactctcgttcatagcgacactagcgtgccacatatcattcaaagaacaagctagctccttctctaaactttttagttcttctgagtaattggtcatttaactataactacctcctaactatgaactactcttctaattcctactgcgtatactttaacatctccgctagccgtttctacattaaactctcccataacatactcttcatatctactatctgtactatcaatggcaaagtcttttctaagctcattgggatgtggtcgtttctctgtctgatacctctgccaatcagggtaatcacgagaaagaatgtcaaatactttatcatgaaatactttaccatgaaagttcattctaattcacccccttgtgcttttaagaataaccctgtataaaagctactgtaaacctcaagcaattcattctcacttaaattactgaaagcagaatatgcttctggatacttcattctacctcctgtggattctaaaaaacctactgtttctgttacatgagttaaaccacctactgcgtataattcgttaaaccaattaaggaacaatgtctgtggttcatttaaccacgttggctctacctcttttttctttaactcttttttgatataccgaattgcattgtttaaataaacttcttcggtgtcacggcttttatttcctagtagtactcgttcaatttcttcaattgcactctctaaatatcccattaattattcctccattccttttttaaatcttaattgcaaccatgtagcggttaccccgattactaatactataatgtctagtagtacgtttcgtacaatccctagccagtccccttcatgtaccatgaaaggtaaggtagctattgtgtataagatactgaacgtgataagcaaccacataatcaccattaagataactacaaaccactcaactaatttctcatctttactcatctagtttaccctcgtttaatttctcttcgtcttcttcaataagtagttgtgtgattgtgattatcccacgcatatttttaatatgtttagcaaattttttgcggtgaataccgaatgcaatagcatcataagcacctttagcaccttctgctagaaaaataacccaaatcagagctagaaaccatgtggctacattatctggtcttacatgcttagcatagctcacaaaggctactacagctactgacatgataacacgcagtatcccattaaacattacccaaaatgatgcttctctcatttcgtaactttccaacattgaaagcttttgtctagtcagtgtattcttaagttcttctaattcgttcatgattaagtctccttcttagtagtatcttaaagtttctcttccattcccatagatactagttcttcataacaatgttcacagatatgtttatctttatatgggttaccttcgttctcaaactcaaagtagtcatcccacatatcaaagtagctatcacaacgtccacaacgagtgccctctagtcctgtttcaatcataaagtcttctccaccacaatgttcacactctgcgcaatcttcttcttcatactcttctccacagtcttcacaagtagccaacactaaccagtctgcctcctgcgttacatcaataagtcccattgcaattagtgctttatctttttcttccattgccatttcctctacttttttattcataattgtcctcctcgtttaattatctttttcacttctaaaattactttgtagacctctacaaatatataagctagcccacctaacagtactgcaacatatagcaaatctattgataacaccataggtagggcaaaccaccctgcatttgttaaaaatgaataactatagtaacctactaaaacacctgcaaaggttaaaacaaaaacaataaaagctactttcacagttgttttaataatagctcctgtgacctgtcttacaatgttatctatcttttcagccttatccatagttacacacctagctgttcctctaaaccagttaaatcaatattgtagtagtcaataaaatctaataagttatctgaataaaactctacgatttcactagcatccattgcttcaatcagctctgtttctgtcaagtcttcaaaatatgctaaatcttcttgagaaaggttgtcttttgcatccgtcaaccagtctgtcattacttgtaacttcttaagcggatgtgctactttcagttctgcttctacatgttcttttactaattgtttatgttcttctaaaaactcaatatccataatatccattttattttcctcttttcttagataattttgcaaaatcttttagtaaattacttgggatgtttaacacagtaactagtgtaccttcactgctaacaataaagcatttaccattatataaaatacatgtttgagggtacttagagttaaatactcttgatactacccatttctttagacgacctgttagctcatgttgtcggtagcctcgttctaacgctaagtaaaattgtcggtctacagagctcttattcactcctacacgttcacgtacacgtttttctccatgtttgcttacttgactcatttgcttccctcctttatcttatgaacttagtttaccttatagtttgttaaatgtcaataggtttatttaaaaaaaaataatataatttgcaacccccaattaatgagggtcgcaatattgtgtttcttacttattcaacaattttttaactactagcgccactacaactgttaatgttacactaccaaatccaacaagcccatagtctaactctgtacctgtagatggtaactctttagcaggttctgtagtgctaatatgagtttccttaacttccttagactctgtagaactagaagcagaatctgtgtagcctgctacttggaaagtttctgtagtttcaactgtctctgaaccagtagaagtgtcttcattggtcactgtttgctctgtagagctttctactgaactttctgtagaatctaccgtagaagacgtagaatcagttgtagagctttctgcagtggttgtatccgtagtagaagacccatctgttcctgtagtcgtatcaacagtggatgattcttctgtagaagtttctgtagtaacagtagtattagtgaacttcacaggaactgttagtgtagcttctcgttcacggtctttaacgaatacgtagatagctgatgtgtccatcccttgttttccttttacttggaaagttactgagccataaccatctgcatctaggtcacctaagtattccgtgttgttattgtacacacctacctgtgcaccttcccagaacttacctgtagataatgttgctttaacatctacagtgactacttggtctcgttctaaatatacaccttcacggctaactggctccccattaatagatgctgtaacgttcattacagtttgtttaatttctgtttcgtcactaaagatagctgacattacacgaggcatatctccctcatatccagaagttaatgcaaagatgctagttgttacttggtcaggacgaccattaactgggaaggtaacctctgcattaccaaattcatcaactttactcttatagtctaccccattattacgaataatcgcataagcattttcgggtgtaggttcattgttccatgtcgctacatgtagtttaaaagtaactacttgccctcgttctactacatagttcccattatataagtctaatccatctgctactacatcaatatttgttacgtatgccataataaatctctcctcttctttatcttataaacttagtttacatcatgttttgttatttgtcaatacatttaactatttttttttattttatttcttctgtacctgtataaggtagctttaacgctcctttatagtctttcagagcttcttctacttttggtttagctttagattttggctcatcaatataaattgcaatatcaccttgtggtttaaatataatttttctaccgttatcccctacacacccattatggcagtagtaccgtcctcgtgtatacatgtatgtttccaacccacattcagggcaagtactatggtgtcggcatctgatggctcctagtgtaaaagaactataaatatactcttctttcgtagcgccactaggtgaagttcctgtcaaacataaatctttttcaaagtcttccccaccgtcatggtagttgaacgttggtaggtctcccccacttaaactatttaaataacaaagtgtatagtggatagcttcatgtctggctacatccattaaaatatcaagcgctctttgggctaagggtgtttcttgtaatagaataacttctttaacaaatctaccatttatctctatacagtgcatgtttgtgttagtgaattggacaaaagaacctagggttcttcttagcttaccattgattacaacatctatttcaggtattttattaccatggtagtctccataagcatttacaataaactcattaacatatttagcaattttgcctgaccatagttctttctctttcttagtcaatgccaaactctttacattcttaaattcgtacatttctatgtctgatttaacatagctgtcagctttaacaacactaaacatacttacacctcatatagccaatttactcgtttgtcttcatctgatacactgtcatagatattatacaagtaaatgtcaatttcttcatcaattctactattaatttcttctctaatagcctttaatatttttaaaattagcctgccttctttaactccttggaagttatctattgcaaacaggtcaccggcttttttataaagaccatcataaaggacatctagctcctccgtaggtcgattttcgattgtttggtacaaatagttgtaatactctaggctccctctcatgatacctcctccttaatctgtaaagctaatagctaattctcggtgtagtgctttgctcaacttcatcataatacgtaatgttggtagtcgtttaccttgttcaattaagattaatgttgagtggtctacttttgctcgttttgcaagttctaatcgagatagctcttggctctctctaatttgtttaattttatctcctagttcacaagcaaatactgcctcgtcctctgttaattttgcatatactttcttttgttcattcatgtgtatctctcctcttctttatcttataaacttagtttacatcattttttgttatttgtcaatagttttatttaattttttttctaactaattaattctagtgagaaatcattttgaagttagcttgttcttaattaattgtagtttagctccccttaccagattagggctaaacaattcagaaccactgtctaatctaggtaactcaaggtctaacattgctaagagtatgtctacttcttctttggaaaaagagactgtaatcatatcggatgggttgttgttctccattccctttatcaattcttcactcatctctatttcatctaaaataggtgcaatctctttcataaagtaagactctaacttttcattctccatttgtagcacactccttagcgaactcttcatactctttcctcatctgttcttgaaattctggttcttctaatagttctagtaaagcattcatcttctctagcatagacattgtgtcaaagtcatatcctctagcttctaacgctcttttcgtataagacatattgcttacctcctatctataaatacatattaacataaaaagagactttatgcaagcccctttttctaaattatttatttaatttttaaatctggtgcgctactgttcttttccggcttattgtacactaaggtatctgagtctaaataaagaactgatgaggtgtcttttaccttatctgctatacctaggaaacgagtccaccagttggtgtttatatagttctctgtaaacacaattttaggtttaatctctcctttagtagatagtatagtagtaccttctgcatcaatagctaccaaaccgccatctacattaatctcaaactgttcttttttagcctctacaactactttactgttctcatagatagggtgtagctcatggcttttcgttaccccatgacttggtgcaacatcgaaagtaattccaaccactgctagtgtgaaaaaagcagctacagcagagcctaccacaatactaaataaccatgaagcccagtctaggtacccataactatcatacattccccataaaacaagaccacctataaatactgctaaaacaataaaaaacaacatattaaatcctccaattttttttaaaataatagtaaacactgcttagttatttcggtgactacagtaagtgcgacacccgtaaaaagcaaagagtgtgcacctttagttatatcattttgactacctataagaagccctaattcaattagggctacagaagccatcccaaacaaatatagtaagaacatggatagcattgtaataatgctaaatataaaaagaatcaagtaagttcctccctaataaaagattaggtagtagtttaacaccactaccttaatcattgttatttctttgtacgacctctacgtggcactcgatatttgtctaaaagatagaacaaaccatttttatataatttgtatttagtataaatatctactaaacgagtaccattcttgtagtctcgaatgactgcgtttaacatatctttgtcattttccacatgagccactcgttctgctactttagaagatttaacgtctgccccatttgcatataagatagagtataaagctcccatactaattccgtatttacgagtaacgacatacgtaggtagaccatttgaccagtctgccaatagcttagcagtgtcttctgtagatagtgagaagtcatccttaccctcttcttcctcttcgactactacagttgtttcttctgattctgattcttcttctagtactgtatttaaaagattggctaagaagtttacaatgccattaattgcatcttgttcttctttacgagtttcctctttatttttaattgtaaagtctaaatcagacaacccgtaagtcccataagctagttcaatggacaagataagatagatagctgttacgtcccaaccataacctctattgctagcccgtaacaatgattccatttcttctaacatatttaggcttcctgcgtagaataaaccttgctctactgggtcggtacagccgtccatatctgcttccatagtgtccttgatacggataattgtagttacaattgcatgtctatcaacgtcttccgctaactcttctgcttttaaataagcttctaattctggtttaactgtatggtcaaactcttttagagaatctttagccattctcaaccctttttcagtttgtgggtgattctctacacgttcaagtaatttgtctcctgaacgtactgcatagcttaacatttggaaagctctggtatcctcacaagtctctgtgtggttgctatctgtttcttcctcttgttcaaatgatgaatcaatagtagagtaagaagccatacccatcgcctccacagttttacgtaataactcttgattatcttttaatttgttattgacttccaaaagttcctcagcatgttcgttctcaaacatggcaagctcgtctacttcttttgtaaagtcttcaacaccatcatcacctgtatttgtaaagatgtcatctaatgactctttattatctgtttcagagctatctccgtctttagagtcgtctaatgtcccagttaattcttctattctaaccaaaccttctgttctaatcaggttacctgctccgacaacataatggttaacttcgttaaagctatcggtaactcctgcatatagcaaatgtcttgcaacattacctaatagaacctctgctacatctaacttgaatctctcaaagttaagctgtaactctttaaggtgttcatcatcttctgtattattaagaacagctgtagtaaaaatagttgaccaatccataatcacatcagctaaattgtgtgtgattacatcaaagtactcttcctctgaatagaattttttaagttctctaccggcatacaaggtaacaatatggtctgtagtagacatgctatctgttactttctctaattcgtctagcacacggaaaggtacctcatggatagtgtctagtgacaagcaattaactaagacttcccaagcttgcttcatgtagtcaaacccaagagtttcaaaaaggtcttctccaacattatgtgatacagctctcattaagccacctaaagataagtctaattcatgtctaggtactgccatttttttcaaaacatcttttgataattcaattgtcatatctacatgatgttttttagggtcgtattctcctcttagttcttcaatagcatctctaaagtacattaacatcaatgctagctcttttgcatctttaaccttaacaattacttcctcaccaattacataaaacgtaccatttgtttcaataaactttaaggcatcttcataggtagctaactgatttacttttgtagtcttaatttcaggttctgcgccttgtgtcttacgcatatcatctaataagttatcccatacaggggtgtattcaccagtgtaagcatacactcctttgtctaaatcataaaaaacaatgttacggacaatatttgtcttagctgatgaatcgtgcaaagtcagttttgcatacttcgaccgctctttatcaaattcaatagtaactttgtcccctttatctaaccactttaatgtgtctaaaatatttgccatcatggcacctccaaaatttattatcaatatccttgatatttatactataacaaagttatagatatttgtcaacaataaatttaaaaaaaaagaggcaggcttacaacctgtctcttctaatctcactctctgcatatatttcgttatctctacttaacttgtagtaccacttattgttagcatcgtctttgtatacactgattacaatggcggaatctttgatagagtcctctttacttgtgtagtaaacagtgtcccctatcataaatttagggagttcatccgtatttaaatcatcatagataaaacatgggtatgtgctaagggcattaattggcaaatcgtatatctgccaatatcctgttctccaataaccatccgcaggggttgtatcataacttatctgcgtaacaggtacaacaccttctgagaaaatccttactagcacatctaacttacgtctttgttctagcccttttacaataagggtcttcccttcaagtaatgcatgtaaactttcttctaaggttaacgttttataatttacctctctcaaattttcaaccattataaccctcctaaattatatccatacctgatgcttgcaacgctttagtgattctttggtcttcctctgttaaatctaagttttcatagaactctttctctgctgattcaattgcgttgaatacctcttcatgctgttcttctggaataaactgtagaataatctcagtcattgcttgttctttagcagactgtcttaatcttagctctcttaaacctgttagacttagaccttggaactggttagctgttatcttagcttttacttcaattgctttcatagcaagtggggcttcaacatagtctacctcttttagagagttaaaacctttttgtatgattgtgtctaacacctctacatcattgtatatctgttccacttgcccgaatgtgttatcgtatgtttcattaggaagtggtgttacctctttagacttaatatcaataattttacctgattttctacgtttgtcaagtagtgattccaagtctactcctgtttctaaagactctcttcgtttctctttgtacctagataatgcgggtttattaatttcaaaatcgaacttctctttacaaaaagcaataatgaaatcgtatggtttaccctcgtctaacgcctcgtctaccttagatacaagcaatttattattgtatagattcagtaatactgaactgttattcacttcttttttagccattttagttataaactccttcaacttttaattatatgaaatatcatttttaagggtcgaaaccctttgttttattagcttctgtaactaatatagcagaacatctgtaaaatttcatatgtaattgaaacaagcaaaaaccccacatattagtagggtttttgtaactttaacagctaaatcattatgaaatatttgtcggtcaatatcagctatctaaaataagttgttttacctaataatgaaataacaactgtttagtgtacttttttcattgtgaattggtctatacacacctcatatttcacctttttaaacaaatgagtacttaccaatttctgctttgactagccatttgtctaagaactcttgctccttatttgtcaattcacggttagctagtccccttgcttgtactagtttattattttttacctcaaaagtaactagactagtgtctggctcttgcttgtctctcataaacaaaatacgagtagaaccttctgccacttgacgagcataagtagcaacacagtggtgtaaactagaaccttctttagttaagtcttccacactatcaggaactttaacaatatagttacctctaactgatacttcttctaaatctttatatttagctacgctacttgcaaagttattattcaatacctcatctttcattgttcggtaattcatattaataatgtcatgagcagtcttaagagctttagggtacttaacaggtaccgcattcatataatggtgtgctctcaagtagtctctgtatgtgtacctagcctctgcttgtgaagtaagtccttgctcaacgtatacttgatagtagagataacgaacagttctatagaagtctaggtttgcaaccatagaaatatcataagtagttgtactgttatattcatcgttaactactcctgcaataatatggtcgtatataggttcccactctatttggataatatctttactacgagagataccatattgttcgtctagttctttagtaaactgataaaggttgtacaataccccaccaaaacgagcataggcttgtcgtagcttagcaacctctttctctgccttcttcatagcagtaggagattcaggggtttttacaatacttctgttatccactgacttatggaaacgactagccattttttggtagaatgtccactctagtccgccttcacaaataatcttgaagatagactttgggatacctaagtgcttagtcggatttgtttgttccttgtctaaatagttcacattctttataggttcataccatttatttctacttttcccttcacctacaacaactactcgataagcgtaagccaaagcaaagtctttaggaacaccgctcttgtaaattaattcaatcttgttatacttcatcaatcggtcaaagaaacgagatactttaccgatttgttcgtctttcacccgactaactcgttcatacacggacacatacatatctgaaccagacattgtatctaattgttcaagaatattctttgctacgtgcccagaaaacatgttactaatgttattttctgagaagtttagtggtttaccattcttaatcagcacagctaccttatctcgtaggttatattctagctcatatacacgagatacctctcgttctaaggtctcctcttggtctccatagtgtactttacttactaccgcatttaagatgaacccataaggcataatcttatggataaacaagtgactttctaggtagaaacgtcctttcttatctgtgccacgctctgtataagcaatgtctcctgtaccaaagtcataatcgtggtctaattctagcataattttggcagattcttctactttatctgttacttttccttttctattaaatatatctaagatttctttattttgttctaacttaatcataatatcctcctaatagtacatctatcaatcgacctgttaaactatattgtctaactgattgagcgtaaccatctgataacccatggtctttaatactatttaaaatctcttctctatccgcctctaatagcacttgtgcagtattaataagtgtcttagcctgtgctgactcacagtcttccctaaactctctaatgatatttgcaataataggagagaacgtgagaccttcacgaatagattccccgtccgctagcacaactaataagtccataaaataacgaataaggatagcacattttaccgctactcccatatcgtcattctcaatagccttatccatacgtgcttttgtggctttaatacgaccattaattcctctaaacaacttctctggtgctaaataagtaaatgtttctcttctgtattctagttctttcatatacgtaactaaacgagtacgcttattgtcaataccttcaataggctctccgaagaaactttgggagcttgtggcatttaacatttgtaaagcctctaaatctccattttcaatacggtaaacatattccatgatagacatatattttacttctactttgttaccatcaatttctgtttcttgcttatgaattgttgtaggatacattaaatcataaggagatggcatgtacacaaccatataattgtagtcgctatcttctgtgtagtaccctgcgtatcggctacctagctcatacacagctaatacatcacgcatttgccctaaatatcgtctctctcctaaagtacctaaccaagtaagtgctcccacatataccttttctttgtagtccatgttaattctctccctctataccgtttattttttattcaactgtagttatcttagcacatacaaaaaaggaagtcaacatatattgtcaacttcctttaaaattttatccaattagtaatgtaactttttcgtctagcaagtagatagtcaccttatattctcccggtctagccgacttaatagttacattgcggatgtcttcctccttaaaagcaattgctcctgtatcacaagaaatatgcacccaaggaacagactcaatcatcacaaattgcattaccatatcgtcaaacatagggaacaaggttgttcctgtgctacctcctaaaacagacactgcaccgattacttgttctagtacagaaccgtttgcgataaactgtaatactttaattccctctactcttgctagtgcttcggtgtaagtatgcacacctagcaatgtttccccatcatacaattttttacggaataccagtgctaacgtacttgttacaccgctagaagactcatcctccaatacatattgcatatgtttacgccctctaattgatacttctgtataaaaaacttttggagaggacatatataggttaaatggaatctcttgtaggttagagaaagcagagtctgctaggctaacatctactgcaaaatcactattgattaaaggtttctcaatagtatcatctaggaactctccataacccccataagctgtgcttgcttcttttagtaaacttaatgcattaatctgtctttctcctactggtctgaatgaaatagttcctgcaactgtttctttcttaaggtctacctcaacatcttcaaaagttgttgtaataggaatatctgtagtcattattgagcttgtgtagcgtgcccctagcaatactgattccttatctgtgataatattataaaattttcttagtgttacttctttcccttttttaagcatagcttacctccatctaatagtaaattattttacaatttctacgtctacatactctccatagttccacccagaaccacttccgtagtccccttcaataactatccgtgcatcattggtaaaaatgcctatcctaactgttgaacaaccataacattcttcactattatcttcaacttttgtaataatgttatctgtgaaatcaaaaccatctggaagaataacctcaaaatcattatacccgcaacagtctggctctgggttacaagacacttttagacgtgacccatcgtttaatacaaggcttgcttcacatccatcctccgcatattctagggctataacacttttaccaattagctcatcataaatactcatagtagtcctccatctatctagttgttttagacttattgctcttccgtctatctgctttacgtttctgagatttggttgcgttagtataccctacactacgtacattaccgcctctaagcttcctctgtacgtagttcttctctaggtatataaatgttaaccctacctctctaaaacgtctctgagctcgtatagacagcttacctacaggttccttaccaatgctaacataataaactgtgtctaaagatgctagggtttgcccctctttctttttacgtagcttaatcatatctgcaggtacaccagtgtagctagacactaagtcttttccatactctaattttaacataatatctttttctgtcaagttctgatattctttaacaaatcctacttggtagtagccttttgtggacagccgttttgtgttactaccacttgttctgcccccagtacctacatagttcttaccaactacagtattccaagaaccatattttgcacctttaaaccccatctaggtcacccctattctcttttgctttaaaatgtcttaacttaattgctttctctgggtgattcctattatggataaacacatcacagaacactcttgtagcctcttcggtaccacctactctatttattaaattattaatgagttcaggtttagtcattcttctagcagttgtagatttaacatagagcccagataccccaccagaacggctgtttgttttcataaccttagctccattagcttctagcctatccccataccgtctgaatgtaccaagcattcttattaaatcttctttccacaagatatttaacatatgataagctgattttttaggggaacggctaggttgtttatactcacctaacattgcctctcccatgaactctgtgtaagctaggatacctacgtgtttatgattatgtctagcaagtatttgctctactttaggtacatggttatcgtggcataatacatatacatagtcgcacaccttttcataatcagatagttgtttatttaatctttttgtcgagtcacgttcagtttttatctctatgccaatgagccctcgtttttctgtgaagactaggcaatcacatattgtgttaccttgcatgattgctttctcaaaaacaatagcgctctttcctatatccccgaatattctttgcttatttaaaattaattcttgaatatctgcctcataaaattgtttatctgtcattatagtgtctcacttctaaattgtggtctttgaccatctaggttaaaccatatctgtttctgtgacaaataaccctttgttatagactccacgtctacatatgccttgtcccacttaatcgagctcaaaccgataaaaacccctgtattatctccataggctatgcccggttttccatcaggtagtagaatatgtctgtcttcttcacgttcgtagaaacccttaccttcgattcctgtgaatgtatcgattccattaactactggggctaggaataatggtggttttgtttcaactcttcgttgttcctttatcgaccgtcctttaatcttttgtgctagctcacccagatatgttgtgcttttgaatctaggggtcactttttcttcaatgtaatctttttggatagagtctccaatctcttttactttatagacagatactacatcagtacctgttagctcctcagctagttcaacaatataaacaggggtttccttcacttcattattaacctttgttttaatagataagaccccacgtctgattaatgttggtgggttgtttacatctattaatgatgtctccccattagggcttattacttctgtaaaattcatacttagtcacctcattattcaatatagcaaaaaagagactagttagtctctcttttaccttttcgttcttctaactgcttaatatattgtttttgaacataacgaacaattgcagttctaataacaacttcaataattcttgctgtaaaaggaactacaaataccattagtgtgaacaaagcaatacctgttacgttaagttgcaagctagttggtatgatgaagataagagaaattgctatagctattcttaaaaggtagttaatgcctaaaaatacataagaactacgtggcaaaggagcctctccttcttcaatcatgtatctttctttacgcttagtattagtaaaggcagttatgccttcacctacgtatgcaatagccaataaaatataaactaaattaccttccatacgagtacctcctataagaatactggcatgaattgtggtgtatcacgctcagggtctaataccataattagctgtgcagggcttgttgttggcaagttattctctgctgagtagttgtttgctcccataggagagccgacatacacatggaatcttgaaaagtcttcttgaataatacgtgttgtatggatatgccccatgattaaatagtcaataacttcatctttgatatgtttagggattttaacatccacttttttgccctcagtgtcaccatgagttactttaatacgtttacctgcaactgtatctttaaaactataaacgtcacttctgttgtctacaagtttaatatctttgtttaatgcccctaattcttgtaagaaatgaagttggtctactacaagataagccacattatcattatgaattttatcattcttgttaccttggaaacggtcgtggttaccaccaaccataccgaaagacacttcaaattctgccttagctagtaaattgagtgtatcagcaagaactctaattcctttagcaatctgctccgtagcagggaactctgcttcaaatgcttggttaacattacgcatattaatgtgttcaataatatccccaatatggtaaacatgaattttcttaatatcctgctccatagccacttggaatacttcttctaataacttttcaagtcgttctttaaagatttcaaagttgtatccccctgtgtcaatgttattaacaaaagcaccaatatgccaatcagataggcacagaataagttctcggtctgaggatactttcggtgcctttaaatttttattgatatattttaaactagacatgtcttttagttcagttaataagtatgtcttcaaaccactaattagatggtcaagataagctccctcacgttgtaacttaccgaactctcggttacgggctagcactgcttgattttctactacgtaagaagacagtacatcaaacgatgatttatcatcttgtaacaatactttacgaacagcctctagcgtaattttgcctgccatgctagcttcgtaaagttcatgtagctctttatcttcttcaatttccttaactagtgcctttaagctatacaggttctccttatctaagtcaaactcctgtgctagtttattaaatttgccaacgtgtactcgattaaagctccgttgtaaactacctataataattgtttgtaaaattcttggttcatctaatttgtctaataacttctctaaaggttcgttgtttttcaaaaatttacactcctatcaaaatttatctatctttattataccacaaagttaaactataagatttcagaaagcacacttaacagtgtgttatttacagcttcactaggtttacctaagaagacggttgccgttgtgtctttccctttaccattatgaactttataggcaacctctccggcattcataccatagctccttacactaaacatgtcgtcaccttttgtgtgcctaccaataaagacaataactttttgatagttatgtgacttataaaaattaattaatttatgggctacttcgttaacatactgttctgcataaccgaaacaaaccactgttccattaatgacgtttgcagacatcttccctattgttcttagcacatattcctgcatatttatgttcaaggcatctacaagtacttcttgttctttcagaatggtgtctagctctttactaggctctgtcaccttatataggttctctccaagcaaatctacaagaagaacccaagagatagttctgtctgtaacttcataacgattgtagttgtctgtctcttctataatatctttggaaaacactaatgcctctttagcaagagccactacattttgttcatcctcttttgtacctgtgttagtgaacttagttaggtcaattattagttctacaaaatgaccaacggggcttataacctcgtctacaaatgaacgaaagctaccttcattacgataggtgtccccaaatgttgctaaatggtacaccttgctaaacgggttgtccatagatgacagtgccgtgtctaacgcacctcgttggctttcaaagaaaggaaaccctactatgataacagcatcatgctcccctatgtggttatgaatatcaggtaaattgaatttattaagaaattctacgtctacattatttttataaaatctacgtagcagggtgttcatgacatagccttgggtgttcttttttgtaataagttttatctttgtcatttacttatacctcactgtctctctttaaaaaacgtatgtctgtacgttcagtgtctctacgtccatttaagaaccctaaaaggtctttaaacgatatatcttggtcgcatttacctaggagctctttatagtattcttcaataggtttatccgcctctacaaacccactaatgatttctttgccatctgggtcaagtacgctaacatagaaaaatccattctcttcatctaataaaatatcgtaccctttataagtatctcgtattccttgtttaatacatttttccttattgtcgttcatgtggttacctccttatacagtataagtatagtatataactaaactacaacattgtcaacactatatacaaaaaaaaaagagaccttaacgggtctcttataatgaaactattctgttttgtccccatatacaaatggttcatctgctaggaacacattgtaaacacctagcttaaatctgtcatcagcaaatgctaagaacgcttgtaaaacttgtttttcttttacatctttagttttaagtttctctgtagcaactacagggtagttatatgggtcttctaatgtacgagtaatgaagaatatttcattatcatcagttataataattggttcatcagaaggtttaaatgtgttatccttttgattatagattaattcattaactgtgtagtctgttcctttttttgtaacagcatatgcatatccgcctgctactgtacctgtaaacccatctgacaatgttggtacaataaggtatttaccatctatcgctacttctaatgtctctggttttaataggtcttcaatatttaatatttctttagccattttctttcctcttttctatgttattcagttggttctactggttctgggtctgatacggtaacatgtactttacctacatgactaccttcctcagttgttgctataatgtctgcttcacctaatgctaaagctacaacgtcaccgcttgcattaacactagctatggtatcactactagaagaccaagtaaatttaggattcgttgcattatcaggtacaatactagcagtaaatttgtatgtcttacctttttctaaagcagattctccactaacattaattcctgtaacttttatgtactcctctatctgaactgtacatttatcagtgtgttctccatcctcagtagttacggtaacaattgcagtacctgccttatctttacttgtgacagttgctttattgtcagagactgttaaccctaaaagttctggtttatctactgagaaactaacttttttgtttgtagctccctctggtaaaaccgtagcagttagttctaatgtccgttctgtgtacattttagcggtctgtttgtctaatgatacactgctaactagaacaatatcagggttgttttctaacgataggttacctgttagaaagatattgtagctaccaaatgagaataggtcatttacaaaaagtttaaacgcttgaagggtacgcttgtctttgtccatgccttcctctactgtgagctcatctactactgcatgattatatgggtctatagttgttttacaaacatagtatactttatttttattagtgtatacaactaattcatctgacctaataaatttgttaagcttatcattccaaaatacagggtaaatataagtttcctcagatttttggtctatctcgtagttatagtgaccagatacggttccagtgtacccatccgaaatagtctctaccacaaggaattttccaaatagattaggcaattctaatttgcttatctcgtctatcttcatgtcgaagctcctttgtattaactaaggaggggttacacccctcgtacttagtctctgtttttgaagtcttgtctagttccgactacttcttttcctgcttcttcttttgctttaccataagaagttacagcgttgtttttgtaccagttgtagatggcaagacctacagatagtactaacattaaaccttggttcatttgttcgtctgtgaatggtaaaggttcccaaccaaatattgtagtcaaaccagttgccacaaatgagattcccatcacaattaaacgaataaatggtgcatacttgccagtgttgttttgttccactaaaaaatacctcctttatgtttctactgctaatatagcagacataaaggaggtattactctaatctgttaaatttattaagacaaactcacgaacattttttatagcatcttttacggctttcttagacaccccgtatttttctgacaattcagtaataatacgagcatcccttactgttccttgaattaactgctgaaatactgctaattcaatttctgaaaaattggcttttgataataacgattcaataagttctgcatcctcaatatctgcctgagagctatcgtcaatccctaacaataactctacttcattatcttgggtgcctagacgttctctagcggtatctcggaaccgtccttttacatagctgtgtcttactcgtaaattcagagcttttttaatatatcccggaaagtctacctctccatttatatcatactcttttgttaactttataaattgctcatcaatatagctcttgagctccttacgtgaagcctcactattgaacgagtctttatgagtgttatagactttatatctaaggttactatacttatggtataatcggtctacatctctagggaacactcctttaccatgagtctctgtaataaaccgattaccattacgtatttctttttcctctttttgaacatctctactcaaaagggttgtccccctcaaacactaccttaacttgtctcttaaccttctgagcatcgtgcccataggctccatatacatcgcagaagtggatagtgtgtggtatttttaactccataccataaaataagactcgttggtaacctaaacaccaacgattacctacatattctatctctataacaacagagttcgcactacttaagtcagagttctctattaggactctcttaccttctctcattgcctttaagaacatgtctcttattggttgtagtgaactaggtgtaccactgttgttcatagtcatagtacgtttagaggagtcttgatgtttttctaatatttcaaaaaatgggttaggtgctagtcctttgcgcaaaataagttcctctcctttatagctaataaaggggctaaatgcccccttatctgttagtctacctgtttaagtaaatctgaaacatctgttcctgtttcggggacttcttctttagtagcttggttaggttgttccttatgattttccatatattctttagtaaattcatataatggaattttgcatacatccacgtcctcattattaaatggagcataacgatgtgggaatgagcgtaccatcattttagcaaatagttcgtctcgaacaggtcttccctcttctgaacgtaaataagctacccaagctttatctgaattaaatttaatctcttgtcctgcatccgttgtataagacttccatgtgcctccactaatcaaggcatactgcttattagttgcaaaacatgaacgataaatgttttcctctaagttcagacctgtgtcggacattaagtacatttccgctttttggttaggacgggacacttttgattttgctgttttaacacgcaggatatgtcctacataggtctcttcctctgcaccgaatgcgtttagctctttattcttaatctggcttgctttaactacttctaaccgtaagctagcccagtggtgtaaagcgtgtcctccgggagagtctactccaccaaacatactacctagctcgtcacgtgcttggttaatagcaataaataatgcttttgaatttgtcattaaaggtgcaatgatttgagcaaactgtgccattgcttttgccttaattccgggctgttggttacctacacctttttctaactctttctcagaagctgtttgcgctactgagtcccaaataattaatacaggttttccgagcttactgaatactggtaggagtcgttgtaactcttctgctacacgttctaccgtgattgtatccttactaccgtctttgttctgtttaggttctaccatgaataccccgcctgcactaaagtctactcctaattctgctaagcgttctgggtctgcggtaccctcaacgtcaatccatacagttggtacatctagcatctgtgctacctttgtcagatgaactgctaaagtggacttaccactagagttcttaccaaagacctcgaccatacgtccaaatggcaatcctccacctaaaatgtaatcaagctgtggaatcattgttggtagtctgtctgatacattagagaacttcgtgtccgtaaatgtagttagacctacttcttttgtcaaatcgatagttccaaaatctatttcttcactttttctttttcttgccaatattgttcactcctactaaatattttggatagttaccacagaattaaaaggggcagtatgcccctcctaactgtttcattattgttaaacgttgcctaccatacctgctaataggtcgtctacactaggtgtgttatttggctgttggttaacaactggttggctgactggctctggtgtattagtcacaggttgctcaggtacagcattttgctgtggtgatacatcaggcatggactgcatattgaaaggcatatccgcatcagaaatggtagaagggtctgtcactggttgttcattgaaagaacccatcggttgttgctgttgtacaggttgttgtacaggttgttggttaaaccctccattaatgtttggaggtgttcccatgttcgttgcattaaaacctgtagcaggctgttgtggttggctttgctgtggcgaccaaccggtattttgttgcattggttgtacaggttgttggttaaattgtggttgtacaggctgttgattaaattgaggctgtactggttgctgattaaattgtggagcttgggttccttgactttgagctccttgatgaataggttccttcccatctactacatcaataaagtactcaataaactcacgattgtactctacagaaggtgtagcttggtatttcaagtcttctaataagttttcccaaccttgtggtaatgcacctaagctacgattactaatcacttgtacattgtacgaagtaggtttgttactacgtggtggttttgtaattgaaatagggaacgcatctgcagaagagatgaagctgtattgtgctacttcttctggtacatcaggtgaaaactgtggacgtaacattgggttacttaagctctcattaatagccatgcaggctgtttgtggtaacttaagtaaacgaaccattagctgaccattagaatctgtttcgtagctaacttctcctgtttgttggttaataattaactgcactacgttaacgtagtaacgttgacgaggttttgtattacggctataagggtttggtacacgattttctgcgttccaacgaatcatagcttgttctaagatgtcttctgcattagggtgttctgaaaagataaagttgctctttaaatctttaccattacgattacgagttgttaagaatgcctcacgagcgctaacattatagttttctcccggtactgcaggaggtaacacacgtacatatagagttgatggttgaccattttctgctttagggaagtataattctttatgttttaaatgatttgatgggtagtctactgcttcacgttctccaatattttgttgttttaattgttgttgtaattgttcttgaaaattcatttaaatttctccatttctatttttttttatattatattatttttttatcactaattttactgttctaaattccattgaaagtctgagaaaccattatttaccttatttttcattgggtctggcatactaaccttttggctatactcatattccctacgaaggtctgctccatactgaataagcatgtcttttctttgttcaaaagctttaactatatattgcagttgtctcacgttaaatgataggttaactatactctgtctaagtttgtaatagtcttcatctaacataatctgtgcctcaatctggtcttttgtaggtttagctactccttgattgattaaggcaacccgagaaggctcatatagttctgctttcttcttctctgcttttaactcatagttttcttgatacatgcgcaagcgctctagtatcgaagtccaataagtatacttagcaggttgctctaaaaatgctgttctcacattaaattcattgacttgtaactctgtttgtaaatcatgtgtcacaacttctccgttatcatcaataattttaattgttttgaactctaaatcatctactcgtaaattaagcacactctctaactcccccttcttgtattaatacagaaaacttgtctgccatattataataacatactatacgtcaatagtcaagctatttttaacatttttttattaaattgtcatttgtttatatgactctattgcatcttggataactttgataccttggtctttttgctcttctgagataagtttgttatcatagtaatctttaatcttagcttggtctttgaagtattttacgtatcctcggtaagaagcaaactcgttgataatttctgcatcataatcaaccatatcattgtaattctccccaatctctacatctgccacaatagggtaacgcatctctttaccttcccactcaatcattaagaaatcaattggcaagttctccataatatatttagccacttttgccatctcgtccacttcatcacgtgggcaatcaataacaagtgagtcatgtacagtgataacaatacgtgaacgcttattgtgttttctgatatactcgtcaatgtataccattgataggttagttaagtaggcaccagttccttgaatgattgtatttactgatttacgcattgcgccattaaatactttcttatctttagagaatgagtcacgcaatagtctacgatgtccttgtagtgtttctacgtagccataacgttgagcaaattgatgtgtctcttcaataaattgtttaatacttggcttacttgcaaagtactcttcaaaaatacgttctgcttcttctacagttacacctaacttaggtgcaattgaaaaaggactttccagttataccctcggtttcccgatatttattaggggcttagactatatcacgcccctagtcgttactctaggagccttaccatttccacctgttggtgtactctactcagttatattgtaggtgtcctctcgacagctactcacttttcgatagtcgttagaggttttcagatttaataaacttccaccctttatgttttgttctctctcctcttaagcatctagctacacatttagaatttagcccatacttttccatgtcaggagctttatagaaatagtaggtgtcacccaatgggctaatcgctatgtgtggttgtatccctttcttaggtttatcaccttcataccagaatacccatagcttatgatgcttgtatctaccattaattacttgaactatattggaatgattaaggttgtgctctctgcaaaacttgtcaatgttgtctataacataatattgaccatttgggtcaatagcacagacattcttgtagttcattgatagtaggcttttgttatcattaagtggcatccatgtacagttagctatactatactcggctcctccttttaagtctttatctaaggatagccctgttgtgaggaatgtatctttgtcccaaccatctactaaatctacagttgctaggaacccatttaaggtctcccactccttactaatagttacacctctacctccgtataactcatatgatttagcatttttattgagacatcgtctgcgcatgtctttatgcacagttcttaattttctatataaagggttgttgtctgttcttgttccaatttccataataccactcctttagtggagtttattaaatcgtctttactacgggattgcctctatccttttcaggacttaggttctcttatcagcttattggttattcagctatgcccgtttaggtaagtttaatcgagtagcttacgctacaagacggcaattgtgcttaccgtaagcgatgccgaagttgacactctttgcacgttgtcgcatatcttttggtacattatctttaggtaacttccatacaagagatgcagttgcctcatgcatatcttcaccattaataaatgcttgtgtcattgctttatcttttgcaatcaaacctaaaatacgcatttctagggcggagtagtctagttgtagcatcccgcctccctcaaatcttgttctaaataaacgtttaattgggtatttatagtcaaaacgtctagggtctccaaccttactacttaattgttgcatattcgggttgtttgcactcaaccgtgtagattctgtacctgtactgttatatgaaccatgaatcatgccatctttgttagatgctaatttaggtaacttttgagcaaagttattttttagtgtgtttactttagaatactctagcaaaagttcagcaacatgtttagcctctggataattttctgcaatataagctagtgcatgtttatctgttttataatctctccatgttaactgatgttctggtaccccattgtcaaaagtttcttttttaatagattctttatcataaggtagtgtaagtcccattactttaaatagcagtctacctttatgttcagacgaacttggtttaaaatgtactttgttctcactatcagtaattttatatttatctcgtagtctggcaacagaggcatctctatctttgacaggctttgtcatttctttaagcccctctttatagaacatcatatgttcatcttctacttcttttacttctggtagctttctaatttcattaagcaaccgatgttcttcttctgtataaatattctctactaattctgcgtattcagagtctagtagaacaccagaagcctcgatgtgtgctaatgttgctgttaatctaggataaaagcctagccataattccaacatcttaggatgttcctcaattcgtttatatagaacattatagatacgtagacagcaatcgacatctccgctagcatatgggtgcataatttctaatggaatccagtcgtaattaaagttaccaccgtcaatctcattaataagtttaatatccgatttcttaggtactacaactttttcttttactggtttaatgatggatttagtaggtttaccaataagcttagcttctttaacttcttgcttatatctatccattgctatcttgaactcacactctactcgttctttttcagcttctttaaaagcatcaatctctgctttcttacgagcaatatagtcctctttatactgttttttatactgctctaacggttcgtcatagcctcccatatctgttaattcatatgcaatatcggatagccgttttgaagtgtctactttctgggaaacaattagatagtaagcaatttttgtatctctgttgtcttcaaactctttaaatccttttgtgttcattaagaatctaatatcgaactgaatattttgtcctacttttggttgctgtctgtccgctaaaaacttctcaaacaagtcatatatttcgttaagttcttctggactccatttagcttcatggtgctctaacggtatagtcacaccttgcccctctagccaactcatagacattacaagaggcttagctcctaataagtcaccacgtaaactatttgtctccaagtcccatgcagttactggcttcgttttgtctaaaaaaccaaaaatttgtcttacacgttcaatgttatcaacaaattcgtaatctacttttttagggataaatgcttgttccccttcttgaacaaacttacccaatgtatcaatatccgccatgataaagttctcaatgtttgggttagctgtccagtattccatactaaacataggtagtacccaagtttcaaaagagtctactacttcgtctacctcaagtccctgctcatcagccgttctcagtatgttctcattcgtaattgttttcttagttggtactcctctattctttgtgatagaagtacttcctaataaatttttacaacccatctcccccattgggatgataatatctggtttgtatttcattaaacgtctgagtaaccgttcctctggttctttacgctgtcttaacgtaggtgtcttatacttaattggtttccctgttctagggttaactttctgtacttccggaatcatgtcgtacacatagtctacagcatagtctctacctgtcagccctattgtatttaacagacccgtgagttttttacccatttctgttcttttaaaatcaactgtttgtccgttctttccattatggacatgattttctctaatgtactcttgtaagaatagaattttcaattacacttttctcctctctgttgctctatgacactagtatactttgtattatgttatttgtcaatatagaaagagcaaaaaagagagctttatttgctctctttattataagtgtctattgactctttcaacttggataatggtacaaatttaactacatatttctcaggttgtataaaacttttagagtttataccatcccatgctaccttctctggcttttttttaatgtttagtttccataatttgtggttctttacagaaataccttgtgatactgcttgagctactacttcatcttctacttctaaaatcttcaatatatctttcattcgataaccagtatggtgagagattgttcgagctatgtctgttcggttaatcgttttatctttactcatcctgattaccctttctctttaatgcacgctctctgttagcattacgtttaattttttccttctgaatatatctgtctattttattgtagtcctcatcactatcacacactagccaaatgttcatagcccaaatagataaggaagtttgtacatacttaaagaatttatatttgtactctgatttaacctcatagtggctacctactttatgatagtattcttcatgtctagtatcgaactcttcttctgtaagacaagggaacgatatttgaatcttgtctacatttgtttttagaggatgtagtgcaaataacacatcatacgggctaacatcaggtagtactactgggcagtcaatcgttacagggcaagcagtgaaagctagttgaacattttcaatctctctgttctcatagctattttttgtaggatagattacttcacttgttaaattcttagatatttgctcaattatgtagtatggtacaattgtatctgtgtaataagacacagaccccgcatgtttaaataatttctgaataagtctgtcgttcgtatacttttcttgataaagtatataagatttttcatttgttctatccatgtagcctataggggttttcaatgggttgcttgctagttcgtccatctccctctctatctcttcttcattcaatcggttaactttaattttttttgctatatcttcttttttcattaaattaatattaaagtaccggtcagtgttataaatattaagtcttttcactctgggtgcttttgtcattataaaatgctccttctactttttaatttccctatctctattataccataaatataaaagatagcttttcgtagaaagagcattatattgtactaagttgtaataaattttaaatagttttctgtattttcaagaacagttttcacatcacctgaactactgtttagctccattaaggctgaaataatatagttttgttgttctttattgtaactctgtgctgtttgaaataaaaatttatcaataataggttcttctggaatttctattagctgagcatctctgatatggctattcttaaataggaaattctctaaaaattccttacatttacctatatagctacttggtgactgtgttaaaaccagttgtgcccgtttcaagtaccaagaacttttgttaaggtcttcttgtagctttcccttgaaaggtgctcgacaaatatacttaataacatttcctaaatggtatctaatttccgcagggtacttagcagtaatatattctataacctctattacctctatttctccttgcgtataatgactagggctattaacgttgtctatcatctactctagccctcccgtagttttttagaagcttgtttaaattcttttttaagtcttttaaggagttcagcatgctcattgctatcttgaggtagtttcttccctatagctacaacatgtgcagtttctaagctgaatactcgtaaatcaatgtcgtatgtcaatgtatcaataacgtagtcttcccgctcacctgtccagttagctgagaaactaacagatatacttgaggtttctttcattacactatctgattcgagatacgactgtaccgttgctagagcttcctgatatgttttagccatactattctcctcctaaataagaattaactagtgcatctagtgaatcctctgcatccatttgtttagcatcatccattatggttgtttcataaactagactatcttcccagttatagtcactaggaaaataaaataaaaagtagtctttgtttttgctaccataaggcttaaagctagttattgcatctaaaactgtagtaaatgtcttgtaacaatgagtgtccttgtagtcgtctacatatgttatgttagagaccatcaaagggtaacctcttttttcaatctcattaacaagtttttcattatatgccatagtaacaaaattctttgctctgtttctagtaaatataagtagtgggatatttttcgtttcttttgcatcccccacaacttgtgcccaccagttcttaatttctttgttatttaagaataagttttcaatcgtccagtcttcacgatttttacattctataacaaatgggaagttagcatcgctagggactacaatgtccccggcaacgttattactagaagcccagtgaagccccccagaagcaggtactcggttaaactcatatccccaccactctgtgagttctttagccatttgacgttcaaaacgtccgcctttgttacgacttttcttgcctactgcgctgtagtctgtcattcagctaacctctttcatattatttttctttattttctttaatagactctaactctttttggattttttcttgaataaggcttaactctttttcgtactctgctttagcctctaagcggttctcttccgttacacctagttttgttaataaattgtcaaatacagcctgctgttcaattaaagaagaaatataaccatcattccgtttgcccataaattcgacaatcgtagttacatcggataatgatactgaacgtttacttaagcgttctaactgtttttggtggtttttatcatgaacctgccccgtagctaaccactcagcccacactctcatgtctgcttccgataattccccgatacgtttgttgcgtttactctctttactcatatgtttcgtcctccattaaactttcaatatttttgaactctttaagaatctctttttcaagctcttctgtaattgtaccactgtcttttaattttttcaatacaatttttaaattaatatcttgcataaggttataattctctaccataaatgcggtgttgcgtgctactgtgcttaacgctccttctagctcttgatatgtcacatacttttgaccatgttctttctctagtaaaacatactcatctgttttttcttcaatcactaaattctgcatctgattaaaataatcaacgaaattcattttcatacctccgtagtaggctgttaccatcctctattttgcgaacttctaaattttcaaatgtctttgcccatatttctcgtgagcacttatcaaaaaagtcatgaacagtaatgtactcgtcacatacacgagaaactacacctatttgatttgttactgagagtgtgtcttttactctttttgcatcgaaccctctaatagtctttttgtaacctactatgtcccctactctaatttgctctttatcaactaggtgtttaactgttactacttcttctttcttggtgtacatgctagtcctccttattctttaatgtgaacttatatttattatctgtcatccctacataagttaagctatagtttggataagtcattttaataagcttcactattttatcgtaagaggtacctaaataaggctctcttaaatcaaaagttacactgcactggtgtttttctaacataacagaaaagctacccataacgtacaataatggtggtttaaattctgggatgagcatagttataccaccccagatgaaccatatgcgccttctcctcgttcagttgtatctaacgctgatacttcaacaaactgtgtgtcgtatcgtggtagtaagaaagcttggcataaacgagtgccttttgggataaatagtgttcccgcaaccacatactcatcagcattagatattctaacttctcctccataaactagctgtagctcctcacttagcttctctagctgtttttcataaagagcattaaagcttggatacattgaccgagctactgttactgggatgttaataagtttaccttcttcattgattgttaaaacgttcttagaaacagctcttgcatctaattgtaaagagaacgtatttttaagtggtaaaccaacctctccacgatattctccttccactacaccttgtgtgtttcctaaagtgattggatatttcatcataccacctcgagggttaatgaataaaccatattgtttggggtcaaacgatgtttttattccagtagaaataacatttgaacctaatctcccgggaataactactccatcgtctgctgtgaataagtcatgtgctacatcattttctcttcctcgttttggtttcttagcgccttcacggattgtatattttactacagcaccttctggtacaaatagatagaacccagtatggtttccgtagtcccaacgaacaaccccatcctcagtaccttcaataattctaagggtaccacacttaaagtcatttgctttccaccagtcacgaactgactgctctgcttctttcatcgagttaaatgttccatgataagtacgctttgtctcatcctcatccataatatttctgctatttagaaaatatactgatactcttcccatccttgttagcctcctatagttattcttattcaaattctcttaatacgtcaatcgcttttaatgcttcacttagcttactgatttctatagatatttcgttttcttctatcttaatttgttgttgcttgtagaataaacaaagtttcttttcttttagttccttaattcttgcttctactttcttttctaactctgttttatgtggtctttctagtaattcaaaagacccgcttgcactaccatttaatcgagctactatttctttagcacagtattggttaccatctcctgacttggtaaataaaattcctttttcacccttatagatttcataggtttgtcctgtagcccaccatggaaagctcataccatctttgcagtgaagtaacatcccttcttcaaggtcttcttctgtgtaatctgcatccttcatcaccttagaaacaacctcaaattctgcgttccctgtattaccatttaaacgtactaagatgtcctcatctaggctctcaattccataatcatcaaaaatacatagtgaacctgactcttttttagttacttcatagattttattcgtagtccaaaaactgtagtttttactgtccgtgcaacgtaatttcatacctgcatgtaaatcttctagtgtatatctcattttttccctctcctatctataacattaattttattaaccctactccatctgcttttacagagtagttatttataatattccaagcctcttctctccctagactatttgcatcttgagttgttgggcttataactacataagtctcgttatgtctcttatatagtttctctgctaacttcttaatttcttttttagcatctttatctaaatagataaatattttttgctcttcattcacactatctaaaataagttcaacttgctcgtcagtcacctgcttaccaaaagtacctaccccagactcaccaactgttaatgcatcaggaacaccttctgttataactatctgaggggtttgactagctctattaatattaaatatagtgtccttttttgaatgctcaccctctttacttggagcgttcaaagacttaataaaactttgcccaatagctctagtattccaatattgatacttgccatcttttccatgagttaaaaaaactaagtggtctttaagtctgactgacttaccattttctaatggtacccaagaatcctttacatacccaatattgtgtatgtaaatatcattcaatgtaaatcccctcttattacagtataataagaaagggtatgcttctggctctctcaggttctggcttagtagtttaaaaccatctggcaatgggggtgcgactaactcttttttagcttgtttagtttcttcttttggcttacctaacgaacctagaagaagtaataagtattcctcatcagttaacttatctgatttaggcacataatttttattattaaacctatagccatattcttctaatatctctagcgcttctttaaaagacacgttgtaatacttcattacaaaagatactgggttacctgaactaccgcacttaaaacaatgccataagttattctttgggtgcccttcttctaccctaacataaaacttatacttagtatcgtagtcgcaaaaaggacaattgtacctgtagtcccttactgcatatttaggtgaacctaattcgttagataacaagtctgtaaacatttaagccactactttcctcataatatttaacattgtatacacattaccaaatttttcataaacactatttaaagagtctagtgacctgcttaacacataagtagccttttcctctctgtctaaccgcatatactcagatatgtccttatacgcttgtttaagttcatctgagtctggcttgctcaattgtagagtatctactttatttatgagctcttcctctactttaactttttggtttgtataaagtaaatcaagaatatccagtactttagaaatactatcatctacttcttttaatgtataatcgtcttttaaaggtgtaacaatgtcatattgttcagcaaagttaaacaactctgcattctcatttgccacttcttcattataccattcattctcattaaagcaagctatttttgaaataactctctttttattatccccaacaccgtatctagctactaatgttttgtctgagtctaacaatctcaaatatagtttttttcgagaagtactatactgatactcatatcttgggcaacaagggtaccaatcgaatagtgtctctaatgggaagctaaatagtttcccttcattactactacttgtaggtacaaatatagtaccttcgtttaattctctacttaatgtataaatcttcatgtaatcacctatttactttcttcaaggtaagaacgaccttgaacttttttcatagtgattacgttctcaaacaatggcttcaagttctccgagtgagttattacgaaaatactactaatgttcttttggcgttctttcaatattttaataacgttctcacaaccaattgcatctaaaccatcaaaacactcatcatacagacctaaattaacagcaatgtttgcttttgactgaactaaatcttgaatagcaaaagatattgctaagtcaattctcttcttctctccctcggagtttgcttgatatgtgtttccaccagagccattcactacctctaagtcaaacttatcagctaaactaccatctgctttctctgtttgggtactaaagtttatttcaatatctgaacctgaaagggtagataagtaatggtttgctctttcatttaagaaaggtgtaactaggtcaagtacctcagagcgtatgcctttattagagaaaatttcttgtgcaatagttttatattgactagcttcttcttcggcatctaactttaattgattaatgttatttagttcatcttctatttctcggtctttgtcatagtcatactgaggtttaggtatttcttgtagtcttgctaacatatccttaatgctgtccctattattaatagatgtgttcaatgtgttctctaatgtatctatctcatgatacaacgcattcatttcaccattaagtttatgataaatattagtatgttcttgaatagcttttcttgcttcctcttgtttagctcttgcttgctctaacagaggggaatacactgctaatgcactattaagcttttcgataaacacttctttatccgctatttctctttgaatgcgttgaatttcttgttctctatgctgtgggctcaataaagcaccgcataggtagcaatttgtatttgttttaacatcttctagttctttttttaatttctcaatagcagtatcattctgtgtttttgcactagacaatctactaacgttagaatactgtgtttcaacttctacatcaatttcacgaacgtctgcaggggaagttaattgtgccatctgctctcttagcttttctaattcgggttcacgtagtgctcttccctctgataaattcttctcactttgtttaatagctaattctgcattagctaactcgctctctttttgttgaatcagcttttctgtattctcgtattgttgtaaagcactattgtaagattgagttaacccatctttttcatatgttttagcaatgtactgtctgttcagctcttctgcaagagctagtgctttttgtgctctttctttagcaacatcctgtgcatatcggtaaacaccaatatcggctaaattctctaagatttgttttttacccttatcagttgctgtagcaaatatttctacgttaccctgaccatacatgatactattagcataagtcaggtaatcaataccgaacacatcttgaatctttttgtcagtgtctgctacagatttctgagttatatcatttgttccttggaaaaatctagtagtatttttatgtttagagtgtttacgataacgttcgatacgatatggtaccccatctttttcaaattctaaaatcactgacatgtttttctttgcttgtttattaattacagcatcagctttcaaaccactaggggtagctccatacaatgcataagtaacggtagacagtaaagtacttttacctgagccattactctgaaatgtttcgtttgtatcatttataccttcgataagtacgagtccttggttgtctaaatcaatagatacagagccaatggacatatagttctctgcgctaactcgtttaaactttaacatcttttcctcccaaaatattaattaattatttcaataaacatattatacttcaaaatactaataaagtcaatagtacaacgcaaaaaagcctagatatatctaggctgaacaattattacactcccattgcctctctcaaacaatcgagggctttgtctttaattatttttgcgttagggtactttttactaataaatgcattgacaacttcgctaggtgtcgaacctgctgttatgtttatacgtggctcaacatggtagtccttctggacttttattctaatattacttaagttgttctcttcttttattctctttacagcctcaacctcgtccacattaccaataaactggatataagatttctccatagaacctgttgttgatgggctatcagctgttactgtttcaaacggagtatagtcagttttaataaatttttgttcccacttcttaccctctatagtaatatcataaaaacctttttcttgaccttcgtccgcaaaagaagtctgtagggtattgcccacataaaacacgttagataggtttcctaaaaattgtcgtttatgatagtgacccaatgtaactatgtcaaacttgtcagggtataagtcagctactttaaaagcaccaccaagtgtatgagaatactttccagtagatgagccatctacaccaatgtgtgcacttagaatattaactgtttcatggtctaaattgtcagcttgttcttttatccaagctttcatctcttcgacctcttccccatagctaacagcatacaacgtataatcatctttaacaatcttgtttaacgtagaacaaactgtaacatttggtaaagcactaaatggggctaaagcactatctgaatatagagagttagttaccttatcatgattaccactgaccatgataacatcgacatcggggtaactactaattacttgaaataacatattaaatattctaacatcaatagatactcgtttatggaacatgtccccattaaataaaacatccccatttttgtttcgtgcatagttcatcaagttgtctaatatagtaatttgctcttttgccctatcagttccatactctgggtctggttttgaaaaatctttaaaaaagtgcatatgaaaatctgaaaaattaataatatgtttcattaattccaacctcctgaaagggtattattcaaattacttatctttttattaacatcgtttgctgtgtagttattctcatcagaatggctacttgctctattcatcgcattatctgctaacagtgcttcatgttctgctcgttcttctggtgtttcatctctaatagtcatagtttctgggagtactttaaaatacaacattttatcataggcaatcccactattattacgtaacttatctacataagctctcaaatatccgttactaaactcctctgatgtttggtttaatgtaaaaataagttctactgcgttcattttacgtttagaaccttcgatagctcctgcatttttaatatcttgaccatagcttgctcggttcaactgagataacgtccaacaaacaaaatcatattcttgtgcaatcgcacgaatatcttcatacagtttacctcctgcatctgattcaccattacttccgcttgcatgagggtttttcattaaatctgggtagtcaataatgacaacatcaatttgctgtcctttacgaatcattacatcggagattaactgggataaaccgctaggtgttagttcttgtggtttatatttacgaatccaaaggttcccccaattcatctgttctttgcccgctccatacgcttgttggatttgggtatacaagtctttattcaattcaccatcaactaagatgttcttcttactttgttgtgataataattgttcaaaacgaacaatcatacggtctactttttcctctaaaggaacataaagaacatttaaaccacgtacaacatagtttctagcttggttaactgcccaagtagttttaccaccaccagtaggtgcaacaaccatcccgacttctccacgagctagcccaccatcggaaatagcatcgatagctgtgaaaccagttgggtatttgttttgttgcaagtttgcaagtaactccatctttttatctacgtcatcgaagaagtctagcaattctgaattattgcctgctgtctcgatagtaagaatgtctctcaagtcatccattagctgttgaatattactatcagagcctagtgtgccttcatttgttacagatttcatgattgcttctcgtgttagtaccttacgaacgtagttctgaatgttctcactgataacttctgagtctacatcttcgttttctagttcgtataagtcgctaactacctcaaagactttgttttgagcttccaaacttttgttttgttttaacatcttatcttctactagtgttaataaactctgttcctctaagctctcatcatgtgttctgtaatataagttgattgtagaaacaatcatctcatatccagaatctttaaactcatccattggaattttacttagaatttcctttgcaaagaatggctcacttaatgctctataaataacttgtttttgaatttgactcatcttactatccgtccctctcccatttatatttcttatttgtcgtcacgcatatctttttggttctcccagttacttttgtcaatctctaaaatctcttgttcagtcattaagacatcatagattctatttacatctaacatacctacagaatctaaaggaattttctcctcaccaaattctttaatgtacatacctaacttagatatatttgttttaaacccatagtagtccgctaacatgaacattgttcttgagaagttctgtgagccccaccaagacatacttaattttctaccttgtttaataaacatttcagcctcatcatttgttacattagataatcgtgactggttacctaacaataaataaagtagttcttcgtcttcctcattcattaataaactgcgtgcagagtttatctgaatagggaacgctaaggcatactgtgttgaagttagcccacgtttactagaaaagtttgcaacttgttctttcaagaagttagcaatactttctttagcttcactagatacgtcagattcacctaacgatttaagtgttgctgtataataagagtacaacgtacctgcttttttgtcaatatcttttttatattctaagtcacaaatagcagtatctaactgtgacaagtctttctgtggcatattgtaagcattcaatagtcctgaaacaattggataagttgcaccataataagtagcttctgaattgatagacgtatacaatccatattggttctgtaacattctgtagaacagttttctatttgtgtaagcctctttagcctctgcacataacaaagtatttacatagggtgtagcccctactcgtgctttacctacagaaatcaaaaactctgttctatcaaattgcactgttaaataaactaaaggattgatattgttctcttcacaatatttagctagctctacaaattttttgtactgaggtgtcccaacaaatcttctaggcaacacatcgtaattgtaagagctttcgtatgctcgtagttgttgcttagcctttttctcatctaaatttttcagtctattatatttttcataaggaatgattgtcacataagcattatacattcttgaaattaaataacctttaaagtacgcttctggttcgtcaaaactatcgaaaaaacttctagtaacaacgttaaactcaattttatcatttaaaatgtcttcacgttttttaagtttttcactaagtatacgtgcctcagctatctccgcttttgttctatatctgcgttttggtactttagttggggctttagggaagacttgttctctaatctcctttgcttgtttagtatcagacgtgataggattttcctttggctcaaaattaagcatatctgtgttaaacacgactaaagtccctccattcattcctctttttgtttcggtttttattaaacctagttcctctaattgattaatatatcttgtaattgttctagggttttttccagtctcttttgccatatccgttttacggataggggaggctactgtttcaccccatctctgagatttatccgcaactctctctaaaaagtctgctatacctttgtttaatttaaatttttcttttaattcgatatttaggcatggactttgcaattgtatgccaccctttctttattatagttatttaggaataggaatatcaacaatttcaaattgctcatcttcatatatttttcttctctgctctgagtgttttttcaaatgtttatttgttaaatcataaaaatcgaatacttctactttgttttctcctgtttttttcttacgtaaaccccgaccaacacgttgtagtgtctgtcttaaggatttacctcctgcaccaagaattaatgtatcaatccctgaaatgtcgacaccttcatcgataatcgtagaagaaatcattactttaagtgcaccatcacgcatgtcctgaattttttcatcccttaaatcattttcaagttgcccattaataaagtagtgagctactccctcttcttttagtaagtcactaatagcctctccgtgctctatacgattaattataattaaaatcccattacctttatcatacatcttcttagtcaactttgcaataagataatttctatattcattatctactatacctttttggtaagcatccatatatgtagtagtttgaatatctacaggggctataattgggaatattgttattttaggtttagctgagtgacctaattcaataagcgtattattagataccttagttgtaatctccccaaagatagcttgaagtctttgccaaagcacatgatttgtacggtcaatcgagccagtaagtgccattctatactgagcattattgcaagatgttaacactttgtaccaagtatctgaacttgtatgatgagcctcatccacaatcattacagatatggaatctaaaaactccatagcctcattatacttcttaagaactttaccatttttcttttcaacaatcttttgatattccgcttggtaccctttcattttcataataacctgcttattcgtaccacatgaataaaaaatttcttctagttcatgtttcaactgcaagtctgcttttgtcttaacttgaaaattatttaggtatccctcaagcaaacttctttggttaaaccctctttcaaatttcggggcaatatccttagcaattttcttgtacattctttctttagcagtcagcttaagtttagcctctgggtctgcggatattgcagatacaatcgtaggtatcattacaaaagtgacttgctgaatgtctttcttacctgcaccaaaagcgcctacctttatcccaagtcttttctcaatacggtcaattgactgtgaaaaaatagaagaactattcgtaaaaaatgcaatacgttctcctgactctaggtaaggggttatttgttgtataagccctgatgctatttctgttttacctccaccagtactaacgtttacaatacctattctgttcttaataacttgctccacagattcatattgatagtcccttaatgttatcttgtcattattatccccattaagcactatctccttatccatgtcctcgactgacatgaactcatcaggtctatcatcaattatctcaaactgaaaaatataacctgactttgataaagttgattggaggttccctagtattgtttctacatgaggtagtaaccctgtaggaaacgtgtcattttctttatcgaaaaaatcaataataccatcccagtaacctgctttatatgcaggactaaattgataaccgtctgctttaactcctaattctgaatgcattaccttatggatttctggtctaaggtaattttccgaatcttcataccttatctgagtatgcataatatctactactagtctcatttattaataaatctcctttaagttagattagtatacatgctccttgacatacccaccttagtataatcaatatattcattatatcacatagtaggtaattgtcaaggtgtatacagagttgatttgtcctattttatatttttcgtagtcttattatatattatttatatattatatattttatatatagttatttaaatatattagttaataatataagggaaccagaaatcctaaataggacaatttagaaactattttaataattcatctttcagttgtttaatatctgctagctcttttgacaactcatctttgatagctttaacattagctagttctttctctagctctgttctctcttgttttatagattccatagcatttttatgctcttgttcagctatagttggtttaaatataagcgcacctgagcttatttgtctgtgtgtttctggcaattttatccctcctaacaaaaaaaaatagactctatatgtctattataacatatagagtctattatacttattctgttgctatttatctgtcatattagtgagtaagcgtctcaccctcgggcgcaaaaatgaattctgggtcgtcatatcaagacggaatttaatggacttaagtgtcccacccgtattaatcttttcaacatagtctacacgaacaaagtcttgtgtacgttgtgttgttgtaggctgtactgtaaatgtcttccatgttttaccttcgtctgtactatatttagcagtcacaacagttccggcaggtgtaaattgttcatatgacattttaaccgtgttgtatggagcctctgttaaatctattgtacgagatacatagctacctttcaatgctgttaaaaatcctgcgaacataatgtcgtccaaggatagcattggtgagatgtattggttagctttaaatgttgctctaagcttagcctcacgagctaactggttaacatctaagtctacatagttagcaataggtaaccaaggtttgtcatctactgttgtacctgcgggctcattatcaaggactaatttcatatcccatctacaaccagtgtttgcaggtgttaggtaggtagacattaacactatgcggtctacattaacgttcttcataacatcgaactcaagtacagcttcttcgttaaatttagccgtgtatacagtgaacttcaaatcagacagttggtgaatagaccatgcacttgcgttagacgaactatataagacaccttccaagtaagggtttgatgttactgtttgtgttggttcgtctactctattttgacccatagttgcaatccacattgtgtatttatcagagtcagtaatgaatactaaacagtattcttgacctgctttacacattagtgggtcatcaaagctgattttcgtaggaacacttgcatcatctgatactttgatttcagaaggttttagcactctttcagcatacacagttttattaggttgcccaccttctgatattccacgtacttgacaaataatatttgtactgttatccttagatgcaaagaatacgtcaaaacttgttgcaactctgttagtgttaaaactgaatgattgtgccaaagggtcaaccaagttaatggtaacatgggtttttatgataatatcctcagttgtttttagcgtaccttgcgcagtgaacgtagtacttgctaagtttgtatcgtttctaacagatacttcacgagtaccacaacgaactcctgcaggtattttaaatacccctttaaatgttccgtctgcatttgccataccagtgccttctgtggcacctttacggtaaccagaagatggggtaacaggtactctcaagccatcaaatgttacgtataggttattagcattaggttgcaagttctctgcgtaaacctcaacatcaatttgtctcataaactcaatcatcgactctttggtttgctgaccaccactggttaaagtagaaccggataacccttgtttacgttggtcttctgtaccagaggcacctccccaagattgtccggggtcgagcgtgatgttagataccatgttcatctcatcgtttgtccaagatgctccaccatgtctccaccattgacgaacagtcatagtagatgtttcttctttggtaactgtgatacgttcatcttcaatccagttatctgcagaagggtttagctttaaggcaccctgtttgttaaaggtgttgtatgggttaacgttcattgcttccgtagcaaatggctgtctaatacccacttcttcggtaaacggtgctgttactagtctaccccacacatgagcttcactagaaccttcgataattgttgggactgttttattaatttctgcataaggtaatgttatttctgctgtgtcaaagtcaaaagcaattgtggcatctgggtgactaacgtcatatttatctagtgagataaacgcatccgagaatactccacgtagatagatagggttttctcctgccattgccggttggtctaagtagaacactgcttggttatactccatgttttccacacgaacagataacttctgtagctcttccattgttagacgtgtgattgtccattgtttagcatcagcagtatttgagtctgggtaaactactactgtaccaatagctaaaactaatgggtctacatggttaggtgcctctactagtcttaaagcgttaggttgtcctttatggacagtgaaattcccgttatggtctaatacaattaggtctttacgagctaagaagtatttatagtctacgttaacaagtgattcatcgactggtttagaacctgtcatctcgttaaagtcgatataccattccctagcatcaccttcaccagtaattacaactttgtaatctgtattttcaatcattgttttattataaacatattgaacgaagtaggttccccctgcaggaggctcttgccctgtaggtgcccaagatattgcttgaccatttactaattggaagtcttcaccttgcttgtactcctgtgctcctgaaccctcagtccatacacggataacttttgttacagaggtgtttttaaggtaatcggtacctccacctacaaccccacgagaaacctgttctttagcaacttcaacttgtgcagttacacggtctacagaagatactggggaattacctaatttatttttacgagttgcattgctatagtagagaccttcgttattgattgtttctaattctcttgatttttcaatatcaatacgagtagttgtaggtttatctactttaaaacctaaaacatatgcccgacctgaatcgacaactaactgaattttattgttagggtctgttgggtgaacttctgtgtacatatcaaaaccacgaacacgatatgaaccagactcatcataagttctttctgctagtattttattaatcttgtccatctcaggagtatctgggttaatgtataactcaccgttaacaaagtggtaaatgtttgatgcagaatcatcgttaactgctagtactacatcttcgtccaatcggtcagccccttctgagaaatggctaggaacaccacttgtttggtctaacagtgatgggtcgtcttctgctgtaataactttttgtacaagtttaacacctacatactccgtaccctctccggtaatatcgatactctgttcgtcaaagttacgcattttaccgcctaagaatactttaccttttttaatagtaagcttcttatcttgtagtacgtattccataccggtttgaatgtcaccgtcactaaatatggattcagccacattgcctaaatctccttgaataattgactgtaactcgtttaactcggcttgttgtaacggcttatctggtttaaataataaacctgaaaatcgtttaccttggtaatatctattgttataaggctctttacctaaatcatcatttgtaatatttttagccatctatgtcacccttcctattgtttaactgacttcttattttccattgtaataataaatttctcttttaaaattacatcacttgttctattttgttgctgtctattttcaaaaaattgcaaaatacctgcatctgttacatctgtaggtaacaaagctctcttttctgaacttgccttagacactaagtcagtatgaatacctacttgcctaaatgttcctgttggtagctcatctcctgtaatttctgcttctacatacaccatccacgcttgctctttatagccatcctcatctggaattagtgtaaattttcttgagccataggacaccacagggtatttagaatcatcacttggtagatattctctacataaagatactcttgctacctttttataaccgataacctctgttagagtagtcgtagtagggtctggtgcaggaggggcatcttcattagtccaaggagtggttttaccaattgctaggtacatactgtcttgtttattttttaattggagtgaggcatacactcgtgaattgttagttgcaatagccacattatttacctcttttctttatcattcaattgttaatatagcatgtttattaaaaaataggacacaactaggtgccctatttatattataacatattatttgctattacccttctataggaacgagttccgttagtgattccacagatgattgtagtcctatactgtacccctcacctaaacgatacttgaatagtacatttagcatgtctagttcaactgttacatctttatctgtttcgttatcaaacacgtaacgaacaaataatagcttgtaatcatttagatagtctgtgatacggtttgctactaagttgaatgtacgcatacgtaagtctaagctatccgtgtttaacttgtgccatgcactcgtagaaaaatcgtatagttcaaccgagaaactgattggtgaacttggacttactagagccttaagtgttgtagagatgttaaagttttcaaacacggtatttaatgcttggtcttctcctaggtcagtaataagtttttcaaattctatagcataattttcttcgataaatgtcttaatatcaaacaccatatatgcctttttatttgttatatccccatttactgtccatgtatacttagtcgggtctaacgtaggtgtttctgtatcttctatcagagaacctacgtagtcgtgggaatcaatgttaggagttctggtgtcacctttttcaatttttaaccaatcaatttgacatgtgcctaatgttgatgatgggtactgaaaaatacgtaaatcgctaggattagttgacgaaacatttcttggtgtgaatgttagtgaccatacgtctgtcaatccctcaactggatttagttttccgtaataagtagttccttccccaatgttatataccataaacgtttgactggctggctttattgcttttatggtaattgtataagtttctcctacaacaaaaggctctaccattctagcgttatataagagataattgctagattttattggaaactcaacggacttattagcaatattctcacccaaagccaccttacccagataatatggtgcatcgagtaaatttggctggtatggggtggctgttgagccttcttcaatcttaatttttcttagtctaactttaccggtcggtttaagaatttgaatgtctctaaataatggataccattcatttggtgcgtcagaggtgtattttaccgtttgtgttccgataagcttctgccatttattcaattcatttttaggtactgtcgttgtctgcaaaacaacgttaccaccaggcaagtatgcgtgttgtaggcgtattccagacgggtcgccagtgaagcccgtttctaacattatttcacaactaatagtatactgtttcccttctattaccggcgtttgcgctccggtagcgtacttcgatagttgatttgtgccatctaaggtaaacacaatctcatcattatcatccgcgactgttgcgccagtacctgaagaaaaacttgaagcattaagtttagcggtaaggttaggattccccgaataatcatagcccccgaagtcagtgctgttggagtataacgtatggacattctttgttgcaggtacttgaatagaagctgttcttccatccttttcttttgtttgattatacatatcagcacctgattctcctagagctgtttttaagtcactcatagagtcagtaggttttggtgtgtaatcaagcaatgttgtacttgctaagtgaatgtagcctcttccgtggtcaaatgctcctgcaagcacatcttccccgtttagcatagagttatttgtatggaagatgttgttctccacaacttgtgtacgttggtcagacaggttaatattacctcgtaggtcatagtataaaccgttaaggaactctaagtctgtcttattcgtcacgtctaggtatacatatgggctttctacagttgttttattcttatttaagcttgtgtctatccttagatagaatagaactcccgcaggtttgaaagctttaataatctctacaatttcaggtggaaacggtctatcaattgaaatatcgataattgcaaaacggtagtagtgacccattaaatggtcgtcaccgtttaattttgacttatttgtgtagaaaatgtttctccaaggttcatatatttggatgactgcatcattgtcgttaaggaaatccaataacgcatcaatgatagcaggaattgtggctctttttagtaaaagttctcttataattctttttctatagtattcgtcattccaatcatctttacgatatacgccaaaccagtccccccaagtatctaggtactcacctgtagctgtttctaatgaagaatgaatcttactagcaatggtctcctgctctgcctgagttaactcatagttcaacgcattaagcactgcaaagttagtatcatcatacttgtttggtttcttaccacgtctcaacagagggtggatatttttataaaagttactcacattatattcctcctaatttatatcagttctacaataacctctcctgcacgaattatctcttcgtccctaacagccatgttcccttccatgttgttaattttacagtcatagattaaaacatcgtcaatattcattataacctgaattaggtcagctaaaattaatggttgtgaaacaacttgactatttaagtagtttcttattgtaagctccactctttcttctaatttactatttattctcgatttgtcacttatagtaacagttgcactcacttgtacattggattttattacagggaatacatccagtttaatgcctgcaggtctgtagtcctcaatagctacttttattttatctagtgtttcttgttttaaatcaccgtttaaatcatgtgcgtatattctaacgtaacctacttcttctttgatataaactcctgcaacttcctctacttgacgtgtaccgtagtctaaggcttttatcgttgctctaccacgtgattccacaaatgcatgaaatctttttttaacggattctatactttcctcatcagtacctgttaaaaaatcaaactcattatacactgtttttaagtttgatatattgttaattacccgattgattcttcctttaggtatgttcccttttgtacctacagttgtacaataaacttctacctttgctgttataacaccttctggaattatgtagtcttgcatcgtttggaatgttaacgtgctaggggcaccagacaagctagagtcgaatgttgttcctgttggtatatacacaggggtctgagtaaccgtatggaactctagtgttaccataccatacgctctttttgcttctcttttcctaaagtcaaaagcatttaggactccttgttcaattccccataaaatattctctctacctaaaatatagtactgctctaactccatagaaacagcttcataaatagaacgaatagtagaaccaactgaaaaatcgtttatctcgtgtgtgtttatcatagttacatcaattagtctacctagtatctctgaaattttctttagtctcattaaaccttatcctttctatacaataattagagcccccgagttatcgttttgaacaacaaactcaaattgctctctagtagactgtaaggttgctctatagttaccaacatatcggtcttcttcaatataatggttcactagaacgcattctgctactcgtgtatctttaagtactgtatcgcatacttctatactaattattttcatttgttcaattgttgttttaccgaacattaagtgcaagtcactaccatactctgggtgcaacataagagaccccttagctgtcatcaatcttgatatagtggcttgcttaatattgtctgccccactagctatcttcaagtccccatgcccattatgtgttattgcaaacacttcgtcacttgttccatgattctcatagtaatctgtatcacttgtcatatccaaatctttacctaaagctaagcctaataagaaatccatatctctttggtttaatgagtttgtgttaatgtccaataagttaccttcatcaggtataaaaagagtgtctccaagtgtagctagatgttctaagttgctcattttttcttgcatagtatctactatgtaagggtaaactaagtcgttgtactccgcaatttttacccaattatctacagagcctgtttctttttgtgctatggattgcattgtatccccatatacaattaaatgctttttatatcgtgccatttactggtaaacctccttgtattaaaggaatttgctcctgaatatatcctagagcgacatccatagattgtaagtccatcactatgtctgtatagtcttttgctccccctaaacagtcaattaagtatttaacattttctctagtctgctttacatctgattcgttaatcattttaaacatctctggttcttcttcgatagcatatataagagcaaaggattctaatactacaatttgtgctagttcgtaaatacgtggagatgaaagcttaagcccactttttaaaaatttgtaggttaaggagtctttagacaaagccttatttgtttcccctatatcgttatctctaacaatatctaatagtgtactagatattttattaactcgtgtatgaggggtgtagaaccttgatataaagtttggttggtcgtcaataacattggctacaacttcaccgctaatatcgacatttaaatatctaaaaaatcttagcaaatctgcggattgttctactttttccatgagttaccctccatatccgattattttttttaactcatttacaccatagttataggctcctgatgttactgcaggatttactgctgtatttggcataacacttctcctatattcatcatgaagtatttgagcaggtgttctagtgtcctgttgtgcattgtatgtccgtccaatgctaggacttacattacctatttgagggcttacttgagctctctcagagggctgtcctgcctctcttaacacgactaaacttaaggtgtaattaaataataatggctgttctgcagaacgttcaatggatagtccttctggcgctaaatgaacaacaaaatatttatcgtctgtgaaattataaaatttcatctctacactagacctattaccattaccaccctgttttgcgtaattatctattatttcttctaattcttttagtctgtccgccccatttttacctctgctgtctaccctgaatcctgttgtaccagaaaattggatagtttctatatctttaccaaagtcttctgttataatgtttgatttagttttaaatatagtaacacgatgtggcttattatatttatattgagttgggtttacttgaaatcgataccaacctttattcgggtcgttacctacttgaatcgcaatacgttttaatgtaccgcccataccatctgattgtcccatagtaaccgctcctttcgttccttaatatagtacaatatagtataaaaaagagggtagcacaaactaccctcatttatattagtttaaattagtagccctatgattaaacctattgttactccaattactaaattaactatccttgtttttcgttcttttttgccatctcttccacgattttctccacatcttctctaattagtgccggaactttctctaaaggatagttctcagggtctttgattacataagatgcatataacattgataagtatgggtacatagaaatatcacctcctttaattaatagtagcttacttgttcatttccgagaacatgtttattgcatccattaaggctagctcattaaattctactttttgtcttaattctttattttctttaattaaactatctatactttgctcgtcttgaggttcattgttctcttgctctggtaacaactcatcaggtacagaactccaagaaatttctttggcttcttctatagacttagcattttcaatcataggaagcaatttattatttaatttattttccctagactcaactacttttttaatatatctagtcatagtagtatattgggacttatcaaatttaagtatcaattgagaaccatctgatgtagcataagcagttattccaacataatcttgaattttattgtctagtagagttattttattaataaactcatcatcgctaataacatcaaataagtaggtagtgcctgatattgtataaggaactttttgcaatatgtaggtctcttcacagtcccttttaaacttactggctactgctagtaactctacttttaataacttttcttcacttaggactagctctccatctacatactgaaaacaatttgaatagtaatgtgaaaagaatggagaatccattggcaaatctacttcatagacaggttcatcactattgagtgacgttccccagttactaacaaaatctttatcatctagtgttacaaatattttttgagtttcttctaccattctgatacctcccttataacaatgccattgttaactcctgtcccattattaacattaccaccaatacgaccgtcagttatatacatatatttaaatgttatcgtagttccatgacctattatagccctaaaccctcttgtgttaaactggtctagtacagacttgtgaataaatacctgagttatatcccaaggttgccctgcacctgactcatatgcggagaaggtcaaaacccatccgtttcggcactctgttaattttttgctaggtaaaacttcttgtttttcgtttaaataggcagcccctttccaaagctgtttagtaggaaacgtcatcgtaccagtgtcagaaacttcaaatataggattagagtcattgttaacatagaatccattgtccctcatataaacgtagtcatagtcctcccactttaaggagtttcttggtttacctgatttaataaagtgaactccttcacctccgttatttgatgtatctgtcgagaaccatatgccattgactgctgatatatcactgttgtttaaggatattcctactaaaccattctcctgccacttatctctttctatctctaataagcctcttagctcattattgttattgtcaacaaaggcattacctaaaaacagacttcgattatcggcatcaataaacatgatagggttacccggtttaacagtagctgttctagtaaacgtactaaacttatcagttacgctcaacctaacttcgtatgtattggtattatctaattctattggtgtattttttactgttaccatactaccattagctgtaaaatttaaagccgtttctgctccataagtaccaacacctgctacttttgttgcgtaggttactgattggatagcatttttgtttacaccacctatagttataggagaccatgtactattgacattaatatctgtagttgtctcaaagttatttctacgtgtgactgtaaagtccatatttggattttcatagggataaatgtctacatgttttgtccagttagacttaaacccacgactgtcagttactgtgactattagtgtatctgatgtgtggttagcaggtgcccctaattctacaactgtttctgcatttgaatagttgattgtttttgatgtaccacctaaagacacgtcaattgttttcaaagtagagtgaccattagccgttgcaaagttagcagggatggttactttgatattagacttaccttgaagagctacctgattgttacctgtaagcccgacagagacactacttgtgtcttcataagtaggggtaccattaatgcttggtacatacttgtttttaggtatagttatatctattctctgtcctgtccatggagtcctgtattgtactccactatacttacttgttacagttacttgaccccatgaggaatccgcatttggtattttcttcaatatattgtctacatctgtttgagttaatgtatacgacattgtgttccctgctgaatttgtggcaataggtatgttgagcccactaatgttaaataccccatcataagtaaagtttgcatcagaccgatagttacttagtgatacattgattttctctcccacatttaaggttatcttacctcccgatgcttgtgcattcattggggggacaatcttgtatgttctttcctgactaactacatcatctaaccctatagtccagagtttaaccttgccttgccaactagttctattgtttaatgcagtaaaaatagttttgttatttgctactgtaaaactaaacgctccactagtgcctacatcattaaggtttgtcaagtacgtccaatggtcatcattgtttaatgtggggttatctctgttagccacccaagcagtcagattatgtttgaatgtagagctctgcctatttattgttattggtaagttccccggtaaatttaatgtagggacagggtttaatgaacttcttctaggtattctatttaggtctactgaaaaattaggtatagttatggtaccataatatgttggtacaccgttatcagtaaaagtaacgtttacaaagtagctacctccaatagttattcttttagaaccgtcattattatggttaactctccactctttagctagtaatagtttcttttggaaagcatttaacatagaggaagctttttctgtctgtgtagtcccttctacggtaactttggcatcaccgatagcgggagcatcaagtctaccccatttatccatagattgtaggtatagccatacgctgattgttgagtagttccctgcaatgttttgatttgcacgccactctaaaactaatctatgtcgggcaaaggctgtatactttgttccatttaaagccatatagtctcatcgtcctttcgtgtccttctctatatagtataacacaaattaaccgaaaacgacttgtaagtgaggttttcggttaaaaagttaaattatttattcaaatgatggtacaaatgcccaacctctaattctttttgtatctgttactgtattcatagcaaatacagacactggttccatagttatacgtttttcagcctttaacattttaacttctgttacctgcccatttaaagtgaaaatacgttctatgttaccatcaatacgagagtagcctgaaaactcttggggggtaatcattgtgtacccatctgtttgattgtttttaacagttaccccattaatatcaatcttaacattcgtattgtatatctcacttggatatggttgccatttaagaggtatattcccgatattgtacatgatacctgatataataacagatgcctcttgtccattttctacaaacatttctattttagtagaggtattcgtaggagtgaatacaagtttgtattgttgaaatcctactggtataggttgtgttgcatcctctacacctaaagtataagttagggcatctccttcataaactctaataccacatttaaagtcagctacaggattgtttgttgttactttcatatagaaagatagagcatactcaaggttctcccctatttcagggagtgccacagactgtgttagtgaagagctagctcttttggagctaacaaacccagaaccaaaccctaggttagctaactcctctgtttgagtagtttcaacaataccaccggtaaacgctccatcttccttgttgggcacccaagtagtagcttgtgacccttcctctatttttagctctctccaataagaacgatgtgtccagtgttctagtatatttttaggatagacatcaaggtacccactagtgttaattgtcaccgttttttcatatctcttccatacagttaaatcttctttactgctgttaccattgactgtccactcttcaatagcaaattgagaccattggtcggtgttctcatcagggaatacacgtatagcaaaaataggtctatcactcatgtaagttatcgggtcttctttatagtcgaagcttatcgtgtatgtttcaccggaactaacttttattgggtgaggtatattacctatttctttagtagcagaacgtgacggcattgaggctagtatgttgcttgttggcttgtcatcttctggtggaagtatttcaaattgaagtattgaatatccaccccatcgaccaaagcctagattccatgtggagttagctaacaagtttttccctactgtaccatcccagaagtctaacccagagaatcctaaagagtttttaagcatgttaacccctccggcttgttgaacagagaagttaaagctgtctttaagctgttgtagttcggagttagttacataaggggtaaaatcaataccgtttataccttctttgattagtcggttatagtcctcttttattgagtctaaatcttcataggtagctaagttatctaggtcagtagtgttagcctttgtatcgaacacactagtaaaatcctcagaacctagtacagtatttataatgctatcggaagatattctctgctctgcctgtgctacccagtcagctaagtttgtgccgtcttcaccccaaggtaaatctttaacctcttctggtgttaatgcaacccacccagtttctgaatctacccaacgcttaggttggttaggtacttttgacaagtctacccatataagcccttgcgatgggttagtaggggctgttggagaataaatccatgcgccttccttaccattaatctgaccttctatatgttcatcagttagcctccatgtataatctgaatagttatcactatctttcatagaggtacctacatacatgtaagtttttccttcaaccttttctcttgtaaaatcaagaccctgtaaatcgttagcccaagcagtgtatgttttgatattctgaacaacattgtttatgtcctcttgaatatctatatcagactttttccaaggagttggaatgatacctacttctagttgaacattaccgaatcgaaactctttaacattagtgtcggctgactcaatacgtattaacacaccttttgttaagcctgttacttttctttgagccacaacacgtttcaaaccatcacttacttgtataggcgtactattagagcctacccagttatccccttctaaagtgtagatagctagtctagcagagccaccacctattacctgcacattcgcagaagctgttagccactgaccttcttttacttctttcatacgataatcagctaaaccgccaccagcaatttctttatcagaagtacctacagtataatcgaaaggttgggtcgttgcatcaccttttacagctaccatgctgtcaatgaactctgtggttgacgaacctaatgtgtagtaaccaacgtttttaatcatcagtctaggcaacatattgtctccggaagagaaccctacatagttgtacccaccaatattttgtaaattatcattgatagtatctgcagtttcttgtgccttgtctgctacattttttgctacgtcagatattctgtttagcaaagtagttcttttgtcataatagttgttaaacgtatttcttaaagtagaaccaactactacagaagttgttgtcatgtcagctagtattggttttaagtactcgtctagtgcgttatatgctgttgtatactgttgagaagacaccccaaatttagttgcttggtctaagtttattggatactctgttttaatctgttcccattgtaagagtatttgttttttctcattcggtgctaacatattgtcattcgacatgtcagctattgcgttatttgcattttcagcatcatttttagcattttcagcatcttgttgagctttcttggcttgttcttctacttttttaatgtattcctctaaagcagtttggtcaccttttatgtcttcccatgaagggctgtaatcagtagcctttgtacctgcctcgaatttgatattcacatagtttgctttcttataacttaccctagcatagactgcattatcaggtgacacatactttttactaaaatccccagagtctgccacagcttccccagaaataaatgttctatttttatcgtaccagtctgttataatcatgtttgtgccagtgttaccataaagtgtagcaatatatggtgttttttgattaactttaatgtagtctgacaccactgagttatctactgcagtacctacagttccatcattctcgtttagtaaacctgctgtctgtgtctttataacaaataagttagttccacctaatgttagattgttaatagaatcgtctacaattcctttaacctcactagcagttactttttgactaatttgttcagcttgtatttcgatttgtgccttattcgattgtacaccgtcttctacagtttgtactctagtagctagtaaagtaatagcttctgatgtttgagtaatgtttgtttccattacagatattttattaccataggcatctagctctgttttagctgagtccgttacttcttttagtaaagcaatcttacttgcgtaatagtccgagaatgtcgctctaaggcgttctccgtctactatactagtttcatccatgtctttaaacaaaggctctacaaacagctctagggctttatatttagctgtatactctgctgtgtctactgcgtacttctctgcttgtgcaattgttgtagggtattccttaacaattacatcccattcctttttaagttggtatttttcagaaggtgttagcttgttatcacttgctatatcagatataagtttgttagcattatctgcatcaatcttagcttgtgaagcatctgccattgcttgtgttgcatctagtgatgcttgggatgcttttttcatagcctctgtgataccgtcttttagttttttactaatagaatttagtaaagctattcttgctgtatagtaagaattaaacgttttgcggagtgttgctccgtctaccgagctagttgattccatgtcagccaatataggggtaacaaatagctctaatgaattatacttagcagtgtagtctttactatcaacctcgtaggtttctgcttgttcgagatagcttggatattcattttttataatatcccattcttttaataaatctattttttcacttggagtcagtctgttatcactagacatatctgcaatcactttatttgctttgtcagcatcttcttgtgcttttttaatcgcatcagacatatctacaaaagaactgtcaatcatatcctgaacttctgtctttgttacttttgattcaactttacctgctacaattgttatttctgtagaaagttctgatatttttccattttgttcttctacaccattagctataatctctaattgaccatttgtttccttaatctgtatagacaacccatttagtttgtcatatacatcggataagtctacatcggctgtaaacagtttcccttgtgaatagataccgtcttttcgtacttctaaatccatatccccattacgtaaataaacgaacccctcattgttgattcctagctcacttatttctatgccaccatcaatattaatagagtcttgttttcttaatttaactctgccatctggtgtcatttcaaatagtgtgcgccaatcctcttctttgttcatcatagaagttctataagtaccatctgggttaatatgaattagtaaatcgtgcaaatctggttttccatcatcatcaagcaccccttgatgcttaaacagtacatttggtgctctacctttcataggctctattaggtcaccattattatagtatgatgtctctaagtcctcatacttagtaccataacctgcatcagttgtagaggagttagctacttcttttgtatcaaaagcaataaatgatttaccagaaaaagtaacgacacgtcctccttctccatcaacgctatcgtatgtcaatgaagggtacaaactaaatttttggtgcatatctccaattaactctaaatctttagggtctgaatttgaaaattgtgttcttgaaagttgcttgcttacatcgttattattataaacactaattacaataggcatgtctttatcggaattaataaaaccaactaatactactgtccctactgctatcgggttaacctgtccataaggctgtccaacgatatttctaccgccaaattccataggaagtcttgcagaaaaacgtccctcatttgcataggaattttgaaatacttctttatgctgtaaagctagtaggtctactgtattatatttatagtttacttgaattacttgtgctaatagtagcgtattaatttgttgtccttctttatatactcttttaatttctgagcctagacctgcttgaaatctacgttttaccaatttattcatcctctcatcatgtgtcctcattatctattatagcacaataactaaaaactacctatacaaaaaaaataaccctaaaaaatagggttatttatatctacgtacatttccgtcaaaagcgttccaccaataacctttagacatgtcgacaataacaatacctgtttttggggattcatccatgttaccttttccattacaagagaccatcttgccctcaccacaatatatccctacatgactagctctgttaaaccaaactaaatctcctaccttcatcttatcaaatagctctaaagacttctgacctctagttgagatagtttgtaattggttatcataaagtagtgaacgagtggtaaccattctagctccaccagatagtgatatgcctgcatggtagaaacaccaccaaacaaatcctgcactgtctgttgctataatatcttcctcaaatgggtctttatctgtattagctccaaaattgaaggcagattggaatgtggactctgttttcgagtgagctcttccccatttagttgcttctgtggcaatgttattcccataaagtattggaagctttttaacttctacttttggtttcttttcaacaactgttttagtttctacttgcttaacaactttcggctcttctggttctaccggagtgctacgcatcatacggaacccgaatggagtagccatggctctagcaccaactgtctttttctttccagaacctgaaccagatttatcgtcccagtctatactacgccaattgtaccctccaccattcataattttagttgtttttccagttacattattttttatagaaaatacatcgtataaatgagaaggattttttataaagtaaactcctgcactgcttgtgctccaaccttgccatttacctttagacacctcgaagtgggcatggtctcctgatgcttgtcctgcagttcctgagtgccctattacttcccctttagctttcttatcccctactttgttgcttgcccagttgttgtcgtgtacgcataatagtgttacgtaagtaacactgccatctacacatttaacaggcttttgagattgccatgcaacccctgagtaagagtcagtgtacacacattcacagtcaaatggtgcatagtaaggtttatgaggagtaccatctgaaaagtctattgcaagtgctcccatgtggctgtatccaccattctctccctgagttacattaattacgtctaatgggaataccgctagctgagttcctgcacctgctgtgtactctttacctgagctacccccactatcagagcctccatcatttttgctgtttgcctctgcttgttcgtctagtagtgcttgcaaggttttctcacctaacatacctccggagaagtcctcagatttaccccataaatgagtgaatctatcgtttcctccattttgtaaacctctagtgaccccaagagtagtgatatacccttgtgtataggagaacgagtgttctactgattctatgtagtattcccacaactcaccattttgctcatcttgaacaaacaaacggttacctaatctatatttagggtcacctttaactacaatgtccccactgtagaaattagcgttttcacagtaccaatttgctagtcttttagtgaactcggaaacaaatcgtttatctgaaccagtaacggttgagctatcacttgggttattctccattatttcttcatattttagtttacctaagttaccttgtgctttgtattcactagcaatagatgtggcttggttaggtatcaaattgaagtagttcattaatttctcttttatgctagatactcctaggtttttatctaacccattaaggaaatccctaactgcggtatatgtaggttctaccttaccattaccctcttctatattttcttcggttattcctgtaaatttagaaaagtcttccttacttaatgcttgatttgtaatatagtggtctataattttatctgcttcgttatctgttatacgtctatctacctgtgttattgatgttcttacattactttttttaactcttagcacatcaacaggatacccttttagatagtttaagacaagagcatactctctgtcaaacatctgtttagctgttgagccatcactctctttactatcattatcactttctggttttgtgttagctttatccccactgttactcttatctataattgccccttctaaatacctattaggtacttctagtttcttatagccatatttagaaactaaatcagggaatacttgtggtttagaacctaacatcatagagtcagtcccatacaagttattaatagatatgttaaatatagagtaagcttctgtatcgttaactgctacagattcagatattacttctgcagatgttactgtgtaggtttgtaatttttcccagtcagatttatcaaatggggttctacgcataatcattctgcattttccctctggtgttgcatcgaaatacaattcgttaaacggtttagcggttacatcatctataagttgctttaaagaaccttcatagtttatgtaaggggtactatctattagtctctccgcctctgtccaactatctagttcccactctaagaacttatctataccttgaccattaaaattaaatttcatatattgtaaaaatcttttcattaggctttctgctatttgacttgcactactaccagacatttttataccttcttgtgcatcgtcaggtaaccaacctaaatctgtcaaaactacacttacttcctgaataacacctaagtcaaactgcattagcgcctttgcaaaagactgccctgttatcctgtacatcttagagttttccccatagtcaccctctagtctaacctcagatattaaaccaactagtagtacagggttatccgattttttagtggaggaagtatctgggtctattttaagaataactgcatcattagcatttagtactctatcccaatacacatcccctgctaatacaaaggagaacactgcactatcgtcttctaaagcattttttgtagttaaactaattacagactctgccataaaattattgcttaccttctctgcttcaccattgttgttattacgtccagtaagcccatcttttgcatcataaactatatggtaattgtctgtctctgtaaaaaaagttaaatcaaatctcgggtatcttaactctactgacattataaacctccttagagaaaagtagagacattaagcctctacactctcctgtattcttttgcgaagaaatctgtgaaggtggcatcgagtgttttgctgataattcctgctgacttatcaccaatttctctagctatactatcacttgttcctccaccttgtatggtgacattaactgatacctcatttgtatttgtcatcgtaggagctgaacctgattgagctccccctgttgcatttattttagaaattgaagctccccaatcatgaccagaaccgttacctgttaatcggaagttttgagctgtttttctagtagcctctgctagtaaacgtggggctgttggctgactagggtttataacatactcatcttgacctgctacctctcccagtagtgcatactctgggctagttatgtgtccgcccatcgctctagttttattaccagtaggaccccatcctgtgctaccgttttgataacggctatcccaagaatttaggtcatttttccaattactattattaaagaaagctagcaattggtcatacccgctcattatgttgtcatgaccgtctactttgtatgcatcaaaagtactttgaacatactgtagtaaacctttagctccgccattgccattgaagttgttctcatcaattatctgttgaacagcactctcgtcaccactagactccgcctgaatcaatctaagtattgtatctatttcatcgtcagaaacatctactcccattttcttagatgcttgtctaatggctttctcccagtctcctccaaagtctttactagaagcatctgaacccattccgtcagaagaatctttagagcttcctgagttaccgaatatgccattttgattttttgcttggtttagtatacgttgagctctatctaataacttagaatagatagatagattagcagactcttctgcgttattctctgcacgtgttttctcagataggactcgtttgttagctgtgtctctggcttcctgtactcctgatttactctcttgtccagaagcaacatcttgccctgcagttaagtctgcttcttccgcatgagcttcttctcctccgaagaatttacgaacaccatcaacgaatttaccgcctagtttagaacctgctagagtacctcctatggcgccaatacctccaccaatagcccctccgatacctgtaccaagaccgggtgcaataaatgtaccaatcattgcccctaacttagctcctccgatacctccaccaatagctccaacactctctcctaccgcctttgttttatcatctgcggttgcaatatctaatagagaagaacctatcgctagaggggttgctaccttacctaaaaacttacctgctttacctagcccaccaagaaccttactgcccattccggcaccttttagtccttttgctccttcaacagccttagctccgacacccattgctgagtccttagctacactacctacatttttaataccttggaaccatccgccagagcctttacctgcagaaaatgcctctttagctgattttaaaaatccgccccctgcagttactcctccacctgtactaaaagtagaacgagttccttttttgattaaactagataaaccaccaatagagcctgatgtggctagtgaaacagccatagctcctaagcctgctcctaatgcatataacgcagggggaagtgcccctaatttagagttagtctctcttaagatgtcccccatatcattaatctgtgaagcttgtttctctgtaacagcttctgaacggttagcggttccttctttagagtttgcgtagtcttctatgttcttatcatatttgccttttcctgtagattggctttcattcattattctatctacgttatcctgacttaaaccaccgttggcataagcttcccaaattgcatctatttggtcattcttagcatctgtaccgaatagttcatgtaaggcactaccaaaagcatacttctgagacttttcgtcaccgggaactgattgttggacactaccaataattttctgtaagttctctggtgttgccccttgttcttgcatagcttttaagtcgtacattcctgctagtccttggaattgtgtccctttacctaaaagtaaagatgccttattgttccatacactaccttgaatacctgcagataaacttgacattaactgcgccccttgttcaccttgaacagcacgcttacctgtttggtttagcattgttaacattgccatttgttctttaagctcttgattacttccgttacgaccagtaaagctttgctcacttaatgttttaagagcttctagttgttctttctcacgacctgccatccctgagcgttggatagcacctaagaaaccttcttgaatattcttaatttggtctctaccactaatagctccactcttcatttggctattcatgaagtcacttaaagtctcattgtctactggaactgcccttgaaccctctgctaaggctcttgtacttcctgctaagtcctctttacttgtaaaaccaatattactcaatgcatcttcttggaattgtaacatgtcagcacctttatagcctaactgtttctcaatacccatctcttgcatactttttcttaatgcacggaaatcactagtacctgtacgttgtcctaaagatatagatgcatcacgcatacctgcattagcagttgctccttttgcgtataaaccacctatggcggcggcggtacctcctagcatagccattgatatagaaggtgcacgctcagctagaacaccacgaatactctttctatcagcatctacttttatgtcagatgcatttagtgaagcttccttagattttaagttctctacagtctctttaatagagtctgctaggcgttttctagcttcaatttctttctcaatttcttttatctcagcctgaatactagtttctctattcttacgaacttgtctatctaaatttgtattacttctagtctctgctagctcttcttgtctctgtttatatcgttctcgtagctcttgctgtctgcttttattttctgaacctaggctttcaaatacaccagtacgtctaaggtcacgcttcatactctctgactgattgtaagtcattcttcctgttgcgcttgcttggtttgcacgatggttaaccctagagttcaccctgtttagttcctgttgtgccctagagatatttcttatcgctctttcttgctctgttaactcatcacgaaatgccttttgttgccttagtgtactagcatttatgtaattagggttagcttgcattctagcatgattttgaaattctttttgtgtcttgttaaatcttctagttagttcttctgcatccgatacagcacgtttatatgctgtcatgtctcccatgctttctgcatttatcttctgattatcaaaagctctatctagttctttttttgccttgataagcttttgcatttcctcaacagctttatcaatgtctttctgacttgtatggaaaaaattctgggtgccctttgcactctttgaatcaattttatccattaacttgtctatttcattaagttttgcaacagcatccccagtcattgcctcaacataaaaacgataattgttgctcatttccacacttcctttacaagtaaagggcagggaataccctacccttaaatttgaaattcgtcatctaatgatgttggctcgacatctttgttaaacaaatcgatagcctcctgaatatctccttgtttcaactgtgagttaaactctaggttcttacgttcctcttctaactcaattgatgatttctcttgccatttgtttccaccatgtttctcaatacgtctagcctcttccattgcttttttaacattgttagcaataagttcgttaatcgtatcttcttcaattgttgttccaccttctgcacggatagcatcaacttcttggcttgcctcccaacgagctttaagtttagccatgtcttcctcacttgtgatttcactaagtttacgagcaatttcttcctcatcgtcaccttcacgaattggggagaactcatcatgtggtttatcataccatgaatcgtcatagtcttggaactcactttctaactgcattcctttagctagtcgttcttgttcttctatgtctctttccatgttatataaaatccactctacttggtcacttgtgagattttgccacgcagggtcactcggtagtaccttaaattttttcattattgcccatagatttctactatagctatttctaacaagggcttttattccgcctagttctttaactctagtatcgaaaggagtttagccattcaccatagtcctttgcaattactcctaatacatataaattataaattttttcatcatcagctagtacttctggcacctctaccccacatactcgaatagttgctaacatttggtaactttggaaaataaagttgctaactgccattcccataccttctaaataggcttctcttcttgcctgaattttaccgttttcgataatattaggtgcttttattttaatatcaaattctaaaccatattcttttaagttataatgcttagaaaatacatcgttaacacccggaataattctatctaactgtttacgttcttctgcatcattgtaatatttcaaaatatttacttcttcatccgtcaagttttctccacgttgcaacttttctattaattgctcacgtgataaatttggtgtaggtgtaaataccttttcattattttctgtcattattatttcctcctagtttactcataaattaatatagcactatctttattataccacactatctagttatttgtatttatagtggcaaaaaaaatagacctatatctgaattaggtctatttaaacttatactagttaatctattgttaagttttcttctctttttatttcttcaacacgtttcacaacgtcttcataaataactgatggtacattttctatgtaacctgagtctctaactacatgttttgcatacaattctactaaatatggatatttcactttaggtacctccttttattaaagccgactctgttacaaagtaagctcagtctacgttgtgttaaatcatatttcctacaagtttccttacctgtatgtgttttatagtaatcaataatatcctcattaggtatactatcagaataaacaactgaaactagtctctttttaattggtactatctcggataaaacattagctacttcgtttatgctatttgcagtgtatgggataacaacaagattaacacctgcttgactacaccattgctcctttagtttgtcacgttcctgtaaatcttgaagtttacccttatagtaacctccggtgtcttcatagtgctgtttcccattatattcaatataggtattgtcatcaggtaagtaaaaatcaaaaaataatgttctaccaccaataactatctttttctgttcttcaaagttaataccattctctttaagcaaacttctaaccatgcgctccccaatactacgtttaacatctaacggacaattgcagttctcctgataagcaaaatggtaccagactgtctcccactcattaccttggggagataacaaagtacacttacctcgaacagctaatttttcagggtagttaataattgtgtatccccgagactctgcgtattctctgaaatatcgtcttttctcactttctgtaagaatgtctatagacatatcattgacttctcccctataatctttttgcgtgatagtgtttaatctgacctcacctttatagcctttatatttgcccttagtgattaatacagctactggggtatctctgtttatgtactctcctatcatgactagtccatatttagcaaaacgttgtctgacatactcttgcttgctttcttcggtcaacccattcagagagggaactctgcctgcgactatactatcaaatgttgttttaccagtcaaacccgcaaattcaccagtcttccatatatagtaaataggttctttattacccttaaaattagcattaagtaatacattgttagcttgtccccaattatctataatagagttcttctgttccatgctgtatacttttttcatattggaacctcctagttattaaacttcaactaatgtaattataccataaaataaacccctaataaatggggtttatttctgtacatcttacttattttacttttgcacttgtaaggtaagtgaattgggttgattctgatgtaacttcgttagctgtgaatgactctgagtaagaaattgcagagcaaccacgataagctactacgatttctttagttaaattatccatcattacgatgtcgataatatcacgttgtaagatgtcttcacctaacgctgtaattcctaaacttgctaagtcttcttttttcatacgcatacgttctaaagtaattgtaccttcatatttcaagtatacgtgttcttgtggcataatactaccgatttcatagataccttgtgtaccatattggcgctcacctgatgcagattgcgcacgaccgataattttattaccaatcattaggtaaactgtgttacctgtgtggactgtttgatttccaacactagccatgtatatatcactcctatattttattcattatgtaagaggaggggcaactgcccctacactctatgctgttaagatttgttgtttgtatactaactgaacagtaatcttattcaagctacggataggcattacagtcatactgattgatgccacgtcaccttctaagacaacttgtacttcttccggtgtgtaatcttggatttcacgagcacgttttttgttatctaagaatgattggataaagtttttgattaagcttgcgcttgtgtcaatcactttagttccgatgaagttgttatctagttcaattttcaattctgaaactaagaagtcgtttgcttcaccaacagacatttcatttttaactgggtcagatttgtcattataagtagttacgtcttgtactacacggaatgcagttaaagtacggttacgtacaaactcgatagaaattactccactttcgtttaacatgtctaactggctactttcaaatacacggtctacagatgttacgttgaagtgtttgaaagtgattgcttcaccaacttctaaaccacttgcaatacctgcaatttgtgaagccatcatgtaacccggtaatttaagcaaacgtccgtcatccattttacgagttccagagaagcctactaaagaagctctagggtcacgtaagtttgttgcacgagtaatgctttcctctactgtttcattagtgcctccaccaacgataatacgcattgggtcaccattgtctgtacggtctttaacaaaagctaaagcctcagaatgaactgcttgtttatctgttaatggtactaagtagtaaccaccttcattagctagtaatgggaatttattagcccaagactcaggagcagttccgtcagaaccacctgttaagtttgttaattcaaagtcttctacaggttttgtagcatctacagctacagttacatagtcattgtattctagctgtttagcaatgtctcctgctaaagctcctacgaatacagcctctgtcttaacatctactttgtctactgcctctaaagcatcagtaggtaagtttttgtcacctatagggaagaatttagcctcccaatctggtaaactattgattgcactaactaagacatttgtctcagagtaaacaccttgacctaactcatacttcattacttctgttgtactttctggttcagcacctacatttaaagttaatgttgttgctttcttactgattttatcttgtgcaattgtaaagttggcttgagcttcactacctttatattggatagagaaaattttacctaagttatcgaaaactttcttataaccatctttagaaaatgcaacagtcaatctttttgtgtgtgttaaattgttgtcttctaatgctacttgaatttcatttgcatctaccccataaattgttgaagcaaatgttaaaccaccttttgtaagagttgcgtttttagcatcttcaacacgaactgctaaaatgtctcctgctgatgcggtattaacgtcagatgcattccatgctaactcaatagcatctagcaaatctccactacgtagtacttgtttagcttgttgataattacggaaacgataaacagtatctggtttaccgcctttagcagagccgactaacattaatgttttgtcagagctacttgatgccccaccaataccgcttgtgtctacggtaatctcagtatgtggacgtgatactttttttcttgggaattgttctacagccatttattatttaacttcctttcctatatacttttctaaaaatggaacaaaatcagcatcctcgtgttggtattctctacctgccattagtgctctgaaacccgctacctgatatttatttagtccatataatacttttgcataatctaaaaatgtatcgacatgaacataacctttaggttttttaggttgctcctttttaacttctttttttactactttctctgtttctttagccactaggatttcatcctcctttttgtaataatctctgtgattctttgttgtaaatcaaatccaatagaattagtaactctgtagtttacagtacaaggtctaccaaacacaagtgtttcacctgattctatgacaacttgcatatcaccaaaatgtagtgtttgcaacatataccctgttttttcatctaaactgtctctcatggtgattaatataattctagcaattgcatctaaacacctagcagtatctatattagaacttattccaattatactaacattatcgttagattggtatcctttgtatacacctgcgacatcatcatccgatactttgcttatatacgacacgttaatggatataccctctaactcctcattgtatgaaaaatcaaatacaggtttgttatcttcaatcctaaaatggtcgctttctgcaaaacttatatctgagctgtttagatagtccgctactggttttgatgtatttataattagcttgtcaccttctctaataatagtggcttcctcactaattacattaccttctcgatactcgtagcctccttggattcctccaatagacttgctatcttcttcgctactaccgagagttactacaaatcgagcaaatgaatccagatgttcttgtgggaatgaataggacatctctacttctctcttaggtggcttaccatctatagagcaataagtgtttttaaaagattctaaagcttctttgtccatcccctttaaagcttcgtctataatatagcactctgatagaattatccttagcctttcttcaaattctttatacaagtaagtgtctaaacttggtatcatacactatcctcctaaattctttagtttccatttcatgagcctgtctatgtttcttagcattgtcttactcatatcatcatcattaacgtttctacgatttattatccaactattcgcaggagaattagcgttaactgtacggaaagctacataggtgttacgtgtacctttaccccaactctgtggtataacagttacattagtagatttaggtttgtagttaatactagacactgatgggctctgctttcgtctatcatacagatagtccattttaacagttacaggttttgtgcctactggaactgctctcaactcatcgtataatcgtcttgacatgtttcgtgttttcatactaatagggatgtataggtaccattccccttttttattatatttctttttattagaacgtgcaaagaaaggtttcaaatctataaccccattttttgccatcctagactctgttactatcaaatagctaggcattctctgtacagaagacccattacgtagagcaacatcttgcgcatcacttagtacttcatcaaccactgcacgagttagattgccaagtattgcttttgaatcagtaaacaatacgggtctctgtccttttctagccatttaatttacctccaaagaagcctcctgattttgtagctttacgcttagggtctaccatagcttcacttgtatcttttttgccctctagctcttccatacggcttgctgtatctatgtctaatgaaaatggttcactatcaataaacacgtcctcacgcttcaataagagctttttaggtagtgactcaaattgtgtttgtttaacaccgaatgtggtatactggtatctgctttcttttaacaggtctataacaatatatctaagtgtaacagacatatttatggatatattagtatctatcaaagattcatttgggtaaatagtatttgtgtcatagtccattctaaaatcgactccatcaactaagatacgtccgtctttaccataaatatcttctatactgttcacatcatagcttaaaaatagtccattagctactcttcttttatttacattaaaaataaagctttgatatattttaacttcggggactgttattctgtccctaaaggttatcttagaatctagctcagtcgttccaatagcggttcctgtgtctagtagtcctaaatcttgattagagatacctttctcttgactctgtatagccattcttaattttactgcaggttggtaagcaatccctcttcccctacagatggggcaatccgacttaggtgagagggtcattgggttacgacatgtacatagccatgctctttcccacaaaacgtcaatacctctattttcaatgaagctagccataccttttgtttcaaattcaaccctactagtatttaattgcttgttagggttacctgtttcacctgctccaccaaatctaattggtttttcacccatttgttaacccccttttttaaataatacctaagttcattccaaagtaagataccaatgatttttctaattcttgtatgtcttcgtcaatttgtcgaatatctgcagaagccccaccatacatagcagactgtgttgtttgtatactttcactaataccatctactgttagtgacttactagcaatacctgcaccaattataagtctaccccattgttggaatatctcttttaacgcatattttattacaagctgttctaagtccgcagggcactcccagtcttcattgtatcctctatttttcctaggtagcattcctgccacatagtcaatatgaatcatttgaggagcatcaacttgtcctcctgatggaggcattcctgctaattgtgggtaccctgagaacgtcatttcataaccaaactgtgttccagtctgcatgagggatgttgggtacatttgtatatgacctgctagtgcatacactttccaccaattagacgggtatctgtatagccctctaccattaacttctaattgaagcttttctgcttgaataataggtcttttaaacacatgagtatacatgtaagagttgaaatctgattggtggtaatcatggtgttctcctcttataagtctaggcataatagcaatatctagtcgtttctctgctttagctattgctgaatctatgatatgttcatagaaagtatctcctaaaggctcccctgtttcagggttaacaacttctacaccaaacatgtattgtttcagcgtagcaggtgtccaaccataatcagcaggctgtatatcatcaattttgtctaaatctatatgcttagggttcccatgtgcatatggttggtattggtatgtattgtccccttgttcctcgtatccgtagtctgggtacatataaatacctccttagtcttcctcttttttagattttgtagttttcttagcaggcgctttcttagcctttttaggtgcctcgtctgctttttctgcagtagcttctttttctttagctttaggtgcatctggtatatattgtatatatggaactttagtccctaaatgttcttgctgttctactgttaggtctgtggtttccccattatggtcaaatgttacctcaccaaaagctgttgttactgttttatttattaagatttctgattttaacatattatttcctccatacatgtattttataataaaaatagggaacagtattaaccgtcccctattccatttttattcaattttgcttagtcttctaaagattagcgttcgttaaatacgttacctgttgcaatatatttaacgttcttaatgcgagcccatttcttaggtgcacgtaatgctaaagctccataccataatactgcgaatgttacagaagcattaacttgtgctaatggtaagcgcatcattggtagtaactcgaacaagtgaactactgatggagttaattccccaacgaatacgtccgctgtttcaggaatttcatcgttcacatcgataaatgtgataactccgtcaactgctttacttgcaggtactctagcgatttggtagaacaatcctgtttccaaacctttacggtaaattgcaacatattgtggacgagcttgatacatgttattaatagtgatttctaatttaacttgttttttcttgtcgtcaattactacagatgcaacatcagatggtgcagattctgcatcgtcagatactactacaactttgtattctgtgtcgattgttaaatcttcatcacggaatttaccttttgttcctgcttctaaagtagctttaacagttgctttttgaggagcattagggagttgcatactattttcatctaaaatttgttctaattccattacagttgaaccgtgtaaacgaatgaaaccacgtgcagagttaaaacctttaacgttgaatcccattgtagcgttttgaccgttgtcgctaattacttgaacttggcggtctaattgttggttaacgaagtctgcttgaacaccgataggcatataagcatctgttggtgtaccataacctttaccaactaatactgatgcttggttaagtaaagcttctgttaagctagcacctttagcatctagtacattatgtttgtcaattagtttagctaaaccatcgaactctaaacctgaacctgcatctgggttttctgataagtcagagtcaccgtagaaagaagcccattcaattgttttagcaacaactgagatagcatcgtctgtcaagatacgcattgggtcttcaatgttgttaactaaacctgttgcgatactcatatttttagtatcagaaacgtatttcatgttaactgttttttgacgtaagttagggtctgaaattggtgctacaccgatttcacgagtaaaacgagtatgacctacacgtccatgtgctagatatacgtcatatttagctacagttgaagttgccggacgtttagtgatgtcacggtaaaatgataagtctccgtctgcccaagttagcattgtgatttggtcgtctaagaactctcgtcttaaagcggcggcatcagtttgagattctggtgtaataccatatcctgtagtgaagcctttaattacttcttcctgtacggaagttaattgtcgttctgtattctttttctctgtcattatgtataaatcacctttctttatattcctctcaatataagacctgaccctgtggaggttcaggagggaatgtcaggtcttttattgtttctgctattaatatagcagtacaattaaaaatcgttcactatatagtaaatttatttatatttattgataaacccttcaattttagctaggtcatcatttgttctagcagttccccaagtgtgtctaactgattctaattcaccacgttgagctactccgctaacagacacacgtttatatgtttccataaagtctggtctaatagcctgtactctatcacggaatggtacttctttttcaggttctgctgtgtcttgttcaccatcaacaactacttctacagttggttcttcggtagctacctcttcctcaactgctacagacttattaacaaacccaacagccttgccttctgagtcctcttttcctgttttaatttcagggttatcaagtaaagatttctcaacagactccgcttcgtctttttctttagcttctttagcttctttaaactctctatgaatttcgtctaacttttcacttagacctaacatatttttaaagatagcttccatagctcctaagatttcagaagatttgataacttgttcgacttcggtagacttctcgatgtcttctttgtcttctttgtcttttttatctttttctttctttttgtcttcgtcttcttcgtcttcctcctcttcttcatctttattttctttagttttatcagatttttcaacagtttccgctgattcttcaacaggttcctcaactgctacttcttcaacagtttctggctcttcctgttctgcttcaactgtttcagtaactccggattcttctccgtcattgcctgcagtctcctcagattcattaggcttttcttccggctcaacctcagcaccttcttcaactttttctggttcagtaggttcttctacagactttgtaatttcctcatctgtagtaggttttactttttcatttaactgtgcatcaaaatcttctataatatcgttcaatgttttagccactatttcttacccctttcttttttcaataaagtttgttgcttctttcaaggaaatccctctacttaattgtaacataaatttagataattcattttcggaagtgtctacttctatatccttagccacttcacgtaaaataaactctttctctgcaggttttttactatatagccctgcaatagttcttactgcatacgtcaaattagaaatgtcctctttaaacatctctcttctaagtgcgcctgcatctacttgtgcatctggtgctgtgccatatcctgtagtccaagatttaactagtgtctcccatgtagctcttgggttagccggatgtgtcgttaatgcaacattatgtattttaacaccttctatgattctgttatcttgtgcatttctactaactactgcaccctcaatagaaaaacctaactgacgagatattcctgatttctggattgtattagctagcttccacatagattgtgcgtacttgttatcctttaatagcttagcctctacgaataaccctttgtccacatcaacataacaattgctagttggggcacctataataaactcagcatcatttttatgctcatagtttatccaaccattttcaataaaataagaaatatcaatgccctgtggtaaaataatatctccttgtaaatctaaatctggggtagttgcatatccttgcacataccattcaccatcattcacagattcagacttctgtatagaattttcaatatccagtggtacgaaaatactatatttttctctaacttctgacaatttgcttttcaccacctaaacgttttactgtatttaatatagcagaacatgtactatctagtaacacaaacaaagttgagggggagctagccccctccattatcttttattctttttttataccatcagtaccatgcttagttgagttggtgttttcttccgattttaactgaccgtccttgccaacattgtcgtctaccttaccattaactttcttagacttaccgtttaaaccttgttgttcatcctgaaatgaaacatctgtatcattttgactgtctttaggttcttctggttctggttgttctacatctccaccagatagctctaatagtctatttaagcggtcttgacttctttggtattctagctgttcttcttgtaacgcttgcccaatagcttgtatgtgaacaccatttagtataacgtctccgcctttaattggttctaaacctttatcatgtcttatctcgttaactgttctgaatactttaccttcttgctcaattattttaagcttatctagttgagcacttaggtctcctcccctaaattggaattggtatttttctccaaattccgcaacaatatatgtgttaacggtgtcctctataaatcttaaaaggggttgtagccctttattctgagaagcctgcattttctcttttgagtttccctcatttagggagcctcctttagaacctgttgcaccaccattattagggaagtttatctctgcagggtcaattccatataaagcagatataacattaattaagtagttaagccatttttcaaattgcatatcattggcagagggtgtcatattgacgaatttgacatcttctgctgaaactacaggtatttgccatgagccatttatccctgctagtgagcttctccactctctacggaaaatgtctagtgcttgttgagactgctgttgccctgtttttacatgtaagatacctctggtagttcctccatgtgagaagaatctatcgttaaatacctctgtattttcatgggcaataaactgcttcaacgcaatttctaactctgggtaaccatactgacctacttcgatgtctgctcttgggttacgtactgcgaatgctagttctctctcattaaatttagcaacaattctgttatcaacaacctgaacaaatctttcaccgtttttaattagtttcccttcaccatttgttgctaagaaaattgtagttgggtctacagtatcaaatttgataaagttaccatctttatcaaataccttttcaaagtttacttggtcatacatatatgttgcccgaacaagttttttacaaaaagttgtaaagttatctctattagggtctctaaattgtgctgtgttctctaaaaaactttcaatacgcttaatattagcaatgtcatgtgaagtaggttctgcttcaatatcttttaaacgtatctcgtaaccaacacctgtttcagaatttcttgcgggcttacaatacatcgatacttggtttgaccgtgtattaataatagcatttaatattatgttattaccaaactttttaagtgtcttatgtaagtcctgagagttacgtatagacggtttagttttatacccggggtttactgacatctctccaataattggttgcatatatgcagtggtcttaccattcatacctttttcaatactttccatactgtacggctcgttattgaccacattggtgtctatttcccttaaatgcatcttataattttccatattcctagtttcttctatggacgatttttctatacctagtgctttatctaaccattttggcaactgttacacctaccttatacataaatttttatccttgtcttatcgtttaagcatcttccattaggtatttcgatttctttctcttccatatcattataaaagaagcttttttgaccatcgttaactgtttcatagtcataaattacagggttaccttttctatctaataaaagtatctctctgctatctaacaatcttaaaattctatagctttctccgtttattgttgtatgtatctggacgtgatttccaccatactcatagtatgagtggataattaaaatccagtcaccaacaaacccattctttttagcaacttttttaaccatctgccatgtctcatcttgaatataacccatgtttcccctccttaaatggttttaacataagtctattataccacataactaatcataaccattttatattactatttactatatattaatatagcataaaaaaaaagaccccttaataaaggggtctttattgctatttaattttagtaccaaccatttgctaaccagaaagattttgcgttctcccatgagccatagcgacctgctacatactcatcagctactcgttcttggttggcaggtgaatagtcaccatttaaataagaggcagatagttggtaacgaccaatatactgaccatttgttgcatcataagaaccactagattcacgttgtgcaatccactcttttgctgagctattgtcagcaggtacctctactacaggggcaggggcttctgctacaactacaggttgctctacaacttcttgttctacaactacaggttgttcaggtgttactggttcgttatattgtacttcaatatctggttcagtggtattcacttgttccactagcttgccattttcatctaaaggtaatagcaatttgtcacctacgaaaataacattagggtcagctacaacacctttattaactttatggatattagtgaaatctaagttgtacttagctccaatttctgatagtgtgtctcctgactctaccttccaatatgtcttaccattctctgttacttcttctgccttagcttcggttcctaaacctactactaaacctaaacttaataaacttaaacctaaaatactagttttcttcaaaataaaattctcctcaatgttttctttatttgtgttgcttacaagaactaatataacatgtaattgttacagccaaaagaacaagcggttacatagccattacatattattacgaatattacaaagaaagaaaaaagaccaccgtaagatggtcttaaatctagtattaagattttttagtaatacctactactggacatactgtctctaatctgttataccatggtgcatttttattccattctttttgactgtaaaatggaatgtcttttccgttattttttctgtaaatatcctcaattactttcatttcatctggatgagatactctgcgacagtttaccccattacagaacataactgtccatgcatcaccattccactctagtactcctgtttttgagttaataggtctttcgtataaacaatacatagttgccactccttctttatttttattattacttggtttgcttggtgtaggcttacttggctccggcttaactgcattgttagagcctcctgccatgaactgttgagcttgacgtacaacttcgtctacatccatacctccggggcatgctgttgcagttacttcgttatgccctaacacgtgttgtcggtcacatggaataccataacgtgtacaaatatctgccactaaacgagcacaatttgtaattgttctagggtctacagaccaattaggtgctcctgacgagtttacattttcaatacctattgaacgttgattagggttagcaatcttaggaacgtctataccacctgtgcctccggcatggaatgctgaatactgctcaccgacacatccaataatttctgttggtgtacattcataatgagcagacgtacctgcgccaccgcctaatagccatgtgttcatagcaacatctttatttgttgttgcattgtgatgtaggataatggtgtcgatagtgataccattacgactacctgcgttcattgggttaggatttacacttgtaatcaagctactaaatacttctcctgccatctattaaacctccttagttagtcataaaaaagaaacgagttctatttcatgcatatctaatatagcacaaaatagaactcgttaatttaatttttatagatgttcaactaagaatctttctacaccgtcaatgtcagttttatctgaataggtagcaacacattctacaatggttgttagttctccgttgttaagtttaacttgttgcttgcctttctctaaagaaataggtaaacctaaatctttattcctatttatcatccccgcacacataaggaatgattctactgcgttatcccctttttttattagatagtagggcacaggaatcataacttgacttgtatattctaagttatcgggcattgcatctcttttttctttaagcaatcttaaattttttataagagctttccccaaagaatttttaatttccattatgagctttcagcccctctctcatactgttaactagctccattaaatcatatttttctgcgacttcatctgttaattcattagccaaagcctcagttactcttaatgtgttaagcacgtcatcttttgtttttagtacttccatagctctttctcgttcttcttctaacttagcttgacgttctttacgctcttcttcttgcttagccatatattcttttgtaccaaatacaatatttacacgcacttcttgtgcctgtagtatagagctacaccctgtttgtaattcatcaatgatagcctcacctagtggtaaaactttaccatccttaacagcttcgacaaatctcttgtttatctctagttcatttaaatttttatctacagctaatctaaataaattctcatattcaactgggtattctgctctgattacttggaaccctaaaaaataacctgatgtgtagtctccttctggcgggttttgttgtaagaactggtagttctcatacaaatctttaactaacttacccatcttctctacaaaaatatcgtagtcctcatcagtagctaccttattagttaattcatctagtttcttattattcataattaattccatattatttatttctcctttcaatatatacattatatcctttgttatatctgttactttgtctgttagtccctcttttgctagcatctttaagttataaaattctctaactaccttacttactagtaaattaaaaattaaaaaaataatagttgaaaatactagtataatgactaacgtagcacttatgactaaggctcccctagaagactacgcagtgagctcatcgacacaatatactctgtcttatttcgtttgtacccatataagctaattagttcctctacatcaggtgcattatctgtgcttatcttagcccctagacctaatttccaagcctttaatgtaagctgtgaaaaagccaacatcttcataccctcaacagcatcatctgacatcgagaaagacatttttaaataagaaccttctctagtaaaccctatcactcctcgtgtaacagaatcttgttcaagaataagataatcagtgcactgcgtatcaaaattatttccaaattttttagtgtctgctagcaattttgctatctttttagggtctaacttctcttcttcatcactacctaagctaactttatgtagttccttggcattaactggttctaactcttttttctttttactctctttgtctaaagggttactattgttgctgtccgaactgcctgtatcttcataaatagttgtagtcatagttttgccatttctaacaacttttcttttaacaggtttcagcttagacaagtctttttgaccaccttttactaaatacatatctttagaatcacagctatctgcatatagatacatgtcataaaaattgtcaaacccatacgctttgacaatatgttgatattcgtttgtcagcaattcgttattttttataacgtacccataacttaaagacttatttattagtccttcaattgtacttttacttatttttacattgttgtctaccataactatccccctaactatatttattataccacaaaaatatatatcaaacaatacaaaatgccttattcagcttgactaaaactaaataaggcaatctacctatacaaattctgtaaatatatcaggtttcgtaggtgctaattgagttgacacccaatcagagtcaaatccatactcttttactcccgtaaatacattcattaggtactcataccctaacatagagtaaacacttgcttgtgagtagtggtcgtcaccttttctgccgataatttgtctaaaacctgtcgatgtcttttcgtcctcaatatcacgtataacaacattcttccagtgctctttatataaattaagctctgtatctggttttgagtagtaacctatttctccttctttcatcttgttaatgtaacgtttattctgcattaacttgtcagcggatactttatttgcttgtgggctccaagtaggtactagattaccagtagatttaggggttgatgggtagacacacccaaaaactcgttcttttccataaatttgcataagtttagcaactttatcccctgagtctcctacgtcagctacgattatatctggattgtaaggagctagctgtaattttatagactgtatgtctacatctattgcattagggtctagcgggttggacttacctacagaaaaaagttttatcaaatctaccgtaccatttgttttaacaccatgtatagatacccaatgtctgttaccccagtcaataccaacagatataaatttataatcacctctgtctttagcaggttctactaaatagtttctcttatggctatcaacgtcagagtcgttaacagttaatttcaagtctgcgtaaggatacgagctaccgctactttcgtagtattttgttactgctcttggcagtcggactagactatatcatcaacctactttttggtttaggttgctctgctttacactacaaatttgatttccttttctaagtatgccgataccttttctattgtatcgagtgagtagggtacctccactaatagcaacccatgctcacttgcgtattttcgttttattttatcgctttcctgttgaagtttaaaccgttcagctccaccgaagtgagctattggttcatagtgttgtataccttgatattcaatcagcaactgctgactaggtatataaaagtcataggataatggttttttaagttctaaattagggaaggtttgctgttctgtaaatcccaaacctatatttgttagatacttccgaatcaatgcttcacctctcgatgacttacacctaggacacatattatggtgtagcaaatgacttgctttgatttcccactcattaccacagcggttgtgacgtaccaaaacttttttcttgctacccatgtactcagataataatgtgtattctttcggaaagcgttcctctaattttttgataaactgctctgtagagtacctctgctcctgtgctgtcgtattccttacgcacgtagggcatcttgttcctcgagataaaaaactattcggagaaactctccatgagttaccacatattctatgttggattgctactttgttagtagctcctgtatactctcctacaacgatgtactcattatccgttaagtttgtaacctcttctgcgaactgctcttgtgtcttttgcttagctcctcgtagcttatttctacaatgagggcatcggttaccatccaaaaaaccttttggtttaactagataagtagtgttacacgttgtgtgagttatagctatctttgttttgttatttacatattcaccaacaactgtatattcattgttagtaagttcagctacccgagctgtaaatgtttcagtggtttgtttctttgccatctaatcacctctcagtaattataatagcatacttagaatgtcttgtagttcttatctagttctcactaaccccatttggttagctacctagaagtttagtcgttaggcatttacaagcttacgcttgatttagcacgggattccctctttccttttcaggaggcagggtctcttatcagcttattggttattcagctttgcccgtttaacagagttttcgacaactgtttccagttgaaggggcgttattcacccagagtatagttataaaaggcttgtttagacaatgattgaagttctttggtctttaactggtcggcagttacccatactgcgttcatttgggaaatcatatatcctctagtacctagaccattttttgttctatcaggatatttaggtacccatacaccgttgtaccatctgtctaacggttctccacacttttgacaaacaaactggaatgagccgtcaactactgtcttagcaacaacatcgaccccttttgggttaacacaaagaatgttacctctactctctacaggagcctctggtgtatatgcatcataactcatttcgttataataattacatttctcacatttatgtaaataccaatgttggtctgaccctttaaagagcccatgtatccccatatctggggcacttggggtgctccatctgttaactatcttataaggtgatgaagacatagactccaacgcagaagcctctgctaatgcaggtacacggtcatactcgtccatagatagatagtcaatatcgacaccctctacagcgcccggttttgaacttgaacgaaaatacaaaaaactatttcttatttttttagcttttaatgagttaacttcttggtctacaattgtgctgtagtacccattctgtaaaacagggtctaaccttgtctgtacaaatttagtcatttgctcattcgttggaaatgtataaagacatttaacagcatcataactatgtgtatctgcaaactgtaacatagaaccaacacccatctctgatagccctagctgtcttgatttaattattgccttgtctctatgttggtcatttattatctttggttgccaaggtcggtgtgcctgtgcttttgttgcatcccttcctgatataataaaagttattgggtgattacgcacagagtgatttctcaataggtacgttgatggggtcagcatatctgttatataagctaactgctccgttgtaattttatccgtaccatacatctctttagccattttagctatatgttgtgcttttgtcgtcattagaaggtcgcctcgttctctctgttcatctgtagctctctgttaactaagacttcctcaagttggtcatctggaaggctggctaactcatcgagagaaattagctcctcttcttcaccgtccttaataatcttttctgtgctaaccttttcagatatgatgtctttatgctcagatgtaagtgcaggtagagttcctgtaccttcttgcattccatcatttatattattaacctgtaaataaatctgaaatagccttgttaaatctgcaatatcatcaacttctatagcacctgatgtaacattagccataaattgattgataagttttattgaagcttgattaaatgtgtctcttagttgttgctctggtgttctttctaactttttttgttttgcattatctttaagtctatctgccatacccattagctagctttcccctttcttactagaatacgcataacaagaccttatatcctcacagacatttagtctaacaatgtctgaaatcttacagctaatatggtctactactactgttggtacgcctgttcttaatacgagcttagatagcggtcttccacaaactgcacatattcttgttgcactacttatgcgtttgtcccctctttttatagttgttgttttactagattgactaaaaatatatctacgtttatccttaatgtctttattggtaatagacacccatcccacctcctaataggctatctttttatgatagctttaatcttctgcaagtcactgattgcttctttaagttgattctctacgttctcttttttctttttattagtataccaatcaaacagaatactagatagcaatgcacatgaggcaccgccaattaatgcctgaaatggggcattttgttctacaataacatatttagagaacagcttacttaaagtaaccatgactaatgcagagatggtaataaaaataatttttttaataattcgtttgctattatccatttttaccactctcccgtctacatttttagtctctatattaatttagcacacctgtttgctaacagctcagtgtactatattaatagtagctctaagggagaggagggaacaacataatgtatgaaacagcactggtctgtgtactgttactacaaatgtttgtttacttgatgtcctactatgtcactcgtgcactacagtataagaaactacaagcagaactgagccctttggcatcatacaagttcactttcgtgctctatggaattagtataggtagtctatacttgttttttaatgggtactatatacctatagtctacacggtaggcgctattataggtatattagtctgtctgtggtttacatctgactaatatagagcaacataacttacagggaggagcatatatgaacgattaccaagtaaatgaccttgttttaagactacaacgactggaagataaactccaacatggagagctaaccgagaagggcattaaacaggacaatgtagaactgaatagtgcggttagtgagttgagagatattgtcaatagtcttgataagcgacttgctgttcacgaagaaaaatatgcacacctaacctaccagattactaagttggaagaaactatagatgcactggaaggagtaaacgataaagagttcgaccacaagagggatattgtagagaacgtttttatgattgtcttaggagcagttgtcacttacgtgttcagcatgtttaagtaatttgtaaaatgtgttattatagtatatagtattattataacatattttgaaaggagaagtatcatgaattaccctaaaagagagaaagttgtagaggtttcattaacatctgggacatattctgtgttccctagaagactcggtgtaacaactaatgatgcaatgtcaattgtaaatggtgcaatgaaaggtgcagagttgccaatgattccagttcataaactagctgatagagattctgagttaacttatgtaaatgcattccagatacagacagctactgagaacgtagtagatgttccagaaagaatcactagcttgtatacgaaaccagaggatgaaacaccagaagatgaggaagtcagattgggaacaattaataactatttctcattaagataagagggcttaactgctctcttttttttgtttaagtaaaatgtcctattttaaatttttgtctttcttatat

SEQ ID NO: 3 - DNA, 20 bp, artificial sequence
  synthetic sequence
gatggagacacggaagctgt

SEQ ID NO: 4 - DNA, 20 bp, artificial sequence
  synthetic sequence
cggctttccccgtatacctc

SEQ ID NO: 5 - DNA, 20 bp, artificial sequence
  synthetic sequence
gccaagcttctcacacttcc

SEQ ID NO: 6 - DNA, 20 bp, artificial
  synthetic sequence
ccacctttttgtcaggtcgt